SEQUENCE LISTING (925 sequences)

SEQ ID NO: 1 - DNA, 32 bp, Artificial Sequence
  Synthetic DNA TRBV2
ntcaaatttcactctgaagatccggtccacaa

SEQ ID NO: 2 - DNA, 32 bp, Artificial Sequence
  Synthetic DNA TRBV3-1
ngctcacttaaatcttcacatcaattccctgg

SEQ ID NO: 3 - DNA, 27 bp, Artificial Sequence
  Synthetic DNA TRBV4-1
ncttaaaccttcacctacacgccctgc

SEQ ID NO: 4 - DNA, 27 bp, Artificial Sequence
  Synthetic DNA TRBV(4-2, 4-3)
ncttattccttcacctacacaccctgc

SEQ ID NO: 5 - DNA, 27 bp, Artificial Sequence
  Synthetic DNA TRBV5-1
ngctctgagatgaatgtgagcaccttg

SEQ ID NO: 6 - DNA, 27 bp, Artificial Sequence
  Synthetic DNA TRBV5-3
ngctctgagatgaatgtgagtgccttg

SEQ ID NO: 7 - DNA, 27 bp, Artificial Sequence
  Synthetic DNA TRBV(5-4, 5-5, 5-6, 5-7, 5-8)
ngctctgagctgaatgtgaacgccttg

SEQ ID NO: 8 - DNA, 23 bp, Artificial Sequence
  Synthetic DNA TRBV6-1
ntcgctcaggctggagtcggctg

SEQ ID NO: 9 - DNA, 21 bp, Artificial Sequence
  Synthetic DNA TRBV(6-2, 6-3)
ngctggggttggagtcggctg

SEQ ID NO: 10 - DNA, 22 bp, Artificial Sequence
  Synthetic DNA TRBV6-4
nccctcacgttggcgtctgctg

SEQ ID NO: 11 - DNA, 21 bp, Artificial Sequence
  Synthetic DNA TRBV6-5
ngctcaggctgctgtcggctg

SEQ ID NO: 12 - DNA, 22 bp, Artificial Sequence
  Synthetic DNA TRBV6-6
ncgctcaggctggagttggctg

SEQ ID NO: 13 - DNA, 23 bp, Artificial Sequence
  Synthetic DNA TRBV6-7
ncccctcaagctggagtcagctg

SEQ ID NO: 14 - DNA, 22 bp, Artificial Sequence
  Synthetic DNA TRBV6-8
ncactcaggctggtgtcggctg

SEQ ID NO: 15 - DNA, 22 bp, Artificial Sequence
  Synthetic DNA TRBV6-9
ncgctcaggctggagtcagctg

SEQ ID NO: 16 - DNA, 25 bp, Artificial Sequence
  Synthetic DNA TRBV7-1
nccactctgaagttccagcgcacac

SEQ ID NO: 17 - DNA, 24 bp, Artificial Sequence
  Synthetic DNA TRBV7-2
ncactctgacgatccagcgcacac

SEQ ID NO: 18 - DNA, 27 bp, Artificial Sequence
  Synthetic DNA TRBV7-3
nctctactctgaagatccagcgcacag

SEQ ID NO: 19 - DNA, 25 bp, Artificial Sequence
  Synthetic DNA TRBV7-4
nccactctgaagatccagcgcacag

SEQ ID NO: 20 - DNA, 24 bp, Artificial Sequence
  Synthetic DNA TRBV7-6
ncactctgacgatccagcgcacag

SEQ ID NO: 21 - DNA, 25 bp, Artificial Sequence
  Synthetic DNA TRBV7-7
nccactctgacgattcagcgcacag

SEQ ID NO: 22 - DNA, 25 bp, Artificial Sequence
  Synthetic DNA TRBV7-8
nccactctgaagatccagcgcacac

SEQ ID NO: 23 - DNA, 24 bp, Artificial Sequence
  Synthetic DNA TRBV7-9
ncaccttggagatccagcgcacag

SEQ ID NO: 24 - DNA, 29 bp, Artificial Sequence
  Synthetic DNA TRBV9
ngcactctgaactaaacctgagctctctg

SEQ ID NO: 25 - DNA, 23 bp, Artificial Sequence
  Synthetic DNA TRBV10-1
ncccctcactctggagtctgctg

SEQ ID NO: 26 - DNA, 24 bp, Artificial Sequence
  Synthetic DNA TRBV10-2
nccccctcactctggagtcagcta

SEQ ID NO: 27 - DNA, 24 bp, Artificial Sequence
  Synthetic DNA TRBV10-3
ncctcctcactctggagtccgcta

SEQ ID NO: 28 - DNA, 25 bp, Artificial Sequence
  Synthetic DNA TRBV(11-1, 11-3)
nccactctcaagatccagcctgcag

SEQ ID NO: 29 - DNA, 27 bp, Artificial Sequence
  Synthetic DNA TRBV11-2
nctccactctcaagatccagcctgcaa

SEQ ID NO: 30 - DNA, 25 bp, Artificial Sequence
  Synthetic DNA TRBV(12-3, 12-4, 12-5)
nccactctgaagatccagccctcag

SEQ ID NO: 31 - DNA, 29 bp, Artificial Sequence
  Synthetic DNA TRBV13
ncattctgaactgaacatgagctccttgg

SEQ ID NO: 32 - DNA, 25 bp, Artificial Sequence
  Synthetic DNA TRBV14
nctactctgaaggtgcagcctgcag

SEQ ID NO: 33 - DNA, 29 bp, Artificial Sequence
  Synthetic DNA TRBV15
ngataacttccaatccaggaggccgaaca

SEQ ID NO: 34 - DNA, 27 bp, Artificial Sequence
  Synthetic DNA TRBV16
nctgtagccttgagatccaggctacga

SEQ ID NO: 35 - DNA, 25 bp, Artificial Sequence
  Synthetic DNA TRBV17
ncttccacgctgaagatccatcccg

SEQ ID NO: 36 - DNA, 25 bp, Artificial Sequence
  Synthetic DNA TRBV18
ngcatcctgaggatccagcaggtag

SEQ ID NO: 37 - DNA, 23 bp, Artificial Sequence
  Synthetic DNA TRBV19
ncctctcactgtgacatcggccc

SEQ ID NO: 38 - DNA, 27 bp, Artificial Sequence
  Synthetic DNA TRBV20-1
ncttgtccactctgacagtgaccagtg

SEQ ID NO: 39 - DNA, 24 bp, Artificial Sequence
  Synthetic DNA TRBV23-1
ncagcctggcaatcctgtcctcag

SEQ ID NO: 40 - DNA, 25 bp, Artificial Sequence
  Synthetic DNA TRBV24-1
nctccctgtccctagagtctgccat

SEQ ID NO: 41 - DNA, 22 bp, Artificial Sequence
  Synthetic DNA TRBV25-1
nccctgaccctggagtctgcca

SEQ ID NO: 42 - DNA, 22 bp, Artificial Sequence
  Synthetic DNA TRBV27
nccctgatcctggagtcgccca

SEQ ID NO: 43 - DNA, 24 bp, Artificial Sequence
  Synthetic DNA TRBV28
nctccctgattctggagtccgcca

SEQ ID NO: 44 - DNA, 32 bp, Artificial Sequence
  Synthetic DNA TRBV29-1
nctaacattctcaactctgactgtgagcaaca

SEQ ID NO: 45 - DNA, 29 bp, Artificial Sequence
  Synthetic DNA TRBV30
ncggcagttcatcctgagttctaagaagc

SEQ ID NO: 46 - DNA, 36 bp, Artificial Sequence
  Synthetic DNA TRBJ1-1
nttacctacaactgtgagtctggtgccttgtccaaa

SEQ ID NO: 47 - DNA, 34 bp, Artificial Sequence
  Synthetic DNA TRBJ1-2
nacctacaacggttaacctggtccccgaaccgaa

SEQ ID NO: 48 - DNA, 34 bp, Artificial Sequence
  Synthetic DNA TRBJ1-3
nacctacaacagtgagccaacttccctctccaaa

SEQ ID NO: 49 - DNA, 32 bp, Artificial Sequence
  Synthetic DNA TRBJ1-4
nccaagacagagagctgggttccactgccaaa

SEQ ID NO: 50 - DNA, 32 bp, Artificial Sequence
  Synthetic DNA TRBJ1-6
nctgtcacagtgagcctggtcccgttcccaaa

SEQ ID NO: 51 - DNA, 26 bp, Artificial Sequence
  Synthetic DNA TRBJ2-1
ncggtgagccgtgtccctggcccgaa

SEQ ID NO: 52 - DNA, 32 bp, Artificial Sequence
  Synthetic DNA TRBJ2-2
nccagtacggtcagcctagagccttctccaaa

SEQ ID NO: 53 - DNA, 27 bp, Artificial Sequence
  Synthetic DNA TRBJ2-3
nactgtcagccgggtgcctgggccaaa

SEQ ID NO: 54 - DNA, 23 bp, Artificial Sequence
  Synthetic DNA TRBJ2-4
nagagccgggtcccggcgccgaa

SEQ ID NO: 55 - DNA, 23 bp, Artificial Sequence
  Synthetic DNA TRBJ2-5
nggagccgcgtgcctggcccgaa

SEQ ID NO: 56 - DNA, 24 bp, Artificial Sequence
  Synthetic DNA TRBJ2-6
ngtcagcctgctgccggccccgaa

SEQ ID NO: 57 - DNA, 24 bp, Artificial Sequence
  Synthetic DNA TRBJ2-7
ngtgagcctggtgcccggcccgaa

SEQ ID NO: 58 - DNA, 65 bp, Artificial Sequence
  Synthetic DNA TRBV2
caagcagaagacggcatacgagctcttccgatcttcaaatttcactctgaagatccggtccacaa

SEQ ID NO: 59 - DNA, 65 bp, Artificial Sequence
  Synthetic DNA TRBV3-1
caagcagaagacggcatacgagctcttccgatctgctcacttaaatcttcacatcaattccctgg

SEQ ID NO: 60 - DNA, 60 bp, Artificial Sequence
  Synthetic DNA TRBV4-1
caagcagaagacggcatacgagctcttccgatctcttaaaccttcacctacacgccctgc

SEQ ID NO: 61 - DNA, 60 bp, Artificial Sequence
  Synthetic DNA TRBV(4-2, 4-3)
caagcagaagacggcatacgagctcttccgatctcttattccttcacctacacaccctgc

SEQ ID NO: 62 - DNA, 60 bp, Artificial Sequence
  Synthetic DNA TRBV5-1
caagcagaagacggcatacgagctcttccgatctgctctgagatgaatgtgagcaccttg

SEQ ID NO: 63 - DNA, 60 bp, Artificial Sequence
  Synthetic DNA TRBV5-3
caagcagaagacggcatacgagctcttccgatctgctctgagatgaatgtgagtgccttg

SEQ ID NO: 64 - DNA, 60 bp, Artificial Sequence
  Synthetic DNA TRBV(5-4, 5-5, 5-6, 5-7, 5-8)
caagcagaagacggcatacgagctcttccgatctgctctgagctgaatgtgaacgccttg

SEQ ID NO: 65 - DNA, 56 bp, Artificial Sequence
  Synthetic DNA TRBV6-1
caagcagaagacggcatacgagctcttccgatcttcgctcaggctggagtcggctg

SEQ ID NO: 66 - DNA, 54 bp, Artificial Sequence
  Synthetic DNA TRBV(6-2, 6-3)
caagcagaagacggcatacgagctcttccgatctgctggggttggagtcggctg

SEQ ID NO: 67 - DNA, 55 bp, Artificial Sequence
  Synthetic DNA TRBV6-4
caagcagaagacggcatacgagctcttccgatctccctcacgttggcgtctgctg

SEQ ID NO: 68 - DNA, 54 bp, Artificial Sequence
  Synthetic DNA TRBV6-5
caagcagaagacggcatacgagctcttccgatctgctcaggctgctgtcggctg

SEQ ID NO: 69 - DNA, 55 bp, Artificial Sequence
  Synthetic DNA TRBV6-6
caagcagaagacggcatacgagctcttccgatctcgctcaggctggagttggctg

SEQ ID NO: 70 - DNA, 56 bp, Artificial Sequence
  Synthetic DNA TRBV6-7
caagcagaagacggcatacgagctcttccgatctcccctcaagctggagtcagctg

SEQ ID NO: 71 - DNA, 55 bp, Artificial Sequence
  Synthetic DNA TRBV6-8
caagcagaagacggcatacgagctcttccgatctcactcaggctggtgtcggctg

SEQ ID NO: 72 - DNA, 55 bp, Artificial Sequence
  Synthetic DNA TRBV6-9
caagcagaagacggcatacgagctcttccgatctcgctcaggctggagtcagctg

SEQ ID NO: 73 - DNA, 58 bp, Artificial Sequence
  Synthetic DNA TRBV7-1
caagcagaagacggcatacgagctcttccgatctccactctgaagttccagcgcacac

SEQ ID NO: 74 - DNA, 57 bp, Artificial Sequence
  Synthetic DNA TRBV7-2
caagcagaagacggcatacgagctcttccgatctcactctgacgatccagcgcacac

SEQ ID NO: 75 - DNA, 60 bp, Artificial Sequence
  Synthetic DNA TRBV7-3
caagcagaagacggcatacgagctcttccgatctctctactctgaagatccagcgcacag

SEQ ID NO: 76 - DNA, 58 bp, Artificial Sequence
  Synthetic DNA TRBV7-4
caagcagaagacggcatacgagctcttccgatctccactctgaagatccagcgcacag

SEQ ID NO: 77 - DNA, 57 bp, Artificial Sequence
  Synthetic DNA TRBV7-6
caagcagaagacggcatacgagctcttccgatctcactctgacgatccagcgcacag

SEQ ID NO: 78 - DNA, 58 bp, Artificial Sequence
  Synthetic DNA TRBV7-7
caagcagaagacggcatacgagctcttccgatctccactctgacgattcagcgcacag

SEQ ID NO: 79 - DNA, 58 bp, Artificial Sequence
  Synthetic DNA TRBV7-8
caagcagaagacggcatacgagctcttccgatctccactctgaagatccagcgcacac

SEQ ID NO: 80 - DNA, 57 bp, Artificial Sequence
  Synthetic DNA TRBV7-9
caagcagaagacggcatacgagctcttccgatctcaccttggagatccagcgcacag

SEQ ID NO: 81 - DNA, 62 bp, Artificial Sequence
  Synthetic DNA TRBV9
caagcagaagacggcatacgagctcttccgatctgcactctgaactaaacctgagctctctg

SEQ ID NO: 82 - DNA, 56 bp, Artificial Sequence
  Synthetic DNA TRBV10-1
caagcagaagacggcatacgagctcttccgatctcccctcactctggagtctgctg

SEQ ID NO: 83 - DNA, 57 bp, Artificial Sequence
  Synthetic DNA TRBV10-2
caagcagaagacggcatacgagctcttccgatctccccctcactctggagtcagcta

SEQ ID NO: 84 - DNA, 57 bp, Artificial Sequence
  Synthetic DNA TRBV10-3
caagcagaagacggcatacgagctcttccgatctcctcctcactctggagtccgcta

SEQ ID NO: 85 - DNA, 58 bp, Artificial Sequence
  Synthetic DNA TRBV(11-1, 11-3)
caagcagaagacggcatacgagctcttccgatctccactctcaagatccagcctgcag

SEQ ID NO: 86 - DNA, 60 bp, Artificial Sequence
  Synthetic DNA TRBV11-2
caagcagaagacggcatacgagctcttccgatctctccactctcaagatccagcctgcaa

SEQ ID NO: 87 - DNA, 58 bp, Artificial Sequence
  Synthetic DNA TRBV(12-3, 12-4, 12-5)
caagcagaagacggcatacgagctcttccgatctccactctgaagatccagccctcag

SEQ ID NO: 88 - DNA, 62 bp, Artificial Sequence
  Synthetic DNA TRBV13
caagcagaagacggcatacgagctcttccgatctcattctgaactgaacatgagctccttgg

SEQ ID NO: 89 - DNA, 58 bp, Artificial Sequence
  Synthetic DNA TRBV14
caagcagaagacggcatacgagctcttccgatctctactctgaaggtgcagcctgcag

SEQ ID NO: 90 - DNA, 62 bp, Artificial Sequence
  Synthetic DNA TRBV15
caagcagaagacggcatacgagctcttccgatctgataacttccaatccaggaggccgaaca

SEQ ID NO: 91 - DNA, 60 bp, Artificial Sequence
  Synthetic DNA TRBV16
caagcagaagacggcatacgagctcttccgatctctgtagccttgagatccaggctacga

SEQ ID NO: 92 - DNA, 58 bp, Artificial Sequence
  Synthetic DNA TRBV17
caagcagaagacggcatacgagctcttccgatctcttccacgctgaagatccatcccg

SEQ ID NO: 93 - DNA, 58 bp, Artificial Sequence
  Synthetic DNA TRBV18
caagcagaagacggcatacgagctcttccgatctgcatcctgaggatccagcaggtag

SEQ ID NO: 94 - DNA, 56 bp, Artificial Sequence
  Synthetic DNA TRBV19
caagcagaagacggcatacgagctcttccgatctcctctcactgtgacatcggccc

SEQ ID NO: 95 - DNA, 60 bp, Artificial Sequence
  Synthetic DNA TRBV20-1
caagcagaagacggcatacgagctcttccgatctcttgtccactctgacagtgaccagtg

SEQ ID NO: 96 - DNA, 57 bp, Artificial Sequence
  Synthetic DNA TRBV23-1
caagcagaagacggcatacgagctcttccgatctcagcctggcaatcctgtcctcag

SEQ ID NO: 97 - DNA, 58 bp, Artificial Sequence
  Synthetic DNA TRBV24-1
caagcagaagacggcatacgagctcttccgatctctccctgtccctagagtctgccat

SEQ ID NO: 98 - DNA, 55 bp, Artificial Sequence
  Synthetic DNA TRBV25-1
caagcagaagacggcatacgagctcttccgatctccctgaccctggagtctgcca

SEQ ID NO: 99 - DNA, 55 bp, Artificial Sequence
  Synthetic DNA TRBV27
caagcagaagacggcatacgagctcttccgatctccctgatcctggagtcgccca

SEQ ID NO: 100 - DNA, 57 bp, Artificial Sequence
  Synthetic DNA TRBV28
caagcagaagacggcatacgagctcttccgatctctccctgattctggagtccgcca

SEQ ID NO: 101 - DNA, 65 bp, Artificial Sequence
  Synthetic DNA TRBV29-1
caagcagaagacggcatacgagctcttccgatctctaacattctcaactctgactgtgagcaaca

SEQ ID NO: 102 - DNA, 62 bp, Artificial Sequence
  Synthetic DNA TRBV30
caagcagaagacggcatacgagctcttccgatctcggcagttcatcctgagttctaagaagc

SEQ ID NO: 103 - DNA, 60 bp, Artificial Sequence
  Synthetic DNA TRBJ1-1
aatgatacggcgaccaccgagatctttacctacaactgtgagtctggtgccttgtccaaa

SEQ ID NO: 104 - DNA, 58 bp, Artificial Sequence
  Synthetic DNA TRBJ1-3
aatgatacggcgaccaccgagatctacctacaacagtgagccaacttccctctccaaa

SEQ ID NO: 105 - DNA, 56 bp, Artificial Sequence
  Synthetic DNA TRBJ1-4
aatgatacggcgaccaccgagatctccaagacagagagctgggttccactgccaaa

SEQ ID NO: 106 - DNA, 56 bp, Artificial Sequence
  Synthetic DNA TRBJ1-6
aatgatacggcgaccaccgagatctctgtcacagtgagcctggtcccgttcccaaa

SEQ ID NO: 107 - DNA, 50 bp, Artificial Sequence
  Synthetic DNA TRBJ2-1
aatgatacggcgaccaccgagatctcggtgagccgtgtccctggcccgaa

SEQ ID NO: 108 - DNA, 56 bp, Artificial Sequence
  Synthetic DNA TRBJ2-2
aatgatacggcgaccaccgagatctccagtacggtcagcctagagccttctccaaa

SEQ ID NO: 109 - DNA, 51 bp, Artificial Sequence
  Synthetic DNA TRBJ2-3
aatgatacggcgaccaccgagatctactgtcagccgggtgcctgggccaaa

SEQ ID NO: 110 - DNA, 47 bp, Artificial Sequence
  Synthetic DNA TRBJ2-4
aatgatacggcgaccaccgagatctagagccgggtcccggcgccgaa

SEQ ID NO: 111 - DNA, 47 bp, Artificial Sequence
  Synthetic DNA TRBJ2-5
aatgatacggcgaccaccgagatctggagccgcgtgcctggcccgaa

SEQ ID NO: 112 - DNA, 48 bp, Artificial Sequence
  Synthetic DNA TRBJ2-6
aatgatacggcgaccaccgagatctgtcagcctgctgccggccccgaa

SEQ ID NO: 113 - DNA, 48 bp, Artificial Sequence
  Synthetic DNA TRBJ2-7
aatgatacggcgaccaccgagatctgtgagcctggtgcccggcccgaa

SEQ ID NO: 114 - DNA, 284 bp, Artificial Sequence
  Synthetic DNA TRBV1*01
gatactggaattacccagacaccaaaatacctggtcacagcaatggggagtaaaaggacaatgaaacgtgagcatctgggacatgattctatgtattggtacagacagaaagctaagaaatccctggagttcatgttttactacaactgtaaggaattcattgaaaacaagactgtgccaaatcacttcacacctgaatgccctgacagctctcgcttataccttcatgtggtcgcactgcagcaagaagactcagctgcgtatctctgcaccagcagccaaga

SEQ ID NO: 115 - DNA, 290 bp, Artificial Sequence
  Synthetic DNA TRBV2*01
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcagtgaagc

SEQ ID NO: 116 - DNA, 288 bp, Artificial Sequence
  Synthetic DNA TRBV2*03
gaacctgaagtcacccagactcccagccatcaggtcacacagatgggacaggaagtgatcttgcgctgtgtccccatctctaatcacttatacttctattggtacagacaaatcttggggcagaaagtcgagtttctggtttccttttataataatgaaatctcagagaagtctgaaatattcgatgatcaattctcagttgagaggcctgatggatcaaatttcactctgaagatccggtccacaaagctggaggactcagccatgtacttctgtgccagcagtgaa

SEQ ID NO: 117 - DNA, 287 bp, Artificial Sequence
  Synthetic DNA TRBV3-1*01
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataataaggagctcattataaatgaaacagttccaaatcgcttctcacctaaatctccagacaaagctcacttaaatcttcacatcaattccctggagcttggtgactctgctgtgtatttctgtgccagcagccaaga

SEQ ID NO: 118 - DNA, 279 bp, Artificial Sequence
  Synthetic DNA TRBV3-1*02
gacacagctgtttcccagactccaaaatacctggtcacacagatgggaaacgacaagtccattaaatgtgaacaaaatctgggccatgatactatgtattggtataaacaggactctaagaaatttctgaagataatgtttagctacaataacaaggagatcattataaatgaaacagttccaaatcgattctcacctaaatctccagacaaagctaaattaaatcttcacatcaattccctggagcttggtgactctgctgtgtatttctgtgccagc

SEQ ID NO: 119 - DNA, 287 bp, Artificial Sequence
  Synthetic DNA TRBV3-2*01
gacacagccgtttcccagactccaaaatacctggtcacacagatgggaaaaaaggagtctcttaaatgagaacaaaatctgggccataatgctatgtattggtataaacaggactctaagaaatttctgaagacaatgtttatctacagtaacaaggagccaattttaaatgaaacagttccaaatcgcttctcacctgactctccagacaaagctcatttaaatcttcacatcaattccctggagcttggtgactctgctgtgtatttctgtgccagcagccaaga

SEQ ID NO: 120 - DNA, 287 bp, Artificial Sequence
  Synthetic DNA TRBV3-2*02
gacacagccgtttcccagactccaaaatacctggtcacacagatgggaaaaaaggagtctcttaaatgagaacaaaatctgggccataatgctatgtattggtataaacaggactctaagaaatttctgaagacaatgtttatctacagtaacaaggagccaattttaaatgaaacagttccaaatcgcttctcacctgactctccagacaaagttcatttaaatcttcacatcaattccctggagcttggtgactctgctgtgtatttctgtgccagcagccaaga

SEQ ID NO: 121 - DNA, 285 bp, Artificial Sequence
  Synthetic DNA TRBV3-2*03
gacacagccgtttcccagactccaaaatacctggtcacacagacgggaaaaaaggagtctcttaaatgagaacaaaatctgggccataatgctatgtattggtataaacaggactctaagaaatttctgaagacaatgtttatctacagtaacaaggagccaattttaaatgaaacagttccaaatcgcttctcacctgactctccagacaaagttcatttaaatcttcacatcaattccctggagcttggtgactctgctgtgtatttctgtgccagcagccaa

SEQ ID NO: 122 - DNA, 287 bp, Artificial Sequence
  Synthetic DNA TRBV4-1*01
gacactgaagttacccagacaccaaaacacctggtcatgggaatgacaaataagaagtctttgaaatgtgaacaacatatggggcacagggctatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaaga

SEQ ID NO: 123 - DNA, 258 bp, Artificial Sequence
  Synthetic DNA TRBV4-1*02
cacctggtcatgggaatgacaaataagaagtctttgaaatgtgaacaacatatggggcacagggcaatgtattggtacaagcagaaagctaagaagccaccggagctcatgtttgtctacagctatgagaaactctctataaatgaaagtgtgccaagtcgcttctcacctgaatgccccaacagctctctcttaaaccttcacctacacgccctgcagccagaagactcagccctgtatctctgcgccagcagccaa

SEQ ID NO: 124 - DNA, 287 bp, Artificial Sequence
  Synthetic DNA TRBV4-2*01
gaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttattccttcacctacacaccctgcagccagaagactcggccctgtatctctgtgccagcagccaaga

SEQ ID NO: 125 - DNA, 282 bp, Artificial Sequence
  Synthetic DNA TRBV4-2*02
gaaacgggagttacgcagacaccaagacacctggtcatgggaatgacaaataagaagtctttgaaatgtgaacaacatctggggcataacgctatgtattggtacaagcaaagtgctaagaagccactggagctcatgtttgtctacaactttaaagaacagactgaaaacaacagtgtgccaagtcgcttctcacctgaatgccccaacagctctcacttatgccttcacctacacaccctgcagccagaagactcggccctgtatctctgtgccagcacc

SEQ ID NO: 126 - DNA, 287 bp, Artificial Sequence
  Synthetic DNA TRBV4-3*01
gaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagccaaga

SEQ ID NO: 127 - DNA, 282 bp, Artificial Sequence
  Synthetic DNA TRBV4-3*02
gaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgggttgaaaacaacagtgtgccaagtcgcttctcacctgaatgccccaacagctctcacttatcccttcacctacacaccctgcagccagaagactcggccctgtatctctgcgccagcagc

SEQ ID NO: 128 - DNA, 282 bp, Artificial Sequence
  Synthetic DNA TRBV4-3*03
gaaacgggagttacgcagacaccaagacacctggtcatgggaatgacaaataagaagtctttgaaatgtgaacaacatctgggtcataacgctatgtattggtacaagcaaagtgctaagaagccactggagctcatgtttgtctacagtcttgaagaacgtgttgaaaacaacagtgtgccaagtcgcttctcacctgaatgccccaacagctctcacttattccttcacctacacaccctgcagccagaagactcggccctgtatctctgcgccagcagc

SEQ ID NO: 129 - DNA, 231 bp, Artificial Sequence
  Synthetic DNA TRBV4-3*04
aagaagtctttgaaatgtgaacaacatctggggcataacgctatgtattggtacaagcaaagtgctaagaagccactggagctcatgtttgtctacagtcttgaagaacgggttgaaaacaacagtgtgccaagtcgcttctcacctgaatgccccaacagctctcacttattccttcacctacacaccctgcagccagaagactcggccctgtatctctgcgccagcagc

SEQ ID NO: 130 - DNA, 286 bp, Artificial Sequence
  Synthetic DNA TRBV5-1*01
aaggctggagtcactcaaactccaagatatctgatcaaaacgagaggacagcaagtgacactgagctgctcccctatctctgggcataggagtgtatcctggtaccaacagaccccaggacagggccttcagttcctctttgaatacttcagtgagacacagagaaacaaaggaaacttccctggtcgattctcagggcgccagttctctaactctcgctctgagatgaatgtgagcaccttggagctgggggactcggccctttatctttgcgccagcagcttgg

SEQ ID NO: 131 - DNA, 285 bp, Artificial Sequence
  Synthetic DNA TRBV5-1*02
agggctggggtcactcaaactccaagacatctgatcaaaacgagaggacagcaagtgacactgggctgctcccctatctctgggcataggagtgtatcctggtaccaacagaccctaggacagggccttcagttcctctttgaatacttcagtgagacacagagaaacaaaggaaacttccttggtcgattctcagggcgccagttctctaactctcgctctgagatgaatgtgagcaccttggagctgggggactcggccctttatctttgcgccagcgcttgc

SEQ ID NO: 132 - DNA, 286 bp, Artificial Sequence
  Synthetic DNA TRBV5-3*01
gaggctggagtcacccaaagtcccacacacctgatcaaaacgagaggacagcaagtgactctgagatgctctcctatctctgggcacagcagtgtgtcctggtaccaacaggccccgggtcaggggccccagtttatctttgaatatgctaatgagttaaggagatcagaaggaaacttccctaatcgattctcagggcgccagttccatgactgttgctctgagatgaatgtgagtgccttggagctgggggactcggccctgtatctctgtgccagaagcttgg

SEQ ID NO: 133 - DNA, 286 bp, Artificial Sequence
  Synthetic DNA TRBV5-3*02
gaggctggagtcacccaaagtcccacacacctgatcaaaacgagaggacagcaagtgactctgagatgctctcctatctctgggcacagcagtgtgtcctggtaccaacaggccccgggtcaggggccccagtttatctttgaatatgctaatgagttaaggagatcagaaggaaacttccctaatcgattctcagggcgccagttccatgactattgctctgagatgaatgtgagtgccttggagctgggggactcggccctgtatctctgtgccagaagcttgg

SEQ ID NO: 134 - DNA, 286 bp, Artificial Sequence
  Synthetic DNA TRBV5-4*01
gagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagcttgg

SEQ ID NO: 135 - DNA, 282 bp, Artificial Sequence
  Synthetic DNA TRBV5-4*02
gagactggagtcacccaaagtcccacacacctgatcaaaacgagaggacagcaagtgactctgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattataactctgagctgaatgtgaacgccttggagctggacgactcggccctgtatctctgtgccagcagc

SEQ ID NO: 136 - DNA, 234 bp, Artificial Sequence
  Synthetic DNA TRBV5-4*03
cagcaagtgacactgagatgctcttctcagtctgggcacaacactgtgtcctggtaccaacaggccctgggtcaggggccccagtttatctttcagtattatagggaggaagagaatggcagaggaaacttccctcctagattctcaggtctccagttccctaattatagctctgagctgaatgtgaacgccttggagctggacgactcggccctgtatctctgtgccagcagc

SEQ ID NO: 137 - DNA, 192 bp, Artificial Sequence
  Synthetic DNA TRBV5-4*04
actgtgtcctggtaccaacaggccctgggtcaggggccccagtttatctttcagtattatagggaggaagagaatggcagaggaaactcccctcctagattctcaggtctccagttccctaattatagctctgagctgaatgtgaacgccttggagctggacgactcggccctgtatctctgtgccagcagc

SEQ ID NO: 138 - DNA, 286 bp, Artificial Sequence
  Synthetic DNA TRBV5-5*01
gacgctggagtcacccaaagtcccacacacctgatcaaaacgagaggacagcaagtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagcttgg

SEQ ID NO: 139 - DNA, 282 bp, Artificial Sequence
  Synthetic DNA TRBV5-5*02
gacgctggagtcacccaaagtcccacacacctgatcaaaacgagaggacagcacgtgactctgagatgctctcctatctctgggcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagc

SEQ ID NO: 140 - DNA, 282 bp, Artificial Sequence
  Synthetic DNA TRBV5-5*03
gacgctggagtcacccaaagtcccacacacctgatcaaaacgagaggacagcaagtgactctgagatgctctcctatctctgagcacaagagtgtgtcctggtaccaacaggtcctgggtcaggggccccagtttatctttcagtattatgagaaagaagagagaggaagaggaaacttccctgatcgattctcagctcgccagttccctaactatagctctgagctgaatgtgaacgccttgttgctgggggactcggccctgtatctctgtgccagcagc

SEQ ID NO: 141 - DNA, 286 bp, Artificial Sequence
  Synthetic DNA TRBV5-6*01
gacgctggagtcacccaaagtcccacacacctgatcaaaacgagaggacagcaagtgactctgagatgctctcctaagtctgggcatgacactgtgtcctggtaccaacaggccctgggtcaggggccccagtttatctttcagtattatgaggaggaagagagacagagaggcaacttccctgatcgattctcaggtcaccagttccctaactatagctctgagctgaatgtgaacgccttgttgctgggggactcggccctctatctctgtgccagcagcttgg

SEQ ID NO: 142 - DNA, 286 bp, Artificial Sequence
  Synthetic DNA TRBV5-7*01
gacgctggagtcacccaaagtcccacacacctgatcaaaacgagaggacagcacgtgactctgagatgctctcctatctctgggcacaccagtgtgtcctcgtaccaacaggccctgggtcaggggccccagtttatctttcagtattatgagaaagaagagagaggaagaggaaacttccctgatcaattctcaggtcaccagttccctaactatagctctgagctgaatgtgaacgccttgttgctaggggactcggccctctatctctgtgccagcagcttgg

SEQ ID NO: 143 - DNA, 286 bp, Artificial Sequence
  Synthetic DNA TRBV5-8*01
gaggctggagtcacacaaagtcccacacacctgatcaaaacgagaggacagcaagcgactctgagatgctctcctatctctgggcacaccagtgtgtactggtaccaacaggccctgggtctgggcctccagttcctcctttggtatgacgagggtgaagagagaaacagaggaaacttccctcctagattttcaggtcgccagttccctaattatagctctgagctgaatgtgaacgccttggagctggaggactcggccctgtatctctgtgccagcagcttgg

SEQ ID NO: 144 - DNA, 238 bp, Artificial Sequence
  Synthetic DNA TRBV5-8*02
aggacagcaagcgactctgagatgctctcctatctctgggcacaccagtgtgtactggtaccaacaggccctgggtctgggcctccagctcctcctttggtatgacgagggtgaagagagaaacagaggaaacttccctcctagattttcaggtcgccagttccctaattatagctctgagctgaatgtgaacgccttggagctggaggactcggccctgtatctctgtgccagcagc

SEQ ID NO: 145 - DNA, 287 bp, Artificial Sequence
  Synthetic DNA TRBV6-1*01
aatgctggtgtcactcagaccccaaaattccaggtcctgaagacaggacagagcatgacactgcagtgtgcccaggatatgaaccataactccatgtactggtatcgacaagacccaggcatgggactgaggctgatttattactcagcttctgagggtaccactgacaaaggagaagtccccaatggctacaatgtctccagattaaacaaacgggagttctcgctcaggctggagtcggctgctccctcccagacatctgtgtacttctgtgccagcagtgaagc

SEQ ID NO: 146 - DNA, 287 bp, Artificial Sequence
  Synthetic DNA TRBV6-2*01
aatgctggtgtcactcagaccccaaaattccgggtcctgaagacaggacagagcatgacactgctgtgtgcccaggatatgaaccatgaatacatgtactggtatcgacaagacccaggcatggggctgaggctgattcattactcagttggtgagggtacaactgccaaaggagaggtccctgatggctacaatgtctccagattaaaaaaacagaatttcctgctggggttggagtcggctgctccctcccaaacatctgtgtacttctgtgccagcagttactc

SEQ ID NO: 147 - DNA, 287 bp, Artificial Sequence; identical to SEQ ID NO: 146
  Synthetic DNA TRBV6-3*01

SEQ ID NO: 148 - DNA, 287 bp, Artificial Sequence
  Synthetic DNA TRBV6-4*01
attgctgggatcacccaggcaccaacatctcagatcctggcagcaggacggcgcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcagtgactc

SEQ ID NO: 149 - DNA, 287 bp, Artificial Sequence
  Synthetic DNA TRBV6-4*02
actgctgggatcacccaggcaccaacatctcagatcctggcagcaggacggagcatgacactgagatgtacccaggatatgagacataatgccatgtactggtatagacaagatctaggactggggctaaggctcatccattattcaaatactgcaggtaccactggcaaaggagaagtccctgatggttatagtgtctccagagcaaacacagatgatttccccctcacgttggcgtctgctgtaccctctcagacatctgtgtacttctgtgccagcagtgactc

SEQ ID NO: 150 - DNA, 287 bp, Artificial Sequence
  Synthetic DNA TRBV6-5*01
aatgctggtgtcactcagaccccaaaattccaggtcctgaagacaggacagagcatgacactgcagtgtgcccaggatatgaaccatgaatacatgtcctggtatcgacaagacccaggcatggggctgaggctgattcattactcagttggtgctggtatcactgaccaaggagaagtccccaatggctacaatgtctccagatcaaccacagaggatttcccgctcaggctgctgtcggctgctccctcccagacatctgtgtacttctgtgccagcagttactc

SEQ ID NO: 151 - DNA, 287 bp, Artificial Sequence
  Synthetic DNA TRBV6-6*01
aatgctggtgtcactcagaccccaaaattccgcatcctgaagataggacagagcatgacactgcagtgtacccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagttactc

SEQ ID NO: 152 - DNA, 282 bp, Artificial Sequence
  Synthetic DNA TRBV6-6*02
aatgctggtgtcactcagaccccaaaattccgcatcctgaagataggacagagcatgacactgcagtgtgcccaggatatgaaccataactacatgtactggtatcgacaagacccaggcatggggctgaagctgatttattattcagttggtgctggtatcactgacaaaggagaagtcccgaatggctacaacgtctccagatcaaccacagaggatttcccgctcaggctggagttggctgctccctcccagacatctgtgtacttctgtgccagcagt

SEQ ID NO: 153 - DNA, 282 bp, Artificial Sequence
  Synthetic DNA TRBV6-6*03
aatgctggtgtcactcagaccccaaaattccgcatcctgaagataggacagagcatgacactgcagtgtgcccaggatatgaaccataactacatgtactggtatcgacaagacccaggcatggggctgaagctgatttattattcagttggtgctggtatcactgataaaggagaagtcccgaatggctacaacgtctccagatcaaccacagaggatttcccgctcaggctggagttggctgctccctcccagacatctgtgtacttctgtgccagcagt

SEQ ID NO: 154 - DNA, 285 bp, Artificial Sequence
  Synthetic DNA TRBV6-6*04
aatgctggtgtcactcagaccccaaaattccgcatcctgaagataggacagagcatgacactgcagtgtacccaggatatgaaccatgaatacatgtactggtatcgacaagacccaggcatggggctgaagctgatttattattcagttggtgctggtatcactgataaaggagaagtcccgaatggctacaatgtctccagatcaaccacagaggatttcccgctcaggctggagttggctgctccctcccagacatctgtgtacttctgtgccagcagtcga

SEQ ID NO: 155 - DNA, 282 bp, Artificial Sequence
  Synthetic DNA TRBV6-6*05
aatgctggtgtcactcagaccccaaaattccgcatcctgaagataggacagagcatgacactgcagtgtgcccaggatatgaaccataactacatgtactggtatcgacaagacccaggcatggggctgaagctgatttattattcagttggtgctggtatcactgacaaaggagaagtcccgaatggctacaacgtctccagatcaaccacagaggatttcccgctcaggctggagttggctgctgcctcccagacatctgtgtacttctgtgccagcagc

SEQ ID NO: 156 - DNA, 287 bp, Artificial Sequence
  Synthetic DNA TRBV6-7*01
aatgctggtgtcactcagaccccaaaattccacgtcctgaagacaggacagagcatgactctgctgtgtgcccaggatatgaaccatgaatacatgtatcggtatcgacaagacccaggcaaggggctgaggctgatttactactcagttgctgctgctctcactgacaaaggagaagttcccaatggctacaatgtctccagatcaaacacagaggatttccccctcaagctggagtcagctgctccctctcagacttctgtttacttctgtgccagcagttactc

SEQ ID NO: 157 - DNA, 284 bp, Artificial Sequence
  Synthetic DNA TRBV6-8*01
aatgctggtgtcactcagaccccaaaattccacatcctgaagacaggacagagcatgacactgcagtgtgcccaggatatgaaccatggatacatgtcctggtatcgacaagacccaggcatggggctgagactgatttactactcagctgctgctggtactactgacaaagaagtccccaatggctacaatgtctctagattaaacacagaggatttcccactcaggctggtgtcggctgctccctcccagacatctgtgtacttgtgtgccagcagttactc

SEQ ID NO: 158 - DNA, 287 bp, Artificial Sequence
  Synthetic DNA TRBV6-9*01
aatgctggtgtcactcagaccccaaaattccacatcctgaagacaggacagagcatgacactgcagtgtgcccaggatatgaaccatggatacttgtcctggtatcgacaagacccaggcatggggctgaggcgcattcattactcagttgctgctggtatcactgacaaaggagaagtccccgatggctacaatgtatccagatcaaacacagaggatttcccgctcaggctggagtcagctgctccctcccagacatctgtatacttctgtgccagcagttattc

SEQ ID NO: 159 - DNA, 290 bp, Artificial Sequence
  Synthetic DNA TRBV7-1*01
ggtgctggagtctcccagtccctgagacacaaggtagcaaagaagggaaaggatgtagctctcagatatgatccaatttcaggtcataatgccctttattggtaccgacagagcctggggcagggcctggagtttccaatttacttccaaggcaaggatgcagcagacaaatcggggcttccccgtgatcggttctctgcacagaggtctgagggatccatctccactctgaagttccagcgcacacagcagggggacttggctgtgtatctctgtgccagcagctcagc

SEQ ID NO: 160 - DNA, 290 bp, Artificial Sequence
  Synthetic DNA TRBV7-2*01
ggagctggagtctcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagcttagc

SEQ ID NO: 161 - DNA, 290 bp, Artificial Sequence
  Synthetic DNA TRBV7-2*02
ggagctggagtctcccagtcccccagtaacaaggtcacagagaagggaaaggatgtagagctcaggtgtgatccaatttcaggtcatactgccctttactggtaccgacagaggctggggcagggcctggagtttttaatttacttccaaggcaacagtgcaccagacaaatcagggctgcccagtgatcgcttctctgcagagaggactggggaatccgtctccactctgacgatccagcgcacacagcaggaggactcggccgtgtatctctgtgccagcagcttagc

SEQ ID NO: 162 - DNA, 290 bp, Artificial Sequence
  Synthetic DNA TRBV7-2*03
ggagctggagtctcccagtcccccagtaacaaggtcacagagaagggaaaggatgtagagctcaggtgtgatccaatttcaggtcatactgccctttactggtaccgacagaggctggggcagggcctggagtttttaatttacttccaaggcaacagtgcaccagacaaatcagggctgcccagtgatcgcttctctgcagagaggactggggaatccgtctccactctgacgatccagcgcacacagcaggaggactcggccgtgtatctctgtaccagcagcttagc

SEQ ID NO: 163 - DNA, 288 bp, Artificial Sequence
  Synthetic DNA TRBV7-2*04
ggagctggagtttcccagtcccccagtaacaaggtcacagagaagggaaaggatgtagagctcaggtgtgatccaatttcaggtcatactgccctttactggtaccgacagagcctggggcagggcctggagtttttaatttacttccaaggcaacagtgcaccagacaaatcagggctgcccagtgatcgcttctctgcagagaggactgggggatccgtctccactctgacgatccagcgcacacagcaggaggactcggccgtgtatctctgtgccagcagctta

SEQ ID NO: 164 - DNA, 290 bp, Artificial Sequence
  Synthetic DNA TRBV7-3*01
ggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagcttaac

SEQ ID NO: 165 - DNA, 290 bp, Artificial Sequence
  Synthetic DNA TRBV7-3*02
ggtgctggagtctcccagacccccagtaacaaggtcacagagaagggaaaagatgtagagctcaggtgtgatccaatttcaggtcatactgccctttactggtaccgacaaagcctggggcagggcccagagtttctaatttacttccaaggcacgggtgcggcagatgactcagggctgcccaaagatcggttctttgcagtcaggcctgagggatccgtctctactctgaagatccagcgcacagagcagggggactcagccgtgtatctccgtgccagcagcttaac

SEQ ID NO: 166 - DNA, 288 bp, Artificial Sequence
  Synthetic DNA TRBV7-3*03
ggtgctggagtctcccagacccccagtaacaaggtcacagagaagggaaaagatgtagagctcaggtgtgatccaatttcaggtcatactgccctttactggtaccgacaaagcctggggcagggcccagagtttctaatttacttccaaggcacgggtgcggcagatgactcagggctgcccaaagatcggttctttgcagtcaggcctgagggatccgtctctactctgaagatccagcgcacagagcagggggactcagccgcgtatctccgtgccagcagctta

SEQ ID NO: 167 - DNA, 285 bp, Artificial Sequence
  Synthetic DNA TRBV7-3*04
ggtgctggagtctcccagacccccagtaacaaggtcacagagaagggaaaatatgtagagctcaggtgtgatccaatttcaggtcatactgccctttactggtaccgacaaagcctggggcagggcccagagtttctaatttacttccaaggcacgggtgcggcagatgactcagggctgcccaacgatcggttctttgcagtcaggcctgagggatccgtctctactctgaagatccagcgcacagagcggggggactctgccgtgtatctctgtgccagcagc

SEQ ID NO: 168 - DNA, 231 bp, Artificial Sequence
  Synthetic DNA TRBV7-3*05
tgggagctcaggtgtgatccaatttcaggtcatactgccctttactggtaccgacaaagcctggggcagggcccagagcttctaatttacttccaaggcacgggtgcggcagatgactcagggctgcccaacgatcggttctttgcagtcaggcctgagggatccgtctctactctgaagatccagcgcacagagcggggggactcagccgtgtatctctgtgccagcagc

SEQ ID NO: 169 - DNA, 290 bp, Artificial Sequence
  Synthetic DNA TRBV7-4*01
ggtgctggagtctcccagtccccaaggtacaaagtcgcaaagaggggacgggatgtagctctcaggtgtgattcaatttcgggtcatgtaaccctttattggtaccgacagaccctggggcagggctcagaggttctgacttactcccagagtgatgctcaacgagacaaatcagggcggcccagtggtcggttctctgcagagaggcctgagagatccgtctccactctgaagatccagcgcacagagcagggggactcagctgtgtatctctgtgccagcagcttagc

SEQ ID NO: 170 - DNA, 288 bp, Artificial Sequence
  Synthetic DNA TRBV7-5*01
ggtgctggagtctcccagtccccaaggtacgaagtcacacagaggggacaggatgtagctcccaggtgtgatccaatttcgggtcaggtaaccctttattggtaccgacagaccctggggcagggccaagagtttctgacttccttccaggatgaaactcaacaagataaatcagggctgctcagtgatcaattctccacagagaggtctgaggatctttctccacctgaagatccagcgcacagagcaagggcgactcggctgtgtatctctgtgccagaagcttag

SEQ ID NO: 171 - DNA, 289 bp, Artificial Sequence
  Synthetic DNA TRBV7-5*02
ggtgctggagtctcccagtccccaaggtacgaagtcacacagaggggacaggatgtagctcccaggtgtgatccaatttcgggtcaggtaaccctttattggtaccgacagaccctggggcagggccaagagtttctgacttccttccaggatgaaactcaacaagataaatcagggctgctcagtgatcaattctccacagagaggtctgaggatctttctccacctgaagatccagcgcacagagcaagggcgactcggctgtgtatctctgtgtcagaagcttagc

SEQ ID NO: 172 - DNA, 290 bp, Artificial Sequence
  Synthetic DNA TRBV7-6*01
ggtgctggagtctcccagtctcccaggtacaaagtcacaaagaggggacaggatgtagctctcaggtgtgatccaatttcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgctgtgccagcagcttagc

SEQ ID NO: 173 - DNA, 285 bp, Artificial Sequence
  Synthetic DNA TRBV7-6*02
ggtgctggagtctcccagtctcccaggtacaaagtcacaaagaggggacaggatgtagctctcaggtgtgatccaatctcgggtcatgtatccctttattggtaccgacaggccctggggcagggcccagagtttctgacttacttcaattatgaagcccaacaagacaaatcagggctgcccaatgatcggttctctgcagagaggcctgagggatccatctccactctgacgatccagcgcacagagcagcgggactcggccatgtatcgctgtgccagcagc

SEQ ID NO: 174 - DNA, 290 bp, Artificial Sequence
  Synthetic DNA TRBV7-7*01
ggtgctggagtctcccagtctcccaggtacaaagtcacaaagaggggacaggatgtaactctcaggtgtgatccaatttcgagtcatgcaaccctttattggtatcaacaggccctggggcagggcccagagtttctgacttacttcaattatgaagctcaaccagacaaatcagggctgcccagtgatcggttctctgcagagaggcctgagggatccatctccactctgacgattcagcgcacagagcagcgggactcagccatgtatcgctgtgccagcagcttagc

SEQ ID NO: 175 - DNA, 285 bp, Artificial Sequence
  Synthetic DNA TRBV7-7*02
ggtgctggagtctcccagtctcccaggtacaaagtcacaaagaggggacaggatgtaactctcaggtgtgatccaatttcgagtcatgtaaccctttattggtatcaacaggccctggggcagggcccagagtttctgacttacttcaattatgaagctcaaccagacaaatcagggctgcccagtgatcggttctctgcagagaggcctgagggatccatctccactctgacgattcagcgcacagagcagcgggactcagccatgtatcgctgtgccagcagc

SEQ ID NO: 176 - DNA, 290 bp, Artificial Sequence
  Synthetic DNA TRBV7-8*01
ggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcagcttagc

SEQ ID NO: 177 - DNA, 290 bp, Artificial Sequence
  Synthetic DNA TRBV7-8*02
ggtgctggagtctcccagtcccctaggtacaaagtcgcaaagagaggacaggatgtagctctcaggtgtgatccaatttcgggtcatgtatcccttttttggtaccaacaggccctggggcaggggccagagtttctgacttatttccagaatgaagctcaactagacaaatcggggctgcccagtgatcgcttctttgcagaaaggcctgagggatccgtctccactctgaagatccagcgcacacagaaggaggactccgccgtgtatctctgtgccagcagcttagc

SEQ ID NO: 178 - DNA, 288 bp, Artificial Sequence
  Synthetic DNA TRBV7-8*03
ggtgctggagtctcccagtcccctaggtacaaagtcgcaaagagaggacaggatgtagctctcaggtgtgatccaatttcgggtcatgtatcccttttttggtaccaacaggccctcgggcaggggccagagtttctgacttatttccagaatgaagctcaactagacaaatcggggctgcccagtgatcgcttctttgcagaaaggcctgagggatccgtctccactctgaagatccagcgcacacagcaggaggactccgccgtgtatctctgtgccagcagccga

SEQ ID NO: 179 - DNA, 288 bp, Artificial Sequence
  Synthetic DNA TRBV7-9*05
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctctctccaccttggagatccagcgcacagagcagggggactcggccatgtatctctgtgccagcaccaaa

SEQ ID NO: 180 - DNA, 288 bp, Artificial Sequence
  Synthetic DNA TRBV7-9*06
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctctttccaccttggagatccagcgcacagagcagggggactcggccatgtatctctgtgccagcacgttg

SEQ ID NO: 181 - DNA, 285 bp, Artificial Sequence
  Synthetic DNA TRBV7-9*03
gatactggagtctcccaggaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagc

SEQ ID NO: 182 - DNA, 290 bp, Artificial Sequence
  Synthetic DNA TRBV7-9*01
gatactggagtctcccagaaccccagacacaagatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagcttagc

SEQ ID NO: 183 - DNA, 288 bp, Artificial Sequence
  Synthetic DNA TRBV7-9*02
gatactggagtctcccagaaccccagacacaacatcacaaagaggggacagaatgtaactttcaggtgtgatccaatttctgaacacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagagcagggggactcggccatgtatctctgtgccagcagctta

SEQ ID NO: 184 - DNA, 207 bp, Artificial Sequence
  Synthetic DNA TRBV7-9*07
cacaaccgcctttattggtaccgacagaccctggggcagggcccagagtttctgacttacttccagaatgaagctcaactagaaaaatcaaggctgctcagtgatcggttctctgcagagaggcctaagggatctttctccaccttggagatccagcgcacagaggagggggactcggccatgtatctctgtgccagcagcagcagt

SEQ ID NO: 185 - DNA, 288 bp, Artificial Sequence
  Synthetic DNA TRBV7-9*04
atatctggagtctcccacaaccccagacacaagatcacaaagaggggacagaatgtaactttcaggtgtgatccaatttctgaacacaaccgcctttattggtaccgacagaaccctgggcagggcccagagtttctgacttacttccagaatgaagctcaactggaaaaatcagggctgctcagtgatcggatctctgcagagaggcctaagggatctttctccaccttggagatccagcgcacagagcagggggactcggccatgtatctctgtgccagcagctct

SEQ ID NO: 186 - DNA, 279 bp, Artificial Sequence
  Synthetic DNA TRBV8-1*01
gaggcagggatcagccagataccaagatatcacagacacacagggaaaaagatcatcctgaaatatgctcagattaggaaccattattcagtgttctgttatcaataagaccaagaataggggctgaggctgatccattattcaggtagtattggcagcatgaccaaaggcggtgccaaggaagggtacaatgtctctggaaacaagctcaagcattttccctcaaccctggagtctactagcaccagccagacctctgtacctctgtggcagtgcatc

SEQ ID NO: 187 - DNA, 271 bp, Artificial Sequence
  Synthetic DNA TRBV8-2*01
gatgctgggatcacccagatgccaagatatcacattgtacagaagaaagagatgatcctggaatgtgctcaggttaggaacagtgttctgatatcgacaggacccaagacgggggctgaagcttatccactattcaggcagtggtcacagcaggaccaaagttgatgtcacagaggggtactgtgtttcttgaaacaagcttgagcatttccccaatcctggcatccaccagcaccagccagacctatctgtaccactgtggcagcacatc

SEQ ID NO: 188 - DNA, 286 bp, Artificial Sequence
  Synthetic DNA TRBV9*01
gattctggagtcacacaaaccccaaagcacctgatcacagcaactggacagcgagtgacgctgagatgctcccctaggtctggagacctctctgtgtactggtaccaacagagcctggaccagggcctccagttcctcattcagtattataatggagaagagagagcaaaaggaaacattcttgaacgattctccgcacaacagttccctgacttgcactctgaactaaacctgagctctctggagctgggggactcagctttgtatttctgtgccagcagcgtag

SEQ ID NO: 189 - DNA, 282 bp, Artificial Sequence
  Synthetic DNA TRBV9*03
gattctggagtcacacaaaccccaaagcacctgatcacagcaactggacagcgagtgacgctgagatgctcccctaggtctggagacctctctgtgtactggtaccaacagagcctggaccagggcctccagttcctcattcaatattataatggagaagagagagcaaaaggaaacattcttgaacgattctccgcacaacagttccctgacttgcactctgaactaaacctgagctctctggagctgggggactcagctttgtatttctgtgccagcagc

SEQ ID NO: 190 - DNA, 286 bp, Artificial Sequence
  Synthetic DNA TRBV9*02
gattctggagtcacacaaaccccaaagcacctgatcacagcaactggacagcgagtgacgctgagatgctcccctaggtctggagacctctctgtgtactggtaccaacagagcctggaccagggcctccagttcctcattcactattataatggagaagagagagcaaaaggaaacattcttgaacgattctccgcacaacagttccctgacttgcactctgaactaaacctgagctctctggagctgggggactcagctttgtatttctgtgccagcagcgtag

SEQ ID NO: 191 - DNA, 287 bp, Artificial Sequence
  Synthetic DNA TRBV10-1*01
gatgctgaaatcacccagagcccaagacacaagatcacagagacaggaaggcaggtgaccttggcgtgtcaccagacttggaaccacaacaatatgttctggtatcgacaagacctgggacatgggctgaggctgatccattactcatatggtgttcaagacactaacaaaggagaagtctcagatggctacagtgtctctagatcaaacacagaggacctccccctcactctggagtctgctgcctcctcccagacatctgtatatttctgcgccagcagtgagtc

SEQ ID NO: 192 - DNA, 282 bp, Artificial Sequence
  Synthetic DNA TRBV10-1*02
gatgctgaaatcacccagagcccaagacacaagatcacagagacaggaaggcaggtgaccttggcgtgtcaccagacttggaaccacaacaatatgttctggtatcgacaagacctgggacatgggctgaggctgatccattactcatatggtgttcacgacactaacaaaggagaagtctcagatggctacagtgtctctagatcaaacacagaggacctccccctcactctggagtctgctgcctcctcccagacatctgtatatttctgcgccagcagt

SEQ ID NO: 193 - DNA, 287 bp, Artificial Sequence
  Synthetic DNA TRBV10-2*01
gatgctggaatcacccagagcccaagatacaagatcacagagacaggaaggcaggtgaccttgatgtgtcaccagacttggagccacagctatatgttctggtatcgacaagacctgggacatgggctgaggctgatctattactcagcagctgctgatattacagataaaggagaagtccccgatggctatgttgtctccagatccaagacagagaatttccccctcactctggagtcagctacccgctcccagacatctgtgtatttctgcgccagcagtgagtc

SEQ ID NO: 194 - DNA, 217 bp, Artificial Sequence
  Synthetic DNA TRBV10-2*02
aaggcaggtgaccttgatgtgtcaccagacttggagccacagctatatgttctggtatcgacaagacctgggacatgggctgaggctgatctattactcagcagctgctgatattacagataaaggagaagtccccgatggctacgttgtctccagatccaagacagagaatttccccctcactctggagtcagctacccgctcccagacatctgtg

SEQ ID NO: 195 - DNA, 273 bp, Artificial Sequence
  Synthetic DNA TRBV10-3*03
gatgctggaatcacccagagcccaagacacaaggtcacagagacaggaacaccagtgactctgagatgtcaccagactgagaaccaccgctacatgtactggtatcgacaagacccggggcatgggctgaggctaatccattactcatatggtgttaaagatactgacaaaggagaagtctcagatggctatagtgtctctagatcaaagacagaggatttcctcctcactctggagtccgctaccagctcccagacatctgtgtacttctgt

SEQ ID NO: 196 - DNA, 273 bp, Artificial Sequence
  Synthetic DNA TRBV10-3*04
gatgctggaatcacccagagcccaagacacaaggtcacagagacaggaacaccagtgactctgagatgtcaccagactgagaaccaccgctacatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgt

SEQ ID NO: 197 - DNA, 287 bp, Artificial Sequence
  Synthetic DNA TRBV10-3*01
gatgctggaatcacccagagcccaagacacaaggtcacagagacaggaacaccagtgactctgagatgtcaccagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgagtc

SEQ ID NO: 198 - DNA, 287 bp, Artificial Sequence
  Synthetic DNA TRBV10-3*02
gatgctggaatcacccagagcccaagacacaaggtcacagagacaggaacaccagtgactctgagatgtcatcagactgagaaccaccgctatatgtactggtatcgacaagacccggggcatgggctgaggctgatccattactcatatggtgttaaagatactgacaaaggagaagtctcagatggctatagtgtctctagatcaaagacagaggatttcctcctcactctggagtccgctaccagctcccagacatctgtgtacttctgtgccatcagtgagtc

SEQ ID NO: 199 - DNA, 290 bp, Artificial Sequence
  Synthetic DNA TRBV11-1*01
gaagctgaagttgcccagtcccccagatataagattacagagaaaagccaggctgtggctttttggtgtgatcctatttctggccatgctaccctttactggtaccggcagatcctgggacagggcccggagcttctggttcaatttcaggatgagagtgtagtagatgattcacagttgcctaaggatcgattttctgcagagaggctcaaaggagtagactccactctcaagatccagcctgcagagcttggggactcggccatgtatctctgtgccagcagcttagc

SEQ ID NO: 200 - DNA, 290 bp, Artificial Sequence
  Synthetic DNA TRBV11-3*01
gaagctggagtggttcagtctcccagatataagattatagagaaaaaacagcctgtggctttttggtgcaatcctatttctggccacaataccctttactggtacctgcagaacttgggacagggcccggagcttctgattcgatatgagaatgaggaagcagtagacgattcacagttgcctaaggatcgattttctgcagagaggctcaaaggagtagactccactctcaagatccagcctgcagagcttggggactcggccgtgtatctctgtgccagcagcttaga

SEQ ID NO: 201 - DNA, 285 bp, Artificial Sequence
  Synthetic DNA TRBV11-3*02
gaagctggagtggttcagtctcccagatataagattatagagaaaaagcagcctgtggctttttggtgcaatcctatttctggccacaataccctttactggtaccggcagaacttgggacagggcccggagcttctgattcgatatgagaatgaggaagcagtagacgattcacagttgcctaaggatcgattttctgcagagaggctcaaaggagtagactccactctcaagatccagcctgcagagcttggggactcggccgtgtatctctgtgccagcagc

SEQ ID NO: 202 - DNA, 269 bp, Artificial Sequence
  Synthetic DNA TRBV11-3*03
ggtctcccagatataagattatagagaagaaacagcctgtggctttttggtgcaatccaatttctggccacaataccctttactggtacctgcagaacttgggacagggcccggagcttctgattcgatatgagaatgaggaagcagtagacgattcacagttgcctaaggatcgattttctgcagagaggctcaaaggagtagactccactctcaagatccagccagcagagcttggggactcggccatgtatctctgtgccagcagc

SEQ ID NO: 203 - DNA, 290 bp, Artificial Sequence
  Synthetic DNA TRBV11-2*01
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgaggactcggccgtgtatctctgtgccagcagcttaga

SEQ ID NO: 204 - DNA, 285 bp, Artificial Sequence
  Synthetic DNA TRBV11-2*03
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccaacctgcaaagcttgaggactcggccgtgtatctctgtgccagcagc

SEQ ID NO: 205 - DNA, 285 bp, Artificial Sequence
  Synthetic DNA TRBV11-2*02
gaagctggagttgcccagtctcccagatataagattatagagaaaaggcagagtgtggctttttggtgcaatcctatatctggccatgctaccctttactggtaccagcagatcctgggacagggcccaaagcttctgattcagtttcagaataacggtgtagtggatgattcacagttgcctaaggatcgattttctgcagagaggctcaaaggagtagactccactctcaagatccagcctgcaaagcttgagaactcggccgtgtatctctgtgccagcagt

SEQ ID NO: 206 - DNA, 290 bp, Artificial Sequence
  Synthetic DNA TRBV12-1*01
gatgctggtgttatccagtcacccaggcacaaagtgacagagatgggacaatcagtaactctgagatgcgaaccaatttcaggccacaatgatcttctctggtacagacagacctttgtgcagggactggaattgctgaattacttctgcagctggaccctcgtagatgactcaggagtgtccaaggattgattctcagcacagatgcctgatgtatcattctccactctgaggatccagcccatggaacccagggacttgggcctatatttctgtgccagcagctttgc

SEQ ID NO: 207 - DNA, 290 bp, Artificial Sequence
  Synthetic DNA TRBV12-2*01
gatgctggcattatccagtcacccaagcatgaggtgacagaaatgggacaaacagtgactctgagatgtgagccaatttttggccacaatttccttttctggtacagagataccttcgtgcagggactggaattgctgagttacttccggagctgatctattatagataatgcaggtatgcccacagagcgattctcagctgagaggcctgatggatcattctctactctgaagatccagcctgcagagcagggggactcggccgtgtatgtctgtgcaagtcgcttagc

SEQ ID NO: 208 - DNA, 290 bp, Artificial Sequence
  Synthetic DNA TRBV12-4*01
gatgctggagttatccagtcaccccggcacgaggtgacagagatgggacaagaagtgactctgagatgtaaaccaatttcaggacacgactaccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttagc

SEQ ID NO: 209 - DNA, 288 bp, Artificial Sequence
  Synthetic DNA TRBV12-4*02
gatgctggagttatccagtcaccccggcacgaggtgacagagatgggacaagaagtgactctgagatgtaaaccaatttcaggacatgactaccttttctggtacagacagaccatgatgcggggactggagttgctcatttactttaacaacaacgttccgatagatgattcagggatgcccgaggatcgattctcagctaagatgcctaatgcatcattctccactctgaggatccagccctcagaacccagggactcagctgtgtacttctgtgccagcagttta

SEQ ID NO: 210 - DNA, 290 bp, Artificial Sequence
  Synthetic DNA TRBV12-3*01
gatgctggagttatccagtcaccccgccatgaggtgacagagatgggacaagaagtgactctgagatgtaaaccaatttcaggccacaactcccttttctggtacagacagaccatgatgcggggactggagttgctcatttactttaacaacaacgttccgatagatgattcagggatgcccgaggatcgattctcagctaagatgcctaatgcatcattctccactctgaagatccagccctcagaacccagggactcagctgtgtacttctgtgccagcagtttagc

SEQ ID NO: 211 - DNA, 290 bp, Artificial Sequence
  Synthetic DNA TRBV12-5*01
gatgctagagtcacccagacaccaaggcacaaggtgacagagatgggacaagaagtaacaatgagatgtcagccaattttaggccacaatactgttttctggtacagacagaccatgatgcaaggactggagttgctggcttacttccgcaaccgggctcctctagatgattcggggatgccgaaggatcgattctcagcagagatgcctgatgcaactttagccactctgaagatccagccctcagaacccagggactcagctgtgtatttttgtgctagtggtttggt

SEQ ID NO: 212 - DNA, 287 bp, Artificial Sequence
  Synthetic DNA TRBV13*01
gctgctggagtcatccagtccccaagacatctgatcaaagaaaagagggaaacagccactctgaaatgctatcctatccctagacacgacactgtctactggtaccagcagggtccaggtcaggacccccagttcctcatttcgttttatgaaaagatgcagagcgataaaggaagcatccctgatcgattctcagctcaacagttcagtgactatcattctgaactgaacatgagctccttggagctgggggactcagccctgtacttctgtgccagcagcttagg

SEQ ID NO: 213 - DNA, 282 bp, Artificial Sequence
  Synthetic DNA TRBV13*02
gctgctggagtcatccagtccccaagacatctgatcagagaaaagagggaaacagccactctgaaatgctatcctatccctagacacgacactgtctactggtaccagcagggcccaggtcaggacccccagttcttcatttcgttttatgaaaagatgcagagcgataaaggaagcatccctgatcgattctcagctcaacagttcagtgactatcattctgaactgaacatgagctccttggagctgggggactcagccctgtacttctgtgccagcagc

SEQ ID NO: 214 - DNA, 290 bp, Artificial Sequence
  Synthetic DNA TRBV14*01
gaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgagtccggtatgcccaacaatcgattcttagctgaaaggactggagggacgtattctactctgaaggtgcagcctgcagaactggaggattctggagtttatttctgtgccagcagccaaga

SEQ ID NO: 215 - DNA, 285 bp, Artificial Sequence
  Synthetic DNA TRBV14*02
gaagctggagttactcagttccccagccacagcgtaatagagaagggccagactgtgactctgagatgtgacccaatttctggacatgataatctttattggtatcgacgtgttatgggaaaagaaataaaatttctgttacattttgtgaaagagtctaaacaggatgaatccggtatgcccaacaatcgattcttagctgaaaggactggagggacgtattctactctgaaggtgcagcctgcagaactggaggattctggagtttatttctgtgccagcagc

SEQ ID NO: 216 - DNA, 287 bp, Artificial Sequence
  Synthetic DNA TRBV15*01
gatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacacagccatgtacctgtgtgccaccagcagaga

SEQ ID NO: 217 - DNA, 282 bp, Artificial Sequence
  Synthetic DNA TRBV15*03
gatgccatggtcatccagaacccaagataccgggttacccagtttggaaagccagtgaccctgagttgttctcagactttgaaccataacgtcatgtactggtaccagcagaagtcaagtcaggccccaaagctgctgttccactactataacaaagattttaacaatgaagcagacacccctgataacttccaatccaggaggccgaacacttctttctgctttctagacatccgctcaccaggcctgggggacgcagccatgtaccagtgtgccaccagc

SEQ ID NO: 218 - DNA, 282 bp, Artificial Sequence
  Synthetic DNA TRBV15*02
gatgccatggtcatccagaacccaagataccaggttacccagtttggaaagccagtgaccctgagttgttctcagactttgaaccataacgtcatgtactggtaccagcagaagtcaagtcaggccccaaagctgctgttccactactatgacaaagattttaacaatgaagcagacacccctgataacttccaatccaggaggccgaacacttctttctgctttcttgacatccgctcaccaggcctgggggacgcagccatgtacctgtgtgccaccagc

SEQ ID NO: 219 - DNA, 290 bp, Artificial Sequence
  Synthetic DNA TRBV16*01
ggtgaagaagtcgcccagactccaaaacatcttgtcagaggggaaggacagaaagcaaaattatattgtgccccaataaaaggacacagttatgttttttggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctacgaagcttgaggattcagcagtgtatttttgtgccagcagccaatc

SEQ ID NO: 220 - DNA, 290 bp, Artificial Sequence
  Synthetic DNA TRBV16*02
ggtgaagaagtcgcccagactccaaaacatcttgtcagaggggaaggacagaaagcaaaattatattgtgccccaataaaaggacacagttaggttttttggtaccaacaggtcctgaaaaacgagttcaagttcttgatttccttccagaatgaaaatgtctttgatgaaacaggtatgcccaaggaaagattttcagctaagtgcctcccaaattcaccctgtagccttgagatccaggctacgaagcttgaggattcagcagtgtatttttgtgccagcagccaatc

SEQ ID NO: 221 - DNA, 285 bp, Artificial Sequence
  Synthetic DNA TRBV16*03
ggtgaagaagtcgcccagactccaaaacatcttgtcagaggggaaggacagaaagcaaaattatattgtgccccaataaaaggacacagttatgttttttggtaccaacaggtcctgaaaaacgagttcaagttcttggtttccttccagaatgaaaatgtctttgatgaaacaggtatgcccaaggaaagattttcagctaagtgcctcccaaattcaccctgtagccttgagatccaggctacgaagcttgaggattcagcagtgtatttttgtgccagcagc

SEQ ID NO: 222 - DNA, 287 bp, Artificial Sequence
  Synthetic DNA TRBV17*01
gagcctggagtcagccagacccccagacacaaggtcaccaacatgggacaggaggtgattctgaggtgcgatccatcttctggtcacatgtttgttcactggtaccgacagaatctgaggcaagaaatgaagttgctgatttccttccagtaccaaaacattgcagttgattcagggatgcccaaggaacgattcacagctgaaagacctaacggaacgtcttccacgctgaagatccatcccgcagagccgagggactcagccgtgtatctctacagtagcggtgg

SEQ ID NO: 223 - DNA, 290 bp, Artificial Sequence
  Synthetic DNA TRBV18*01
aatgccggcgtcatgcagaacccaagacacctggtcaggaggaggggacaggaggcaagactgagatgcagcccaatgaaaggacacagtcatgtttactggtatcggcagctcccagaggaaggtctgaaattcatggtttatctccagaaagaaaatatcatagatgagtcaggaatgccaaaggaacgattttctgctgaatttcccaaagagggccccagcatcctgaggatccagcaggtagtgcgaggagattcggcagcttatttctgtgccagctcaccacc

SEQ ID NO: 224 - DNA, 287 bp, Artificial Sequence
  Synthetic DNA TRBV19*01
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacagatagtaaatgactttcagaaaggagatatagctgaagggtacagcgtctctcgggagaagaaggaatcctttcctctcactgtgacatcggcccaaaagaacccgacagctttctatctctgtgccagtagtataga

SEQ ID NO: 225 - DNA, 287 bp, Artificial Sequence
  Synthetic DNA TRBV19*02
gatggtggaatcactcagtccccaaagtacctgttcagaaaggaaggacagaatgtgaccctgagttgtgaacagaatttgaaccacgatgccatgtactggtaccgacaggtcccagggcaagggctgagattgatctactactcacacatagtaaatgactttcagaaaggagatatagctgaagggtacagcgtctctcgggagaagaaggaatcctttcctctcactgtgacatcggcccaaaagaacccgacagctttctatctctgtgccagtagtataga

SEQ ID NO: 226 - DNA, 282 bp, Artificial Sequence
  Synthetic DNA TRBV19*03
gatggtggaatcactcagtccccaaagtacctgttcagaaaggaaggacagaatgtgaccctgagttgtgaacagaatttgaaccacgatgccatgtactggtaccgacaggacccagggcaagggctgagattgatctactactcacacatagtaaatgactttcagaaaggagatatagctgaagggtacagcgtctctcgggagaagaaggaatcctttcctctcactgtgacatcggcccaaaagaacccgacagctttctatctctgtgccagtagc

SEQ ID NO: 227 - DNA, 291 bp, Artificial Sequence
  Synthetic DNA TRBV20-1*05
ggtgctgtcgtctctcaacatccgagcagggttatctgtaagagtggaacctctgtgaagatcgagtgccgttccctggactttcaggccacaactatgttttggtatcgtcagttcccgaaaaagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaga

SEQ ID NO: 228 - DNA, 291 bp, Artificial Sequence
  Synthetic DNA TRBV20-1*07
ggtgctgtcgtctctcaacatccgagcagggttatctgtaagagtggaacctctgtgaagatcgagtgccgttccctggactttcaggccacaactatgttttggtatcgtcagttcccgaaaaagagtctcatgcagatcgcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctaga

SEQ ID NO: 229 - DNA, 291 bp, Artificial Sequence
  Synthetic DNA TRBV20-1*04
ggtgctgtcgtctctcaacatccgagcagggttatctgtaagagtggaacctctgtgaagatcgagtgccgttccttggactttcaggccacaactatgttttggtatcgtcagttcccgaaaaagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagt

SEQ ID NO: 230 - DNA, 288 bp, Artificial Sequence
  Synthetic DNA TRBV20-1*06
ggtgctgtcgtctctcaacatccgagtagggttatctgtaagagtggaacctctgtgaagatcgagtgccgttccctggactttcaggccacaactatgttttggtatcgtcagttcccgaaaaagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgct

SEQ ID NO: 231 - DNA, 288 bp, Artificial Sequence
  Synthetic DNA TRBV20-1*02
ggtgctgtcgtctctcaacatccgagcagggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgct

SEQ ID NO: 232 - DNA, 293 bp, Artificial Sequence
  Synthetic DNA TRBV20-1*01
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagaga

SEQ ID NO: 233 - DNA, 288 bp, Artificial Sequence
  Synthetic DNA TRBV20-1*03
ggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctgcaaggccacatacgagcaaggcgtcgagaaggacaagtttctcatcaaccatgcaagcctgaccttgtccactctgacagtgaccagtgcccatcctgaagacagcagcttctacatctgcagtgct

SEQ ID NO: 234 - DNA, 290 bp, Artificial Sequence
  Synthetic DNA TRBV21-1*01
gacaccaaggtcacccagagacctagacttctggtcaaagcaagtgaacagaaagcaaagatggattgtgttcctataaaagcacatagttatgtttactggtatcgtaagaagctggaagaagagctcaagtttttggtttactttcagaatgaagaacttattcagaaagcagaaataatcaatgagcgatttttagcccaatgctccaaaaactcatcctgtaccttggagatccagtccacggagtcaggggacacagcactgtatttctgtgccagcagcaaagc

SEQ ID NO: 235 - DNA, 288 bp, Artificial Sequence
  Synthetic DNA TRBV22-1*01
gatgctgacatctatcagatgccattccagctcactggggctggatgggatgtgactctggagtggaaacggaatttgagacacaatgacatgtactgctactggtactggcaggacccaaagcaaaatctgagactgatctattactcaagggttgaaaaggatattcagagaggagatctaactgaaggctacgtgtctgccaagaggagaaggggctatttcttctcagggtgaagttggcccacaccagccaaacagctttgtacttctgtcctgggagcgcac

SEQ ID NO: 236 - DNA, 290 bp, Artificial Sequence
  Synthetic DNA TRBV23-1*01
catgccaaagtcacacagactccaggacatttggtcaaaggaaaaggacagaaaacaaagatggattgtacccccgaaaaaggacatacttttgtttattggtatcaacagaatcagaataaagagtttatgcttttgatttcctttcagaatgaacaagttcttcaagaaacggagatgcacaagaagcgattctcatctcaatgccccaagaacgcaccctgcagcctggcaatcctgtcctcagaaccgggagacacggcactgtatctctgcgccagcagtcaatc

SEQ ID NO: 237 - DNA, 288 bp, Artificial Sequence
  Synthetic DNA TRBV24-1*01
gatgctgatgttacccagaccccaaggaataggatcacaaagacaggaaagaggattatgctggaatgttctcagactaagggtcatgatagaatgtactggtatcgacaagacccaggactgggcctacggttgatctattactcctttgatgtcaaagatataaacaaaggagagatctctgatggatacagtgtctctcgacaggcacaggctaaattctccctgtccctagagtctgccatccccaaccagacagctctttacttctgtgccaccagtgatttg

SEQ ID NO: 238 - DNA, 287 bp, Artificial Sequence
  Synthetic DNA TRBV25-1*01
gaagctgacatctaccagaccccaagataccttgttatagggacaggaaagaagatcactctggaatgttctcaaaccatgggccatgacaaaatgtactggtatcaacaagatccaggaatggaactacacctcatccactattcctatggagttaattccacagagaagggagatctttcctctgagtcaacagtctccagaataaggacggagcattttcccctgaccctggagtctgccaggccctcacatacctctcagtacctctgtgccagcagtgaata

SEQ ID NO: 239 - DNA, 287 bp, Artificial Sequence
  Synthetic DNA TRBV26*01
gatgctgtagttacacaattcccaagacacagaatcattgggacaggaaaggaattcattctacagtgttcccagaatatgaatcatgttacaatgtactggtatcgacaggacccaggacttggactgaagctggtctattattcacctggcactgggagcactgaaaaaggagatatctctgaggggtatcatgtttcttgaaatactatagcatcttttcccctgaccctgaagtctgccagcaccaaccagacatctgtgtatctctatgccagcagttcatc

SEQ ID NO: 240 - DNA, 287 bp, Artificial Sequence
  Synthetic DNA TRBV27*01
gaagcccaagtgacccagaacccaagatacctcatcacagtgactggaaagaagttaacagtgacttgttctcagaatatgaaccatgagtatatgtcctggtatcgacaagacccagggctgggcttaaggcagatctactattcaatgaatgttgaggtgactgataagggagatgttcctgaagggtacaaagtctctcgaaaagagaagaggaatttccccctgatcctggagtcgcccagccccaaccagacctctctgtacttctgtgccagcagtttatc

SEQ ID NO: 241 - DNA, 287 bp, Artificial Sequence
  Synthetic DNA TRBV28*01
gatgtgaaagtaacccagagctcgagatatctagtcaaaaggacgggagagaaagtttttctggaatgtgtccaggatatggaccatgaaaatatgttctggtatcgacaagacccaggtctggggctacggctgatctatttctcatatgatgttaaaatgaaagaaaaaggagatattcctgaggggtacagtgtctctagagagaagaaggagcgcttctccctgattctggagtccgccagcaccaaccagacatctatgtacctctgtgccagcagtttatg

SEQ ID NO: 242 - DNA, 290 bp, Artificial Sequence
  Synthetic DNA TRBV29-1*01
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgttgaaga

SEQ ID NO: 243 - DNA, 288 bp, Artificial Sequence
  Synthetic DNA TRBV29-1*02
agtgctgtcatctctcaaaagccaagcagggatatctgtcaacgtggaacctccctgacgatccagtgtcaagtcgatagccaagtcaccatgatgttctggtaccgtcagcaacctggacagagcctgacactgatcgcaactgcaaatcagggctctgaggccacatatgagagtggatttgtcattgacaagtttcccatcagccgcccaaacctaacattctcaagtctgactgtgagcaacatgagccctgaagacagcagcatatatctctgcagcgttgaa

SEQ ID NO: 244 - DNA, 231 bp, Artificial Sequence
  Synthetic DNA TRBV29-1*03
acgatccagtgtcaagtcgatagccaagtcaccatgatattctggtaccgtcagcaacctggacagagcctgacactgatcgcaactgcaaatcagggctctgaggccacatatgagagtggatttgtcattgacaagtttcccatcagccgcccaaacctaacattctcaactctgactgtgagcaacatgagccctgaagacagcagcatatatctctgcagcgcgggc

SEQ ID NO: 245 - DNA, 284 bp, Artificial Sequence
  Synthetic DNA TRBV30*02
tctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctcctcctcagtgactctggcttctatctctgtgcctggagtgt

SEQ ID NO: 246 - DNA, 282 bp, Artificial Sequence
  Synthetic DNA TRBV30*05
tctcagactattcatcaatggccagcgaccctggtgcagcctgtgggcagcccgctctccctggagtgcactgtggagggaacatcaaaccccaacctatactggtaccgacaggctgcaggacggggcctccagctgctcttctactccgttggtattggccagatcagctctgaggtgccccagaatctctcagcctccagaccccaggaccggcagttcatcctgagttctaagaagctccttctcagtgactctggcttctatctctgtgcctgggga

SEQ ID NO: 247 - DNA, 284 bp, Artificial Sequence
  Synthetic DNA TRBV30*01
tctcagactattcatcaatggccagcgaccctggtgcagcctgtgggcagcccgctctctctggagtgcactgtggagggaacatcaaaccccaacctatactggtaccgacaggctgcaggcaggggcctccagctgctcttctactccgttggtattggccagatcagctctgaggtgccccagaatctctcagcctccagaccccaggaccggcagttcatcctgagttctaagaagctccttctcagtgactctggcttctatctctgtgcctggagtgt

SEQ ID NO: 248 - DNA, 276 bp, Artificial Sequence
  Synthetic DNA TRBV30*04
actattcatcaatggccagcgaccctggtgcagcctgtgggcagcccgctctctctggagtgcactgtggagggaacatcaaaccccaacctatactggtaccgacaggctgcaggcaggggcctccagctgctcttctactccattggtattgaccagatcagctctgaggtgccccagaatctctcagcctccagaccccaggaccggcagttcattctgagttctaagaagctcctcctcagtgactctggcttctatctctgtgcctggagt

SEQ ID NO: 249 - DNA, 448 bp, Artificial Sequence
  Synthetic DNA TCRBJ1S1
ttgaaaaaggaacctaggaccctgtggatggactctgtcattctccatggtcctaaaaagcaaaagtcaaagtgttcttctgtgtaatacccataaagcacaggaggagatttcttagctcactgtcctccatcctagccagggccctctcccctctctatgccttcaatgtgattttcaccttgacccctgtcactgtgtgaacactgaagctttctttggacaaggcaccagactcacagttgtaggtaagacatttttcaggttcttttgcagatccgtcacagggaaaagtgggtccacagtgtcccttttagagtggctatattcttatgtgctaactatggctacaccttcggttcggggaccaggttaaccgttgtaggtaaggctgggggtctctaggaggggtgcgatgagggaggactctgtcctgggaaatgtcaaa

SEQ ID NO: 250 - DNA, 448 bp, Artificial Sequence
  Synthetic DNA TCRBJ1S2
gccagggccctctcccctctctatgccttcaatgtgattttcaccttgacccctgtcactgtgtgaacactgaagctttctttggacaaggcaccagactcacagttgtaggtaagacatttttcaggttcttttgcagatccgtcacagggaaaagtgggtccacagtgtcccttttagagtggctatattcttatgtgctaactatggctacaccttcggttcggggaccaggttaaccgttgtaggtaaggctgggggtctctaggaggggtgcgatgagggaggactctgtcctgggaaatgtcaaagagaacagagatcccagctcccggagccagactgagggagacgtcatgtcatgtcccgggattgagttcaggggaggctccctgtgagggcgaatccacccaggcttcccagaggctctgagcagtcacagctgagc

SEQ ID NO: 251 - DNA, 450 bp, Artificial Sequence
  Synthetic DNA TCRBJ1S3
gattttataggaggccactctgtgtctctttttgtcacctgcctgagtcttgggcaagctctggaagggaacacagagtactggaagcagagctgctgtccctgtgagggaagagttcccatgaactcccaacctctgcctgaatcccagctgtgctcagcagagactggggggttttgaagtggccctgggaggctgtgctctggaaacaccatatattttggagagggaagttggctcactgttgtaggtgagtaagtcaaggctggacagctgggaacttgcaaaaaggggctggaatccagacggagcctttgtctctagtgcttaggtgaaagtgtatttttgtcaggaaggcctatgaggcagatgaggaggggatagcctccctctcctctcgactattttgtagactgcctgtgccaagttaggttcccctactgagagatg

SEQ ID NO: 252 - DNA, 451 bp, Artificial Sequence
  Synthetic DNA TCRBJ1S4
cagaagagggaacttgggggatcacacggggcctaattggtctgctgaccaccgcattttgggttgtaccattgtctacccctctacccaccagggttaaaattctactaaggaacaggagaggacctggcaggtggacttggggaggcaggagtggaaggcagcaggtcgcggttttccttccagtctttaatgttgtgcaactaatgaaaaactgttttttggcagtggaacccagctctctgtcttgggtatgtaaaagacttctttcgggatagtgtatcataaggtcggagttccaggaggaccccttgcgggagggcagaaactgagaacacagccaagaaaagctcataaaatgtgggtcagtggagtgtgtggtggggccccaagagttctgtgtgtaagcagcttctggaaggaagggcccacaccagctcctctggggttt

SEQ ID NO: 253 - DNA, 450 bp, Artificial Sequence
  Synthetic DNA TCRBJ1S5
gatagtgtatcataaggtcggagttccaggaggaccccttgcgggagggcagaaactgagaacacagccaagaaaagctcataaaatgtgggtcagtggagtgtgtggtggggccccaagagttctgtgtgtaagcagcttctggaaggaagggcccacaccagctcctctggggtttgccacactcatgatgcactgtgtagcaatcagccccagcattttggtgatgggactcgactctccatcctaggtaagttgcagaatcagggtggtatggccattgtcccttgaaggcagagttctctgcttctcctcccggtgctggtgaggcagattgagtaaaatctcttaccccatggggtaagagctgtgcctgtgcctgcgttccctttggtgtgtcttggttgactcctctatttctcttctctaagtcttcagtccataatctgc

SEQ ID NO: 254 - DNA, 453 bp, Artificial Sequence
  Synthetic DNA TCRBJ1S6
atggctctgcctctcctaagcctcttcctcttgcgccttatgctgcacagtatgcttaggcctttttcctaacagaatccctttggtccagagccatgaatccaggcagagaaaggcagccatcctgctgtcagggagctaagacttgccctctgactggagatcgccgggtgggttttatctaagcctctgcagctgtgctcctataattcacccctccactttgggaacgggaccaggctcactgtgacaggtatgggggctccactcttgactcgggggtgcctgggtttgactgcaatgatcagttgctgggaagggaattgagtgtaagaacggaggtcagggtcaccccttcttacctggagcactgtgccctctcctcccctccctggagctcttccagcttgttgctctgctgtgttgcctgcagttcctcagctgtagagctcc

SEQ ID NO: 255 - DNA, 449 bp, Artificial Sequence
  Synthetic DNA TCRBJ2S1
aatccactgtgttgtcccccagccaagtggattctcctctgcaaattggtggtggcctcatgcaagatccagttaccgtgtccagctaactcgagacaggaaaagataggctcaggaaagagaggaagggtgtgccctctgtctgtgctaagggaggtggggaaggagaaggaattctgggcagccccttcccactgtgctcctacaatgagcagttcttcgggccagggacacggctcaccgtgctaggtaagaagggggctccaggtgggagagagggtgagcagcccagcctgcacgaccccagaaccctgttcttaggggagtggacactgggcaatccagggccctcctcgagggaagcggggtttgcgccagggtccccagggctgtgcgaacaccggggagctgttttttggagaaggctctaggctgaccgtactgggtaa

SEQ ID NO: 256 - DNA, 451 bp, Artificial Sequence
  Synthetic DNA TCRBJ2S2
ctgtgctcctacaatgagcagttcttcgggccagggacacggctcaccgtgctaggtaagaagggggctccaggtgggagagagggtgagcagcccagcctgcacgaccccagaaccctgttcttaggggagtggacactgggcaatccagggccctcctcgagggaagcggggtttgcgccagggtccccagggctgtgcgaacaccggggagctgttttttggagaaggctctaggctgaccgtactgggtaaggaggcggttggggctccggagagctccgagagggcgggatgggcagaggtaagcagctgccccactctgagaggggctgtgctgagaggcgctgctgggcgtctgggcggaggactcctggttctgggtgctgggagagcgatggggctctcagcggtgggaaggacccgagctgagtctgggacagcagagcgg

SEQ ID NO: 257 - DNA, 449 bp, Artificial Sequence
  Synthetic DNA TCRBJ2S3
gggcgggatgggcagaggtaagcagctgccccactctgagaggggctgtgctgagaggcgctgctgggcgtctgggcggaggactcctggttctgggtgctgggagagcgatggggctctcagcggtgggaaggacccgagctgagtctgggacagcagagcgggcagcaccggtttttgtcctgggcctccaggctgtgagcacagatacgcagtattttggcccaggcacccggctgacagtgctcggtaagcgggggctcccgctgaagccccggaactggggagggggcgccccgggacgccgggggcgtcgcagggccagtttctgtgccgcgtctcggggctgtgagccaaaaacattcagtacttcggcgccgggacccggctctcagtgctgggtaagctggggccgccgggggaccggggacgagactgcgctcgggttt

SEQ ID NO: 258 - DNA, 450 bp, Artificial Sequence
  Synthetic DNA TCRBJ2S4
gacagcagagcgggcagcaccggtttttgtcctgggcctccaggctgtgagcacagatacgcagtattttggcccaggcacccggctgacagtgctcggtaagcgggggctcccgctgaagccccggaactggggagggggcgccccgggacgccgggggcgtcgcagggccagtttctgtgccgcgtctcggggctgtgagccaaaaacattcagtacttcggcgccgggacccggctctcagtgctgggtaagctggggccgccgggggaccggggacgagactgcgctcgggtttttgtgcggggctcgggggccgtgaccaagagacccagtacttcgggccaggcacgcggctcctggtgctcggtgagcgcgggctgctggggcgcgggcgcgggcggcttgggtctggtttttgcggggagtccccgggctgtgctctggggc

SEQ ID NO: 259 - DNA, 448 bp, Artificial Sequence
  Synthetic DNA TCRBJ2S5
ccccggaactggggagggggcgccccgggacgccgggggcgtcgcagggccagtttctgtgccgcgtctcggggctgtgagccaaaaacattcagtacttcggcgccgggacccggctctcagtgctgggtaagctggggccgccgggggaccggggacgagactgcgctcgggtttttgtgcggggctcgggggccgtgaccaagagacccagtacttcgggccaggcacgcggctcctggtgctcggtgagcgcgggctgctggggcgcgggcgcgggcggcttgggtctggtttttgcggggagtccccgggctgtgctctggggccaacgtcctgactttcggggccggcagcaggctgaccgtgctgggtgagttttcgcgggaccacccgggcggcgggattcaggtggaaggcggcggctgcttcgcggcacccggtccgg

SEQ ID NO: 260 - DNA, 453 bp, Artificial Sequence
  Synthetic DNA TCRBJ2S6
cagtgctgggtaagctggggccgccgggggaccggggacgagactgcgctcgggtttttgtgcggggctcgggggccgtgaccaagagacccagtacttcgggccaggcacgcggctcctggtgctcggtgagcgcgggctgctggggcgcgggcgcgggcggcttgggtctggtttttgcggggagtccccgggctgtgctctggggccaacgtcctgactttcggggccggcagcaggctgaccgtgctgggtgagttttcgcgggaccacccgggcggcgggattcaggtggaaggcggcggctgcttcgcggcacccggtccggccctgtgctgggagacctgggctgggtccccagggtgggcaggagctcggggagccttagaggtttgcatgcgggggtgcacctccgtgctcctacgagcagtacttcgggccgggcaccaggct

SEQ ID NO: 261 - DNA, 447 bp, Artificial Sequence
  Synthetic DNA TCRBJ2S7
tgactttcggggccggcagcaggctgaccgtgctgggtgagttttcgcgggaccacccgggcggcgggattcaggtggaaggcggcggctgcttcgcggcacccggtccggccctgtgctgggagacctgggctgggtccccagggtgggcaggagctcggggagccttagaggtttgcatgcgggggtgcacctccgtgctcctacgagcagtacttcgggccgggcaccaggctcacggtcacaggtgagattcgggcgtctccccaccttccagcccctcggtccccggagtcggagggtggaccggagctggaggagctgggtgtccggggtcagctctgcaaggtcacctccccgctcctggggaaagactggggaagagggagggggtggggaggtgctcagagtccggaaagctgagcagagggcgaggccacttttaat

SEQ ID NO: 262 - DNA, 296 bp, Artificial Sequence
  Synthetic DNA IGHV1-46*02
caggtgcagctggtgcagtctggggctgaggtgaagaagcctggggcctcagtgaaggtttcctgcaaggcatctggatacaccttcaacagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaga

SEQ ID NO: 263 - DNA, 260 bp, Artificial Sequence
  Synthetic DNA IGHV1/OR15-5*01
agaagcctggggcctcagtgaaggtctcctgcaaggcttctggatacaccttcaccagctactgtatgcactgggtgcaccaggtccatgcacaagggcttgagtggatgggattggtgtgccctagtgatggcagcacaagctatgcacagaagttccaggccagagtcaccataaccagggacacatccatgagcacagcctacatggagctaagcagtctgagatctgaggacacggccatgtattactgtgtgaga

SEQ ID NO: 264 - DNA, 294 bp, Artificial Sequence
  Synthetic DNA IGHV1/OR15-5*03
caggtacagctggtgcagtctggggctgaggtgaagaagcctggggcctcagtgaaggtctcctgcaaggcttctggatacaccttcaccaactactgtatgcactgggtgcgccaggtccatgcacaagggcttgagtggatgggattggtgtgccctagtgatggcagcacaagctatgcacaaaagttccaggccagagtcaccataaccagggacacatccatgagcacagcctacatggagctaagcagtctgagatctgaggacacggccatgtattactgtgtgaga

SEQ ID NO: 265 - DNA, 296 bp, Artificial Sequence
  Synthetic DNA IGHV1/OR15-9*01
caggtacagctgatgcagtctggggctgaggtgaagaagcctggggcctcagtgaggatctcctgcaaggcttctggatacaccttcaccagctactgtatgcactgggtgtgccaggcccatgcacaagggcttgagtggatgggattggtgtgccctagtgatggcagcacaagctatgcacagaagttccagggcagagtcaccataaccagggacacatccatgggcacagcctacatggagctaagcagcctgagatctgaggacacggccatgtattactgtgtgagaga

SEQ ID NO: 266 - DNA, 260 bp, Artificial Sequence
  Synthetic DNA IGHV1-c*01
ggaagtctggggcctcagtgaaagtctcctgtagtttttctgggtttaccatcaccagctacggtatacattgggtgcaacagtcccctggacaagggcttgagtggatgggatggatcaaccctggcaatggtagcccaagctatgccaagaagtttcagggcagattcaccatgaccagggacatgtccacaaccacagcctacacagacctgagcagcctgacatctgaggacatggctgtgtattactatgcaaga

SEQ ID NO: 267 - DNA, 296 bp, Artificial Sequence
  Synthetic DNA IGHV1-NL1*01
caggttcagctgttgcagcctggggtccaggtgaagaagcctgggtcctcagtgaaggtctcctgctaggcttccagatacaccttcaccaaatactttacacggtgggtgtgacaaagccctggacaagggcatnagtggatgggatgaatcaacccttacaacgataacacacactacgcacagacgttctggggcagagtcaccattaccagtgacaggtccatgagcacagcctacatggagctgagcngcctgagatccgaagacatggtcgtgtattactgtgtgagaga

SEQ ID NO: 268 - DNA, 296 bp, Artificial Sequence
  Synthetic DNA IGHV1-58*01
caaatgcagctggtgcagtctgggcctgaggtgaagaagcctgggacctcagtgaaggtctcctgcaaggcttctggattcacctttactagctctgctgtgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagctgagcagcctgagatccgaggacacggccgtgtattactgtgcggcaga

SEQ ID NO: 269 - DNA, 296 bp, Artificial Sequence
  Synthetic DNA IGHV1-58*02
caaatgcagctggtgcagtctgggcctgaggtgaagaagcctgggacctcagtgaaggtctcctgcaaggcttctggattcacctttactagctctgctatgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagctgagcagcctgagatccgaggacacggccgtgtattactgtgcggcaga

SEQ ID NO: 270 - DNA, 275 bp, Artificial Sequence
  Synthetic DNA IGHV1-69*03
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgatgacacggc

SEQ ID NO: 271 - DNA, 233 bp, Artificial Sequence
  Synthetic DNA IGHV1-69*07
agaagcctgggtcctcggtgaaggtctcctgcaaggcttctggaggcaccttcagcagctatgctatcagctgggtgcgacaggcccctggacaagggcttgagtggatgggaaggatcatccctatctttggtacagcaaactacgcacagaagttccagggcagagtcacgattaccgcggacgaatccacgagcacagcctacatggagctgagcagcctgagatctgag

SEQ ID NO: 272 - DNA, 296 bp, Artificial Sequence
  Synthetic DNA IGHV1-69*12
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaga

SEQ ID NO: 273 - DNA, 294 bp, Artificial Sequence
  Synthetic DNA IGHV1-69*05
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccacggacgaatccacgagcacagcctacatggagctgagcagcctgagatctgaggacacggccgtgtattactgtgcgaga

SEQ ID NO: 274 - DNA, 296 bp, Artificial Sequence
  Synthetic DNA IGHV1-69*13
caggtccagctggtgcagtctggggctgaggtgaagaagcctgggtcctcagtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaga

SEQ ID NO: 275 - DNA, 296 bp, Artificial Sequence
  Synthetic DNA IGHV1-69*01
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgagaga

SEQ ID NO: 276 - DNA, 296 bp, Artificial Sequence
  Synthetic DNA IGHV1-69*06
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacaaatccacgagcacagcctacatggagctgagcagcctgagatctgaggacacggccgtgtattactgtgcgagaga

SEQ ID NO: 277 - DNA, 294 bp, Artificial Sequence
  Synthetic DNA IGHV1-69*02
caggtccagctggtgcaatctggggctgaggtgaagaagcctgggtcctcggtgaaggtctcctgcaaggcttctggaggcaccttcagcagctatactatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgaga

SEQ ID NO: 278 - DNA, 296 bp, Artificial Sequence
  Synthetic DNA IGHV1-69*08
caggtccagctggtgcaatctggggctgaggtgaagaagcctgggtcctcggtgaaggtctcctgcaaggcttctggaggcaccttcagcagctatactatcagctgggtgcgacaggcccctggacaagggcttgagtggatgggaaggatcatccctatccttggtacagcaaactacgcacagaagttccagggcagagtcacgattaccgcggacaaatccacgagcacagcctacatggagctgagcagcctgagatctgaggacacggccgtgtattactgtgcgagaga

SEQ ID NO: 279 - DNA, 296 bp, Artificial Sequence
  Synthetic DNA IGHV1-69*04
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagaga

SEQ ID NO: 280 - DNA, 296 bp, Artificial Sequence
  Synthetic DNA IGHV1-69*11
caggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtacagcaaactacgcacagaagttccagggcagagtcacgattaccgcggacgaatccacgagcacagcctacatggagctgagcagcctgagatctgaggacacggccgtgtattactgtgcgagaga

SEQ ID NO: 281 - DNA, 296 bp, Artificial Sequence
  Synthetic DNA IGHV1-69*09
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaaggatcatccctatccttggtatagcaaactacgcacagaagttccagggcagagtcacgattaccgcggacaaatccacgagcacagcctacatggagctgagcagcctgagatctgaggacacggccgtgtattactgtgcgagaga

SEQ ID NO: 282 - DNA, 296 bp, Artificial Sequence
  Synthetic DNA IGHV1-69*10
caggtccagctggtgcagtctggggctgaggtgaagaagcctgggtcctcagtgaaggtctcctgcaaggcttctggaggcaccttcagcagctatgctatcagctgggtgcgacaggcccctggacaagggcttgagtggatgggagggatcatccctatccttggtatagcaaactacgcacagaagttccagggcagagtcacgattaccgcggacaaatccacgagcacagcctacatggagctgagcagcctgagatctgaggacacggccgtgtattactgtgcgagaga

SEQ ID NO: 283 - DNA, 294 bp, Artificial Sequence
  Synthetic DNA IGHV1-f*01
gaggtccagctggtacagtctggggctgaggtgaagaagcctggggctacagtgaaaatctcctgcaaggtttctggatacaccttcaccgactactacatgcactgggtgcaacaggcccctggaaaagggcttgagtggatgggacttgttgatcctgaagatggtgaaacaatatacgcagagaagttccagggcagagtcaccataaccgcggacacgtctacagacacagcctacatggagctgagcagcctgagatctgaggacacggccgtgtattactgtgcaaca

SEQ ID NO: 284 - DNA, 233 bp, Artificial Sequence
  Synthetic DNA IGHV1-f*02
agaagcctggggctacagtgaaaatctcctgcaaggtttctggatacaccttcaccgactactacatgcactgggtgcaacaggcccctggaaaagggcttgagtggatgggacttgttgatcctgaagatggtgaaacaatatatgcagagaagttccagggcagagtcaccataaccgcggacacgtctacagacacagcctacatggagctgagcagcctgagatctgag

SEQ ID NO: 285 - DNA, 296 bp, Artificial Sequence
  Synthetic DNA IGHV1-24*01
caggtccagctggtacagtctggggctgaggtgaagaagcctggggcctcagtgaaggtctcctgcaaggtttccggatacaccctcactgaattatccatgcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgagcagcctgagatctgaggacacggccgtgtattactgtgcaacaga

SEQ ID NO: 286 - DNA, 294 bp, Artificial Sequence
  Synthetic DNA IGHV7-4-1*01
caggtgcagctggtgcaatctgggtctgagttgaagaagcctggggcctcagtgaaggtttcctgcaaggcttctggatacaccttcactagctatgctatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatctgcagcctaaaggctgaggacactgccgtgtattactgtgcgaga

SEQ ID NO: 287 - DNA, 274 bp, Artificial Sequence
  Synthetic DNA IGHV7-4-1*03
caggtgcagctggtgcaatctgggtctgagttgaagaagcctggggcctcagtgaaggtttcctgcaaggcttctggatacaccttcactagctatgctatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcacgctaaaggctgaggacactg

SEQ ID NO: 288 - DNA, 296 bp, Artificial Sequence
  Synthetic DNA IGHV7-4-1*02
caggtgcagctggtgcaatctgggtctgagttgaagaagcctggggcctcagtgaaggtttcctgcaaggcttctggatacaccttcactagctatgctatgaattgggtgcgacaggcccctggacaagggcttgagtggatgggatggatcaacaccaacactgggaacccaacgtatgcccagggcttcacaggacggtttgtcttctccttggacacctctgtcagcacggcatatctgcagatcagcagcctaaaggctgaggacactgccgtgtattactgtgcgagaga

SEQ ID NO: 289 - DNA, 296 bp, Artificial Sequence
  Synthetic DNA IGHV7-81*01
caggtgcagctggtgcagtctggccatgaggtgaagcagcctggggcctcagtgaaggtctcctgcaaggcttctggttacagtttcaccacctatggtatgaattgggtgccacaggcccctggacaagggcttgagtggatgggatggttcaacacctacactgggaacccaacatatgcccagggcttcacaggacggtttgtcttctccatggacacctctgccagcacagcatacctgcagatcagcagcctaaaggctgaggacatggccatgtattactgtgcgagata

SEQ ID NO: 290 - DNA, 289 bp, Artificial Sequence
  Synthetic DNA IGHV7-40*03
ctgcagctggtgcagtctgggcctgaggtgaagaagcctggggcctcagtgaaggtctcctataagtcttctggttacaccttcaccatctatggtatgaattgggtatgatagacccctggacagggctttgagtggatgtgatggatcatcacctacactgggaacccaacgtatacccacggcttcacaggatggtttgtcttctccatggacacgtctgtcagcacggcgtgtcttcagatcagcagcctaaaggctgaggacacggccgagtattactgtgcga

SEQ ID NO: 291 - DNA, 296 bp, Artificial Sequence
  Synthetic DNA IGHV5-51*01
gaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaca

SEQ ID NO: 292 - DNA, 245 bp, Artificial Sequence
  Synthetic DNA IGHV5-51*05
aaaagcccggggagtctctgaagatctcctgtaagggttctggatacagctttaccagctactggatcggctgggtgcgccagatgcccaggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatg

SEQ ID NO: 293 - DNA, 296 bp, Artificial Sequence
  Synthetic DNA IGHV5-51*02
gaggtgcagctggtgcagtctggagcagaggtgaaaaagcccggggagtctctgaagatctcctgtaagggttctggatacagctttaccagctactggaccggctgggtgcgccagatgcccgggaaaggcttggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagaca

SEQ ID NO: 294 - DNA, 294 bp, Artificial Sequence
  Synthetic DNA IGHV5-51*03
gaggtgcagctggtgcagtctggagcagaggtgaaaaagccgggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgaga

SEQ ID NO: 295 - DNA, 294 bp, Artificial Sequence
  Synthetic DNA IGHV5-51*04
gaggtgcagctggtgcagtctggagcagaggtgaaaaagccgggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagcccatcagcaccgcctacctgcagtggagcagcctgaaggcctcggacaccgccatgtattactgtgcgaga

SEQ ID NO: 296 - DNA, 294 bp, Artificial Sequence
  Synthetic DNA IGHV5-a*01
gaagtgcagctggtgcagtctggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgaga

SEQ ID NO: 297 - DNA, 294 bp, Artificial Sequence
  Synthetic DNA IGHV5-a*03
gaagtgcagctggtgcagtccggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccacgtcaccatctcagctgacaagtccatcagcactgcctacctgcagtggagcagcctgaaggcctcggacaccgccatgtattactgtgcgaga

SEQ ID NO: 298 - DNA, 294 bp, Artificial Sequence
  Synthetic DNA IGHV5-a*04
gaagtgcagctggtgcagtctggagcagaggtgaaaaagcccggggagtctctgaggatctcctgtaagggttctggatacagctttaccagctactggatcagctgggtgcgccagatgcccgggaaaggcctggagtggatggggaggattgatcctagtgactcttataccaactacagcccgtccttccaaggccaggtcaccatctcagctgacaagtccatcagcactgcctacctgcagtggagcagcctgaaggcctcggacaccgccatgtattactgtgcgaga

SEQ ID NO: 299 - DNA, 295 bp, Artificial Sequence
  Synthetic DNA IGHV5-a*02
gaagtgcagctggtgcagtctggagcagaggtgaaaaagcccggggagtctctgaggatctcctgtaagggttctggatacagctttaccagctactggatcagctgggtgcgccagatgcccgggaaaggcttggagtggatggggaggattgatcctagtgactcttataccaactacagcccgtccttccaaggccacgtcaccatctcagctgacaagtccatcagcactgcctacctgcagtggagcagcctgaaggctcggacaccgccatgtattactgtgcgagaca

SEQ ID NO: 300 - DNA, 294 bp, Artificial Sequence
  Synthetic DNA IGHV5-78*01
gaggtgcagctgttgcagtctgcagcagaggtgaaaagacccggggagtctctgaggatctcctgtaagacttctggatacagctttaccagctactggatccactgggtgcgccagatgcccgggaaagaactggagtggatggggagcatctatcctgggaactctgataccagatacagcccatccttccaaggccacgtcaccatctcagccgacagctccagcagcaccgcctacctgcagtggagcagcctgaaggcctcggacgccgccatgtattattgtgtgaga

SEQ ID NO: 301 - DNA, 296 bp, Artificial Sequence
  Synthetic DNA IGHV3-11*01
caggtgcagctggtggagtctgggggaggcttggtcaagcctggagggtccctgagactctcctgtgcagcctctggattcaccttcagtgactactacatgagctggatccgccaggctccagggaaggggctggagtgggtttcatacattagtagtagtggtagtaccatatactacgcagactctgtgaagggccgattcaccatctccagggacaacgccaagaactcactgtatctgcaaatgaacagcctgagagccgaggacacggccgtgtattactgtgcgagaga

SEQ ID NO: 302 - DNA, 294 bp, Artificial Sequence
  Synthetic DNA IGHV3-11*03
caggtgcagctgttggagtctgggggaggcttggtcaagcctggagggtccctgagactctcctgtgcagcctctggattcaccttcagtgactactacatgagctggatccgccaggctccagggaaggggctggagtgggtttcatacattagtagtagtagtagttacacaaactacgcagactctgtgaagggccgattcaccatctccagagacaacgccaagaactcactgtatctgcaaatgaacagcctgagagccgaggacacggccgtgtattactgtgcgaga

SEQ ID NO: 303 - DNA, 296 bp, Artificial Sequence
  Synthetic DNA IGHV3-21*01
gaggtgcagctggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaga

SEQ ID NO: 304 - DNA, 296 bp, Artificial Sequence
  Synthetic DNA IGHV3-21*02
gaggtgcaactggtggagtctgggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagaga

SEQ ID NO: 305 - DNA, 296 bp, Artificial Sequence
  Synthetic DNA IGHV3-48*01
gaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagccgaggacacggctgtgtattactgtgcgagaga

SEQ ID NO: 306 - DNA, 296 bp, Artificial Sequence
  Synthetic DNA IGHV3-48*02
gaggtgcagctggtggagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgcagactctgtgaagggccgattcaccatctccagagacaatgccaagaactcactgtatctgcaaatgaacagcctgagagacgaggacacggctgtgtattactgtgcgagaga

SEQ ID NO: 307 - DNA, 293 bp, Artificial Sequence
  Synthetic DNA IGHV3-h*01
gaggtgcagctggtggagtctgggggaggcttggtaaagcctggggggtccctgagactctcctgtgcagcctctggattcaccttcagtgactactacatgaactgggtccgccaggctccagggaaggggctggagtgggtctcatccattagtagtagtagtaccatatactacgcagactctgtgaagggccgattcaccatctccagagacaacgccaagaactcactgtatctgcaaatgaacagcctgagagccgaggacacggctgtgtattactgtgcgagaga

SEQ ID NO: 308 - DNA, 293 bp, Artificial Sequence
  Synthetic DNA IGHV3-h*02
gaggtgcagctggtggagtctgggggaggcttggtaaagcctggggggtccctgagactctcctgtgcagcctctggattcaccttcagtgactactacatgaactgggtccgccaggctccagggaaggggctggagtgggtctcatccattagtagtagtagtaccatatactacgcagactctgtgaagggccgattcaccatctccagagacaacgccaagaactcactgtatctgcaaatgaacagcctgagagccgaggacacggctgtttattactgtgcgagaga

SEQ ID NO: 309 - DNA, 296 bp, Artificial Sequence
  Synthetic DNA IGHV3-48*03
gaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtttattactgtgcgagaga

SEQ ID NO: 310 - DNA, 292 bp, Artificial Sequence
  Synthetic DNA IGHV3/OR16-8*01
gaggtgcagctggtggagtctgggggaggcttggtacagcctggggggtccctgagactgtcctgtccagcctctggattcaccttcagtaaccactacatgagctgggtccgccaggctccagggaagggactggagtgggtttcatacattagtggtgatagtggttacacaaactacgcagactctgtgaagggccgattcaccatctccagggacaacgccaataactcaccgtatctgcaaatgaacagcctgagagctgaggacacggctgtgtattactgtgtga

SEQ ID NO: 311 - DNA, 292 bp, Artificial Sequence
  Synthetic DNA IGHV3/OR16-9*01
gaggtgcagctggtggagtctggaggaggcttggtacagcctggggggtccctgagactctcctgtgcagcctctggattcaccttcagtaaccactacacgagctgggtccgccaggctccagggaagggactggagtgggtttcatacagtagtggtaatagtggttacacaaactacgcagactctgtgaaaggccgattcaccatctccagggacaacgccaagaactcactgtatctgcaaatgaacagcctgagagccgaggacacggctgtgtattactgtgtga

SEQ ID NO: 312 - DNA, 293 bp, Artificial Sequence
  Synthetic DNA IGHV3-13*01
gaggtgcagctggtggagtctgggggaggcttggtacagcctggggggtccctgagactctcctgtgcagcctctggattcaccttcagtagctacgacatgcactgggtccgccaagctacaggaaaaggtctggagtgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagaga

SEQ ID NO: 313 - DNA, 291 bp, Artificial Sequence
  Synthetic DNA IGHV3-13*03
gaggtgcagctggtggagtctgggggaggcttggtacagcctggggggtccctgagactctcctgtgcagcctgtggattcaccttcagtagctacgacatgcactgggtccgccaagctacaggaaaaggtctggagtgggtctcagctattggtactgctggtgacacatactatccaggctccgtgaagggccaattcaccatctccagagaaaatgccaagaactccttgtatcttcaaatgaacagcctgagagccggggacacggctgtgtattactgtgcaaga

SEQ ID NO: 314 - DNA, 293 bp, Artificial Sequence
  Synthetic DNA IGHV3-13*02
gaggtgcatctggtggagtctgggggaggcttggtacagcctgggggggccctgagactctcctgtgcagcctctggattcaccttcagtaactacgacatgcactgggtccgccaagctacaggaaaaggtctggagtgggtctcagccaatggtactgctggtgacacatactatccaggctccgtgaaggggcgattcaccatctccagagaaaatgccaagaactccttgtatcttcaaatgaacagcctgagagccggggacacggctgtgtattactgtgcaagaga

SEQ ID NO: 315 - DNA, 123 bp, Artificial Sequence
  Synthetic DNA IGHV3-47*02
atactatgcagactccgtgatgggccgattcaccatctccagagacaacgccaagaagtccttgtatcttcaaatgaacagcctgatagctgaggacatggctgtgtattattgtgcaagaga

SEQ ID NO: 316 - DNA, 282 bp, Artificial Sequence
  Synthetic DNA IGHV3-47*03
gaggatcagctggtggagtctgggggaggcttggtacagcctggggggtccctgagaccctcctgtgcagcctctggattcgccttcagtagctatgttctgcactgggttcgccgggctccagggaagggtccggagtgggtatcagctattggtactggtggtgatacatactatgcagactccgtgatgggccgattcaccatctccagagacaacgccaagaagtccttgtatctcaaatgaacagcctgatagctgaggacatggctgtgtattatg

SEQ ID NO: 317 - DNA, 291 bp, Artificial Sequence
  Synthetic DNA IGHV3-47*01
gaggatcagctggtggagtctgggggaggcttggtacagcctggggggtccctgcgaccctcctgtgcagcctctggattcgccttcagtagctatgctctgcactgggttcgccgggctccagggaagggtctggagtgggtatcagctattggtactggtggtgatacatactatgcagactccgtgatgggccgattcaccatctccagagacaacgccaagaagtccttgtatcttcatatgaacagcctgatagctgaggacatggctgtgtattattgtgcaaga

SEQ ID NO: 318 - DNA, 291 bp, Artificial Sequence
  Synthetic DNA IGHV3/OR16-10*01
gaggttcagctggtgcagtctgggggaggcttggtacatcctggggggtccctgagactctcctgtgcaggctctggattcaccttcagtagctatgctatgcactgggttcgccaggctccaggaaaaggtctggagtgggtatcagctattggtactggtggtggcacatactatgcagactccgtgaagggccgattcaccatctccagagacaatgccaagaactccttgtatcttcaaatgaacagcctgagagccgaggacatggctgtgtattactgtgcaaga

SEQ ID NO: 319 - DNA, 291 bp, Artificial Sequence
  Synthetic DNA IGHV3/OR16-10*02
gaggttcagctggtgcagtctgggggaggcttggtacagcctggggggtccctgagactctcctgtgcaggctctggattcaccttcagtagctatgctatgcactgggttcgccaggctccaggaaaaggtctggagtgggtatcagctattggtactggtggtggcacatactatgcagactccgtgaagggccgattcaccatctccagagacaatgccaagaactccttgtatcttcaaatgaacagcctgagagccgaggacatggctgtgtattactgtgcaaga

SEQ ID NO: 320 - DNA, 296 bp, Artificial Sequence
  Synthetic DNA IGHV3-62*01
gaggtgcagctggtggagtctggggaaggcttggtccagcctggggggtccctgagactctcctgtgcagcctctggattcaccttcagtagctctgctatgcactgggtccgccaggctccaagaaagggtttgtagtgggtctcagttattagtacaagtggtgataccgtactctacacagactctgtgaagggccgattcaccatctccagagacaatgcccagaattcactgtctctgcaaatgaacagcctgagagccgagggcacagttgtgtactactgtgtgaaaga

SEQ ID NO: 321 - DNA, 296 bp, Artificial Sequence
  Synthetic DNA IGHV3-64*01
gaggtgcagctggtggagtctgggggaggcttggtccagcctggggggtccctgagactctcctgtgcagcctctggattcaccttcagtagctatgctatgcactgggtccgccaggctccagggaagggactggaatatgtttcagctattagtagtaatgggggtagcacatattatgcaaactctgtgaagggcagattcaccatctccagagacaattccaagaacacgctgtatcttcaaatgggcagcctgagagctgaggacatggctgtgtattactgtgcgagaga

SEQ ID NO: 322 - DNA, 296 bp, Artificial Sequence
  Synthetic DNA IGHV3-64*02
gaggtgcagctggtggagtctggggaaggcttggtccagcctggggggtccctgagactctcctgtgcagcctctggattcaccttcagtagctatgctatgcactgggtccgccaggctccagggaagggactggaatatgtttcagctattagtagtaatgggggtagcacatattatgcagactctgtgaagggcagattcaccatctccagagacaattccaagaacacgctgtatcttcaaatgggcagcctgagagctgaggacatggctgtgtattactgtgcgagaga

SEQ ID NO: 323 - DNA, 296 bp, Artificial Sequence
  Synthetic DNA IGHV3-64*03
gaggtgcagctggtggagtctgggggaggcttggtccagcctggggggtccctgagactctcctgttcagcctctggattcaccttcagtagctatgctatgcactgggtccgccaggctccagggaagggactggaatatgtttcagctattagtagtaatgggggtagcacatactacgcagactcagtgaagggcagattcaccatctccagagacaattccaagaacacgctgtatgtccaaatgagcagtctgagagctgaggacacggctgtgtattactgtgtgaaaga

SEQ ID NO: 324 - DNA, 296 bp, Artificial Sequence
  Synthetic DNA IGHV3-64*05
gaggtgcagctggtggagtctgggggaggcttggtccagcctggggggtccctgagactctcctgttcagcctctggattcaccttcagtagctatgctatgcactgggtccgccaggctccagggaagggactggaatatgtttcagctattagtagtaatgggggtagcacatactacgcagactcagtgaagggcagattcaccatctccagagacaattccaagaacacgctgtatgttcaaatgagcagtctgagagctgaggacacggctgtgtattactgtgtgaaaga

SEQ ID NO: 325 - DNA, 296 bp, Artificial Sequence
  Synthetic DNA IGHV3-64*04
caggtgcagctggtggagtctgggggaggcttggtccagcctggggggtccctgagactctcctgttcagcctctggattcaccttcagtagctatgctatgcactgggtccgccaggctccagggaagggactggaatatgtttcagctattagtagtaatgggggtagcacatactacgcagactcagtgaagggcagattcaccatctccagagacaattccaagaacacgctgtatctgcaaatgaacagcctgagagctgaggacacggctgtgtattactgtgcgagaga

SEQ ID NO: 326 - DNA, 296 bp, Artificial Sequence
  Synthetic DNA IGHV3-16*01
gaggtacaactggtggagtctgggggaggcttggtacagcctggggggtccctgagactctcctgtgcagcctctggattcaccttcagtaacagtgacatgaactgggcccgcaaggctccaggaaaggggctggagtgggtatcgggtgttagttggaatggcagtaggacgcactatgtggactccgtgaagcgccgattcatcatctccagagacaattccaggaactccctgtatctgcaaaagaacagacggagagccgaggacatggctgtgtattactgtgtgagaaa

SEQ ID NO: 327 - DNA, 296 bp, Artificial Sequence
  Synthetic DNA IGHV3-16*02
gaggtgcagctggtggagtctgggggaggcttggtacagcctggggggtccctgagactctcctgtgcagcctctggattcaccttcagtaacagtgacatgaactgggcccgcaaggctccaggaaaggggctggagtgggtatcgggtgttagttggaatggcagtaggacgcactatgtggactccgtgaagcgccgattcatcatctccagagacaattccaggaactccctgtatctgcaaaagaacagacggagagccgaggacatggctgtgtattactgtgtgagaaa

SEQ ID NO: 328 - DNA, 294 bp, Artificial Sequence
  Synthetic DNA IGHV3/OR16-15*02
gaggtgcagctggtggagtctgggggaggcttggtccagcctggggggtccctgagacactcctgtgcagcctctggattcaccttcagtaacagtgacatgaactgggtcctctaggctccaggaaaggggctggagtgggtctcgggtattagttggaatggcggtaagacgcactatgtggactccgtgaagggccaatttaccatctccagagacaattccagcaagtccctgtatctgcaaaagaacagacagagagccaaagacatggccgtgtattactgtgtgaga

SEQ ID NO: 329 - DNA, 294 bp, Artificial Sequence
  Synthetic DNA IGHV3/OR16-16*01
gaggtgcagctggtggagtctgggggaggcttggtccagcctggggggtccctgagacactcctgtgcagcctctggattcaccttcagtaacagtgacatgaactgggtcctctaggctccaggaaaggggctggagtgggtctcggatattagttggaatggcggtaagacgcactatgtggactccgtgaagggccaatttaccatctccagagacaattccagcaagtccctgtatctgcaaaagaacagacagagagccaaggacatggccgtgtattactgtgtgaga

SEQ ID NO: 330 - DNA, 296 bp, Artificial Sequence
  Synthetic DNA IGHV3/OR16-15*01
gaagtgcagctggtggagtctgggggaggcttggtccagcctggggggtccctgagactctcctgtgcagcctctgtattcaccttcagtaacagtgacataaactgggtcctctaggctccaggaaaggggctggagtgggtctcgggtattagttggaatggcggtaagacgcactatgtggactccgtgaagggccaattttccatctccagagacaattccagcaagtccctgtatctgcaaaagaacagacagagagccaaggacatggccgtgtattactgtgtgagaaa

SEQ ID NO: 331 - DNA, 296 bp, Artificial Sequence
  Synthetic DNA IGHV3-19*01
acagtgcagctggtggagtctgggggaggcttggtagagcctggggggtccctgagactctcctgtgcagcctctggattcaccttcagtaacagtgacatgaactgggtccgccaggctccaggaaaggggctggagtgggtatcgggtgttagttggaatggcagtaggacgcactatgcagactctgtgaagggccgattcatcatctccagagacaattccaggaacttcctgtatcagcaaatgaacagcctgaggcccgaggacatggctgtgtattactgtgtgagaaa

SEQ ID NO: 332 - DNA, 296 bp, Artificial Sequence
  Synthetic DNA IGHV3-35*01
gaggtgcagctggtggagtctgggggaggcttggtacagcctgggggatccctgagactctcctgtgcagcctctggattcaccttcagtaacagtgacatgaactgggtccatcaggctccaggaaaggggctggagtgggtatcgggtgttagttggaatggcagtaggacgcactatgcagactctgtgaagggccgattcatcatctccagagacaattccaggaacaccctgtatctgcaaacgaatagcctgagggccgaggacacggctgtgtattactgtgtgagaaa

SEQ ID NO: 333 - DNA, 298 bp, Artificial Sequence
  Synthetic DNA IGHV3-43*01
gaagtgcagctggtggagtctgggggagtcgtggtacagcctggggggtccctgagactctcctgtgcagcctctggattcacctttgatgattataccatgcactgggtccgtcaagctccggggaagggtctggagtgggtctctcttattagttgggatggtggtagcacatactatgcagactctgtgaagggccgattcaccatctccagagacaacagcaaaaactccctgtatctgcaaatgaacagtctgagaactgaggacaccgccttgtattactgtgcaaaagata

SEQ ID NO: 334 - DNA, 294 bp, Artificial Sequence
  Synthetic DNA IGHV3-43*02
gaagtgcagctggtggagtctgggggaggcgtggtacagcctggggggtccctgagactctcctgtgcagcctctggattcacctttgatgattatgccatgcactgggtccgtcaagctccagggaagggtctggagtgggtctctcttattagtggggatggtggtagcacatactatgcagactctgtgaagggccgattcaccatctccagagacaacagcaaaaactccctgtatctgcaaatgaacagtctgagaactgaggacaccgccttgtattactgtgcaaaa

SEQ ID NO: 335 - DNA, 298 bp, Artificial Sequence
  Synthetic DNA IGHV3-9*01
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagata

SEQ ID NO: 336 - DNA, 296 bp, Artificial Sequence
  Synthetic DNA IGHV3-20*01
gaggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtatcactgtgcgagaga

SEQ ID NO: 337 - DNA, 296 bp, Artificial Sequence
  Synthetic DNA IGHV3-74*01
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagaga

SEQ ID NO: 338 - DNA, 294 bp, Artificial Sequence
  Synthetic DNA IGHV3-74*02
gaggtgcagctggtggagtctgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaaga

SEQ ID NO: 339 - DNA, 296 bp, Artificial Sequence
  Synthetic DNA IGHV3-74*03
gaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaacgtacgcggactccgtgaagggccgattcaccatctccagagacaacgccaagaacacgctgtatctgcaaatgaacagtctgagagccgaggacacggctgtgtattactgtgcaagaga

SEQ ID NO: 340 - DNA, 294 bp, Artificial Sequence
  Synthetic DNA IGHV3/OR16-13*01
gaggtgcagctggtggagtctgggggaggcttagtacagcctggagggtccctgagactctcctgtgcagcctctggattcaccttcagtagctactggatgcactgggtccgccaagctccagggaaggggctggtgtgggtctcacgtattaatagtgatgggagtagcacaagctacgcagactccatgaagggccaattcaccatctccagagacaatgctaagaacacgctgtatctgcaaatgaacagtctgagagctgaggacatggctgtgtattactgtactaga

SEQ ID NO: 341 - DNA, 294 bp, Artificial Sequence
  Synthetic DNA IGHV3/OR16-14*01
gaggtgcagctggaggagtctgggggaggcttagtacagcctggagggtccctgagactctcctgtgcagcctctggattcaccttcagtagctactggatgcactgggtccgccaatctccagggaaggggctggtgtgagtctcacgtattaatagtgatgggagtagcacaagctacgcagactccttgaagggccaattcaccatctccagagacaatgctaagaacacgctgtatctgcaaatgaacagtctgagagctgaggacatggctgtgtattactgtactaga

SEQ ID NO: 342 - DNA, 296 bp, Artificial Sequence
  Synthetic DNA IGHV3-30*01
caggtgcagctggtggagtctgggggaggcgtggtccagcctgggaggtccctgagactctcctgtgcagcctctggattcaccttcagtagctatgctatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaga

SEQ ID NO: 343 - DNA, 294 bp, Artificial Sequence
  Synthetic DNA IGHV3-30*08
caggtgcagctggtggactctgggggaggcgtggtccagcctgggaggtccctgagactctcctgtgcagcctctgcattcaccttcagtagctatgctatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgaga

SEQ ID NO: 344 - DNA, 296 bp, Artificial Sequence
  Synthetic DNA IGHV3-30*17
caggtgcagctggtggagtctgggggaggcgtggtccagcctgggaggtccctgagactctcctgtgcagcctctggattcaccttcagtagctatgctatgcactgggtccgccaggctccgggcaaggggctagagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaga

SEQ ID NO: 345 - DNA, 296 bp, Artificial Sequence
  Synthetic DNA IGHV3-30*11
caggtgcagctggtggagtctgggggaggcgtggtccagcctgggaggtccctgagactctcctgtgcagcgtctggattcaccttcagtagctatgctatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaga

SEQ ID NO: 346 - DNA, 296 bp, Artificial Sequence
  Synthetic DNA IGHV3-30*10
caggtgcagctggtggagtctgggggaggcgtggtccagcctgggaggtccctgagactctcctgtgcagcctctggattcaccttcagtagctatgctatgcactgggtccgccaggctccaggcaaggggctagagtgggtggcagttatatcatatgatggaagtaataaatactacacagactccgtgaagggccgattcaccatctccagagacaattccaagaacacgctgtatctgcaaatgaacagcctgagagctgaggacacggctgtgtattactgtgcgagaga

SEQ ID NO: 347 - DNA, 296 bp, Artificial Sequence
  Synthetic DNA IGHV3-30*16
caggtgcagctggtggagtctgggggaggcgtggtccagcctgggaggtccctgagactctcctgtgcagcctctggattcaccttcagtagctatgctatgcactgggtccgccaggccccaggcaaggggctagagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaga

SEQ ID NO: 348 - DNA, 296 bp, Artificial Sequence
  Synthetic DNA IGHV3-30*15
caggtgcagctggtggagtctgggggaggcgtggtccagcctgggaggtccctgagactctcctgtgcagcctctggattcaccttcagtagctatgctatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgagcagcctgagagctgaggacacggctgtgtattactgtgcgagaga

SEQ ID NO: 349 - DNA, 296 bp, Artificial Sequence
  Synthetic DNA IGHV3-30*07
caggtgcagctggtggagtctgggggaggcgtggtccagcctgggaggtccctgagactctcctgtgcagcctctggattcaccttcagtagctatgctatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagccgaggacacggctgtgtattactgtgcgagaga

SEQ ID NO: 350 - DNA, 296 bp, Artificial Sequence
  Synthetic DNA IGHV3-30*04
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaga

SEQ ID NO: 351 - DNA, 296 bp, Artificial Sequence
  Synthetic DNA IGHV3-30*09
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcgccatctccagagacaattccaagaacacgctgtatctgcaaatgaacagcctgagagctgaggacacggctgtgtattactgtgcgagaga

SEQ ID NO: 352 - DNA, 296 bp, Artificial Sequence
  Synthetic DNA IGHV3-30*14
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatcttcaaatgaacagcctgagagctgaggacacggctgtgtattactgtgcgagaga

SEQ ID NO: 353 - DNA, 294 bp, Artificial Sequence
  Synthetic DNA IGHV3-30-3*01
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgaga

SEQ ID NO: 354 - DNA, 296 bp, Artificial Sequence
  Synthetic DNA IGHV3-30-3*02
caggtgcagctggtggagtctgggggaggcgtggtccagcctgggaggtccctgagactctcctgtgcagcgtctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgaaaga

SEQ ID NO: 355 - DNA, 296 bp, Artificial Sequence
  Synthetic DNA IGHV3-30*03
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagaga

SEQ ID NO: 356 - DNA, 296 bp, Artificial Sequence
  Synthetic DNA IGHV3-30*18
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaaga

SEQ ID NO: 357 - DNA, 296 bp, Artificial Sequence
  Synthetic DNA IGHV3-30*06
caggtgcagctggtggagtctgggggaggcgtggtccagcctgggaggtccctgagactctcctgtgcagcgtctggattcaccttcagtagctatggcatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaga

SEQ ID NO: 358 - DNA, 296 bp, Artificial Sequence
  Synthetic DNA IGHV3-30*12
caggtgcagctggtggagtctggggggggcgtggtccagcctgggaggtccctgagactctcctgtgcagcgtctggattcaccttcagtagctatggcatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagccgaggacacggctgtgtattactgtgcgagaga

SEQ ID NO: 359 - DNA, 296 bp, Artificial Sequence
  Synthetic DNA IGHV3-30*19
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaga

SEQ ID NO: 360 - DNA, 296 bp, Artificial Sequence
  Synthetic DNA IGHV3-33*05
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagccgaggacacggctgtgtattactgtgcgagaga

SEQ ID NO: 361 - DNA, 296 bp, Artificial Sequence
  Synthetic DNA IGHV3-30*05
caggtgcagctggtggagtctgggggaggcgtggtccagcctgggaggtccctgagactctcctgtgcagcctctggattcaccttcagtagctatggcatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagctgagggcacggctgtgtattactgtgcgagaga

SEQ ID NO: 362 - DNA, 296 bp, Artificial Sequence
  Synthetic DNA IGHV3-30*13
caggtgcagctggtggagtctgggggaggcgtggtccagcctgggaggtccctgagactctcctgtgcagcctctggattcaccttcagtagctatggcatgcactgggtccgccaggctccaggcaaggggctagagtgggtggcagttatatcatatgatggaagtaataaatactacgcagactccgtgaagggccgattcaccatctccagagacaattccaagaacaggctgtatctgcaaatgaacagcctgagagctgaggacacggctgtgtattactgtgcgagaga

SEQ ID NO: 363 - DNA, 296 bp, Artificial Sequence
  Synthetic DNA IGHV3-33*01
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaattccaagaacacgctgtatctgcaaatgaacagcctgagagccgaggacacggctgtgtattactgtgcgagaga

SEQ ID NO: 364 - DNA, 296 bp, Artificial Sequence
  Synthetic DNA IGHV3-33*04
caggtgcagctggtggagtctgggggaggcgtggtccagcctgggaggtccctgagactctcctgtgcagcgtctggattcaccttcagtagctatggcatgcactgggtccgccaggctccaggcaaggggctagagtgggtggcagttatatggtatgacggaagtaataaatactatgcagactccgtgaagggccgattcaccatctccagagacaattccaagaacacgctgtatctgcaaatgaacagcctgagagccgaggacacggctgtgtattactgtgcgagaga

SEQ ID NO: 365 - DNA, 296 bp, Artificial Sequence
  Synthetic DNA IGHV3-33*02
caggtacagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgcgaagggccgattcaccatctccagagacaattccacgaacacgctgtttctgcaaatgaacagcctgagagccgaggacacggctgtgtattactgtgcgagaga

SEQ ID NO: 366 - DNA, 296 bp, Artificial Sequence
  Synthetic DNA IGHV3-33*03
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatggtatgatggaagtaataaatactatgcagactccgtgaagggccgattcaccatctccagagacaactccaagaacacgctgtatctgcaaatgaacagcctgagagccgaggacacggctgtgtattactgtgcgaaaga

SEQ ID NO: 367 - DNA, 296 bp, Artificial Sequence
  Synthetic DNA IGHV3-30*02
caggtgcagctggtggagtctgggggaggcgtggtccagcctggggggtccctgagactctcctgtgcagcgtctggattcaccttcagtagctatggcatgcactgggtccgccaggctccaggcaaggggctggagtgggtggcatttatacggtatgatggaagtaataaatactatgcagactccgtgaagggccgattcaccatctccagagacaattccaagaacacgctgtatctgcaaatgaacagcctgagagctgaggacacggctgtgtattactgtgcgaaaga

SEQ ID NO: 368 - DNA, 296 bp, Artificial Sequence
  Synthetic DNA IGHV3-52*01
gaggtgcagctggtggagtctgggtgaggcttggtacagcctggagggtccctgagactctcctgtgcagcctctggattcaccttcagtagctcctggatgcactgggtctgccaggctccggagaaggggctggagtgggtggccgacataaagtgtgacggaagtgagaaatactatgtagactctgtgaagggccgattgaccatctccagagacaatgccaagaactccctctatctgcaagtgaacagcctgagagctgaggacatgaccgtgtattactgtgtgagagg

SEQ ID NO: 369 - DNA, 294 bp, Artificial Sequence
  Synthetic DNA IGHV3-52*02
gaggtgcagctggtggagtctgggtgaggcttggtacagcctggagggtccctgagactctcctgtgcagcctctggattcaccttcagtagctcctggatgcactgggtctgccaggctccggagaaggggcaggagtgggtggccgacataaagtgtgacggaagtgagaaatactatgtagactctgtgaagggccgattgaccatctccagagacaatgccaagaactccctctatctgcaagtgaacagcctgagagctgaggacatgaccgtgtattactgtgtgaga

SEQ ID NO: 370 - DNA, 294 bp, Artificial Sequence
  Synthetic DNA IGHV3-52*03
gaggtgcagctggtcgagtctgggtgaggcttggtacagcctggagggtccctgagactctcctgtgcagcctctggattcaccttcagtagctcctggatgcactgggtctgccaggctccggagaaggggctggagtgggtggccgacataaagtgtgacggaagtgagaaatactatgtagactctgtgaagggccgattgaccatctccagagacaatgccaagaactccctctatctgcaagtgaacagcctgagagctgaggacatgaccgtgtattactgtgtgaga

SEQ ID NO: 371 - DNA, 296 bp, Artificial Sequence
  Synthetic DNA IGHV3-7*01
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagaga

SEQ ID NO: 372 - DNA, 294 bp, Artificial Sequence
  Synthetic DNA IGHV3-7*02
gaggtgcagctggtggagtctgggggaggcttggtccagcctggggggtccctgagactctcctgtgcagcctctggattcacctttagtagctattggatgagctgggtccgccaggctccagggaaagggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgaga

SEQ ID NO: 373 - DNA, 296 bp, Artificial Sequence
  Synthetic DNA IGHV3-23*01
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaga

SEQ ID NO: 374 - DNA, 296 bp, Artificial Sequence
  Synthetic DNA IGHV3-23*04
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaaga

SEQ ID NO: 375 - DNA, 296 bp, Artificial Sequence
  Synthetic DNA IGHV3-23*02
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacggagactccgtgaagggccggttcaccatctcaagagacaattccaagaacacgctgtatctgcaaatgaacagcctgagagccgaggacacggccgtatattactgtgcgaaaga

SEQ ID NO: 376 - DNA, 294 bp, Artificial Sequence
  Synthetic DNA IGHV3-23*03
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagttatttatagcggtggtagtagcacatactatgcagactccgtgaagggccggttcaccatctccagagataattccaagaacacgctgtatctgcaaatgaacagcctgagagccgaggacacggccgtatattactgtgcgaaa

SEQ ID NO: 377 - DNA, 294 bp, Artificial Sequence
  Synthetic DNA IGHV3-23*05
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctatttatagcagtggtagtagcacatactatgcagactccgtgaagggccggttcaccatctccagagacaattccaagaacacgctgtatctgcaaatgaacagcctgagagccgaggacacggccgtatattactgtgcgaaa

SEQ ID NO: 378 - DNA, 293 bp, Artificial Sequence
  Synthetic DNA IGHV3-53*01
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgagaga

SEQ ID NO: 379 - DNA, 291 bp, Artificial Sequence
  Synthetic DNA IGHV3-53*02
gaggtgcagctggtggagactggaggaggcttgatccagcctggggggtccctgagactctcctgtgcagcctctgggttcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagccgaggacacggccgtgtattactgtgcgaga

SEQ ID NO: 380 - DNA, 293 bp, Artificial Sequence
  Synthetic DNA IGHV3-66*03
gaggtgcagctggtggagtctggaggaggcttgatccagcctggggggtccctgagactctcctgtgcagcctctgggttcaccgtcagtagcaactacatgagctgggtccgccaggctccagggaaggggctggagtgggtctcagttatttatagctgtggtagcacatactacgcagactccgtgaagggccgattcaccatctccagagacaattccaagaacacgctgtatcttcaaatgaacagcctgagagctgaggacacggctgtgtattactgtgcgagaga

SEQ ID NO: 381 - DNA, 293 bp, Artificial Sequence
  Synthetic DNA IGHV3-53*03
gaggtgcagctggtggagtctggaggaggcttgatccagcctggggggtccctgagactctcctgtgcagcctctgggttcaccgtcagtagcaactacatgagctgggtccgccagcctccagggaaggggctggagtgggtctcagttatttatagcggtggtagcacatactacgcagactctgtgaagggccgattcaccatctccagagacaattccaagaacacgctgtatcttcaaatgaacagcctgagagccgaggacacggccgtgtattactgtgctaggga

SEQ ID NO: 382 - DNA, 293 bp, Artificial Sequence
  Synthetic DNA IGHV3-66*01
gaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagaga

SEQ ID NO: 383 - DNA, 293 bp, Artificial Sequence
  Synthetic DNA IGHV3-66*04
gaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggcagattcaccatctccagagacaattccaagaacacgctgtatcttcaaatgaacagcctgagagccgaggacacggctgtgtattactgtgcgagaca

SEQ ID NO: 384 - DNA, 291 bp, Artificial Sequence
  Synthetic DNA IGHV3-66*02
gaggtgcagctggtggagtctgggggaggcttggtccagcctggggggtccctgagactctcctgtgcagcctctggattcaccgtcagtagcaactacatgagctgggtccgccaggctccagggaaggggctggagtgggtctcagttatttatagcggtggtagcacatactacgcagactccgtgaagggccgattcaccatctccagagacaattccaagaacacgctgtatcttcaaatgaacagcctgagagctgaggacacggctgtgtattactgtgcgaga

SEQ ID NO: 385 - DNA, 292 bp, Artificial Sequence
  Synthetic DNA IGHV3-38*01
gaggtgcagctggtggagtctgggggaggcttggtacagcctagggggtccctgagactctcctgtgcagcctctggattcaccgtcagtagcaatgagatgagctggatccgccaggctccagggaaggggctggagtgggtctcatccattagtggtggtagcacatactacgcagactccaggaagggcagattcaccatctccagagacaattccaagaacacgctgtatcttcaaatgaacaacctgagagctgagggcacggccgcgtattactgtgccagatata

SEQ ID NO: 386 - DNA, 292 bp, Artificial Sequence
  Synthetic DNA IGHV3-38*02
gaggtgcagctggtggagtctgggggaggcttggtacagcctagggggtccctgagactctcctgtgcagcctctggattcaccgtcagtagcaatgagatgagctggatccgccaggctccagggaaggggctggagtgggtctcatccattagtggtggtagcacatactacgcagactccaggaagggcagattcaccatctccagagacaattccaagaacacgctgtatcttcaaatgaacaacctgagagctgagggcacggccgtgtattactgtgccagatata

SEQ ID NO: 387 - DNA, 288 bp, Artificial Sequence
  Synthetic DNA IGHV3-d*01
gaggtgcagctggtggagtctcggggagtcttggtacagcctggggggtccctgagactctcctgtgcagcctctggattcaccgtcagtagcaatgagatgagctgggtccgccaggctccagggaagggtctggagtgggtctcatccattagtggtggtagcacatactacgcagactccaggaagggcagattcaccatctccagagacaattccaagaacacgctgcatcttcaaatgaacagcctgagagctgaggacacggctgtgtattactgtaagaaa

SEQ ID NO: 388 - DNA, 294 bp, Artificial Sequence
  Synthetic DNA IGHV3/OR16-12*01
gaggtgcagctggtagagtctgggagaggcttggcccagcctggggggtacctaaaactctccggtgcagcctctggattcaccgtcggtagctggtacatgagctggatccaccaggctccagggaagggtctggagtgggtctcatacattagtagtagtggttgtagcacaaactacgcagactctgtgaagggcagattcaccatctccacagacaactcaaagaacacgctctacctgcaaatgaacagcctgagagtggaggacacggccgtgtattactgtgcaaga

SEQ ID NO: 389 - DNA, 302 bp, Artificial Sequence
  Synthetic DNA IGHV3-15*01
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacaga

SEQ ID NO: 390 - DNA, 302 bp, Artificial Sequence
  Synthetic DNA IGHV3-15*02
gaggtgcagctggtggagtctgggggagccttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacaga

SEQ ID NO: 391 - DNA, 302 bp, Artificial Sequence
  Synthetic DNA IGHV3-15*04
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattgaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacaga

SEQ ID NO: 392 - DNA, 302 bp, Artificial Sequence
  Synthetic DNA IGHV3-15*05
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagtctgaaaaccgaggacacagccgtgtattactgtaccacaga

SEQ ID NO: 393 - DNA, 302 bp, Artificial Sequence
  Synthetic DNA IGHV3-15*06
gaggtgcagctggtggagtctgggggaggcttggtaaagcctggggggtcccttagactctcctgtgcagcctctggattcactttcagtaacgcctggatgagctgggtccgccaggctccagggaaggggctggagtgggtcggccgtattaaaagcaaaactgatggtgggacaacaaactacgctgcacccgtgaaaggcagattcaccatctcaagagatgattcaaaaaacacgctgtatctgcaaatgaacagcctgaaaaccgaggacacagccgtgtattactgtaccacaga

SEQ ID NO: 394 - DNA, 302 bp, Artificial Sequence
  Synthetic DNA IGHV3-15*07
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacaga

SEQ ID NO: 395 - DNA, 302 bp, Artificial Sequence
  Synthetic DNA IGHV3-15*03
gaggtgcagctggtggagtctgccggagccttggtacagcctggggggtcccttagactctcctgtgcagcctctggattcacttgcagtaacgcctggatgagctgggtccgccaggctccagggaaggggctggagtgggttggccgtattaaaagcaaagctaatggtgggacaacagactacgctgcacctgtgaaaggcagattcaccatctcaagagttgattcaaaaaacacgctgtatctgcaaatgaacagcctgaaaaccgaggacacagccgtgtattactgtaccacaga

SEQ ID NO: 396 - DNA, 302 bp, Artificial Sequence
  Synthetic DNA IGHV3-15*08
gaggtgcagctggtggagtctgcgggaggcttggtacagcctggggggtcccttagactctcctgtgcagcctctggattcacttgcagtaacgcctggatgagctgggtccgccaggctccagggaaggggctggagtgggttggctgtattaaaagcaaagctaatggtgggacaacagactacgctgcacctgtgaaaggcagattcaccatctcaagagatgattcaaaaaacacgctgtatctgcaaatgatcagcctgaaaaccgaggacacggccgtgtattactgtaccacagg

SEQ ID NO: 397 - DNA, 302 bp, Artificial Sequence
  Synthetic DNA IGHV3-72*01
gaggtgcagctggtggagtctgggggaggcttggtccagcctggagggtccctgagactctcctgtgcagcctctggattcaccttcagtgaccactacatggactgggtccgccaggctccagggaaggggctggagtgggttggccgtactagaaacaaagctaacagttacaccacagaatacgccgcgtctgtgaaaggcagattcaccatctcaagagatgattcaaagaactcactgtatctgcaaatgaacagcctgaaaaccgaggacacggccgtgtattactgtgctagaga

SEQ ID NO: 398 - DNA, 165 bp, Artificial Sequence
  Synthetic DNA IGHV3-72*02
accttcagtgaccactacatggactgggtccgccaggctccagggaaggggctggagtgggttggccgtactagaaacaaagctaacagctacaccacagaatacgccgcgtctgtgaaaggcagattcaccatctcaagagatgattcaaagaactcactgtat

SEQ ID NO: 399 - DNA, 300 bp, Artificial Sequence
  Synthetic DNA IGHV3/OR15-7*01
gaggtgcagctggtggagtctgggggaggcttggtccagcctgggggttctctgagactctcatgtgcagcctctggattcaccttcagtgaccactacatgagctgggtccgccaggctcaagggaaagggctagagttggtaggtttaataagaaacaaagctaacagttacacgacagaatatgctgcgtctgtgaaaggcagacttaccatctcaagagaggattcaaagaacacgatgtatctgcaaatgagcaacctgaaaaccgaggacttggccgtgtattactgtgctaga

SEQ ID NO: 400 - DNA, 300 bp, Artificial Sequence
  Synthetic DNA IGHV3/OR15-7*03
gaggtgcagctggtggagtctgggggaggcttggtccagcctgggggttctctgagactctcatgtgcagcctctggattcaccttcagtgaccactacatgagctgggtccgccaggctcaagggaaagggctagagttggtaggtttaataagaaacaaagctaacagttacacgacagaatatgctgcgtctgtgaaaggcagacttaccatctcaagagaggattcaaagaacacgctgtatctgcaaatgagcagcctgaaaaccgaggacttggccgtgtattactgtgctaga

SEQ ID NO: 401 - DNA, 300 bp, Artificial Sequence
  Synthetic DNA IGHV3/OR15-7*02
gaggtgcagctgttggagtctgggggaggcttggtccagcctgggggttctctgagactctcatgtgctgcctctggattcaccttcagtgaccactacatgagctgggtccgccaggctcaagggaaagggctagagttggtaggtttaataagaaacaaagctaacagttacacgacagaatatgctgcgtctgtgaaaggcagacttaccatctcaagagaggattcaaagaacacgctgtatctgcaaatgagcagcctgaaaaccgaggacttggccgtgtattactgtgctaga

SEQ ID NO: 402 - DNA, 302 bp, Artificial Sequence
  Synthetic DNA IGHV3-73*01
gaggtgcagctggtggagtctgggggaggcttggtccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtactagaca

SEQ ID NO: 403 - DNA, 302 bp, Artificial Sequence
  Synthetic DNA IGHV3-73*02
gaggtgcagctggtggagtccgggggaggcttggtccagcctggggggtccctgaaactctcctgtgcagcctctgggttcaccttcagtggctctgctatgcactgggtccgccaggcttccgggaaagggctggagtgggttggccgtattagaagcaaagctaacagttacgcgacagcatatgctgcgtcggtgaaaggcaggttcaccatctccagagatgattcaaagaacacggcgtatctgcaaatgaacagcctgaaaaccgaggacacggccgtgtattactgtactagaca

SEQ ID NO: 404 - DNA, 302 bp, Artificial Sequence
  Synthetic DNA IGHV3-22*01
gaggtgcatctggtggagtctgggggagccttggtacagcctggggggtccctgagactctcctgtgcagcctctggattcaccttcagttactactacatgagcggggtccgccaggctcccgggaaggggctggaatgggtaggtttcattagaaacaaagctaatggtgggacaacagaatagaccacgtctgtgaaaggcagattcacaatctcaagagatgattccaaaagcatcacctatctgcaaatgaagagcctgaaaaccgaggacacggccgtgtattactgttccagaga

SEQ ID NO: 405 - DNA, 302 bp, Artificial Sequence
  Synthetic DNA IGHV3-22*02
gaggtgcagctggtggagtctgggggaggcttggtacagcctggggggtccctgagactctcctgtgcagcctctggattcaccttcagttactactacatgagcggggtccgccaggctcccgggaaggggctggaatgggtaggtttcattagaaacaaagctaatggtgggacaacagaatagaccacgtctgtgaaaggcagattcacaatctcaagagatgattccaaaagcatcacctatctgcaaatgaagagcctgaaaaccgaggacacggccgtgtattactgttccagaga

SEQ ID NO: 406 - DNA, 302 bp, Artificial Sequence
  Synthetic DNA IGHV3-71*01
gaggtgcagctggtggagtccgggggaggcttggtccagcctggggggtccctgagactctcctgtgcagcctctggattcaccttcagtgactactacatgagctgggtccgccaggctcccgggaaggggctggagtgggtaggtttcattagaaacaaagctaatggtgggacaacagaatagaccacgtctgtgaaaggcagattcacaatctcaagagatgattccaaaagcatcacctatctgcaaatgaacagcctgagagccgaggacacggccgtgtattactgtgcgagaga

SEQ ID NO: 407 - DNA, 302 bp, Artificial Sequence
  Synthetic DNA IGHV3-49*03
gaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagaga

SEQ ID NO: 408 - DNA, 302 bp, Artificial Sequence
  Synthetic DNA IGHV3-49*05
gaggtgcagctggtggagtctgggggaggcttggtaaagccagggcggtccctgagactctcctgtacagcttctggattcacctttggtgattatgctatgagctggttccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagaga

SEQ ID NO: 409 - DNA, 302 bp, Artificial Sequence
  Synthetic DNA |IGHV3-49*01
gaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattagaagcaaagcttatggtgggacaacagaatacaccgcgtctgtgaaaggcagattcaccatctcaagagatggttccaaaagcatcgcctatctgcaaatgaacagcctgaaaaccgaggacacagccgtgtattactgtactagaga

SEQ ID NO: 410 - DNA, 302 bp, Artificial Sequence
  Synthetic DNA IGHV3-49*04
gaggtgcagctggtggagtctgggggaggcttggtacagccagggcggtccctgagactctcctgtacagcttctggattcacctttggtgattatgctatgagctgggtccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagaga

SEQ ID NO: 411 - DNA, 302 bp, Artificial Sequence
  Synthetic DNA IGHV3-49*02
gaggtgcagctggtggagtctgggggaggcttggtacagccagggccgtccctgagactctcctgtacagcttctggattcacctttgggtattatcctatgagctgggtccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactagaga

SEQ ID NO: 412 - DNA, 296 bp, Artificial Sequence
  Synthetic DNA IGHV3-25*01
gagatgcagctggtggagtctgggggaggcttgcaaaagcctgcgtggtccccgagactctcctgtgcagcctctcaattcaccttcagtagctactacatgaactgtgtccgccaggctccagggaatgggctggagttggtttgacaagttaatcctaatgggggtagcacatacctcatagactccggtaaggaccgattcaatacctccagagataacgccaagaacacacttcatctgcaaatgaacagcctgaaaaccgaggacacggccctctattagtgtaccagaga

SEQ ID NO: 413 - DNA, 296 bp, Artificial Sequence
  Synthetic DNA IGHV3-25*02
gagatgcagctggtggagtctgggggaggcttggcaaagcctgcgtggtccccgagactctcctgtgcagcctctcaattcaccttcagtagctactacatgaactgtgtccgccaggctccagggaatgggctggagttggtttgacaagttaatcctaatgggggtagcacatacctcatagactccggtaaggaccgattcaatacctccagagataacgccaagaacacacttcatctgcaaatgaacagcctgaaaaccgaggacacggccctctattagtgtaccagaga

SEQ ID NO: 414 - DNA, 294 bp, Artificial Sequence
  Synthetic DNA IGHV3-25*03
gagatgcagctggtggagtctgggggaggcttggcaaagcctgcgtggtccccgagactctcctgtgcagcctctcaattcaccttcagtagctactacatgaactgtgtccgccaggctccagggaatgggctggagttggttggacaagttaatcctaatgggggtagcacatacctcatagactccggtaaggaccgattcaatacctccagagataacgccaagaacacacttcatctgcaaatgaacagcctgaaaaccgaggacacggccctgtattagtgtaccaga

SEQ ID NO: 415 - DNA, 298 bp, Artificial Sequence
  Synthetic DNA IGHV3-63*01
gaggtggagctgatagagtccatagagggcctgagacaacttgggaagttcctgagactctcctgtgtagcctctggattcaccttcagtagctactgaatgagctgggtcaatgagactctagggaaggggctggagggagtaatagatgtaaaatatgatggaagtcagatataccatgcagactctgtgaagggcagattcaccatctccaaagacaatgctaagaactcaccgtatctccaaacgaacagtctgagagctgaggacatgaccatgcatggctgtacataaggtt

SEQ ID NO: 416 - DNA, 294 bp, Artificial Sequence
  Synthetic DNA IGHV3-63*02
gaggtggagctgatagagtccatagagggcctgagacaacttgggaagttcctgagactctcctgtgtagcctctggattcaccttcagtagctactgaatgagctgggtcaatgagactctagggaaggggctggagggagtaatagatgtaaaatatgatggaagtcagatataccatgcagactctgtgaagggcagattcaccatctccaaagacaatgctaagaactcaccgtatctgcaaacgaacagtctgagagctgaggacatgaccatgcatggctgtacataa

SEQ ID NO: 417 - DNA, 298 bp, Artificial Sequence
  Synthetic DNA IGHV3-32*01
gaggtggagctgatagagtccatagaggacctgagacaacctgggaagttcctgagactctcctgtgtagcctctagattcgccttcagtagcttctgaatgagccgagttcaccagtctccaggcaaggggctggagtgagtaatagatataaaagatgatggaagtcagatacaccatgcagactctgtgaagggcagattctccatctccaaagacaatgctaagaactctctgtatctgcaaatgaacactcagagagctgaggacgtggccgtgtatggctatacataaggtc

SEQ ID NO: 418 - DNA, 296 bp, Artificial Sequence
  Synthetic DNA IGHV3-54*01
gaggtacagctggtggagtctgaagaaaaccaaagacaacttgggggatccctgagactctcctgtgcagactctggattaaccttcagtagctactgaatgagctcagattcccaagctccagggaaggggctggagtgagtagtagatatatagtaggatagaagtcagctatgttatgcacaatctgtgaagagcagattcaccatctccaaagaaaatgccaagaactcactctgtttgcaaatgaacagtctgagagcagagggcacggccgtgtattactgtatgtgagy

SEQ ID NO: 419 - DNA, 296 bp, Artificial Sequence
  Synthetic DNA IGHV3-54*04
gaggtacagctggtggagtctgaagaaaaccaaagacaacttgggggatccctgagactctcctgtgcagactctggattaaccttcagtagctactgaatgagctcagattcccaggctccagggaaggggctggagtgagtagtagatatatagtaggatagaagtcagctatgttatgcacaatctgtgaagagcagattcaccatctccaaagaaaatgccaagaactcactctgtttgcaaatgaacagtctgagagcagagggcacggccgtgtattactgtatgtgagt

SEQ ID NO: 420 - DNA, 207 bp, Artificial Sequence
  Synthetic DNA IGHV3-54*02
tagctactgaatgagctcagattcccaggctccagggaaggggctggagtgagtagtagatatatagtacgatagaagtcagatatgttatgcacaatctgtgaagagcagattcaccatctccaaagaaaatgccaagaactcactccgtttgcaaatgaacagtctgagagcagagggcacggccgtgtattactgtatgtgagg

SEQ ID NO: 421 - DNA, 31 bp, Artificial Sequence
  Synthetic DNA >IGHJ4_1
tgaggagacggtgaccagggttccttggccc

SEQ ID NO: 422 - DNA, 31 bp, Artificial Sequence
  Synthetic DNA >IGHJ4_3
tgaggagacggtgaccagggtcccttggccc

SEQ ID NO: 423 - DNA, 31 bp, Artificial Sequence
  Synthetic DNA >IGHJ4_2
tgaggagacggtgaccagggttccctggccc

SEQ ID NO: 424 - DNA, 32 bp, Artificial Sequence
  Synthetic DNA >IGHJ3_12
ctgaagagacggtgaccattgtcccttggccc

SEQ ID NO: 425 - DNA, 32 bp, Artificial Sequence
  Synthetic DNA >IGHJ6_1
ctgaggagacggtgaccgtggtcccttgcccc

SEQ ID NO: 426 - DNA, 31 bp, Artificial Sequence
  Synthetic DNA >IGHJ6_2
tgaggagacggtgaccgtggtcccttggccc

SEQ ID NO: 427 - DNA, 32 bp, Artificial Sequence
  Synthetic DNA >IGHJ6_34
ctgaggagacggtgaccgtggtccctttgccc

SEQ ID NO: 428 - DNA, 32 bp, Artificial Sequence
  Synthetic DNA >IGHJ2_1
ctgaggagacagtgaccagggtgccacggccc

SEQ ID NO: 429 - DNA, 32 bp, Artificial Sequence
  Synthetic DNA >IGHJ5_1
ctgaggagacggtgaccagggttccttggccc

SEQ ID NO: 430 - DNA, 32 bp, Artificial Sequence
  Synthetic DNA >IGHJ5_2
ctgaggagacggtgaccagggttccctggccc

SEQ ID NO: 431 - DNA, 32 bp, Artificial Sequence
  Synthetic DNA >IGHJ1_1
ctgaggagacggtgaccagggtgccctggccc

SEQ ID NO: 432 - DNA, 34 bp, Artificial Sequence
  Synthetic DNA >IGHJSEQ4_1
tgaggagacggtgaccagggttccttggccccag

SEQ ID NO: 433 - DNA, 34 bp, Artificial Sequence
  Synthetic DNA >IGHJSEQ4_3
tgaggagacggtgaccagggtcccttggccccag

SEQ ID NO: 434 - DNA, 34 bp, Artificial Sequence
  Synthetic DNA >IGHJSEQ4_2
tgaggagacggtgaccagggttccctggccccag

SEQ ID NO: 435 - DNA, 35 bp, Artificial Sequence
  Synthetic DNA >IGHJSEQ3_12
ctgaagagacggtgaccattgtcccttggccccag

SEQ ID NO: 436 - DNA, 35 bp, Artificial Sequence
  Synthetic DNA >IGHJSEQ6_1
ctgaggagacggtgaccgtggtcccttgcccccag

SEQ ID NO: 437 - DNA, 34 bp, Artificial Sequence
  Synthetic DNA >IGHJSEQ6_2
tgaggagacggtgaccgtggtcccttggccccag

SEQ ID NO: 438 - DNA, 35 bp, Artificial Sequence
  Synthetic DNA >IGHJSEQ6_34
ctgaggagacggtgaccgtggtccctttgccccag

SEQ ID NO: 439 - DNA, 35 bp, Artificial Sequence
  Synthetic DNA >IGHJSEQ2_1
ctgaggagacagtgaccagggtgccacggccccag

SEQ ID NO: 440 - DNA, 35 bp, Artificial Sequence
  Synthetic DNA >IGHJSEQ5_1
ctgaggagacggtgaccagggttccttggccccag

SEQ ID NO: 441 - DNA, 35 bp, Artificial Sequence
  Synthetic DNA >IGHJSEQ5_2
ctgaggagacggtgaccagggttccctggccccag

SEQ ID NO: 442 - DNA, 35 bp, Artificial Sequence
  Synthetic DNA >IGHJSEQ1_1
ctgaggagacggtgaccagggtgccctggccccag

SEQ ID NO: 443 - DNA, 36 bp, Artificial Sequence
  Synthetic DNA >IGHV1
tgggtgcaccaggtccangnacaagggcttgagtgg

SEQ ID NO: 444 - DNA, 36 bp, Artificial Sequence
  Synthetic DNA >IGHV2
tgggtgcgacaggctcgngnacaacgccttgagtgg

SEQ ID NO: 445 - DNA, 36 bp, Artificial Sequence
  Synthetic DNA >IGHV3
tgggtgcgccagatgccngngaaaggcctggagtgg

SEQ ID NO: 446 - DNA, 36 bp, Artificial Sequence
  Synthetic DNA >IGHV4
tgggtccgccagscyccngngaaggggctggagtgg

SEQ ID NO: 447 - DNA, 36 bp, Artificial Sequence
  Synthetic DNA >IGHV5
tgggtccgccaggctccngnaaaggggctggagtgg

SEQ ID NO: 448 - DNA, 36 bp, Artificial Sequence
  Synthetic DNA >IGHV6
tgggtctgccaggctccngngaaggggcaggagtgg

SEQ ID NO: 449 - DNA, 37 bp, Artificial Sequence
  Synthetic DNA >IGH7_3.25p
tgtgtccgccaggctccagggaatgggctggagttgg

SEQ ID NO: 450 - DNA, 37 bp, Artificial Sequence
  Synthetic DNA >IGH8_3.54p
tcagattcccaagctccagggaaggggctggagtgag

SEQ ID NO: 451 - DNA, 37 bp, Artificial Sequence
  Synthetic DNA >IGH9_3.63p
tgggtcaatgagactctagggaaggggctggagggag

SEQ ID NO: 452 - DNA, 48 bp, Artificial Sequence
  Synthetic DNA >IGHJ4*01/1-48
actactttgactactggggccaaggaaccctggtcaccgtctcctcag

SEQ ID NO: 453 - DNA, 48 bp, Artificial Sequence
  Synthetic DNA >IGHJ4*03/1-48
gctactttgactactggggccaagggaccctggtcaccgtctcctcag

SEQ ID NO: 454 - DNA, 48 bp, Artificial Sequence
  Synthetic DNA >IGHJ4*02/1-48
actactttgactactggggccagggaaccctggtcaccgtctcctcag

SEQ ID NO: 455 - DNA, 50 bp, Artificial Sequence
  Synthetic DNA >IGHJ3*01/1-50
tgatgcttttgatgtctggggccaagggacaatggtcaccgtctcttcag

SEQ ID NO: 456 - DNA, 50 bp, Artificial Sequence
  Synthetic DNA >IGHJ3*02/1-50
tgatgcttttgatatctggggccaagggacaatggtcaccgtctcttcag

SEQ ID NO: 457 - DNA, 63 bp, Artificial Sequence
  Synthetic DNA >IGHJ6*01/1-63
attactactactactacggtatggacgtctgggggcaagggaccacggtcaccgtctcctcag

SEQ ID NO: 458 - DNA, 63 bp, Artificial Sequence
  Synthetic DNA >IGHJ6*02/1-62
attactactactactacggtatggacgtctggggccaagggaccacggtcaccgtctcctcag

SEQ ID NO: 459 - DNA, 63 bp, Artificial Sequence
  Synthetic DNA >IGHJ6*04/1-63
attactactactactacggtatggacgtctggggcaaagggaccacggtcaccgtctcctcag

SEQ ID NO: 460 - DNA, 63 bp, Artificial Sequence
  Synthetic DNA >IGHJ6*03/1-62
attactactactactactacatggacgtctggggcaaagggaccacggtcaccgtctcctcag

SEQ ID NO: 461 - DNA, 53 bp, Artificial Sequence
  Synthetic DNA >IGHJ2*01/1-53
ctactggtacttcgatctctggggccgtggcaccctggtcactgtctcctcag

SEQ ID NO: 462 - DNA, 51 bp, Artificial Sequence
  Synthetic DNA >IGHJ5*01/1-51
acaactggttcgactcctggggccaaggaaccctggtcaccgtctcctcag

SEQ ID NO: 463 - DNA, 51 bp, Artificial Sequence
  Synthetic DNA >IGHJ5*02/1-51
acaactggttcgacccctggggccagggaaccctggtcaccgtctcctcag

SEQ ID NO: 464 - DNA, 52 bp, Artificial Sequence
  Synthetic DNA >IGHJ1*01/1-52
gctgaatacttccagcactggggccagggcaccctggtcaccgtctcctcag

SEQ ID NO: 465 - DNA, 61 bp, Artificial Sequence
  Synthetic DNA >IGHJ2P*01/1-61
ctacaagtgcttggagcactggggcagggcagcccggacaccgtctccctgggaacgtcag

SEQ ID NO: 466 - DNA, 54 bp, Artificial Sequence
  Synthetic DNA >IGHJ1P*01/1-54
aaaggtgctgggggtcccctgaacccgacccgccctgagaccgcagccacatca

SEQ ID NO: 467 - DNA, 52 bp, Artificial Sequence
  Synthetic DNA >IGHJ3P*01/1-52
cttgcggttggacttcccagccgacagtggtggtctggcttctgaggggtca

SEQ ID NO: 468 - DNA, 58 bp, Artificial Sequence
  Synthetic DNA TRBJ1-2
aatgatacggcgaccaccgagatctacctacaacggttaacctggtccccgaaccgaa

SEQ ID NO: 469 - DNA, 284 bp, Artificial Sequence
  Synthetic DNA TRBV2*02
gaacctgaagtcacccagactcccagccatcaggtcacacagatgggacaggaagtgatcttgcactgtgtccccatctctaatcacttatacttctattggtacagacaaatcttggggcagaaagtcgagtttctggtttccttttataataatgaaatctcagagaagtctgaaatattcgatgatcaattctcagttgaaaggcctgatggatcaaatttcactctgaagatccggtccacaaagctggaggactcagccatgtacttctgtgccagcag

SEQ ID NO: 470 - DNA, 33 bp, Artificial Sequence
  Synthetic DNA Jseq 1-1
acaactgtgagtctggtgccttgtccaaagaaa

SEQ ID NO: 471 - DNA, 33 bp, Artificial Sequence
  Synthetic DNA Jseq 1-2
acaacggttaacctggtccccgaaccgaaggtg

SEQ ID NO: 472 - DNA, 33 bp, Artificial Sequence
  Synthetic DNA Jseq 1-3
acaacagtgagccaacttccctctccaaaatat

SEQ ID NO: 473 - DNA, 33 bp, Artificial Sequence
  Synthetic DNA Jseq 1-4
aagacagagagctgggttccactgccaaaaaac

SEQ ID NO: 474 - DNA, 33 bp, Artificial Sequence
  Synthetic DNA Jseq 1-5
aggatggagagtcgagtcccatcaccaaaatgc

SEQ ID NO: 475 - DNA, 33 bp, Artificial Sequence
  Synthetic DNA Jseq 1-6
gtcacagtgagcctggtcccgttcccaaagtgg

SEQ ID NO: 476 - DNA, 33 bp, Artificial Sequence
  Synthetic DNA Jseq 2-1
agcacggtgagccgtgtccctggcccgaagaac

SEQ ID NO: 477 - DNA, 33 bp, Artificial Sequence
  Synthetic DNA Jseq 2-2
agtacggtcagcctagagccttctccaaaaaac

SEQ ID NO: 478 - DNA, 33 bp, Artificial Sequence
  Synthetic DNA Jseq 2-3
agcactgtcagccgggtgcctgggccaaaatac

SEQ ID NO: 479 - DNA, 33 bp, Artificial Sequence
  Synthetic DNA Jseq 2-4
agcactgagagccgggtcccggcgccgaagtac

SEQ ID NO: 480 - DNA, 33 bp, Artificial Sequence
  Synthetic DNA Jseq 2-5
agcaccaggagccgcgtgcctggcccgaagtac

SEQ ID NO: 481 - DNA, 33 bp, Artificial Sequence
  Synthetic DNA Jseq 2-6
agcacggtcagcctgctgccggccccgaaagtc

SEQ ID NO: 482 - DNA, 33 bp, Artificial Sequence
  Synthetic DNA Jseq 2-7
gtgaccgtgagcctggtgcccggcccgaagtac

SEQ ID NO: 483 - DNA, 34 bp, Artificial Sequence
  Synthetic DNA TRBJ1-5
nacctaggatggagagtcgagtcccatcaccaaa

SEQ ID NO: 484 - DNA, 58 bp, Artificial Sequence
  Synthetic DNA TRBJ1-5
aatgatacggcgaccaccgagatctacctaggatggagagtcgagtcccatcaccaaa

SEQ ID NO: 485 - DNA, 26 bp, Artificial Sequence
  TCR gamma primer
ggaggggaaggccccacagtgtcttc

SEQ ID NO: 486 - DNA, 28 bp, Artificial Sequence
  TCR gamma primer
ccaaatcaggctttggagcacctgatct

SEQ ID NO: 487 - DNA, 27 bp, Artificial Sequence
  TCR gamma primer
caaaggcttagaatatttattacatgt

SEQ ID NO: 488 - DNA, 27 bp, Artificial Sequence
  TCR gamma primer
tgaagtcatacagttcctggtgtccat

SEQ ID NO: 489 - DNA, 31 bp, Artificial Sequence
  TCR gamma primer
agtgttgttccactgccaaagagtttcttat

SEQ ID NO: 490 - DNA, 31 bp, Artificial Sequence
  TCR gamma primer
agctttgttccgggaccaaataccttgattt

SEQ ID NO: 491 - DNA, 30 bp, Artificial Sequence
  TCR gamma primer
cttagtcccttcagcaaatatcttgaacca

SEQ ID NO: 492 - DNA, 30 bp, Artificial Sequence
  TCR gamma primer
cctagtcccttttgcaaacgtcttgatcca

SEQ ID NO: 493 - DNA, 33 bp, Artificial Sequence
  TCR gamma primer
atcacgagtgttgttccactgccaaagagtttc

SEQ ID NO: 494 - DNA, 33 bp, Artificial Sequence
  TCR gamma primer
atcacgagctttgttccgggaccaaataccttg

SEQ ID NO: 495 - DNA, 33 bp, Artificial Sequence
  TCR gamma primer
atcacgcttagtcccttcagcaaatatcttgaa

SEQ ID NO: 496 - DNA, 33 bp, Artificial Sequence
  TCR gamma primer
atcacgcctagtcccttttgcaaacgtcttgat

SEQ ID NO: 497 - DNA, 34 bp, Artificial Sequence
  TCR gamma primer
caagcagaagacggcatacgagctcttccgatct

SEQ ID NO: 498 - DNA, 25 bp, Artificial Sequence
  TCR gamma primer
aatgatacggcgaccaccgagatct

SEQ ID NO: 499 - DNA, 27 bp, Artificial Sequence
  IGHJ primer
gctccccgctatccccagacagcagac

SEQ ID NO: 500 - DNA, 27 bp, Artificial Sequence
  IGHJ primer
agactgggagggggctgcagtgggact

SEQ ID NO: 501 - DNA, 28 bp, Artificial Sequence
  IGHJ primer
agagaaaggaggcagaaggaaagccatc

SEQ ID NO: 502 - DNA, 28 bp, Artificial Sequence
  IGHJ primer
cttcagagttaaagcaggagagaggttg

SEQ ID NO: 503 - DNA, 28 bp, Artificial Sequence
  IGHJ primer
tccctaagtggactcagagagggggtgg

SEQ ID NO: 504 - DNA, 28 bp, Artificial Sequence
  IGHJ primer
gaaaacaaaggccctagagtggccattc

SEQ ID NO: 505 - DNA, 36 bp, Artificial Sequence
  IGHV primer
tgggtgcnacaggcccctggacaagggcttgagtgg

SEQ ID NO: 506 - DNA, 36 bp, Artificial Sequence
  IGHV primer
tgggtgcgacaggctcctggaaaagggcttgagtgg

SEQ ID NO: 507 - DNA, 36 bp, Artificial Sequence
  IGHV primer
tgggtgcgccaggcccccggacaaaggcttgagtgg

SEQ ID NO: 508 - DNA, 36 bp, Artificial Sequence
  IGHV primer
tgggtgcgacaggcccccggacaagcgcttgagtgg

SEQ ID NO: 509 - DNA, 36 bp, Artificial Sequence
  IGHV primer
tgggtgcgacaggcccccagacaagcgcttgagtgg

SEQ ID NO: 510 - DNA, 36 bp, Artificial Sequence
  IGHV primer
tgggtgcgacaggctcgtggacaacgccttgagtgg

SEQ ID NO: 511 - DNA, 36 bp, Artificial Sequence
  IGHV primer
tggttgcaacaggcccctggacaagggcttgaaagg

SEQ ID NO: 512 - DNA, 36 bp, Artificial Sequence
  IGHV primer
tgggtgcgacaggccactggacaagggcttgagtgg

SEQ ID NO: 513 - DNA, 36 bp, Artificial Sequence
  IGHV primer
tgggtgcaacagtcccctggacaagggcttgagtgg

SEQ ID NO: 514 - DNA, 36 bp, Artificial Sequence
  IGHV primer
tgggtgcaacaggcccctggaaaagggcttgagtgg

SEQ ID NO: 515 - DNA, 36 bp, Artificial Sequence
  IGHV primer
tgggtgtgacaaagccctggacaagggcatnagtgg

SEQ ID NO: 516 - DNA, 36 bp, Artificial Sequence
  IGHV primer
tgggtgcgacaggcccctggacaagagcttgggtgg

SEQ ID NO: 517 - DNA, 36 bp, Artificial Sequence
  IGHV primer
tgggtgtgacaggcccctgaacaagggcttgagtgg

SEQ ID NO: 518 - DNA, 36 bp, Artificial Sequence
  IGHV primer
tggatgcgccaggcccctggacaaaggcttgagtgg

SEQ ID NO: 519 - DNA, 36 bp, Artificial Sequence
  IGHV primer
tggatgcgccaggcccctggacaaggcttcgagtgg

SEQ ID NO: 520 - DNA, 36 bp, Artificial Sequence
  IGHV primer
tgggtgtgacaggcccctggacaaggacttgagtgg

SEQ ID NO: 521 - DNA, 36 bp, Artificial Sequence
  IGHV primer
tgggtgcaccaggtccatgcacaagggcttgagtgg

SEQ ID NO: 522 - DNA, 36 bp, Artificial Sequence
  IGHV primer
tgggtgcgccaggtccatgcacaagggcttgagtgg

SEQ ID NO: 523 - DNA, 36 bp, Artificial Sequence
  IGHV primer
tgggtgtgccaggcccatgcacaagggcttgagtgg

SEQ ID NO: 524 - DNA, 36 bp, Artificial Sequence
  IGHV primer
tagatctgtcagccctcagcaaaggccctggagtgg

SEQ ID NO: 525 - DNA, 36 bp, Artificial Sequence
  IGHV primer
tggatccgtcagcccccagggaaggccctggagtgg

SEQ ID NO: 526 - DNA, 36 bp, Artificial Sequence
  IGHV primer
tggatccgtcagcccccaggaaaggccctggagtgg

SEQ ID NO: 527 - DNA, 36 bp, Artificial Sequence
  IGHV primer
tggatccgtcagcccccggggaaggccctggagtgg

SEQ ID NO: 528 - DNA, 36 bp, Artificial Sequence
  IGHV primer
tgggtccgccaggctccagggaaagggctggagtgg

SEQ ID NO: 529 - DNA, 36 bp, Artificial Sequence
  IGHV primer
tgggtccggcaagctccagggaagggcctggagtgg

SEQ ID NO: 530 - DNA, 36 bp, Artificial Sequence
  IGHV primer
tggatccgccaggctccagggaaggggctggagtgg

SEQ ID NO: 531 - DNA, 36 bp, Artificial Sequence
  IGHV primer
tgggtccgccaagctacaggaaaaggtctggagtgg

SEQ ID NO: 532 - DNA, 36 bp, Artificial Sequence
  IGHV primer
tgggtccgccaggctccagggaaggggctggagtgg

SEQ ID NO: 533 - DNA, 36 bp, Artificial Sequence
  IGHV primer
tgggcccgcaaggctccaggaaaggggctggagtgg

SEQ ID NO: 534 - DNA, 36 bp, Artificial Sequence
  IGHV primer
tgggtccgccaggctccaggaaaggggctggagtgg

SEQ ID NO: 535 - DNA, 36 bp, Artificial Sequence
  IGHV primer
tgggtccgccaagctccagggaaggggctggagtgg

SEQ ID NO: 536 - DNA, 36 bp, Artificial Sequence
  IGHV primer
ggggtccgccaggctcccgggaaggggctggaatgg

SEQ ID NO: 537 - DNA, 36 bp, Artificial Sequence
  IGHV primer
tgtgtccgccaggctccagggaatgggctggagttg

SEQ ID NO: 538 - DNA, 36 bp, Artificial Sequence
  IGHV primer
tgggtccgccaggctccaggcaaggggctagagtgg

SEQ ID NO: 539 - DNA, 36 bp, Artificial Sequence
  IGHV primer
tgggtccgccaggctccaggcaaggggctggagtgg

SEQ ID NO: 540 - DNA, 36 bp, Artificial Sequence
  IGHV primer
tgggtccgccaggccccaggcaaggggctagagtgg

SEQ ID NO: 541 - DNA, 36 bp, Artificial Sequence
  IGHV primer
tgggtccgccaggctccgggcaaggggctagagtgg

SEQ ID NO: 542 - DNA, 36 bp, Artificial Sequence
  IGHV primer
cgagttcaccagtctccaggcaaggggctggagtga

SEQ ID NO: 543 - DNA, 36 bp, Artificial Sequence
  IGHV primer
tgggtccatcaggctccaggaaaggggctggagtgg

SEQ ID NO: 544 - DNA, 36 bp, Artificial Sequence
  IGHV primer
tgggtccgtcaagctccggggaagggtctggagtgg

SEQ ID NO: 545 - DNA, 36 bp, Artificial Sequence
  IGHV primer
tgggtccgtcaagctccagggaagggtctggagtgg

SEQ ID NO: 546 - DNA, 36 bp, Artificial Sequence
  IGHV primer
tgggttcgccgggctccagggaagggtctggagtgg

SEQ ID NO: 547 - DNA, 36 bp, Artificial Sequence
  IGHV primer
tgggttcgccgggctccagggaagggtccggagtgg

SEQ ID NO: 548 - DNA, 36 bp, Artificial Sequence
  IGHV primer
tggttccgccaggctccagggaaggggctggagtgg

SEQ ID NO: 549 - DNA, 36 bp, Artificial Sequence
  IGHV primer
tgggtctgccaggctccggagaaggggctggagtgg

SEQ ID NO: 550 - DNA, 36 bp, Artificial Sequence
  IGHV primer
tgggtctgccaggctccggagaaggggcaggagtgg

SEQ ID NO: 551 - DNA, 36 bp, Artificial Sequence
  IGHV primer
tgggtccgccagcctccagggaaggggctggagtgg

SEQ ID NO: 552 - DNA, 36 bp, Artificial Sequence
  IGHV primer
tcagattcccaagctccagggaaggggctggagtga

SEQ ID NO: 553 - DNA, 36 bp, Artificial Sequence
  IGHV primer
tcagattcccaggctccagggaaggggctggagtga

SEQ ID NO: 554 - DNA, 36 bp, Artificial Sequence
  IGHV primer
tgggtccgccaggctccaagaaagggtttgtagtgg

SEQ ID NO: 555 - DNA, 36 bp, Artificial Sequence
  IGHV primer
tgggtcaatgagactctagggaaggggctggaggga

SEQ ID NO: 556 - DNA, 36 bp, Artificial Sequence
  IGHV primer
tgggtccgccaggctccagggaagggactggaatat

SEQ ID NO: 557 - DNA, 36 bp, Artificial Sequence
  IGHV primer
tgggtccgccaggctcccgggaaggggctggagtgg

SEQ ID NO: 558 - DNA, 36 bp, Artificial Sequence
  IGHV primer
tgggtccgccaggcttccgggaaagggctggagtgg

SEQ ID NO: 559 - DNA, 36 bp, Artificial Sequence
  IGHV primer
tgggtccgccaagctccagggaaggggctggtgtgg

SEQ ID NO: 560 - DNA, 36 bp, Artificial Sequence
  IGHV primer
tgggtccgccaggctccagggaagggtctggagtgg

SEQ ID NO: 561 - DNA, 36 bp, Artificial Sequence
  IGHV primer
tgggtccgccaggctcaagggaaagggctagagttg

SEQ ID NO: 562 - DNA, 36 bp, Artificial Sequence
  IGHV primer
tgggtccgccaggctccagggaagggactggagtgg

SEQ ID NO: 563 - DNA, 36 bp, Artificial Sequence
  IGHV primer
tgggttcgccaggctccaggaaaaggtctggagtgg

SEQ ID NO: 564 - DNA, 36 bp, Artificial Sequence
  IGHV primer
tggatccaccaggctccagggaagggtctggagtgg

SEQ ID NO: 565 - DNA, 36 bp, Artificial Sequence
  IGHV primer
tgggtccgccaatctccagggaaggggctggtgtga

SEQ ID NO: 566 - DNA, 36 bp, Artificial Sequence
  IGHV primer
tgggtcctctaggctccaggaaaggggctggagtgg

SEQ ID NO: 567 - DNA, 36 bp, Artificial Sequence
  IGHV primer
tggatccggcagcccccagggaagggactggagtgg

SEQ ID NO: 568 - DNA, 36 bp, Artificial Sequence
  IGHV primer
tggatccggcagccaccagggaagggcctggagtgg

SEQ ID NO: 569 - DNA, 36 bp, Artificial Sequence
  IGHV primer
tggatccgccagcccccagggaagggcctggagtgg

SEQ ID NO: 570 - DNA, 36 bp, Artificial Sequence
  IGHV primer
tggatccgccagcncccagggaagggcctggagtgg

SEQ ID NO: 571 - DNA, 36 bp, Artificial Sequence
  IGHV primer
tggatccgccagcacccagggaagggcctggagtgg

SEQ ID NO: 572 - DNA, 36 bp, Artificial Sequence
  IGHV primer
tggatccgccagcccccagggaaggggctggagtgg

SEQ ID NO: 573 - DNA, 36 bp, Artificial Sequence
  IGHV primer
tggatccgccagcccctagggaaggggctggagtgg

SEQ ID NO: 574 - DNA, 36 bp, Artificial Sequence
  IGHV primer
tggatccgccagcccccagggaagggactggagtgg

SEQ ID NO: 575 - DNA, 36 bp, Artificial Sequence
  IGHV primer
tggatccggcagcccccagggaaggggctggagtgg

SEQ ID NO: 576 - DNA, 36 bp, Artificial Sequence
  IGHV primer
tgggtccgccagcccccagggaaggggctggagtgg

SEQ ID NO: 577 - DNA, 36 bp, Artificial Sequence
  IGHV primer
tggatccggcagcccgccgggaagggactggagtgg

SEQ ID NO: 578 - DNA, 36 bp, Artificial Sequence
  IGHV primer
tggatccggcagccgccggggaagggactggagtgg

SEQ ID NO: 579 - DNA, 36 bp, Artificial Sequence
  IGHV primer
tggatccggcagcccgctgggaagggcctggagtgg

SEQ ID NO: 580 - DNA, 36 bp, Artificial Sequence
  IGHV primer
tggatccggcagcccgccgggaaggggctggagtgg

SEQ ID NO: 581 - DNA, 36 bp, Artificial Sequence
  IGHV primer
tgggtgcgccagatgcccgggaaaggcctggagtgg

SEQ ID NO: 582 - DNA, 36 bp, Artificial Sequence
  IGHV primer
tgggtgcgccagatgcccgggaaaggcttggagtgg

SEQ ID NO: 583 - DNA, 36 bp, Artificial Sequence
  IGHV primer
tgggtgcgccagatgcccaggaaaggcctggagtgg

SEQ ID NO: 584 - DNA, 36 bp, Artificial Sequence
  IGHV primer
tgggtgcgccagatgcccgggaaagaactggagtgg

SEQ ID NO: 585 - DNA, 36 bp, Artificial Sequence
  IGHV primer
tggatcaggcagtccccatcgagaggccttgagtgg

SEQ ID NO: 586 - DNA, 36 bp, Artificial Sequence
  IGHV primer
tgcgacaggcccctggacaagggcttgagtggatgg

SEQ ID NO: 587 - DNA, 36 bp, Artificial Sequence
  IGHV primer
tgggtatgatagacccctggacagggctttgagtgg

SEQ ID NO: 588 - DNA, 36 bp, Artificial Sequence
  IGHV primer
tgggtgccacaggcccctggacaagggcttgagtgg

SEQ ID NO: 589 - DNA, 22 bp, Artificial Sequence
  IGHJ primer
ctgaggagacggtgaccagggt

SEQ ID NO: 590 - DNA, 22 bp, Artificial Sequence
  IGHJ primer
ctgaggagacagtgaccagggt

SEQ ID NO: 591 - DNA, 22 bp, Artificial Sequence
  IGHJ primer
ctgaagagacggtgaccattgt

SEQ ID NO: 592 - DNA, 22 bp, Artificial Sequence
  IGHJ primer
ctgaggagacggtgaccagggt

SEQ ID NO: 593 - DNA, 22 bp, Artificial Sequence
  IGHJ primer
ctgaggagacggtgaccagggt

SEQ ID NO: 594 - DNA, 22 bp, Artificial Sequence
  IGHJ primer
ctgaggagacggtgaccgtggt

SEQ ID NO: 595 - DNA, 50 bp, Homo sapiens
gaattattataagaaactctttggcagtggaacaacactggttgtcacag

SEQ ID NO: 596 - DNA, 50 bp, Homo sapiens
gaattattataagaaactctttggcagtggaacaacacttgttgtcacag

SEQ ID NO: 597 - DNA, 47 bp, Homo sapiens
ttattataagaaactctttggcagtggaacaacacttgttgtcacag

SEQ ID NO: 598 - DNA, 62 bp, Homo sapiens
tgggcaagagttgggcaaaaaaatcaaggtatttggtcccggaacaaagcttatcattacag

SEQ ID NO: 599 - DNA, 60 bp, Homo sapiens
ataccactggttggttcaagatatttgctgaagggactaagctcatagtaacttcacctg

SEQ ID NO: 600 - DNA, 60 bp, Homo sapiens
atagtagtgattggatcaagacgtttgcaaaagggactaggctcatagtaacttcgcctg

SEQ ID NO: 601 - DNA, 300 bp, Homo sapiens
tcttccaacttggaagggagaacgaagtcagtcatcaggcagactgggtcatctgctgaaatcacttgtgatcttgctgaaggaagtaacggctacatccactggtacctacaccaggaggggaaggccccacagcgtcttcagtactatgactcctacaactccaaggttgtgttggaatcaggagtcagtccagggaagtattatacttacgcaagcacaaggaacaacttgagattgatactgcgaaatctaattgaaaatgactctggggtctattactgtgccacctgggacggg

SEQ ID NO: 602 - DNA, 297 bp, Homo sapiens
tcttccaacttggaagggagaacgaagtcagtcatcaggcagactgggtcatctgctgaaatcacttgtgatcttgctgaaggaagtaacggctacatccactggtacctacaccaggaggggaaggccccacagcgtcttcagtactatgactcctacaactccaaggttgtgttggaatcaggagtcagtccagggaagtattatacttacgcaagcacaaggaacaacttgagattgatactgcaaaatctaattgaaaatgactctggggtctattactgtgccacctgggac

SEQ ID NO: 603 - DNA, 300 bp, Homo sapiens
tcttccaacttggaagggagaacgaagtcagtcatcaggcagactgggtcatctgctgaaatcacttgtgatcttgctgaaggaagtaccggctacatccactggtacctacaccaggaggggaaggccccacagcgtcttctgtactatgactcctacacctccagcgttgtgttggaatcaggaatcagcccagggaagtatgatacttatggaagcacaaggaagaacttgagaatgatactgcgaaatcttattgaaaatgactctggagtctattactgtgccacctgggatggg

SEQ ID NO: 604 - DNA, 300 bp, Homo sapiens
tcttccaacttggaagggagaacgaagtcagtcatcaggcagactgggtcatctgctgaaatcacttgtgatcttgctgaaggaagtaccggctacatccactggtacctacaccaggaggggaaggccccacagcgtcttctgtactatgactcctacacctccagcgttgtgttggaatcaggaatcagcccagggaagtatgatacttacggaagcacaaggaagaacttgagaatgatactgcgaaatcttattgaaaatgactctggagtctattactgtgccacctgggatggg

SEQ ID NO: 605 - DNA, 300 bp, Homo sapiens
tcttccaacttggaagggagaacaaagtcagtcaccaggccaactgggtcatcagctgtaatcacttgtgatcttcctgtagaaaatgccgtctacacccactggtacctacaccaggaggggaaggccccacagcgtcttctgtactatgactcctacaactccagggttgtgttggaatcaggaatcagtcgagaaaagtatcatacttatgcaagcacagggaagagccttaaatttatactggaaaatctaattgaacgtgactctggggtctattactgtgccacctgggatagg

SEQ ID NO: 606 - DNA, 297 bp, Homo sapiens
tcttccaacttggaagggagaacgaagtcagtcaccaggctgactgggtcatctgctgaaatcacctgtgatcttcctggagcaagtaccttatacatccactggtacctgcaccaggaggggaaggccccacagtgtcttctgtactatgaaccctactactccagggttgtgctggaatcaggaatcactccaggaaagtatgacactggaagcacaaggagcaattggaatttgagactgcaaaatctaattaaaaatgattctgggttctattactgtgccacctgggacagg

SEQ ID NO: 607 - DNA, 300 bp, Homo sapiens
tcttccaacttggaagggagaacgaagtcagtcaccaggcagactgggtcatctgctgaaatcacttgcgatcttactgtaacaaataccttctacatccactggtacctacaccaggaggggaaggccccacagcgtcttctgtactatgacgtctccaccgcaagggatgtgttggaatcaggactcagtccaggaaagtattatactcatacacccaggaggtggagctggatattgagactgcaaaatctaattgaaaatgattctggggtctattactgtgccacctgggacagg

SEQ ID NO: 608 - DNA, 299 bp, Homo sapiens
tcttccaacttggaagggagaacgaagtcagtcaccaggcagactgggtcatctgctgaaatcacttgcgatcttactgtaacaaataccttctacatccactggtacctacaccaggaggggaaggccccacagcgtcttctgtactatgacgtctccactgcaagggatgtgttggaatcaggactcagtccaggaaagtattatactcatacacccaggaggtggagctggatattgagactgcaaaatctaattgaaaatgattctggggtctattactgtgccacctgggacag

SEQ ID NO: 609 - DNA, 300 bp, Homo sapiens
tcttccaacttggaagggagaatgaagtcagtcaccaggccgactgggtcatctgctgaaatcacttgtgaccttactgtaataaatgccgtctacatccactggtacctacagcaggaggggaagaccccacagcatcttctgcactatgaagtctccaactcaagggatgtgttggaatcaggtctcagtcttggaaagtattatactcatacaccgaggaggtggagctggaatttgagactgcaaaatctaattgaaaatgattctggggtctattactgtgccacctggggcagg

SEQ ID NO: 610 - DNA, 300 bp, Homo sapiens
tcttccaacttggaagggagaatgaagtcagtcaccaggccgactgggtcatctgctgaaatcacttgtgaccttactgtaataaatgccgtctacatccactggtacctacagcaggaggggaagaccccacagcatcttctgcactatgatgtctccaactcaagggatgtgttggaatcaggtctcagtcttggaaagtattatactcatacaccgaggaggtggagctggaatttgagactgcaaaatctaattgaaaatgattctggggtctattactgtgccacctggggcagg

SEQ ID NO: 611 - DNA, 300 bp, Homo sapiens
tcttccaacttggaagggggaacgaagtcagtcacgaggccgactaggtcatctgctgaaatcacttgtgaccttactgtaataaatgccttctacatccactggtacctacaccaggaggggaaggccccacagcgtcttctgtactatgacgtctccaactcaaaggatgtgttggaatcaggactcagtccaggaaagtattatactcatacacccaggaggtggagctggatattgatactacgaaatctaattgaaaatgattctggggtctattactgtgccacctgggacagg

SEQ ID NO: 612 - DNA, 311 bp, Homo sapiens
ttatcaaaagtggagcagttccagctatccatttccacggaagtcaagaaaagtattgacataccttgcaagatatcgagcacaaggtttgaaacagatgtcattcactggtaccggcagaaaccaaatcaggctttggagcacctgatctatattgtctcaacaaaatccgcagctcgacgcagcatgggtaagacaagcaacaaagtggaggcaagaaagaattctcaaactctcacttcaatccttaccatcaagtccgtagagaaagaagacatggccgtttactactgtgctgcgtggtgggtggc

SEQ ID NO: 613 - DNA, 308 bp, Homo sapiens
ttatcaaaagtggagcagttccagctatccatttccacggaagtcaagaaaagtattgacataccttgcaagatatcgagcacaaggtttgaaacagatgtcattcactggtaccggcagaaaccaaatcaggctttggagcacctgatctatattgtctcaacaaaatccgcagctcgacgcagcatgggtaagacaagcaacaaagtggaggcaagaaagaattctcaaactctcacttcaatccttaccatcaagtccgtagagaaagaagacatggccgtttactactgtgctgcgtgggatta

SEQ ID NO: 614 - DNA, 309 bp, Homo sapiens
cttgggcagttggaacaacctgaaatatctatttccagaccagcaaataagagtgcccacatatcttggaaggcatccatccaaggctttagcagtaaaatcatacactggtactggcagaaaccaaacaaaggcttagaatatttattacatgtcttcttgacaatctctgctcaagattgctcaggtgggaagactaagaaacttgaggtaagtaaaaatgctcacacttccacttccactttgaaaataaagttcttagagaaagaagatgaggtggtgtaccactgtgcctgctggattaggcac

SEQ ID NO: 615 - DNA, 309 bp, Homo sapiens
cttgggcagttggaacaacctgaaatatctatttccagaccagcaaataagagtgcccacatatcttggaaggcatccatccaaggctttagcagtaaaatcatacactggtactggcagaaaccaaacaaaggcttagaatatttattacatgtcttcttgacaatctctgctcaagattgctcaggtgggaagactaagaaacttgagataagtaaaaatgctcacacttccacttccactttgaaaataaagttcttagagaaagaagatgaggtggtgtaccactgtgcctgctggattaggcac

SEQ ID NO: 616 - DNA, 306 bp, Homo sapiens
gcaggtcacctagagcaacctcaaatttccagtactaaaacgctgtcaaaaacagcccgcctggaatgtgtggtgtctggaataacaatttctgcaacatctgtatattggtatcgagagagacctggtgaagtcatacagttcctggtgtccatttcatatgacggcactgtcagaaaggaatccggcattccgtcaggcaaatttgaggtggataggatacctgaaacgtctacatccactctcaccattcacaatgtagagaaacaggacatagctacctactactgtgccttgtgggaggtg

SEQ ID NO: 617 - DNA, 306 bp, Homo sapiens
gcaggtcacctagagcaacctcaaatttccagtactaaaacgctgtcaaaaacagcccgcctggaatgtgtggtgtctggaataaaaatttctgcaacatctgtatattggtatcgagagagacctggtgaagtcatacagttcctggtgtccatttcatatgacggcactgtcagaaaggaatctggcattccgtcaggcaaatttgaggtggataggatacctgaaacgtctacatccactctcaccattcacaatgtagagaaacaggacatagctacctactactgtgccttgtgggaggtg

SEQ ID NO: 618 - DNA, 285 bp, Homo sapiens
ctcatcaggccggagcagctggcccatgtcctggggcactagggaagcttggtcatcctgcagtgcgtggtccgcaccaggatcagctacacccactggtaccagcagaagggccaggtccctgaggcactccaccagctggccatgtccaagttggatgtgcagtgggattccatcctgaaagcagataaaatcatagccaaggatggcagcagctctatcttggcagtactgaagttggagacaggcatcgagggcatgaactactgcacaacctgggccctg

SEQ ID NO: 619 - DNA, 48 bp, Homo sapiens; identical to SEQ ID NO: 452

SEQ ID NO: 620 - DNA, 48 bp, Homo sapiens; identical to SEQ ID NO: 453

SEQ ID NO: 621 - DNA, 48 bp, Homo sapiens; identical to SEQ ID NO: 454

SEQ ID NO: 622 - DNA, 50 bp, Homo sapiens; identical to SEQ ID NO: 455

SEQ ID NO: 623 - DNA, 50 bp, Homo sapiens; identical to SEQ ID NO: 456

SEQ ID NO: 624 - DNA, 63 bp, Homo sapiens; identical to SEQ ID NO: 457

SEQ ID NO: 625 - DNA, 62 bp, Homo sapiens
attactactactactacggtatggacgtctggggccaagggaccacggtcaccgtctcctca

SEQ ID NO: 626 - DNA, 63 bp, Homo sapiens; identical to SEQ ID NO: 459

SEQ ID NO: 627 - DNA, 62 bp, Homo sapiens
attactactactactactacatggacgtctggggcaaagggaccacggtcaccgtctcctca

SEQ ID NO: 628 - DNA, 53 bp, Homo sapiens; identical to SEQ ID NO: 461

SEQ ID NO: 629 - DNA, 51 bp, Homo sapiens; identical to SEQ ID NO: 462

SEQ ID NO: 630 - DNA, 51 bp, Homo sapiens; identical to SEQ ID NO: 463

SEQ ID NO: 631 - DNA, 52 bp, Homo sapiens; identical to SEQ ID NO: 464

SEQ ID NO: 632 - DNA, 61 bp, Homo sapiens
gctacaagtgcttggagcactggggcagggcagcccggacaccgtctccctgggaacgtca

SEQ ID NO: 633 - DNA, 54 bp, Homo sapiens; identical to SEQ ID NO: 466

SEQ ID NO: 634 - DNA, 52 bp, Homo sapiens; identical to SEQ ID NO: 467

SEQ ID NO: 635 - DNA, 296 bp, Homo sapiens
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaga

SEQ ID NO: 636 - DNA, 276 bp, Homo sapiens
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctaagatctgacgacacggcc

SEQ ID NO: 637 - DNA, 296 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccagtaccagggacacgtccatcagcacagcctacatggagctgagcaggctgagatctgacgacacggtcgtgtattactgtgcgagaga

SEQ ID NO: 638 - DNA, 296 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaga

SEQ ID NO: 639 - DNA, 296 bp, Homo sapiens
  misc_feature (113)..(113): n = A,T,C or G
caggtgcagctggtgcagtctggggctgaggtgaagaagcttggggcctcagtgaaggtctcctgcaaggcttctggatacaccttcaccggctactatatgcactgggtgcnacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagaga

SEQ ID NO: 640 - DNA, 294 bp, Homo sapiens
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggctgggtcaccatgaccagggacacgtccatcagcacagcctacatggagctgagcaggctgagatctgacgacacggccgtgtattactgtgcgaga

SEQ ID NO: 641 - DNA, 296 bp, Homo sapiens; identical to SEQ ID NO: 285

SEQ ID NO: 642 - DNA, 296 bp, Homo sapiens
caggtccagcttgtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgagaga

SEQ ID NO: 643 - DNA, 296 bp, Homo sapiens
caggttcagctggtgcagtctggggctgaggtgaagaagcctggggcctcagtgaaggtttcctgcaaggcttctggatacaccttcactagctatgctatgcattgggtgcgccaggcccccggacaaaggcttgagtggatgggatggagcaacgctggcaatggtaacacaaaatattcacaggagttccagggcagagtcaccattaccagggacacatccgcgagcacagcctacatggagctgagcagcctgagatctgaggacatggctgtgtattactgtgcgagaga

SEQ ID NO: 644 - DNA, 296 bp, Homo sapiens
  misc_feature (295)..(295): n = A,T,C or G
cagatgcagctggtgcagtctggggctgaggtgaagaagactgggtcctcagtgaaggtttcctgcaaggcttccggatacaccttcacctaccgctacctgcactgggtgcgacaggcccccggacaagcgcttgagtggatgggatggatcacacctttcaatggtaacaccaactacgcacagaaattccaggacagagtcaccattactagggacaggtctatgagcacagcctacatggagctgagcagcctgagatctgaggacacagccatgtattactgtgcaagana

SEQ ID NO: 645 - DNA, 296 bp, Homo sapiens
cagatgcagctggtgcagtctggggctgaggtgaagaagactgggtcctcagtgaaggtttcctgcaaggcttccggatacaccttcacctaccgctacctgcactgggtgcgacaggcccccggacaagcgcttgagtggatgggatggatcacacctttcaatggtaacaccaactacgcacagaaattccaggacagagtcaccattaccagggacaggtctatgagcacagcctacatggagctgagcagcctgagatctgaggacacagccatgtattactgtgcaagata

SEQ ID NO: 646 - DNA, 260 bp, Homo sapiens
agaagactgggtcctcagtgaaggtttcctgcaaggcttccggatacaccttcacctaccgctacctgcactgggtgcgacaggcccccagacaagcgcttgagtggatgggatggatcacacctttcaatggtaacaccaactacgcacagaaattccaggacagagtcaccattaccagggacaggtctatgagcacagcctacatggagctgagcagcctgagatctgaggacacagccatgtattactgtgcaaga

SEQ ID NO: 647 - DNA, 296 bp, Homo sapiens
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaga

SEQ ID NO: 648 - DNA, 296 bp, Homo sapiens; identical to SEQ ID NO: 262

SEQ ID NO: 649 - DNA, 296 bp, Homo sapiens
caggtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgctagaga

SEQ ID NO: 650 - DNA, 296 bp, Homo sapiens; identical to SEQ ID NO: 268

SEQ ID NO: 651 - DNA, 296 bp, Homo sapiens; identical to SEQ ID NO: 269

SEQ ID NO: 652 - DNA, 296 bp, Homo sapiens
caggtgcagctggggcagtctgaggctgaggtaaagaagcctggggcctcagtgaaggtctcctgcaaggcttccggatacaccttcacttgctgctccttgcactggttgcaacaggcccctggacaagggcttgaaaggatgagatggatcacactttacaatggtaacaccaactatgcaaagaagttccagggcagagtcaccattaccagggacatgtccctgaggacagcctacatagagctgagcagcctgagatctgaggactcggctgtgtattactgggcaagata

SEQ ID NO: 653 - DNA, 296 bp, Homo sapiens; identical to SEQ ID NO: 275

SEQ ID NO: 654 - DNA, 294 bp, Homo sapiens; identical to SEQ ID NO: 277

SEQ ID NO: 655 - DNA, 275 bp, Homo sapiens; identical to SEQ ID NO: 270

SEQ ID NO: 656 - DNA, 296 bp, Homo sapiens; identical to SEQ ID NO: 279

SEQ ID NO: 657 - DNA, 294 bp, Homo sapiens; identical to SEQ ID NO: 273

SEQ ID NO: 658 - DNA, 296 bp, Homo sapiens; identical to SEQ ID NO: 276

SEQ ID NO: 659 - DNA, 233 bp, Homo sapiens; identical to SEQ ID NO: 271

SEQ ID NO: 660 - DNA, 296 bp, Homo sapiens; identical to SEQ ID NO: 278

SEQ ID NO: 661 - DNA, 296 bp, Homo sapiens; identical to SEQ ID NO: 281

SEQ ID NO: 662 - DNA, 296 bp, Homo sapiens; identical to SEQ ID NO: 282

SEQ ID NO: 663 - DNA, 296 bp, Homo sapiens; identical to SEQ ID NO: 280

SEQ ID NO: 664 - DNA, 296 bp, Homo sapiens; identical to SEQ ID NO: 272

SEQ ID NO: 665 - DNA, 296 bp, Homo sapiens; identical to SEQ ID NO: 274

SEQ ID NO: 666 - DNA, 296 bp, Homo sapiens
caggtgcagctggtgcagtctggggctgaggtgaagaagcctggggcctcagtgaaggtctcctgcaaggcttctggatacaccttcaccagttatgatatcaactgggtgcgacaggccactggacaagggcttgagtggatgggatggatgaaccctaacagtggtaacacaggctatgcacagaagttccagggcagagtcaccatgaccaggaacacctccataagcacagcctacatggagctgagcagcctgagatctgaggacacggccgtgtattactgtgcgagagg

SEQ ID NO: 667 - DNA, 296 bp, Homo sapiens; identical to SEQ ID NO: 267
  misc_feature (136)..(253): n = A,T,C or G

SEQ ID NO: 668 - DNA, 260 bp, Homo sapiens; identical to SEQ ID NO: 266

SEQ ID NO: 669 - DNA, 294 bp, Homo sapiens; identical to SEQ ID NO: 283

SEQ ID NO: 670 - DNA, 233 bp, Homo sapiens; identical to SEQ ID NO: 284

SEQ ID NO: 671 - DNA, 294 bp, Homo sapiens
caggtgcagctggtgcagtctggggctgaggtgaagaagcctggggcctcagtgaaggtctcctgcaaggcttctggatacatcttcaccgactactatatgcactgggtgcgacaggcccctggacaagagcttgggtggatgggacggatcaaccctaacagtggtggcacaaactatgcacagaagtttcagggcagagtcaccatgaccagggacacgtccatcagcacagcctacacggagctgagcagcctgagatctgaggacacggccacgtattactgtgcgaga

SEQ ID NO: 672 - DNA, 296 bp, Homo sapiens
caggtgcagctggtgcagtctggggctgaggtgaagaagcctggggcctcagtgaaggtctcctgcaaggcttctggatacatcttcaccgactactatatgcactgggtgcgacaggcccctggacaagagcttgggtggatgggacggatcaaccctaacagtggtggcacaaactatgcacagaagtttcagggcagagtcaccatgaccagggacacgtccatcagcacagcctgcacggagctgagcagcctgagatctgaggacacggccacgtattactgtgcgagaga

SEQ ID NO: 673 - DNA, 296 bp, Homo sapiens
caggtgcagctggtgcagtctggagctgaggtgaagaagcctagagcctcagtgaaggtctcctgcaaggcttctggttacacctttaccagctactatatgcactgggtgtgacaggcccctgaacaagggcttgagtggatgggatggatcaacacttacaatggtaacacaaactacccacagaagctccagggcagagtcaccatgaccagagacacatccacgagcacagcctacatggagctgagcaggctgagatctgacgacatggccgtgtattactgtgcgagaga

SEQ ID NO: 674 - DNA, 294 bp, Homo sapiens
caggtccaactggtgtagtctggagctgaggtgaagaagcctggggcctcagtgaaggtctcctgcaaggcttctggatacaccttcaccgactactttatgaactggatgcgccaggcccctggacaaaggcttgagtggatgggatggatcaacgctggcaatggtaacacaaaatattcacagaagctccagggcagagtcaccattaccagggacacatcttcgagcacagcctacatgcagctgagcagcctgagatctgaggacacggccgtgtattactgtgcgaga

SEQ ID NO: 675 - DNA, 260 bp, Homo sapiens
agaagcctggggcctcagtgaaggtctcctgcaaggcttctggatacaccttcaccgactactttatgaactggatgcgccaggcccctggacaaaggcttgagtggatgggatggatcaacgctggcaatggtaacacaaaatattcacagaagctccagggcagagtcaccattaccagggacacatctgcgagcacagcctacatgcagctgagcagcctgagatctgaggacacggccgtgtattactgtgcgaga

SEQ ID NO: 676 - DNA, 294 bp, Homo sapiens
caggtccaactggtgtagtctggagctgaggtgaagaagcctggggcctcagtgaaggtctcctgcaaggcttctggatacaccttcaccagctactatatgaactggatgcgccaggcccctggacaaggcttcgagtggatgggatggatcaacgctggcaatggtaacacaaagtattcacagaagctccagggcagagtcaccattaccagggacacatctgcgagcacagcctacatgcagctgagcagcctgagatctgaggacacggccgtgtattactgtgcgaga

SEQ ID NO: 677 - DNA, 294 bp, Homo sapiens
caggaccagttggtgcagtctggggctgaggtgaagaagcctctgtcctcagtgaaggtctccttcaaggcttctggatacaccttcaccaacaactttatgcactgggtgtgacaggcccctggacaaggacttgagtggatgggatggatcaatgctggcaatggtaacacaacatatgcacagaagttccagggcagagtcaccataaccagggacacgtccatgagcacagcctacacggagctgagcagcctgagatctgaggacacggccgtgtattactgtgcgaga

SEQ ID NO: 678 - DNA, 260 bp, Homo sapiens; identical to SEQ ID NO: 263

SEQ ID NO: 679 - DNA, 294 bp, Homo sapiens; identical to SEQ ID NO: 264

SEQ ID NO: 680 - DNA, 296 bp, Homo sapiens; identical to SEQ ID NO: 265

SEQ ID NO: 681 - DNA, 301 bp, Homo sapiens
caggtcaccttgaaggagtctggtcctgcactggtgaaacccacacagaccctcatgctgacctgcaccttctctgggttctcactcagcacttctggaatgggtgtgggttagatctgtcagccctcagcaaaggccctggagtggcttgcacacatttattagaatgataataaatactacagcccatctctgaagagtaggctcattatctccaaggacacctccaagaatgaagtggttctaacagtgatcaacatggacattgtggacacagccacacattactgtgcaaggagac

SEQ ID NO: 682 - DNA, 301 bp, Homo sapiens
caggtcaccttgaaggagtctggtcctgtgctggtgaaacccacagagaccctcacgctgacctgcaccgtctctgggttctcactcagcaatgctagaatgggtgtgagctggatccgtcagcccccagggaaggccctggagtggcttgcacacattttttcgaatgacgaaaaatcctacagcacatctctgaagagcaggctcaccatctccaaggacacctccaaaagccaggtggtccttaccatgaccaacatggaccctgtggacacagccacatattactgtgcacggatac

SEQ ID NO: 683 - DNA, 302 bp, Homo sapiens
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacagccacatattactgtgcacacagacc

SEQ ID NO: 684 - DNA, 124 bp, Homo sapiens
actagtggagtgggtgtgggctggatccgtcagcccccaggaaaggccctggagtggcttgcactcatttattgggatgatgataagcgctacagcccatctctgaagagcaggctcaccatca

SEQ ID NO: 685 - DNA, 210 bp, Homo sapiens
gctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacagcccatctctgaagagcaggctcaccattaccaaggacacctccaaaaaccaggt

SEQ ID NO: 686 - DNA, 297 bp, Homo sapiens
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacaggcacatattactgtgtacgg

SEQ ID NO: 687 - DNA, 301 bp, Homo sapiens
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacggcccatctctgaagagcaggctcaccatcaccaaggacacctccaaaaaccaggtggtccttacaatgaccaacatggaccctgtggacacagccacatattactgtgcacacagac

SEQ ID NO: 688 - DNA, 300 bp, Homo sapiens
cagatcaccttgaaggagtctggtcctacgctggtaaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacggcccatctctgaagagcaggctcaccatcaccaaggacacctccaaaaaccaggtggtccttacaatgaccaacatggaccctgtggacacagccacatattactgtgcacacaga

SEQ ID NO: 689 - DNA, 294 bp, Homo sapiens
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattggaatgatgataagcgctacagcccatctctgaagagcaggctcaccatcaccaaggacacctccaaaaaccaggtggtccttacaatgaccaacatggaccctgtggacacaggcacatattactgtgta

SEQ ID NO: 690 - DNA, 301 bp, Homo sapiens
caggtcaccttgaaggagtctggtcctgcgctggtgaaacccacacagaccctcacactgacctgcaccttctctgggttctcactcagcactagtggaatgcgtgtgagctggatccgtcagcccccaggaaaggccctggagtggcttgcactcatttattgggatgatgataagcgctacagcccatctctgaagagcaggctcaccatcaccaaggacacctccaaaaaccaggtggtccttacaatgaccaacatggaccctgtggacacagccacatattactgtgcacacagac

SEQ ID NO: 691 - DNA, 301 bp, Homo sapiens
caggtcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacggcccatctctgaagagcaggctcaccatcaccaaggacacctccaaaaaccaggtggtccttacaatgaccaacatggaccctgtggacacagccacatattactgtgcacacagac

SEQ ID NO: 692 - DNA, 297 bp, Homo sapiens
cagatcaccttgaaggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttattgggatgatgataagcgctacagcccatctctgaagagcaggctcaccatcaccaaggacacctccaaaaaccaggtggtccttacaatgaccaacatggaccctgtggacacagccacatattactgtgcacgg

SEQ ID NO: 693 - DNA, 301 bp, Homo sapiens
caggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattactgtgcacggatac

SEQ ID NO: 694 - DNA, 290 bp, Homo sapiens
caggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacggccgtgtattactg

SEQ ID NO: 695 - DNA, 290 bp, Homo sapiens
caggtcaccttgaaggagtctggtcctgcgctggtgaaacccacacagaccctcacactgacctgcaccttctctgggttctcactcagcactagtggaatgcgtgtgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatggaccctgtggacacggccgtgtattactg

SEQ ID NO: 696 - DNA, 288 bp, Homo sapiens
caggtcaccttgaaggagtctggtcctgcgctggtgaaacccacacagaccctcacactgacctgcaccttctctgggttctcactcagcactagtggaatgcgtgtgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatggaccctgtggacacagccacgtattac

SEQ ID NO: 697 - DNA, 237 bp, Homo sapiens
tgcgctggtgaaacccacacagaccctcacactgacctgcaccttctctgggttctcactcagcactagtggaatgcgtgcgagctggatccgtcagcccccagggaaggccctggagtggcttgcacgcattgattgggatgatgataaattctacagcacatctctgaagaccaggctcaccatctccaaggacacctccaaaaaccaggtggtccttacaatgaccaacatgga

SEQ ID NO: 698 - DNA, 290 bp, Homo sapiens
caggtcaccttgaaggagtctggtcctgcgctggtgaaacccacacagaccctcacactgacctgcaccttctctgggttctcactcagcactagtggaatgcgtgtgagctggatccgtcagcccccagggaaggccctggagtggcttgcacgcattgattgggatgatgataaattctacagcacatccctgaagaccaggctcaccatctccaaggacacctccaaaaaccaggtggtccttacaatgaccaacatggaccctgtggacacggccgtgtattactg

SEQ ID NO: 699 - DNA, 290 bp, Homo sapiens
caggtcaccttgagggagtctggtcctgcgctggtgaaacccacacagaccctcacactgacctgcaccttctctgggttctcactcagcactagtggaatgtgtgtgagctggatccgtcagcccccggggaaggccctggagtggcttgcactcattgattgggatgatgataaatactacagcacatctctgaagaccaggctcaccatctccaaggacacctccaaaaaccaggtggtccttacaatgaccaacatggaccctgtggacacggccgtgtattactg

SEQ ID NO: 700 - DNA, 290 bp, Homo sapiens
caggtcaccttgagggagtctggtcctgcgctggtgaaacccacacagaccctcacactgacctgcgccttctctgggttctcactcagcactagtggaatgtgtgtgagctggatccgtcagcccccagggaaggccctggagtggcttgcacgcattgattgggatgatgataaatactacagcacatctctgaagaccaggctcaccatctccaaggacacctccaaaaaccaggtggtccttacaatgaccaacatggaccctgtggacacggccgtgtattactg

SEQ ID NO: 701 - DNA, 297 bp, Homo sapiens
cagatcaccttgaaggagtctggtcctacgctggtgaaacccacacagaccctcacgctgacccgcaccttctctgggttctcactcagcactagtggaatgtgtgtgagctggatccgtcagcccccagggaaggccctggagtggcttgcactcattgattgggatgatgataaatactacagcacatctctgaacaccaggctcaccatctccaaggacacctccaaaaaccaggtggtccttacaatgaccaacatggaccctgtggacacaggcacatattactgtgtacgg

SEQ ID NO: 702 - DNA, 301 bp, Homo sapiens
caggtcaccttgaaggagtctggtcctgcgctggtgaaacccacacagaccctcacactgacctgcaccttctctgggttctcactcagcactagtggaatgcgtgtgagctggatccgtcagcccccagggaaggccctggagtggattgcacgcattgattgggatgatgataaatactacagcacatctctgaagaccaggctcaccatctccaaggacacctccaaaaaccaggtggtccttacaatgaccaacatggaccctgtggacacagccacgtattactgtgcacggatac

SEQ ID NO: 703 - DNA, 301 bp, Homo sapiens
cgggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcacgcattgattgggatgatgataaatactacagcacatctctgaagaccaggctcaccatctccaaggacacctccaaaaaccaggtggtccttacaatgaccaacatggaccctgtggacacagccacgtattactgtgcacggatac

SEQ ID NO: 704 - DNA, 301 bp, Homo sapiens
cagatcaccttgaaggagtctggtcctacgctggtgaaacccacacagaccctcacgctgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacatattactgtgcacacagac

SEQ ID NO: 705 - DNA, 301 bp, Homo sapiens
caggtcaccttgagggagtctggtcctgcgctggtgaaacccacacagaccctcacactgacctgcaccttctctgggttctcactcagcactagtggaatgtgtgtgagctggatccgtcagcccccagggaaggccctggagtggcttgcactcattgattgggatgatgataaatactacagcacatctctgaagaccaggctcaccatctccaaggacacctccaaaaaccaggtggtccttacaatgaccaacatggaccctgtggacacagccacgtattattgtgcacggatac

SEQ ID NO: 706 - DNA, 298 bp, Homo sapiens
caggtcaccttgaaggagtctggtcctgcgctggtgaaacccacagagaccctcacgctgacctgcactctctctgggttctcactcagcacttctggaatgggtatgagctggatccgtcagcccccagggaaggccctggagtggcttgctcacatttttttgaatgacaaaaaatcctacagcacgtctctgaagaacaggctcatcatctccaaggacacctccaaaagccaggtggtccttaccatgaccaacatggaccctgtggacacagccacgtattactgtgcatgga

SEQ ID NO: 707 - DNA, 296 bp, Homo sapiens; identical to SEQ ID NO: 301

SEQ ID NO: 708 - DNA, 294 bp, Homo sapiens; identical to SEQ ID NO: 302

SEQ ID NO: 709 - DNA, 293 bp, Homo sapiens; identical to SEQ ID NO: 312

SEQ ID NO: 710 - DNA, 293 bp, Homo sapiens; identical to SEQ ID NO: 314

SEQ ID NO: 711 - DNA, 291 bp, Homo sapiens; identical to SEQ ID NO: 313

SEQ ID NO: 712 - DNA, 302 bp, Homo sapiens; identical to SEQ ID NO: 389

SEQ ID NO: 713 - DNA, 302 bp, Homo sapiens; identical to SEQ ID NO: 390

SEQ ID NO: 714 - DNA, 302 bp, Homo sapiens; identical to SEQ ID NO: 395

SEQ ID NO: 715 - DNA, 302 bp, Homo sapiens; identical to SEQ ID NO: 391

SEQ ID NO: 716 - DNA, 302 bp, Homo sapiens; identical to SEQ ID NO: 392

SEQ ID NO: 717 - DNA, 302 bp, Homo sapiens; identical to SEQ ID NO: 393

SEQ ID NO: 718 - DNA, 302 bp, Homo sapiens; identical to SEQ ID NO: 394

SEQ ID NO: 719 - DNA, 302 bp, Homo sapiens; identical to SEQ ID NO: 396

SEQ ID NO: 720 - DNA, 296 bp, Homo sapiens; identical to SEQ ID NO: 326

SEQ ID NO: 721 - DNA, 296 bp, Homo sapiens; identical to SEQ ID NO: 327

SEQ ID NO: 722 - DNA, 296 bp, Homo sapiens; identical to SEQ ID NO: 331

SEQ ID NO: 723 - DNA, 296 bp, Homo sapiens; identical to SEQ ID NO: 336

SEQ ID NO: 724 - DNA, 296 bp, Homo sapiens; identical to SEQ ID NO: 303

SEQ ID NO: 725 - DNA, 296 bp, Homo sapiens; identical to SEQ ID NO: 304

SEQ ID NO: 726 - DNA, 302 bp, Homo sapiens; identical to SEQ ID NO: 404

SEQ ID NO: 727 - DNA, 302 bp, Homo sapiens; identical to SEQ ID NO: 405

SEQ ID NO: 728 - DNA, 296 bp, Homo sapiens; identical to SEQ ID NO: 373

SEQ ID NO: 729 - DNA, 296 bp, Homo sapiens; identical to SEQ ID NO: 375

SEQ ID NO: 730 - DNA, 294 bp, Homo sapiens; identical to SEQ ID NO: 376

SEQ ID NO: 731 - DNA, 296 bp, Homo sapiens; identical to SEQ ID NO: 374

SEQ ID NO: 732 - DNA, 294 bp, Homo sapiens; identical to SEQ ID NO: 377

SEQ ID NO: 733 - DNA, 296 bp, Homo sapiens; identical to SEQ ID NO: 412

SEQ ID NO: 734 - DNA, 296 bp, Homo sapiens; identical to SEQ ID NO: 413

SEQ ID NO: 735 - DNA, 294 bp, Homo sapiens; identical to SEQ ID NO: 414

SEQ ID NO: 736 - DNA, 296 bp, Homo sapiens; identical to SEQ ID NO: 342

SEQ ID NO: 737 - DNA, 296 bp, Homo sapiens; identical to SEQ ID NO: 367

SEQ ID NO: 738 - DNA, 296 bp, Homo sapiens; identical to SEQ ID NO: 355

SEQ ID NO: 739 - DNA, 296 bp, Homo sapiens; identical to SEQ ID NO: 350

SEQ ID NO: 740 - DNA, 296 bp, Homo sapiens; identical to SEQ ID NO: 361

SEQ ID NO: 741 - DNA, 296 bp, Homo sapiens; identical to SEQ ID NO: 357

SEQ ID NO: 742 - DNA, 296 bp, Homo sapiens; identical to SEQ ID NO: 349

SEQ ID NO: 743 - DNA, 294 bp, Homo sapiens; identical to SEQ ID NO: 343

SEQ ID NO: 744 - DNA, 296 bp, Homo sapiens; identical to SEQ ID NO: 351

SEQ ID NO: 745 - DNA, 296 bp, Homo sapiens; identical to SEQ ID NO: 346

SEQ ID NO: 746 - DNA, 296 bp, Homo sapiens; identical to SEQ ID NO: 345

SEQ ID NO: 747 - DNA, 296 bp, Homo sapiens; identical to SEQ ID NO: 358

SEQ ID NO: 748 - DNA, 296 bp, Homo sapiens; identical to SEQ ID NO: 362

SEQ ID NO: 749 - DNA, 296 bp, Homo sapiens; identical to SEQ ID NO: 352

SEQ ID NO: 750 - DNA, 296 bp, Homo sapiens; identical to SEQ ID NO: 348

SEQ ID NO: 751 - DNA, 296 bp, Homo sapiens; identical to SEQ ID NO: 347

SEQ ID NO: 752 - DNA, 296 bp, Homo sapiens; identical to SEQ ID NO: 344

SEQ ID NO: 753 - DNA, 296 bp, Homo sapiens; identical to SEQ ID NO: 356

SEQ ID NO: 754 - DNA, 296 bp, Homo sapiens; identical to SEQ ID NO: 359

SEQ ID NO: 755 - DNA, 294 bp, Homo sapiens; identical to SEQ ID NO: 353

SEQ ID NO: 756 - DNA, 296 bp, Homo sapiens; identical to SEQ ID NO: 354

SEQ ID NO: 757 - DNA, 298 bp, Homo sapiens; identical to SEQ ID NO: 417

SEQ ID NO: 758 - DNA, 296 bp, Homo sapiens; identical to SEQ ID NO: 363

SEQ ID NO: 759 - DNA, 296 bp, Homo sapiens; identical to SEQ ID NO: 365

SEQ ID NO: 760 - DNA, 296 bp, Homo sapiens; identical to SEQ ID NO: 366

SEQ ID NO: 761 - DNA, 296 bp, Homo sapiens; identical to SEQ ID NO: 364

SEQ ID NO: 762 - DNA, 296 bp, Homo sapiens; identical to SEQ ID NO: 360

SEQ ID NO: 763 - DNA, 296 bp, Homo sapiens; identical to SEQ ID NO: 332

SEQ ID NO: 764 - DNA, 292 bp, Homo sapiens; identical to SEQ ID NO: 385

SEQ ID NO: 765 - DNA, 292 bp, Homo sapiens; identical to SEQ ID NO: 386

SEQ ID NO: 766 - DNA, 298 bp, Homo sapiens; identical to SEQ ID NO: 333

SEQ ID NO: 767 - DNA, 294 bp, Homo sapiens; identical to SEQ ID NO: 334

SEQ ID NO: 768 - DNA, 291 bp, Homo sapiens; identical to SEQ ID NO: 317

SEQ ID NO: 769 - DNA, 293 bp, Homo sapiens
gaggatcagctggtggagtctgggggaggcttggtacagcctggggggtccctgagaccctcctgtgcagcctctggattcgccttcagtagctatgttctgcactgggttcgccgggctccagggaagggtccggagtgggtatcagctattggtactggtggtgatacatactatgcagactccgtgatgggccgattcaccatctccagagacaacgccaagaagtccttgtatcttcaaatgaacagcctgatagctgaggacatggctgtgtattattgtgcaagaga

SEQ ID NO: 770 - DNA, 282 bp, Homo sapiens; identical to SEQ ID NO: 316

SEQ ID NO: 771 - DNA, 296 bp, Homo sapiens; identical to SEQ ID NO: 305

SEQ ID NO: 772 - DNA, 296 bp, Homo sapiens; identical to SEQ ID NO: 306

SEQ ID NO: 773 - DNA, 296 bp, Homo sapiens; identical to SEQ ID NO: 309

SEQ ID NO: 774 - DNA, 302 bp, Homo sapiens; identical to SEQ ID NO: 409

SEQ ID NO: 775 - DNA, 302 bp, Homo sapiens; identical to SEQ ID NO: 411

SEQ ID NO: 776 - DNA, 302 bp, Homo sapiens; identical to SEQ ID NO: 407

SEQ ID NO: 777 - DNA, 302 bp, Homo sapiens; identical to SEQ ID NO: 410

SEQ ID NO: 778 - DNA, 302 bp, Homo sapiens; identical to SEQ ID NO: 408

SEQ ID NO: 779 - DNA, 296 bp, Homo sapiens; identical to SEQ ID NO: 368

SEQ ID NO: 780 - DNA, 294 bp, Homo sapiens; identical to SEQ ID NO: 369

SEQ ID NO: 781 - DNA, 294 bp, Homo sapiens; identical to SEQ ID NO: 370

SEQ ID NO: 782 - DNA, 293 bp, Homo sapiens; identical to SEQ ID NO: 378

SEQ ID NO: 783 - DNA, 291 bp, Homo sapiens; identical to SEQ ID NO: 379

SEQ ID NO: 784 - DNA, 293 bp, Homo sapiens; identical to SEQ ID NO: 381

SEQ ID NO: 785 - DNA, 296 bp, Homo sapiens; identical to SEQ ID NO: 418

SEQ ID NO: 786 - DNA, 296 bp, Homo sapiens
gaggtacagctggtggagtctgaagaaaaccaaagacaacttgggggatccctgagactctcctgtgcagactctggattaaccttcagtagctactgaatgagctcagattcccaggctccagggaaggggctggagtgagtagtagatatatagtacgatagaagtcagatatgttatgcacaatctgtgaagagcagattcaccatctccaaagaaaatgccaagaactcactccgtttgcaaatgaacagtctgagagcagagggcacggccgtgtattactgtatgtgagg

SEQ ID NO: 787 - DNA, 296 bp, Homo sapiens; identical to SEQ ID NO: 419

SEQ ID NO: 788 - DNA, 296 bp, Homo sapiens; identical to SEQ ID NO: 320

SEQ ID NO: 789 - DNA, 298 bp, Homo sapiens; identical to SEQ ID NO: 415

SEQ ID NO: 790 - DNA, 294 bp, Homo sapiens; identical to SEQ ID NO: 416

SEQ ID NO: 791 - DNA, 296 bp, Homo sapiens; identical to SEQ ID NO: 321

SEQ ID NO: 792 - DNA, 296 bp, Homo sapiens; identical to SEQ ID NO: 322

SEQ ID NO: 793 - DNA, 296 bp, Homo sapiens; identical to SEQ ID NO: 323

SEQ ID NO: 794 - DNA, 296 bp, Homo sapiens; identical to SEQ ID NO: 325

SEQ ID NO: 795 - DNA, 296 bp, Homo sapiens; identical to SEQ ID NO: 324

SEQ ID NO: 796 - DNA, 293 bp, Homo sapiens; identical to SEQ ID NO: 382

SEQ ID NO: 797 - DNA, 291 bp, Homo sapiens; identical to SEQ ID NO: 384

SEQ ID NO: 798 - DNA, 293 bp, Homo sapiens; identical to SEQ ID NO: 380

SEQ ID NO: 799 - DNA, 293 bp, Homo sapiens; identical to SEQ ID NO: 383

SEQ ID NO: 800 - DNA, 296 bp, Homo sapiens; identical to SEQ ID NO: 371

SEQ ID NO: 801 - DNA, 294 bp, Homo sapiens; identical to SEQ ID NO: 372

SEQ ID NO: 802 - DNA, 302 bp, Homo sapiens; identical to SEQ ID NO: 406

SEQ ID NO: 803 - DNA, 302 bp, Homo sapiens; identical to SEQ ID NO: 397

SEQ ID NO: 804 - DNA, 165 bp, Homo sapiens; identical to SEQ ID NO: 398

SEQ ID NO: 805 - DNA, 302 bp, Homo sapiens; identical to SEQ ID NO: 402

SEQ ID NO: 806 - DNA, 302 bp, Homo sapiens; identical to SEQ ID NO: 403

SEQ ID NO: 807 - DNA, 296 bp, Homo sapiens; identical to SEQ ID NO: 337

SEQ ID NO: 808 - DNA, 294 bp, Homo sapiens; identical to SEQ ID NO: 338

SEQ ID NO: 809 - DNA, 296 bp, Homo sapiens; identical to SEQ ID NO: 339

SEQ ID NO: 810 - DNA, 298 bp, Homo sapiens; identical to SEQ ID NO: 335

SEQ ID NO: 811 - DNA, 288 bp, Homo sapiens; identical to SEQ ID NO: 387

SEQ ID NO: 812 - DNA, 293 bp, Homo sapiens; identical to SEQ ID NO: 307

SEQ ID NO: 813 - DNA, 293 bp, Homo sapiens; identical to SEQ ID NO: 308

SEQ ID NO: 814 - DNA, 300 bp, Homo sapiens; identical to SEQ ID NO: 399

SEQ ID NO: 815 - DNA, 300 bp, Homo sapiens; identical to SEQ ID NO: 401

SEQ ID NO: 816 - DNA, 300 bp, Homo sapiens; identical to SEQ ID NO: 400

SEQ ID NO: 817 - DNA, 291 bp, Homo sapiens; identical to SEQ ID NO: 318

SEQ ID NO: 818 - DNA, 291 bp, Homo sapiens; identical to SEQ ID NO: 319

SEQ ID NO: 819 - DNA, 294 bp, Homo sapiens; identical to SEQ ID NO: 388

SEQ ID NO: 820 - DNA, 294 bp, Homo sapiens; identical to SEQ ID NO: 340

SEQ ID NO: 821 - DNA, 294 bp, Homo sapiens; identical to SEQ ID NO: 341

SEQ ID NO: 822 - DNA, 296 bp, Homo sapiens; identical to SEQ ID NO: 330

SEQ ID NO: 823 - DNA, 294 bp, Homo sapiens; identical to SEQ ID NO: 328

SEQ ID NO: 824 - DNA, 294 bp, Homo sapiens; identical to SEQ ID NO: 329

SEQ ID NO: 825 - DNA, 292 bp, Homo sapiens; identical to SEQ ID NO: 310

SEQ ID NO: 826 - DNA, 292 bp, Homo sapiens; identical to SEQ ID NO: 311

SEQ ID NO: 827 - DNA, 296 bp, Homo sapiens
caggtgcagctgcaggagtcgggcccaggactggtgaagccttcggacaccctgtccctcacctgcgctgtctctggttactccatcagcagtagtaactggtggggctggatccggcagcccccagggaagggactggagtggattgggtacatctattatagtgggagcacctactacaacccgtccctcaagagtcgagtcaccatgtcagtagacacgtccaagaaccagttctccctgaagctgagctctgtgaccgccgtggacacggccgtgtattactgtgcgagaaa

SEQ ID NO: 828 - DNA, 296 bp, Homo sapiens
caggtgcagctgcaggagtcgggcccaggactggtgaagccttcacagaccctgtccctcacctgcgctgtctctggttactccatcagcagtagtaactggtggggctggatccggcagcccccagggaagggactggagtggattgggtacatctattatagtgggagcatctactacaacccgtccctcaagagtcgagtcaccatgtcagtagacacgtccaagaaccagttctccctgaagctgagctctgtgaccgccgtggacacggccgtgtattactgtgcgagaaa

SEQ ID NO: 829 - DNA, 296 bp, Homo sapiens
caggtgcagctgcaggagtcgggcccaggactggtgaagccttcggacaccctgtccctcacctgcgctgtctctggttactccatcagcagtagtaactggtggggctggatccggcagcccccagggaagggactggagtggattgggtacatctattatagtgggagcacctactacaacccgtccctcaagagtcgagtcaccatgtcagtagacacgtccaagaaccagttctccctgaagctgagctctgtgaccgccgtggacacggccgtgtattactgtgcgagaga

SEQ ID NO: 830 - DNA, 294 bp, Homo sapiens
caggtgcagctgcaggagtcgggcccaggactggtgaagccttcggacaccctgtccctcacctgcgctgtctctggttactccatcagcagtagtaactggtggggctggatccggcagcccccagggaagggactggagtggattgggtacatctattatagtgggagcacctactacaacccgtccctcaagagtcgagtcaccatgtcagtagacacgtccaagaaccagttctccctgaagctgagctctgtgaccgccgtggacaccggcgtgtattactgtgcgaga

SEQ ID NO: 831 - DNA, 287 bp, Homo sapiens
caggtgcagctgcaggagtcgggcccaggactggtgaagccttcggacaccctgtccctcacctgcgctgtctctggttactccatcagcagtagtaactggtggggctggatccggcagcccccagggaagggactggagtggattgggtacatctattatagtgggagcatctactacaacccgtccctcaagagtcgagtcaccatgtcagtagacacgtccaagaaccagttctccctgaagctgagctctgtgaccgccgtggacacggccgtgtattactg

SEQ ID NO: 832 - DNA, 299 bp, Homo sapiens
cagctgcagctgcaggagtccggctcaggactggtgaagccttcacagaccctgtccctcacctgcgctgtctctggtggctccatcagcagtggtggttactcctggagctggatccggcagccaccagggaagggcctggagtggattgggtacatctatcatagtgggagcacctactacaacccgtccctcaagagtcgagtcaccatatcagtagacaggtccaagaaccagttctccctgaagctgagctctgtgaccgccgcggacacggccgtgtattactgtgccagaga

SEQ ID NO: 833 - DNA, 294 bp, Homo sapiens
cagctgcagctgcaggagtccggctcaggactggtgaagccttcacagaccctgtccctcacctgcgctgtctctggtggctccatcagcagtggtggttactcctggagctggatccggcagccaccagggaagggcctggagtggattgggtacatctatcatagtgggagcacctactacaacccgtccctcaagagtcgagtcaccatatcagtagacaggtccaagaaccagttctccctgaagctgagctctgtgaccgctgcggacacggccgtgtattactgtgcg

SEQ ID NO: 834 - DNA, 299 bp, Homo sapiens
cagctgcagctgcaggagtccggctcaggactggtgaagccttcacagaccctgtccctcacctgcgctgtctctggtggctccatcagcagtggtggttactcctggagctggatccggcagccaccagggaagggcctggagtggattgggagtatctattatagtgggagcacctactacaacccgtccctcaagagtcgagtcaccatatccgtagacacgtccaagaaccagttctccctgaagctgagctctgtgaccgctgcagacacggctgtgtattactgtgcgagaca

SEQ ID NO: 835 - DNA, 227 bp, Homo sapiens
tctggtggctccatcagcagtggtggttactcctggagctggatccggcagccaccagggaagggcctggagtggattgggtacatctatcatagtgggagcacctactacaacccgtccctcaagagtcgagtcaccatatcagtagacacgtccaagaaccagttctccctgaagctgagctctgtgaccgccgcagacacggccgtgtattactgtgcgagaga

SEQ ID NO: 836 - DNA, 299 bp, Homo sapiens
caggtgcagctgcaggagtcgggcccaggactggtgaagccttcacagaccctgtccctcacctgcactgtctctggtggctccatcagcagtggtgattactactggagttggatccgccagcccccagggaagggcctggagtggattgggtacatctattacagtgggagcacctactacaacccgtccctcaagagtcgagttaccatatcagtagacacgtccaagaaccagttctccctgaagctgagctctgtgactgccgcagacacggccgtgtattactgtgccagaga

SEQ ID NO: 837 - DNA, 299 bp, Homo sapiens
caggtgcagctgcaggagtcgggcccaggactggtgaagccttcggacaccctgtccctcacctgcactgtctctggtggctccatcagcagtggtgattactactggagttggatccgccagcccccagggaagggcctggagtggattgggtacatctattacagtgggagcacctactacaacccgtccctcaagagtcgagttaccatatcagtagacacgtccaagaaccagttctccctgaagctgagctctgtgactgcagcagacacggccgtgtattactgtgccagaga

SEQ ID NO: 838 - DNA, 290 bp, Homo sapiens
caggtgcagctgcaggagtcgggcccaggactggtgaagccttcacagaccctgtccctcacctgcactgtctctggtggctccatcagcagtggtgattactactggagttggatccgccagcccccagggaagggcctggagtggattgggtacatctattacagtgggagcacctactacaacccgtccctcaagagtcgagttaccatatcagtagacacgtccaagaaccagttctccctgaagctgagctctgtgactgccgcggacacggccgtgtattactg

SEQ ID NO: 839 - DNA, 290 bp, Homo sapiens
caggtgcagctgcaggactcgggcccaggactggtgaagccttcacagaccctgtccctcacctgcactgtctctggtggctccatcagcagtggtgattactactggagttggatccgccagcccccagggaagggcctggagtggattgggtacttctattacagtgggagcacctactacaacccgtccctcaagagtcgagttaccatatcagtagacacgtccaagaaccagttctccctgaagctgagctctgtgactgccgcagacacggccgtgtattactg

SEQ ID NO: 840 - DNA, 228 bp, Homo sapiens
  misc_feature (54)..(54): n = A,T,C or G
ctctggtggctccatcagcagtggtgattactactggagttggatccgccagcncccagggaagggcctggagtggattgggtacatctattacagtgggagcacctactacaacccgtccctcaagagtcgagtcaccatatcagtagacacgtccaagaaccagttctccctgaagctgagctctgtgactgccgcagacacggccgtgtattactgtgccagaga

SEQ ID NO: 841 - DNA, 227 bp, Homo sapiens
tctggtggctccatcagcagtggtgattactactggagttggatccgccagcacccagggaagggcctggagtggattgggtacatctattacagtgggagcacctactacaacccgtccctcaagagtcgagttaccatatcagtagacacgtccaagaaccagttctccctgaagctgagctctgtgactgccgcagacacggccgtgtattactgtgccagaga

SEQ ID NO: 842 - DNA, 299 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtctagttaccatatcagtagacacgtctaagaaccagttctccctgaagctgagctctgtgactgccgcggacacggccgtgtattactgtgcgagaga

SEQ ID NO: 843 - DNA, 299 bp, Homo sapiens
caggtgcagctgcaggagtcgggcccaggactggtgaagccttcacagaccctgtccctcacctgtactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaga

SEQ ID NO: 844 - DNA, 299 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagaga

SEQ ID NO: 845 - DNA, 294 bp, Homo sapiens
caggtgcggctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcg

SEQ ID NO: 846 - DNA, 291 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgaccgcggacgcggccgtgtattactgtgcg

SEQ ID NO: 847 - DNA, 290 bp, Homo sapiens
caggtgcagctgcaggagtcgggcccaggactggtgaagccttcacagaccctgtccctcacctgcactgtctctggtggctccatcagcagtggtagttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactg

SEQ ID NO: 848 - DNA, 290 bp, Homo sapiens
caggtgcagctgcaggagtcgggcccaggactggtgaagccttcacagaccctgtccctcacctgcactgtctctggtggatccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactg

SEQ ID NO: 849 - DNA, 290 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatccgtagacacgtccaagaaccagttctccctgaagctgagctctgtgactgccgcggacacggccgtgtattactg

SEQ ID NO: 850 - DNA, 290 bp, Homo sapiens
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacaagtccaagaaccagttctccctgaagctgagctctgtgaccgccgcggacacggccgtgtattactg

SEQ ID NO: 851 - DNA, 299 bp, Homo sapiens
caggtgcagctgcaggagtcgggcccaggactgttgaagccttcacagaccctgtccctcacctgcactgtctctggtggctccatcagcagtggtggttactactggagctggatccgccagcacccagggaagggcctggagtggattgggtgcatctattacagtgggagcacctactacaacccgtccctcaagagtcgagttaccatatcagtagacccgtccaagaaccagttctccctgaagccgagctctgtgactgccgcggacacggccgtggattactgtgcgagaga

SEQ ID NO: 852 - DNA, 293 bp, Homo sapiens
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagagg

SEQ ID NO: 853 - DNA, 293 bp, Homo sapiens
caggtgcagctacaacagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcgagagg

SEQ ID NO: 854 - DNA, 284 bp, Homo sapiens
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggccgtgtattactg

SEQ ID NO: 855 - DNA, 293 bp, Homo sapiens
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaacaacaacccgtccctcaagagtcgagccaccatatcagtagacacgtccaagaaccagttctccctgaagctgagctctgtgaccgccgcggacacggctgtgtattactgtgcgagagg

SEQ ID NO: 856 - DNA, 293 bp, Homo sapiens
caggtgcagctacagcagtggggcgcaggactgttgaagccttcggagaccctgtccctcacctgcgctgtctatggtgggtccttcagtggttactactggtgctggatccgccagcccctagggaaggggctggagtggattggggaaatcaatcatagtggaagcaccaacaacaacccgtccctcaagagtcgagccaccatatcagtagacacgtccaagaaccagttctccctgaagctgagctctgtgaccgccgcggacacggctgtgtattactgtgcgagagg

SEQ ID NO: 857 - DNA, 284 bp, Homo sapiens
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgggctctgtgaccgccgcggacacggccgtgtattactg

SEQ ID NO: 858 - DNA, 284 bp, Homo sapiens
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcaaccatagtggaagcaccaactacaacccgtccctcaagagtcgagtcaccatatcagtagacacgtccaagaaccagttctccctgaagctgagctctgtgaccgccgcggacacggccgtgtattactg

SEQ ID NO: 859 - DNA, 288 bp, Homo sapiens
caggtgcagctacagcagtggggcgcaggactgttgaagccttcggagaccctgtccctcacctgcgctgtctatggtgggaccttcagtggttactactggagctggatccgccagcccccagggaaggggctggagtggattggggaaatcaatcatagtggaagcaccaactacaacccgtccctcaagagtcgagtcaccatatcagtagacacgtccaagaaccagttctccctgaagctgagctctgtgaccgccgcggacacggctgtgtattactgtgcg

SEQ ID NO: 860 - DNA, 293 bp, Homo sapiens
caggtgcagctgcaggagtcgggcccaggactggtgaagccttcacagaccctgtccctcacctgcgctgtctatggtgggtccttcagtggttactactggagctggatccgccagcccccagggaagggactggagtggattggggaaatcaatcatagtggaagcaccaactacaacccgtccctcaagagtcgagttaccatatcagtagacacgtctaagaaccagttctccctgaagctgagctctgtgactgccgcggacacggccgtgtattactgtgcgagaga

SEQ ID NO: 861 - DNA, 293 bp, Homo sapiens
caggtgcagctgcaggagtcgggcccaggactggtgaagccttcggagaccctgtccctcacctgcgctgtctatggtgggtccttcagtggttactactggagctggatccgccagcccccagggaagggactggagtggattggggaaatcaatcatagtggaagcaccaactacaacccgtccctcaagagtcgaatcaccatgtcagtagacacgtccaagaaccagttctacctgaagctgagctctgtgaccgccgcggacacggccgtgtattactgtgcgagata

SEQ ID NO: 862 - DNA, 293 bp, Homo sapiens
caggtgcagctacagcagtggggcgcaggactgttgaagccttcggagaccctgtccctcacctgcgctgtctatggtgggtccgtcagtggttactactggagctggatccggcagcccccagggaaggggctggagtggattgggtatatctattatagtgggagcaccaacaacaacccctccctcaagagtcgagccaccatatcagtagacacgtccaagaaccagttctccctgaacctgagctctgtgaccgccgcggacacggccgtgtattgctgtgcgagaga

SEQ ID NO: 863 - DNA, 291 bp, Homo sapiens
caggtgcagctacagcagtggggcgcaggactgttgaagccttcggagaccctgtccctcacctgcgctgtctatggtgggtccttcagtggttactactggagctggatccgccagcccccagggaaggggctggagtggattggggaaatcattcatagtggaagcaccaactacaacccgtccctcaagagtcgagtcaccatatcagtagacacgtccaagaaccagttctccctgaagctgagctctgtgaccgccgcggacacggctgtgtattactgtgcgaga

SEQ ID NO: 864 - DNA, 221 bp, Homo sapiens
tatggtgggtccttcagtggttactactggagctggatccgccagcccccagggaaggggctggagtggattggggaaatcaatcatagtggaagcaccaactacaacccctccctcaagagtcgagtcaccatatcagtagacacgtccaagaaccagttctccctgaagctgagctctgtgaccgccgcggacacggctgtgtattactgtgcgagagg

SEQ ID NO: 865 - DNA, 299 bp, Homo sapiens
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcgagaca

SEQ ID NO: 866 - DNA, 299 bp, Homo sapiens
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccacttctccctgaagctgagctctgtgaccgccgcagacacggctgtgtattactgtgcgagaga

SEQ ID NO: 867 - DNA, 290 bp, Homo sapiens
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggccgtgtattactg

SEQ ID NO: 868 - DNA, 196 bp, Homo sapiens
gctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcggacac

SEQ ID NO: 869 - DNA, 294 bp, Homo sapiens
cagctgcagctgcaggagtcgggcccaggactggtgaagccttcggagaccccgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatccgtagacacgtccaagaaccagttctccctgaagctgagctctgtgaccgccgcagacacggctgtgtattactgtgcg

SEQ ID NO: 870 - DNA, 299 bp, Homo sapiens
cggctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatcagtagacacgtccaagaaccagttccccctgaagctgagctctgtgaccgccgcggacacggccgtgtattactgtgcgagaga

SEQ ID NO: 871 - DNA, 299 bp, Homo sapiens
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtatctattatagtgggagcacctactacaacccgtccctcaagagtcgagtcaccatatcagtagacacgtccaagaaccagttctccctgaagctgagctctgtgaccgccgcggacacggccgtgtattactgtgcgagaga

SEQ ID NO: 872 - DNA, 296 bp, Homo sapiens
caggtgcagctgcaggagtcgggcccaggactggtgaagcctccggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattgctgtgcgagaga

SEQ ID NO: 873 - DNA, 296 bp, Homo sapiens
caggtgcagctgcaggagtcgggcccaggactggtgaagccttcggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactgtgcgagaga

SEQ ID NO: 874 - DNA, 287 bp, Homo sapiens
caggtgcagctgcaggagtcgggcccaggactggtgaagcctccggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactg

SEQ ID NO: 875 - DNA, 287 bp, Homo sapiens
caggtgcagctgcaggagtcgggcccaggactggtgaagcctccggggaccctgtccctcacctgcgctatctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactg

SEQ ID NO: 876 - DNA, 287 bp, Homo sapiens
caggtgcagctgcaggagttgggcccaggactggtgaagcctccggggaccctgtccctcacctgcgctgtctctggtggctccatcagcagtagtaactggtggagttgggtccgccagcccccagggaaggggctggagtggattggggaaatctatcatagtgggagcaccaactacaacccgtccctcaagagtcgagtcaccatatcagtagacaagtccaagaaccagttctccctgaagctgagctctgtgaccgccgcggacacggccgtgtattactg

SEQ ID NO: 877 - DNA, 224 bp, Homo sapiens
  misc_feature (59)..(61): n = A,T,C or G
tctggtggctccatcagcagtagtaactggtggagttgggtccgccagcccccagggannnggctggagtggattggggaaatctatcatagtgggagcaccaactacaacccgtccctcaagagtcgagtcaccatgtcagtagacacgtccaagaaccagttctccctgaagctgagctctgtgaccgccgcggacacggccgtgtattactgtgcgagaga

SEQ ID NO: 878 - DNA, 293 bp, Homo sapiens
caggtgcagctgcaggagtcgggcccaggactggtgaagccttcggagaccctgtccctcacctgcactgtctctggtggctccatcagtagttactactggagctggatccggcagcccgccgggaagggactggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagaga

SEQ ID NO: 879 - DNA, 296 bp, Homo sapiens
caggtgcagctgcaggagtcgggcccaggactggtgaagccttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtccgtagacacgtccaagaaccagttctacctgaagctgagctctgtgaccgccgcggacacggccgtgtattactgtgcgagata

SEQ ID NO: 880 - DNA, 296 bp, Homo sapiens
caggtgcagctgcaggagtcgggcccaggactggtgaagccttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtcagtagacacgtccaagaaccagttctacctgaagctgagctctgtgaccgccgcggacacggccgtgtattactgtgcgagata

SEQ ID NO: 881 - DNA, 287 bp, Homo sapiens
caggtgcagctgcaggagtcgggcccaggactggtgaagccttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtcagtagacacgtccaagaaccagttctccctgaagctgagctctgtgaccgccgcggacacggccgtgtattactg

SEQ ID NO: 882 - DNA, 287 bp, Homo sapiens
caggtgcagctgcaggagtcgggcccaggactggtgaagctttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtcagtagacacgtccaagaaccagttctacctgaagctgagctctgtgaccgccgcggacacggccgtgtattactg

SEQ ID NO: 883 - DNA, 287 bp, Homo sapiens
caggtgcagctgcaggagtcgggcccaggactggtgaagctttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtccgtagacacgtccaagaaccagttctacctgaagctgagctctgtgaccgccgcggacacggccgtgtattactg

SEQ ID NO: 884 - DNA, 287 bp, Homo sapiens
caggtgcagctgcaggagtcgggcccaggactggtgaagccttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtccgtagacacgtccaagaagcagttctacctgaagctgagctctgtgaccgctgcggacacggccgtgtattactg

SEQ ID NO: 885 - DNA, 286 bp, Homo sapiens
caggtgcagctgcaggagtcgggcccaggactggtgaagccttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtccgtagacacgtccaggaaccagttctccctgaagctgagctctgtgaccgccgcagacacggccgtgtattact

SEQ ID NO: 886 - DNA, 296 bp, Homo sapiens
caggtgcagctgcaggagtcgggcccaggactggtgaagccttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtcagtagacacgtccaagaaccagttctacctgaagctgagctctgtgaccgccgcggacacggccgtgtattactgtgcgagaga

SEQ ID NO: 887 - DNA, 296 bp, Homo sapiens
caggtgcagctgcaggagtcgggcccaggactggtgaagccttcggagaccctgtccctcatctgcgctgtctctggtgactccatcagcagtggtaactggtgaatctgggtccgccagcccccagggaaggggctggagtggattggggaaatccatcatagtgggagcacctactacaacccgtccctcaagagtcgaatcaccatgtccgtagacacgtccaagaaccagttctccctgaagctgagctctgtgaccgccgtggacacggccgtgtattactgtgcgagaaa

SEQ ID NO: 888 - DNA, 293 bp, Homo sapiens
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaga

SEQ ID NO: 889 - DNA, 293 bp, Homo sapiens
caggtgcagctgcaggagtcgggcccaggactggtgaagccttcggagaccctgtccctcacctgcactgtctctggtggctccgtcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaga

SEQ ID NO: 890 - DNA, 288 bp, Homo sapiens
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccaattctccctgaagctgagctctgtgaccgctgcggacacggccgtgtattactgtgcg

SEQ ID NO: 891 - DNA, 288 bp, Homo sapiens
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattatagtgggagcacctactacaacccgtccctcaagagtcgagtcaccatgtcagtagacacgtccaagaaccagttctccctgaagctgagctctgtgaccgccgcagacacggctgtgtattactgtgcg

SEQ ID NO: 892 - DNA, 288 bp, Homo sapiens
caggtgcagctgcaggagtcgggcccaggactggtgaagccttcggagaccctgtccctcacctgcactgtctctggtggctccatcagtagttactactggagctggatccggcagccgccggggaagggactggagtggattgggcgtatctattatagtgggagcacctactacaacccgtccctcaagagtcgagtcaccatatccgtagacacgtccaagaaccagttctccctgaagctgagctctgtgaccgccgcagacacggctgtgtattactgtgcg

SEQ ID NO: 893 - DNA, 288 bp, Homo sapiens
caggtgcagctgcaggagtcgggcccaggactggtgaagccttcggagaccctgtccctcacctgcactgtcactggtggctccatcagtagttactactggagctggatccggcagcccgctgggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcg

SEQ ID NO: 894 - DNA, 291 bp, Homo sapiens
caggtgcagctgcaggagtcgggcccaggactggtgaagccttcggacaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgaga

SEQ ID NO: 895 - DNA, 237 bp, Homo sapiens
  misc_feature (36)..(214): n = A,T,C or G
tccctcacctgcactgtctctggtggctccatcagnagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgccgcagacncggccgtgtattactgtgcgaga

SEQ ID NO: 896 - DNA, 221 bp, Homo sapiens
  misc_feature (54)..(54): n = A,T,C or G
tctggtggctccatcagtagttactactggagctggatccggcagcccccaggnannngactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagagg

SEQ ID NO: 897 - DNA, 293 bp, Homo sapiens
caggtgcagctacagcagtggggcgcaggactgttgaagccttcggagaccctgtccctcacctgcgctgtctatggtggctccatcagtagttactactggagctggatccggcagcccgccgggaaggggctggagtggattgggcgtatctataccagtgggagcaccaactacaacccctccctcaagagtcgagtcaccatgtcagtagacacgtccaagaaccagttctccctgaagctgagctctgtgaccgccgcggacacggccgtgtattactgtgcgagata

SEQ ID NO: 898 - DNA, 299 bp, Homo sapiens
caggtgcagctgcaggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaga

SEQ ID NO: 899 - DNA, 299 bp, Homo sapiens
caggtgcagctgcaggagtcgggcccaggactggtgaagccttcacagaccctgtccctcacctgcactgtctctggtggctccatcagcagtggtagttactactggagctggatccggcagcccgccgggaagggactggagtggattgggcgtatctataccagtgggagcaccaactacaacccctccctcaagagtcgagtcaccatatcagtagacacgtccaagaaccagttctccctgaagctgagctctgtgaccgccgcagacacggccgtgtattactgtgcgagaga

SEQ ID NO: 900 - DNA, 299 bp, Homo sapiens
caggtgcagctgcaggagtcgggcccaggactggtgaagccttcggagaccctgtccctcacctgcactgtctctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccacttctccctgaagctgagctctgtgaccgctgcggacacggccgtgtattactgtgcgagaga

SEQ ID NO: 901 - DNA, 287 bp, Homo sapiens
caggtgcagctgcaggagtcgggcccaggactggtgaagccttcggagaccctgtccctcacctgcactgtctctggtggctccgtcagcagtggtagttactactggagctggatccggcagcccccagggaagggactggagtggattggatatatctattacagtgggagcaccaactacaacccctccctcaagagtcgagtcaccatatcagtagacacgtccaagaaccagttctccctgaagctgagctctgtgaccgctgacacggccgtgtattactg

SEQ ID NO: 902 - DNA, 297 bp, Homo sapiens
cagctgcagctgcaggagtcgggcccaggactggtgaagccttcggagaccctgtccctcacctgcactgtctctggtggctccatcagcagtagtagttactactggggctggatccggcagcccccagggaagggactggagtggattgggtatatctattacagtgggagcaccaactacaacccctccctcaagagtcgagtcaccatatcagtagacaagtccaagaaccagttctccctgaagctgagctctgtgaccgccgcggacacggccgtgtattactgtgcgaga

SEQ ID NO: 903 - DNA, 227 bp, Homo sapiens
tctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgccgcggacacggccgtgtattactgtgccagaga

SEQ ID NO: 904 - DNA, 227 bp, Homo sapiens
tctggtggctccgtcagcagtggtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaca

SEQ ID NO: 905 - DNA, 299 bp, Homo sapiens
caggtgcagctgcaggagtcgggcccaggactggtgaagccttcggagaccctgtccctcacctgcactgtctctggtggctccgtcagcagtggtggttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgagaga

SEQ ID NO: 906 - DNA, 294 bp, Homo sapiens
caggtgcagctgcaggagtcgggcccaggactggtgaagccttcggagaccctgtccctcacctgcgctgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgaga

SEQ ID NO: 907 - DNA, 294 bp, Homo sapiens
caggtgcagctgcaggagtcgggcccaggactggtgaagccttcggagaccctgtccctcacctgcactgtctctggttactccatcagcagtggttactactggggctggatccggcagcccccagggaaggggctggagtggattgggagtatctatcatagtgggagcacctactacaacccgtccctcaagagtcgagtcaccatatcagtagacacgtccaagaaccagttctccctgaagctgagctctgtgaccgccgcagacacggccgtgtattactgtgcgaga

SEQ ID NO: 908 - DNA, 296 bp, Homo sapiens
caggtgcagctgcaggagtcgggcccaggactggtgaagccttcggagaccctgtccctcacctgcgttgtctctggtggctccatcagcagtagtaactggtggagctgggtccgccagcccccagggaaggggctggagtggattggggaaatctatcatagtgggaaccccaactacaacccgtccctcaagagtcgagtcaccatatcaatagacaagtccaagaaccaattctccctgaagctgagctctgtgaccgccgcggacacggccgtgtattactgtgcgagaga

SEQ ID NO: 909 - DNA, 296 bp, Homo sapiens; identical to SEQ ID NO: 291

SEQ ID NO: 910 - DNA, 296 bp, Homo sapiens; identical to SEQ ID NO: 293

SEQ ID NO: 911 - DNA, 294 bp, Homo sapiens; identical to SEQ ID NO: 294

SEQ ID NO: 912 - DNA, 294 bp, Homo sapiens; identical to SEQ ID NO: 295

SEQ ID NO: 913 - DNA, 245 bp, Homo sapiens; identical to SEQ ID NO: 292

SEQ ID NO: 914 - DNA, 294 bp, Homo sapiens; identical to SEQ ID NO: 300

SEQ ID NO: 915 - DNA, 294 bp, Homo sapiens; identical to SEQ ID NO: 296

SEQ ID NO: 916 - DNA, 295 bp, Homo sapiens; identical to SEQ ID NO: 299

SEQ ID NO: 917 - DNA, 294 bp, Homo sapiens; identical to SEQ ID NO: 297

SEQ ID NO: 918 - DNA, 294 bp, Homo sapiens; identical to SEQ ID NO: 298

SEQ ID NO: 919 - DNA, 305 bp, Homo sapiens
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagaga

SEQ ID NO: 920 - DNA, 305 bp, Homo sapiens
caggtacagctgcagcagtcaggtccgggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagaga

SEQ ID NO: 921 - DNA, 294 bp, Homo sapiens; identical to SEQ ID NO: 286

SEQ ID NO: 922 - DNA, 296 bp, Homo sapiens; identical to SEQ ID NO: 288

SEQ ID NO: 923 - DNA, 274 bp, Homo sapiens; identical to SEQ ID NO: 287

SEQ ID NO: 924 - DNA, 289 bp, Homo sapiens; identical to SEQ ID NO: 290

SEQ ID NO: 925 - DNA, 296 bp, Homo sapiens; identical to SEQ ID NO: 289

(19) United States
(12) Patent Application Publication
(10) Pub. No.: US 2015/0299785 A1
(43) Pub. Date: Oct. 22, 2015

(54) METHOD OF MEASURING ADAPTIVE IMMUNITY

(71) Applicant: Fred Hutchinson Cancer Research Center, Seattle, WA (US)

(72) Inventors: Robert J. Livingston, Seattle, WA (US); Christopher S. Carlson, Kirkland, WA (US); Harlan S. Robins, Seattle, WA (US)

(21) Appl. No.: 14/640,145

(22) Filed: Mar. 6, 2015

Related U.S. Application Data
(63) Continuation of application No. 13/217,126, filed on Aug. 24, 2011, now abandoned.
Application No. 13/217,126 is a continuation-in-part of application No. 12/794,507, filed on Jun. 4, 2010, now abandoned.
(60) Provisional application No. 61/220,344, filed on Jun. 25, 2009.
(60) Provisional application No. 61/376,655, filed on Aug. 24, 2010.
(60) Provisional application No. 61/425,672, filed on Dec. 21, 2010.
(60) Provisional application No. 61/481,653, filed on May 2, 2011.
(60) Provisional application No. 61/492,085, filed on Jun. 1, 2011.

(51) Int. Cl. C12Q 1/68

(52) U.S. Cl. CPC C12Q 1/6874

(57) ABSTRACT

Compositions and methods for measuring adaptive immune receptor (T cell receptor and immunoglobulin) diversity are described, and find uses for assessing immunocompetence and other purposes. Means are provided for assessing the effects of diseases or conditions that compromise the immune system and of therapies aimed to reconstitute it. Lymphoid (B- and T-cell) adaptive immune receptor diversity is quantified by calculating the number of uniquely rearranged. CDR3-containing immunoglobulin (Ig) or T-cell receptor (TCR) variable region-encoding genes from sample cells such as blood cells.

### CROSS-REFERENCE TO RELATED APPLICATIONS

This application is continuation-in-part of U.S. application Ser. No. 12/794,507, filed on Jun. 4, 2010, now pending; which application claims the benefit of U.S. Provisional Application No. 61/220,344, filed on Jun. 25, 2009. This application also claims the benefit of U.S. Provisional Application No. 61/376,655, filed Aug. 24, 2010; U.S. Provisional Application No. 61/425,672, filed Dec. 21, 2010; U.S. Provisional Application No. 61/481,653, filed May 2, 2011; and U.S. Provisional Application No. 61/492,085, filed Jun. 1, 2011. All of the above-mentioned applications are hereby incorporated by reference in their entirety.

### BACKGROUND

1. Technical Field

What is described is a method to measure the adaptive immunity of a patient by analyzing the diversity of T cell receptor genes or antibody genes using large scale sequencing of nucleic acid extracted from adaptive immune system cells.

2. Description of the Related Art

The adaptive immune system protects higher organisms against infections and other clinical insults attributable to foreign substances using adaptive immune receptors, antigen-specific recognition proteins that are expressed by hematopoietic cells of the lymphoid lineage and that are capable of distinguishing self from non-self molecules in the host. B lymphocytes mature to express antibodies (immunoglobulins, Igs) that occur as heterodimers of a heavy (H) a light (L) chain polypeptide, while T lymphocytes express heterodimeric T cell receptors (TCR).

Immunocompetence is the ability of the body to produce a normal immune response (i.e., antibody production and/or cell-mediated immunity) following exposure to a pathogen, which might be a live organism (such as a bacterium or fungus), a virus, or specific antigenic components isolated from a pathogen and introduced in a vaccine. Immunocompetence is the opposite of immunodeficiency or immuno-incompetent or immunocompromised. Several examples would be a newborn that does not yet have a fully functioning immune system but may have maternally transmitted antibody (immunodeficient); a late stage AIDS patient with a failed or failing immune system (immuno-incompetent); a transplant recipient taking medication so their body will not reject the donated organ (immunocompromised); age-related attenuation of T cell function in the elderly; or individuals exposed to radiation or chemotherapeutic drugs. There may be cases of overlap but these terms are all indicators of a dysfunctional immune system. In reference to lymphocytes, immunocompetence means that a B cell or T cell is mature and can recognize antigens and allow a person to mount an immune response.

Immunocompetence depends on the ability of the adaptive immune system to mount an immune response specific for any potential foreign antigens, using the highly polymorphic receptors encoded by B cells (immunoglobulins, Igs) and T cells (T cell receptors, TCRs).

Igs expressed by B cells are proteins consisting of four polypeptide chains, two heavy chains (H chains) and two light chains (L chains), forming an H₂L₂ structure. Each pair of H and L chains contains a hypervariable domain, consisting of a light chain variable (V_{L}) and a heavy chain variable (V_{H}) region, and a constant domain. The H chains of Igs are of several types, μ, δ, γ, α, and ρ. The diversity of Igs within an individual is mainly determined by the hypervariable domain. The V domain of H chains is created by the combinatorial joining of three types of germline gene segments, the V_{H}, D_{H}, and J_{H} segments. Hypervariable domain sequence diversity is further increased by independent addition and deletion of nucleotides at the V_{H}-D_{H}, D_{H}-J_{H}, and V_{H}-J_{H} junctions during the process of Ig gene rearrangement. In this respect, immunocompetence is reflected in the diversity of Igs.

TCRs expressed by αβ T cells are proteins consisting of two transmembrane polypeptide chains (α and β), expressed front the TCRA and TCRB genes, respectively. Similar TCR proteins are expressed in gamma-delta T cells, from the TCRG and TCRD loci. Each TCR peptide contains variable complementarity determining regions (CDRs), as well as framework regions (FRs) and a constant region. The sequence diversity of αβ T cells is largely determined by the amino acid sequence of the third complementarity-determining region (CDR3) loops of the α and β chain variable domains, which diversity is a result of recombination between variable (V_{β}), diversity (D_{β}), and joining (J_{β}) gene segments in the β chain locus, and between analogous V_{α} and H_{α} gene segments in the α chain locus, respectively. The existence of multiple such gene segments in the TCR α and β chain loci allows for a large number of distinct CDR3 sequences to be encoded. CDR3 sequence diversity is further increased by independent addition and deletion of nucleotides at the V_{β}-D_{β}, D_{β}-J_{β}, and V_{α}-J_{α} junctions during the process of TCR gene rearrangement. In this respect, immunocompetence is reflected in the diversity of TCRs.

TCRγδ is distinctive from the αβ TCR in that it encodes a receptor that interacts closely with the innate immune system. TCRγβ, is expressed early in development, has specialized anatomical distribution, has unique, pathogen and small-molecule specificities, and has a broad spectrum of innate and adaptive cellular interactions. A biased pattern of TCRγ V and J segment expression is established early in ontogeny as the restricted subsets of TCRγδ cells populate the mouth, skin, gut, vagina, and lungs prenatally. Consequently, the diverse TCRγ repertoire in adult tissues is the result of extensive peripheral expansion following stimulation by environmental exposure to pathogens and toxic molecules. Therefore, measurement of the TCRγ diversity in the adult is a proxy to the history of environmental exposure.

There exists a long-felt need for methods of assessing or measuring the adaptive immune system of patients in a variety of settings, whether immunocompetence in the immunocompromised, or dysregulated adaptive immunity in malignancies or autoimmune disease. A demand exists for methods of diagnosing a disease state or the effects of aging by assessing the immunocompetence of a patient. In the same way results of therapies that modify the immune system need to be monitored by assessing the immunocompetence of the patient while undergoing the treatment. Additionally, a demand exists for methods to monitor the adaptive immune system in the context of autoimmune disease flares and remissions, in order to monitor response to therapy, or the need to initiate prophylactic therapy pre-symptomatically.

### BRIEF SUMMARY

In certain embodiments the present invention provides a composition comprising (a) a plurality of V-segment oligonucleotide primers that are each independently capable of specifically hybridizing to at least one polynucleotide encoding a human T cell receptor (TCR) V-region polypeptide, wherein each V-segment primer comprises a nucleotide sequence of at least 15 contiguous nucleotides that is complementary to at least one functional TCR Vγ-encoding gene segment and wherein the plurality of V-segment primers specifically hybridize to substantially all functional TCR Vγ-encoding gene segments that are present in a sample that comprises T cells from a human subject; and (b) a plurality of J-segment oligonucleotide primers that are each independently capable of specifically hybridizing to at least one polynucleotide encoding a human T cell receptor (TCR) J-region polypeptide, wherein each J-segment primer comprises a nucleotide sequence of at least 15 contiguous nucleotides that is complementary to at least one functional TCR Jγ-encoding gene segment and wherein the plurality of J-segment primers specifically hybridize to substantially all functional TCR Jγ-encoding gene segments that are present in the sample that comprises T cells from the human subject; wherein the V-segment and J-segment primers are capable of promoting amplification in a multiplex polymerase chain reaction (PCR) of substantially all rearranged TCRγ CDR3-encoding regions in the sample to produce a multiplicity of amplified rearranged DNA molecules from a population of T cells in the sample, said multiplicity of amplified rearranged DNA molecules being sufficient to quantify diversity of the TCRγ CDR3-encoding region in the population of T cells.

In certain embodiments each amplified rearranged DNA molecule in the multiplicity of amplified rearranged DNA molecules is less than 600 nucleotides in length. In certain embodiments each functional TCR Vγ-encoding gene segment comprises a V gene recombination signal sequence (RSS) and each functional TCR Jγ-encoding gene segment comprises a J gene RSS, and wherein each amplified rearranged DNA molecule comprises (i) at least 40 contiguous nucleotides of a sense strand of the TCR Vγ-encoding gene segment, said at least 40 contiguous nucleotides being situated 5′ to the V gene RSS and (ii) at least 30 contiguous nucleotides of a sense strand of the TCR Jγ-encoding gene segment, said at least 30 contiguous nucleotides being situated 3′ to the J gene RSS. In certain embodiments the V-segment oligonucleotide primers comprise one or more of the nucleotide sequences set forth in SEQ ID NOS:601-618. In certain embodiments the J-segment oligonucleotide primers comprise one or more of the nucleotide sequences set forth in SEQ ID NOS:595-600 and 493-496. In certain embodiments either or both of (i) the V-segment oligonucleotide primers comprise one or a plurality of oligonucleotides that exhibit at least 90% sequence identity to one or more of the nucleotide sequences set forth in SEQ ID NOS:601-618, and (ii) the J-segment oligonucleotide primers comprise one or a plurality of oligonucleotides that exhibit at least 90% sequence identity to one or more of the nucleotide sequences set forth in SEQ ID NOS:595-600 and 493-496.

In certain embodiments either or both of (i) the V-segment oligonucleotide primers comprise one or a plurality of oligonucleotides that exhibit at least 95% sequence identity to one or more of the nucleotide sequences set forth in SEQ ID NOS:601-618 and (ii) the J-segment oligonucleotide primers comprise one or a plurality of oligonucleotides that exhibit at least 95% sequence identity to one or more of the nucleotide sequences set forth in SEQ ID NOS:595-600 and 493-496. In certain embodiments diversity of the TCRγ CDR3-encoding region is quantifiable by sequencing the multiplicity of amplified rearranged DNA molecules. In certain embodiments either or both of (i) each V-segment oligonucleotide primer has a 5′ end that is modified with a universal forward primer sequence that is compatible with a DNA sequencer, and (ii) each J-segment oligonucleotide primer has a 5′ end that is modified with a universal reverse primer sequence that is compatible with a DNA sequencer. In certain further embodiments the universal forward primer sequence is set forth in SEQ ID NO:497 and the universal reverse primer sequence is set forth in SEQ ID NO:498. In certain embodiments either or both of (i) the V-segment oligonucleotide primers comprise one or more of the nucleotide sequences set forth in SEQ ID NOS:485-488 and 497, and (ii) the J-segment oligonucleotide primers comprise one or more of the nucleotide sequences set forth in SEQ ID NOS:489-496 and 498.

According to certain other embodiments there is provided a method for quantifying TCRγ CDR3-encoding region diversity in a population of T cells, comprising (a) amplifying DNA extracted from a biological sample that comprises T cells, in a multiplex polymerase chain reaction (PCR) that comprises (i) a plurality of V-segment oligonucleotide primers that are each independently capable of specifically hybridizing to at least one polynucleotide encoding a human T cell receptor (TCR) V-region polypeptide, wherein each V-segment primer comprises a nucleotide sequence of at least 15 contiguous nucleotides that is complementary to at least one functional TCR Vγ-encoding gene segment and wherein the plurality of V-segment primers specifically hybridize to substantially all functional TCR Vγ-encoding gene segments that are present in the sample, and (ii) a plurality of J-segment oligonucleotide primers that are each independently capable of specifically hybridizing to at least one polynucleotide encoding a human T cell receptor (TCR) J-region polypeptide, wherein each J-segment primer comprises a nucleotide sequence of at least 15 contiguous nucleotides that is complementary to at least one functional TCR Jγ-encoding gene segment and wherein the plurality of J-segment primers specifically hybridize to substantially all functional TCR Jγ-encoding gene segments that are present in the sample, wherein the V-segment and J-segment primers are capable of promoting amplification in said multiplex polymerase chain reaction (PCR) of substantially all rearranged TCRγ CDR3-encoding regions in the sample to produce a multiplicity of amplified rearranged DNA molecules from a population of T cells in the sample, said multiplicity of amplified rearranged DNA molecules being sufficient to quantify diversity of the TCRγ CDR3-encoding region in the population of T cells; and (b) determining a relative frequency of occurrence for each unique rearranged DNA molecule in said multiplicity of amplified rearranged DNA molecules, and thereby quantifying TCRγ CDR3-encoding region diversity. In certain further embodiments the step of determining comprises sequencing said multiplicity of amplified rearranged DNA molecules.

In another embodiment there is provided a composition comprising (a) a plurality of V-segment oligonucleotide primers that are each independently capable of specifically hybridizing to at least one polynucleotide encoding a human immunoglobulin heavy chain (IGH) V-region polypeptide, wherein each V-segment primer comprises a nucleotide sequence of at least 15 contiguous nucleotides that is complementary to at least one functional IGH V_{H}-encoding gene segment and wherein the plurality of V-segment primers specifically hybridize to substantially all functional IGH V_{H}-encoding gene segments that are present in a sample that comprises B cells from a human subject; and (b) a plurality of J-segment oligonucleotide primers that are each independently capable of specifically hybridizing to at least one polynucleotide encoding a human immunoglobulin heavy chain (IGH) J-region polypeptide, wherein each J-segment primer comprises a nucleotide sequence of at least 15 contiguous nucleotides that is complementary to at least one functional TCR J_{H}-encoding gene segment and wherein the plurality of J-segment primers specifically hybridize to substantially all functional IGH Jβ-encoding gene segments that are present in the sample that comprises B cells from the human subject; wherein the V-segment and J-segment primers are capable of promoting amplification in a multiplex polymerase chain reaction (PCR) of substantially all rearranged IGH CDR3-encoding regions in the sample to produce a multiplicity of amplified rearranged DNA molecules from a population of B cells in the sample, said multiplicity of amplified rearranged DNA molecules being sufficient to quantify diversity of the IGH CDR3-encoding region in the population of B cells. In certain embodiments each amplified rearranged DNA molecule in the multiplicity of amplified rearranged DNA molecules is less than 600 nucleotides in length.

In certain embodiments each functional IGH VH-encoding gene segment comprises a V gene and each functional IGH JH-encoding gene segment comprises a J gene, and wherein each amplified rearranged DNA molecule comprises (i) at least 40 contiguous nucleotides derived from the IGH VH-encoding gene segment, said at least 40 contiguous nucleotides being situated 5′ to the V gene RSS and (ii) at least 30 contiguous nucleotides of the IGH JH-encoding gene segment, said at least 30 contiguous nucleotides being situated 3′ to the J gene RSS. In certain embodiments the V-segment oligonucleotide primers comprise one or more of the nucleotide sequences set forth in SEQ ID NOS:443-451, 505-588 and 635-925. In certain embodiments the J-segment oligonucleotide primers comprise one or more of the nucleotide sequences set forth in SEQ ID NOS:421-431, 452-467, 499-504 and 619-634. In certain embodiments either or both of (i) the V-segment oligonucleotide primers comprise one or a plurality of oligonucleotides that exhibit at least 90% sequence identity to one or more of the nucleotide sequences set forth in SEQ ID NOS:443-451, 505-588 and 635-925, and (ii) the J-segment oligonucleotide primers comprise one or a plurality of oligonucleotides that exhibit at least 90% sequence identity to one or more of the nucleotide sequences set forth in SEQ ID NOS:421-431, 452-467, 499-504 and 619-634 In certain embodiments either or both of (i) the V-segment oligonucleotide primers comprise one or a plurality of oligonucleotides that exhibit at least 95% sequence identity to one or more of the nucleotide sequences set forth in SEQ ID NOS:443-451, 505-588 and 635-925, and (ii) the J-segment oligonucleotide primers comprise one or a plurality of oligonucleotides that exhibit at least 95% sequence identity to one or more of the nucleotide sequences set forth in SEQ ID NOS:421-431, 452-467, 499-504 and 619-634.

In certain embodiments diversity of the IGH CDR3-encoding region is quantifiable by sequencing the multiplicity of amplified rearranged DNA molecules. In certain embodiments either or both of (i) each V-segment oligonucleotide primer has a 5′ end that is modified with a universal forward primer sequence that is compatible with a DNA sequencer, and (ii) each J-segment oligonucleotide primer has a 5′ end that is modified with a universal reverse primer sequence that is compatible with a DNA sequencer. In certain embodiments the universal forward primer sequence is set forth in SEQ ID NO:497 and the universal reverse primer sequence is set forth in SEQ ID NO:498. In certain embodiments either or both of (i) the V-segment oligonucleotide primers comprise one or more of the nucleotide sequences set forth in SEQ ID NOS:497, 505-588 and 635-925 and, and (ii) the J-segment oligonucleotide primers comprise one or more of the nucleotide sequences set forth in SEQ ID NOS:498, 499-504 and 619-634.

According to certain other embodiments there is provided a method for quantifying IGH CDR3-encoding region diversity in a population of B cells, comprising (a) amplifying DNA extracted from a biological sample that comprises B cells, in a multiplex polymerase chain reaction (PCR) that comprises (i) a plurality of variable (V)-segment oligonucleotide primers that are each independently capable of specifically hybridizing to at least one polynucleotide encoding a human immunoglobulin heavy chain (IGH) V-region polypeptide, wherein each V-segment primer comprises a nucleotide sequence of at least 15 contiguous nucleotides that is complementary to at least one functional IGH V-encoding gene segment and wherein the plurality of V-segment primers specifically hybridize to substantially all functional IGH V-encoding gene segments that are present in the sample, and (ii) a plurality of J-segment oligonucleotide primers that are each independently capable of specifically hybridizing to at least one polynucleotide encoding a human immunoglobulin heavy chain (IGH) J-region polypeptide, wherein each J-segment primer comprises a nucleotide sequence of at least 15 contiguous nucleotides that is complementary to at least one functional IGH J-encoding gene segment and wherein the plurality of J-segment primers specifically hybridize to substantially all functional IGH J-encoding gene segments that are present in the sample, wherein the V-segment and J-segment primers are capable of promoting amplification in said multiplex polymerase chain reaction (PCR) of substantially all rearranged IGH CDR3-encoding regions in the sample to produce a multiplicity of amplified rearranged DNA molecules from a population of B cells in the sample, said multiplicity of amplified rearranged DNA molecules being sufficient to quantify diversity of the IGH CDR3-encoding region in the population of B cells; and (b) determining a relative frequency of occurrence for each unique rearranged DNA molecule in said multiplicity of amplified rearranged DNA molecules, and thereby quantifying IGH CDR3-encoding region diversity. In certain embodiments the step of determining comprises sequencing said multiplicity of amplified rearranged DNA molecules.

Turning to another embodiment, there is provided a composition comprising (a) a plurality of V-segment oligonucleotide primers that are each independently capable of specifically hybridizing to at least one polynucleotide encoding a human T cell receptor (TCR) V-region polypeptide, wherein each V-segment primer comprises a nucleotide sequence of at least 15 contiguous nucleotides that is complementary to at least one functional TCR Vβ-encoding gene segment and wherein the plurality of V-segment primers specifically hybridize to substantially all functional TCR Vβ-encoding gene segments that are present in a sample that comprises T cells from a human subject; and (b) a plurality of J-segment oligonucleotide primers that are each independently capable of specifically hybridizing to at least one polynucleotide encoding a human T cell receptor (TCR) J-region polypeptide, wherein each J-segment primer comprises a nucleotide sequence of at least 15 contiguous nucleotides that is complementary to at least one functional TCR Jβ-encoding gene segment and wherein the plurality of J-segment primers specifically hybridize to substantially all functional TCR Jβ-encoding gene segments that are present in the sample that comprises T cells from the human subject; wherein the V-segment and J-segment primers are capable of promoting amplification in a multiplex polymerase chain reaction (PCR) of substantially all rearranged TCRβ CDR3-encoding regions in the sample to produce a multiplicity of amplified rearranged DNA molecules from a population of T cells in the sample, said multiplicity of amplified rearranged DNA molecules being sufficient to quantify diversity of the TCRβ CDR3-encoding region in the population of T cells.

In certain embodiments each amplified rearranged DNA molecule in the multiplicity of amplified rearranged DNA molecules is less than 600 nucleotides in length. In certain embodiments each functional TCR Vβ-encoding gene segment comprises a V gene recombination signal sequence (RSS) and each functional TCR Jβ-encoding gene segment comprises a J gene RSS, and wherein each amplified rearranged DNA molecule comprises (i) at least 40 contiguous nucleotides of a sense strand of the TCR Vβ-encoding gene segment, said at least 40 contiguous nucleotides being situated 5′ to the V gene RSS and (ii) at least 30 contiguous nucleotides of a sense strand of the TCR Jβ-encoding gene segment, said at least 30 contiguous nucleotides being situated 3′ to the J gene RSS. In certain embodiments the V-segment oligonucleotide primers comprise one or more of the nucleotide sequences set forth in SEQ ID NOS:1-45 and 58-102. In certain embodiments the J-segment oligonucleotide primers comprise one or more of the nucleotide sequences set forth in SEQ ID NOS:46-57, 103-113, 468 and 483-484. In certain embodiments either or both of (i) the V-segment oligonucleotide primers comprise one or a plurality of oligonucleotides that exhibit at least 90% sequence identity to one or more of the nucleotide sequences set forth in SEQ ID NOS: 1-45 and 58-102, and (ii) the J-segment oligonucleotide primers comprise one or a plurality of oligonucleotides that exhibit at least 90% sequence identity to one or more of the nucleotide sequences set forth in SEQ ID NOS: 46-57, 103-113, 468 and 483-484. In certain embodiments either or both of (i) the V-segment oligonucleotide primers comprise one or a plurality of oligonucleotides that exhibit at least 95% sequence identity to one or more of the nucleotide sequences set forth in SEQ ID NOS: 1-45 and 58-102, and (ii) the J-segment oligonucleotide primers comprise one or a plurality of oligonucleotides that exhibit at least 95% sequence identity to one or more of the nucleotide sequences set forth in SEQ ID NOS: 46-57, 103-113, 468 and 483-484.

In certain embodiments diversity of the TCRβ CDR3-encoding region is quantifiable by sequencing the multiplicity of amplified rearranged DNA molecules. In certain embodiments either or both of (i) each V-segment oligonucleotide primer has a 5′ end that is modified with a universal forward primer sequence that is compatible with a DNA sequencer, and (ii) each J-segment oligonucleotide primer has a 5′ end that is modified with a universal reverse primer sequence that is compatible with a DNA sequencer. In certain embodiments the universal forward primer sequence is set forth in SEQ ID NO:497 and the universal reverse primer sequence is set forth in SEQ ID NO:498. In certain embodiments either or both of (i) the V-segment oligonucleotide primer comprises the nucleotide sequence set forth in SEQ ID NOS: 497, and (ii) the J-segment oligonucleotide primers comprise one or more of the nucleotide sequences set forth in SEQ ID NOS:470-482 and 498. In certain embodiments each functional TCR Jβ-encoding gene segment comprises a J gene RSS and each J-segment oligonucleotide primer independently contains a unique four-base tag at a position that is complementary to nucleotide positions +11 through +14 located 3′ of the RSS on a sense strand of the TCR Jβ-encoding gene segment.

In certain other embodiments there is provided a method for quantifying TCRβ CDR3-encoding region diversity in a population of T cells, comprising (a) amplifying DNA extracted from a biological sample that comprises T cells, in a multiplex polymerase chain reaction (PCR) that comprises (i) a plurality of V-segment oligonucleotide primers that are each independently capable of specifically hybridizing to at least one polynucleotide encoding a human T cell receptor (TCR) V-region polypeptide, wherein each V-segment primer comprises a nucleotide sequence of at least 15 contiguous nucleotides that is complementary to at least one functional TCR Vβ-encoding gene segment and wherein the plurality of V-segment primers specifically hybridize to substantially all functional TCR Vβ-encoding gene segments that are present in the sample, and (ii) a plurality of J-segment oligonucleotide primers that are each independently capable of specifically hybridizing to at least one polynucleotide encoding a human T cell receptor (TCR) J-region polypeptide, wherein each J-segment primer comprises a nucleotide sequence of at least 15 contiguous nucleotides that is complementary to at least one functional TCR Jβ-encoding gene segment and wherein the plurality of J-segment primers specifically hybridize to substantially all functional TCR Jβ-encoding gene segments that are present in the sample, wherein the V-segment and J-segment primers are capable of promoting amplification in said multiplex polymerase chain reaction (PCR) of substantially all rearranged TCRβ CDR3-encoding regions in the sample to produce a multiplicity of amplified rearranged DNA molecules from a population of T cells in the sample, said multiplicity of amplified rearranged DNA molecules being sufficient to quantify diversity of the TCRβ CDR3-encoding region in the population of T cells; and (b) determining a relative frequency of occurrence for each unique rearranged DNA molecule in said multiplicity of amplified rearranged DNA molecules, and thereby quantifying TCRβ CDR3-encoding region diversity. In certain embodiments the step of determining comprises sequencing said multiplicity of amplified rearranged DNA molecules.

In certain embodiments of the invention there is provided a composition comprising a multiplicity of V-segment primers, wherein each primer comprises a sequence that is complementary to a single functional V segment or a small family of V segments; and a multiplicity of J-segment primers, wherein each primer comprises a sequence that is complementary to a J segment; wherein the V segment and J-segment primers permit amplification of a TCR CDR3 region by a multiplex polymerase chain reaction (PCR) to produce a multiplicity of amplified DNA molecules sufficient to quantify the diversity of the TCR genes. One embodiment of the invention is the composition, wherein each V-segment primer comprises a sequence that is complementary to a single Vβ segment, and each J segment primer comprises a sequence that is complementary to a Jβ segment, and wherein V segment and J-segment primers permit amplification of a TCRβ CDR3 region. Another embodiment is the composition, wherein each V-segment primer comprises a sequence that is complementary to a single functional Vα segment, and each J segment primer comprises a sequence that is complementary to a Jα segment, and wherein V segment and J-segment primers permit amplification of a TCRα CDR3 region.

Another embodiment of the invention is the composition, wherein the V segment primers hybridize with a conserved segment, and have similar annealing strength. Another embodiment is wherein the V segment primer is anchored at position −43 in the Vβ segment relative to the recombination signal sequence (RSS). Another embodiment is wherein the multiplicity of V segment primers consist of at least 45 primers specific to 45 different Vβ genes. Another embodiment is wherein the V segment primers have sequences that are selected from the group consisting of SEQ ID NOS:1-45. Another embodiment is wherein the V segment primers have sequences that are selected from the group consisting of SEQ ID NOS:58-102. Another embodiment is wherein there is a V segment primer for each Vβ segment.

Another embodiment of the invention is the composition, wherein the J segment primers hybridize with a conserved framework region element of the Jβ segment, and have similar annealing strength. In certain embodiments, the multiplicity of J segment primers consist of at least thirteen primers specific to thirteen different Jβ genes, and in certain embodiments the J segment primers have sequences that are selected from SEQ ID NOS:46-57. In another embodiment the J segment primers have sequences that are selected from SEQ ID NOS:102-113. Another embodiment is wherein there is a J segment primer for each Jβ segment. Another embodiment is wherein all J segment primers anneal to the same conserved motif.

Another embodiment of the invention is the composition, wherein the amplified DNA molecule starts from said conserved motif and amplifies adequate sequence to diagnostically identify the J segment and includes the CDR3 junction and extends into the V segment. Another embodiment is wherein the amplified Jβ gene segments each have a unique four base tag at positions +11 through +14 downstream of the RSS site.

In other embodiments there is provided a composition further comprising a set of sequencing oligonucleotides, wherein the sequencing oligonucleotides hybridize to a regions within the amplified DNA molecules. An embodiment is wherein the sequencing oligonucleotides hybridize adjacent to a four base tag within the amplified Jβ gene segments at positions +11 through +14 downstream of the RSS site. Another embodiment is wherein the sequencing oligonucleotides are selected from the group consisting of SEQ ID NOS:58-70. Another embodiment is wherein the V-segment or J-segment are selected to contain a sequence error-correction by merger of closely related sequences. Another embodiment is the composition, further comprising a universal C segment primer for generating cDNA from mRNA.

In certain other embodiments there is provided a composition comprising a multiplicity of V segment primers, wherein each V segment primer comprises a sequence that is complementary to a single functional V segment or a small family of V segments; and a multiplicity of J segment primers, wherein each J segment primer comprises a sequence that is complementary to a J segment; wherein the V segment and J segment primers permit amplification of the TCRG CDR3 region by a multiplex polymerase chain reaction (PCR) to produce a multiplicity of amplified DNA molecules sufficient to quantify the diversity of antibody heavy chain genes. In certain other embodiments there is provided a composition comprising a multiplicity of V segment primers, wherein each V segment primer comprises a sequence that is complementary to a single functional V segment or a small family of V segments; and a multiplicity of J segment primers, wherein each J segment primer comprises a sequence that is complementary to a J segment; wherein the V segment and J segment primers permit amplification of antibody heavy chain (IGH, Igh or IgH) CDR3 region by a multiplex polymerase chain reaction (PCR) to produce a multiplicity of amplified DNA molecules sufficient to quantify the diversity of antibody heavy chain genes. In another embodiment there is provided a composition comprising a multiplicity of V segment primers, wherein each V segment primer comprises a sequence that is complementary to a single functional V segment or a small family of V segments; and a multiplicity of J segment primers, wherein each J segment primer comprises a sequence that is complementary to a J segment; wherein the V segment and J segment primers permit amplification of antibody light chain (IGL) V_{L} region by a multiplex polymerase chain reaction (PCR) to produce a multiplicity of amplified DNA molecules sufficient to quantify the diversity of antibody light chain genes.

In certain other embodiments there is provided a method comprising selecting a multiplicity of V segment primers, wherein each V segment primer comprises a sequence that is complementary to a single functional V segment or a small family of V segments; and selecting a multiplicity of J segment primers, wherein each J segment primer comprises a sequence that is complementary to a J segment; combining the V segment and J segment primers with a sample of genomic DNA to permit amplification of a CDR3 region by a multiplex polymerase chain reaction (PCR) to produce a multiplicity of amplified DNA molecules sufficient to quantify the diversity of the TCR genes.

One embodiment of the invention is the method wherein each V segment primer comprises a sequence that is complementary to a single functional Vβ segment, and each J segment primer comprises a sequence that is complementary to a Jβ segment; and wherein combining the V segment and J segment primers with a sample of genomic DNA permits amplification of a TCR CDR3 region by a multiplex polymerase chain reaction (PCR) and produces a multiplicity of amplified DNA molecules. Another embodiment is wherein each V segment primer comprises a sequence that is complementary to a single functional Vα segment, and each J segment primer comprises a sequence that is complementary to a Jα segment; and wherein combining the V segment and J segment primers with a sample of genomic DNA permits amplification of a TCR CDR3 region by a multiplex polymerase chain reaction (PCR) and produces a multiplicity of amplified DNA molecules.

Another embodiment is the method further comprising a step of sequencing the amplified DNA molecules. Another embodiment is wherein the sequencing step utilizes a set of sequencing oligonucleotides that hybridize to regions within the amplified DNA molecules. Another embodiment is the method, further comprising a step of calculating the total diversity of TCRβ CDR3 sequences among the amplified DNA molecules. Another embodiment is wherein the method shows that the total diversity of a normal human subject is greater than 1·10⁶ sequences, greater than 2·10⁶ sequences, or greater than 3·10⁶ sequences. In certain other embodiments there is provided a method of diagnosing immunodeficiency in a human patient, comprising measuring the diversity of TCR CDR3 sequences of the patient, and comparing the diversity of the subject to the diversity obtained from a normal subject. Another embodiment is the method wherein measuring the diversity of TCR sequences comprises the steps of selecting a multiplicity of V segment primers, wherein each V segment primer comprises a sequence that is complementary to a single functional V segment or a small family of V segments; and selecting a multiplicity of J segment primers, wherein each J segment primer comprises a sequence that is complementary to a J segment; combining the V segment and J segment primers with a sample of genomic DNA to permit amplification of a TCR CDR3 region by a multiplex polymerase chain reaction (PCR) to produce a multiplicity of amplified DNA molecules; sequencing the amplified DNA molecules; calculating the total diversity of TCR CDR3 sequences among the amplified DNA molecules.

An embodiment of the invention is the method, wherein comparing the diversity is determined by calculating using the following equation:

$$Δ ( t ) = {\sum }_{x} {E ( {n}_{x} )}_{measurement 1 + 2} - {\sum }_{x} {E ( {n}_{x} )}_{measurement 2} = S {\int }_{0}^{∞} {e}^{- λ} ( 1 - {e}^{- λ t} ) d G ( λ )$$

wherein G(λ) is the empirical distribution function of the parameters λ₁, . . . , λ_{S}, nₓ is the number of clonotypes sequenced exactly x times, and

$$E ( {n}_{x} ) = S {\int }_{0}^{∞} ( \frac{{e}^{- λ} {λ}^{x}}{x !} ) d G ( λ ) .$$

Another embodiment is the method wherein the diversity of at least two samples of genomic DNA are compared. Another embodiment is wherein one sample of genomic DNA is from a patient and the other sample is from a normal subject. Another embodiment is wherein one sample of genomic DNA is from a patient before a therapeutic treatment and the other sample is from the patient after treatment. Another embodiment is wherein the two samples of genomic DNA are from the same patient at different times during treatment. Another embodiment is wherein a disease is diagnosed based on the comparison of diversity among the samples of genomic DNA. Another embodiment is wherein the immunocompetence of a human patient is assessed by the comparison.

These and other aspects of the herein described invention embodiments will be evident upon reference to the following detailed description and attached drawings. All of the U.S. patents, U.S. patent application publications, U.S. patent applications, foreign patents, foreign patent applications and non-patent publications referred to in this specification and/or listed in the Application Data Sheet are incorporated herein by reference in their entirety, as if each was incorporated individually. Aspects and embodiments of the invention can be modified, if necessary, to employ concepts of the various patents, applications and publications to provide yet further embodiments.

### BRIEF DESCRIPTION OF THE SEVERAL VIEWS OF THE DRAWINGS

FIG. 1A illustrates the rearrangement and sequencing strategy of the template region of TCRγ (gamma) gene in a T cell, where V and J represent the combinatorial assortment of V and J segments and N represents the addition or deletion of random DNA sequence at the splice junctions. Arrows represent the flanking TCRγ (gamma) V and J primers that amplify the gene region encoding the CDR3 region. The TRGJseq primers are used to sequence 60 bases of the CDR3 region, sufficient to identify the V, J segments and random N nucleotides that comprise the pathogen binding domain of the T cell receptor.

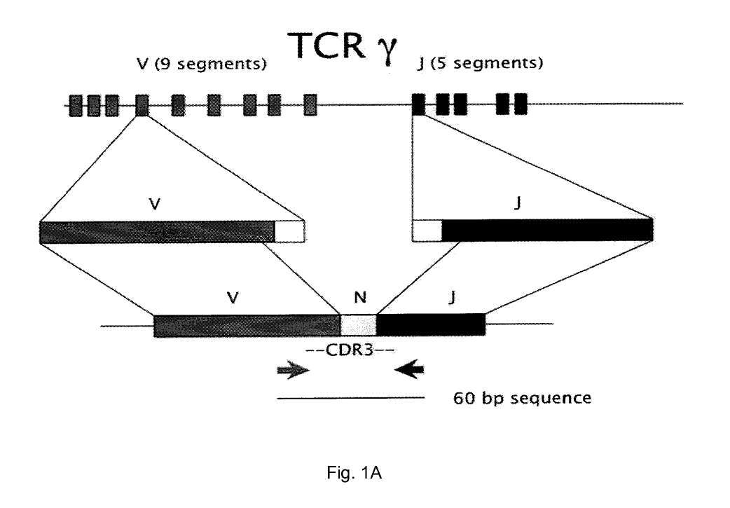

FIG. 1B illustrates the rearrangement and sequencing strategy of the immunoglobulin heavy chain (IGH) gene in a mature B cell, where V, D and J represent the combinatorial assortment of V, D and J segments and N represents the insertion or deletion of random DNA sequence at the splice junctions. Arrows represent the flanking IGH V and J primers that amplify the IGH gene region encoding the CDR3 domain. The IGHJseq primers are used to sequence 100 bases of the CDR3 region, sufficient to identify the V, D, and J segments and random N nucleotides that comprise the pathogen binding domain of the immunoglobulin.

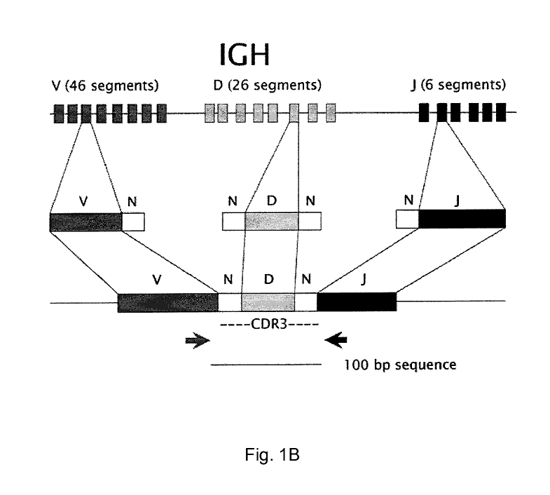

FIG. 2A shows the TCR gamma V-J usage in the peripheral blood of two donors.

FIG. 2B shows the TCR gamma V-J usage in saliva.

FIG. 3A shows the three dimensional representation of the IGTHV and IGHJ usage in 28 million sequences from B cells. The V segments are listed on the X axis, the J segments are listed on the Y axis and the number of observations of each pairing are shown on the Z axis.

FIG. 3B illustrates the lengths of the CDR3 sequences in all IGHV/IGHJ pairings. The CDR3 length is shown on the X axis, the IGHJ segment is listed on the Y axis and the number of observations is listed on Z axis.

### DETAILED DESCRIPTION

The present invention provides, in certain embodiments and as described herein, compositions and methods that are useful for characterizing large and structurally diverse populations of Adaptive Immune Receptors, such as immunoglobulins (Ig) and/or T cell receptors (TCR) that may be present in a biological sample from a subject or biological source, including a human subject. Disclosed herein are unexpectedly advantageous approaches by which partial DNA coding sequences can be readily determined for substantially all Adaptive Immune Receptors (TCR and/or Ig) that may be present in a biological sample, and from which partial sequences the diversity of Adaptive Immune Receptors in the sample can be quantitatively and qualitatively determined. In preferred embodiments, surprising adaptive immune receptor structural diversity can be characterized at the molecular and organismal levels, by determining and quantifying productively rearranged DNA sequences that encode TCR or Ig complementarity determining region-3 (CDR3), such as the CDR3 of a TCRγ or a TCRβ polypeptide chain or the CDR3 of an immunoglobulin heavy chain (referred to herein as IGH, IgH or Igh) polypeptide, along with V-region and/or J-region encoding sequences adjacent to the CDR3 encoding sequences.

In particular, and as explained in greater detail herein, the present embodiments relate in pertinent part to a strategy according to which coding sequences for TCR and/or Ig CDR3-containing regions may be determined for substantially all productively rearranged Adaptive Immune Receptor genes in a sample, such as genes that have been somatically rearranged to promote expression of functional T cell receptors and immunoglobulins. In certain embodiments, there are presently provided determination and quantification of the molecular sequence diversity in a sample of V-region polypeptide-encoding polynucleotide sequences, and in particular, of CDR3-encoding polynucleotides, for substantially all of one or more of the TCR α, β, γ, and δ chains and/or for one or more of Ig H and L chains, that may be present in the sample.

Compositions are provided that comprise a plurality of V-segment and J-segment primers that are capable of promoting amplification in a multiplex polymerase chain reaction (PCR) of substantially all productively rearranged adaptive immune receptor CDR3-encoding regions in the sample for a given class of such receptors (e.g., TCRγ, TCRβ, IgH, etc.), to produce a multiplicity of amplified rearranged DNA molecules from a population of T cells (for TCR) or B cells (for Ig) in the sample. Primers are designed in a manner that provides for the multiplicity of amplified rearranged DNA molecules to be sufficient, upon determination of every DNA sequence that has been amplified, to quantify diversity of the TCR or Ig CDR3-encoding region in the population of T or B cells. Preferably and in certain embodiments, primers are designed so that each amplified rearranged DNA molecule in the multiplicity of amplified rearranged DNA molecules is less than 600 nucleotides in length, thereby excluding amplification products from non-rearranged adaptive immune receptor loci.

In the human genome there are currently believed to be about 70 TCR Vα and about 61 Jα gene segments, about 52 TCR Vβ, about 2 Dβ and about 13 Jβ gene segments, about 9 TCR Vγ and about 5 Jγ gene segments, and about 46 immunoglobulin heavy chain (IGH) V_{H}, about 23 D_{H} and about 6 J_{H} gene segments. Accordingly, where genomic sequences for these loci are known such that specific molecular probes for each of them can be readily produced, it is believed according to non-limiting theory that the present compositions and methods relate to substantially all (e.g., greater than 90%, 91%, 92%, 93%, 94%, 95%, 96%, 97%, 98% or 99%) of these known and readily detectable adaptive immune receptor V-, D- and J-region encoding gene segments.

The TCR and Ig genes can generate millions of distinct proteins via somatic mutation. Because of this diversity-generating mechanism, the hypervariable complementarity determining regions of these genes can encode sequences that can interact with millions of ligands, and these regions are linked to a constant region that can transmit a signal to the cell indicating binding of the protein's cognate ligand.

The adaptive immune system employs several strategies to generate a repertoire of T- and B-cell antigen receptors with sufficient diversity to recognize the universe of potential pathogens. In αβ and γδ T cells, which primarily recognize peptide antigens presented by MHC molecules, most of this receptor diversity is contained within the third complementarity-determining region (CDR3) of the T cell receptor (TCR) α and β chains (or γ and δ chains). Although it has been estimated that the adaptive immune system can generate up to 10¹⁸ distinct TCR αβ pairs, direct experimental assessment of TCR CDR3 diversity has not been possible.

What is described herein is a novel method of measuring TCR and Ig CDR3 diversity that is based on single molecule DNA sequencing, and use this approach to sequence the CDR3 regions in millions of rearranged TCR and Ig genes of T and B cells isolated from peripheral blood and other tissues and bodily fluids such as, but not limited to, skin, colon, and saliva.

The ability of the adaptive immune system to mount an immune response specific for any of the vast number of potential foreign antigens to which an individual might be exposed relies on the highly variable receptors encoded by B cells (immunoglobulins) and T cells (T cell receptors; TCRs). The TCRs expressed by αβ T cells, which primarily recognize peptide antigens presented by major histocompatibility complex (MHC) class I and II molecules, are heterodimeric proteins consisting of two transmembrane polypeptide chains (α and β), each containing one variable and one constant domain. The peptide specificity of a T cells is in large part determined by the amino acid sequence encoded in the third complementarity-determining region (CDR3) loops of the α and β chain variable domains. The CDR3 regions of the β and α chains are formed by rearrangement of (i.e., such that the genes are no longer in their germline configuration) and recombination between noncontiguous variable (V_{β}), diversity (D_{β}), and joining (J_{β}) gene segments in the (chain locus, and between analogous V_{β} and J_{β} gene segments in the α chain locus, respectively. In TCRγ, the CDR3 domain is generated by V-J recombination. (Lefranc, M. P. and Lefranc, G., The T Cell Receptor Facts Book Academic Press 2001, which is herein incorporated by reference in its entirety.) The existence of multiple V, D and J gene segments in the TCR α, β and γ chain loci allows for a large number of distinct CDR3 sequences to be encoded. CDR3 sequence diversity is further increased by template-independent addition and deletion of nucleotides at the V_{β}-D_{β}, D_{β}-J_{β}, and V_{α}-J_{α} junctions during the process of TCR gene rearrangement.

During maturation of the progenitor B cell, the immunoglobulin genes are similarly assembled by rearrangement and recombination via splicing one of each of redundant V, D and J gene segments, where the pathogen-binding CDR3 domain of the antibody is encoded by the V(D)J sequence and hypervariable splice junctions. (Lefranc, M. P. and Lefranc, G., The Immunoglobulin FactsBook Academic Press 2001, which is herein incorporated by reference in its entirety.) Functional TCR and Ig encoding genes thus include those in which the germline DNA has been rearranged so that the relative positions of V, D and J encoding segments are no longer those found in germline DNA, whereby the recombination events that produce the rearranged adaptive immune receptor-(TCR- or Ig-) encoding DNA result in rearranged loci that are capable of productive TCR or Ig expression. For example, a functional TCR is expressed on a T cell surface, and is capable of TCR functions such as antigen recognition and binding and/or T cell activation signal transduction, and is encoded by rearranged functional TCR encoding genes which may comprise TCR V region-encoding and TCR J region-encoding gene segments. As another example, a functional Ig may be expressed on a B cell surface or secreted by cells of the B cell lineage (e.g., B cells or plasma cells), and is capable of Ig functions such as antigen recognition and binding and/or Ig effector functions, and is encoded by rearranged functional Ig encoding genes which may comprise Ig V region-encoding and Ig J region-encoding gene segments.

The sheer magnitude of possible CDR3 regions of these genes created by the splicing of the gene segments is estimated to be greater than one hundred million different sequence combinations and is so great it had not been possible to measure directly. In the absence of a DNA sequencing technology that is capable of directly assessing repertoire size, diversity in the T-cell repertoire has been indirectly assessed by a non-quantitative method to determine the distribution of lengths of TCR chain CDR3-encoding gene regions, a technique that is referred to as TCR “spectratyping.” However, spectratyping is a non-quantitative methodology that does not provide resolution at the level of DNA sequence. In other words, additional experimental methodology beyond spectratyping is desirable to identify and quantify uniquely rearranged CDR3-encoding sequences and to assess biomarkers in the receptor profile or disease state.

PCR-based methods have been previously developed to survey the diversity of the TCR and Ig repertoires in a sample, however these methods are limited in that they only capture single TCR sequences, and therefore are not capable of measuring or estimating the breadth and depth of the TCR and Ig repertoires in the sample. These previously described methodologies are limited because the copy numbers for any specifically identified sequences cannot be applied to quantification of the whole population of TCR or Ig repertoires. In other words, the small subset of a population of B or T cells that is sampled by these methods is insufficient to extrapolate to the whole cell population with any confidence.

Other alternative methods can involve the use of monoclonal antibodies or hybridization techniques to identify the TCR of individual clones, but these methods are unlikely to efficiently identify the rare sequences that may be most responsible for a disease state and/or the magnitude of the TCR repertoire because they are based on known IgH and TCR molecules which may not be associated with a particular disease state.

Thus there still is a need in the art for a platform independent methodology to identify directly mass numbers of individual Ig (heavy and light chain) and TCR (αβ and γδ) sequences on a large scale for use in identifying rare sequences associated with a disease state or abundant malignant clone sequences and thus creating therapeutic, diagnostic, prophylactic or predictive biomarkers.

As noted above, previous attempts to assess the diversity of receptors in the adult human u T cell repertoire relied on examining rearranged TCR α and β chain genes expressed in small, well-defined subsets of the repertoire, followed by extrapolation of the diversity present in these subsets to the entire repertoire, to arrive at an estimate of there being a total of approximately 10⁶ unique TCRβ chain CDR3 sequences per individual, with 10-20% of these unique TCRβ CDR3 sequences expressed by cells in the antigen-experienced CD45RO⁺ compartment. The accuracy and precision of this estimate are severely limited by the need to extrapolate. For instance, based on the degree of diversity observed in a sample yielding on the order of merely hundreds of TCR sequences, extrapolation must be used to project an estimate of the diversity of the entire TCR repertoire. It is possible that the actual number of unique TCR chain CDR3 sequences in the αβ T cell repertoire is significantly larger than 1×10⁶ unique TCRβ CDR3 sequences predicted by prior extrapolation methods.

Recent advances in high-throughput DNA sequencing technology have made possible significantly deeper sequencing than capillary-based technologies. For example, in current high-throughput sequencing methodologies such as those available from Illumina, Inc. (e.g., GeneAnalyzer™ GA2, Illumina, Inc., San Diego, Calif.), a complex library of heterogeneous template DNA molecules that have been modified to carry universal PCR adapter sequences at each end may be hybridized to a lawn of adapter-complementary oligonucleotides that has been immobilized on a solid surface. Solid phase PCR is utilized to amplify the hybridized library, resulting in millions of template clusters on the surface, each comprising multiple (˜1,000) identical copies of a single DNA molecule from the original library. A 30-54 bp interval in the molecules in each cluster is sequenced using reversible dye-termination chemistry. As described herein, appropriate selection of PCR oligonucleotide primers may permit simultaneous sequencing, from amplified genomic DNA, of the independently rearranged TCR or Ig CDR3-encoding regions carried in millions of T or B cells. This approach enables direct sequencing of a significant fraction of the uniquely rearranged TCR and Ig CDR3 regions in populations of T or B cells, which thereby permits estimation of the relative frequency of each CDR3 sequence in the population.

Accurate estimation of the diversity of TCR and Ig CDR3 sequences in the entire T or B cell repertoire from the diversity measured in a finite sample of T or B cells requires an estimate of the number of CDR3 sequences present in the repertoire that were not observed in the sample. TCR or Ig CDR3 diversity in the entire T or B cell repertoire being examined (e.g., TCRβ, TCRγ, IgH, etc.) can be estimated using direct measurements of the number of unique TCR or Ig CDR3 sequences observed in blood samples containing millions of αβ or γδ T cells or B cells.

The results described herein in the Examples identify a lower bound for TCRβ CDR3 diversity in the CD4⁺ and CD8⁺ T cell compartments that is several fold higher than previous estimates. In addition, the results herein demonstrate that there are at least 1.5×10⁶ unique TCRβ CDR3 sequences in the CD45RO⁺ compartment of antigen-experienced T-cells, a large proportion of which are present at low relative frequency. The existence of such a diverse population of TCRβ CDR3 sequences in antigen-experienced cells has not been previously demonstrated.

The diverse pool of TCRβ chains in each healthy individual is a sample from an estimated theoretical space of greater than 10¹¹ possible sequences. However, the realized set of rearranged of TCRs is not evenly sampled from this theoretical space. Different Vβs and Jβs are found with over a thousand-fold frequency difference. Additionally, the insertion rates of nucleotides are strongly biased. This reduced space of realized TCRβ sequences leads to the possibility of shared β chains between people. With the sequence data generated by the methods described herein, the in vivo J usage, V usage, mono- and di-nucleotide biases, and position dependent amino acid usage can be computed. These biases significantly narrow the size of the sequence space from which TCRβ are selected, suggesting that different individuals share TCRβ chains with identical amino acid sequences. Results herein show that many thousands of such identical sequences are shared pairwise between individual human genomes. Similar approaches as described herein pertain to the TCRγ and IgH loci. For example, at least hundreds of pairwise matching IgH sequences were detected just in the naïve B cell subset of the human B cell compartment, exclusive of the memory B cell subpopulation. Without wishing to be bound by theory, it is believed that the effects of antigen-specific selection pressure and somatic hypermutation of immunoglobulins are likely to underlie an even greater incidence of matching IgH sequences in the memory B cell pool.

The results described herein in the Examples further show that there exists diversity between the TCRγ V and J pairings in blood between donors. This result is surprising in view of reports in the literature stating the TCRγ in peripheral blood is restricted to a single dominant V9-JP pair (e.g., Triebel et al., 1988 J Exp Med. 167(2):694-9; PMID 2450164). The methods of the present invention showed that there are 35 pairings, including 32 in the bottom five percent of all sequences. These previously unseen, rare V-J pairings in the blood illustrate the sensitivity of the methods described herein for detecting potential TCRγ biomarkers for disease states.

Additionally, a TCRγ library was amplified and sequenced from saliva. As described in the Examples, results using the methods provided herein showed that the V-J pairings in the saliva TCRγ are distinct from the pattern observed in the blood, specifically a bias in pairings between V1-J1/2, V5-J1/2, and V11-JP1 suggesting the diversity of the TCRγ repertoire in the peripheral tissues exposed to the environment could harbor signals that can be used to monitor a disease state such as an autoimmune disease or an environmentally induced disease.

The present methods are also useful for determining diversity of T or B cell receptor in skin and other body tissues, such as oral, vaginal and intestinal mucosa. Results shown herein in the Examples indicate that the most common V-J pairing observed in skin was V9-JP, which is similar to blood and saliva. The V9-J1 pairing was also found at significant levels in skin, but was not observed in high levels in blood and saliva. The diversity of the TCRγ sequences in colon was distinct from the other tissues that were examined, in that the most prevalent TCRγ V segment observed in colon was the TCRγ V10 segment, and more V-J combinations were observed in colon than in blood, skin, or saliva.

The number of TCR sequences generated by the methods described herein far exceeds the number of all previously known TCRγ sequences prior to this disclosure. Therefore, the present disclosure provides in another embodiment methods for identifying a tissue-specific V-J usage bias in adaptive immune receptors in T cells (i.e., in TCR) or in B cells (e.g., in IgH). In certain embodiments, the present disclosure also provides methods for identifying a tissue-specific V-J usage bias associated with a disease of the tissue. Thus, the present disclosure provides methods for detecting disease by detecting tissue-specific V-J usage bias. By V-J bias is meant a statistically significant difference in the usage of specific V segments, specific J segments, or specific V-J combinations between two individuals, or in different tissues within an individual. This biological bias is distinct from any technical bias in the amplification of specific PCR products. In certain embodiments, By providing compositions and methods for identifying the CDR3-encoding sequences of substantially all productively rearranged TCRγ, TCRβ or IgH genes in a biological sample, the frequency of usage of any particular TCRγ (or TCRβ or IgH) V region-encoding gene and/or of any particular TCRγ (or TCRβ or IgH) J region-encoding gene can be quantified. Because the numbers of V-encoding and J-encoding genes are known for the human TCRγ, TCRβ and IgH loci, determination as described herein of the relative abundance of specific V- and J-encoding sequences in a sample permits, for the first time, accurate characterization of such quantitative biases in the rearrangement of particular V- and J-encoding genes.

The assay technology uses two pools of primers to provide for a highly multiplexed PCR reaction. The first, “forward” pool (e.g., by way of illustration and not limitation, V-segment oligonucleotide primers described herein may in certain preferred embodiments be used as “forward” primers when J-segment oligonucleotide primers are used as “reverse” primers according to commonly used PCR terminology, but the skilled person will appreciate that in certain other embodiments J-segment primers may be regarded as “forward” primers when used with V-segment “reverse” primers) includes an oligonucleotide primer that is specific to (e.g., having a nucleotide sequence complementary to a unique sequence region of) each V-region encoding segment (“V segment) in the respective TCR or Ig gene locus. In certain embodiments, primers targeting a highly conserved region are used, to simultaneously capture many V segments, thereby reducing the number of primers required in the multiplex PCR. Similarly, in certain embodiments, the “reverse” pool primers anneal to a conserved sequence in the joining (“J”) segment. Each primer may be designed so that a respective amplified DNA segment is obtained that includes a sequence portion of sufficient length to identify each J segment unambiguously based on sequence differences amongst known J-region encoding gene segments in the human genome database, and also to include a sequence portion to which a J-segment-specific primer may anneal for resequencing. This design of V- and J-segment-specific primers enables direct observation of a large fraction of the somatic rearrangements present in the adaptive immune receptor gene repertoire within an individual. This feature in turn enables rapid comparison of the TCR and/or Ig repertoires (i) in individuals having a particular disease, disorder, condition or other indication of interest (e.g., cancer, an autoimmune disease, an inflammatory disorder or other condition) with (ii) the TCR and/or Ig repertoires of control subjects who are free of such diseases, disorders conditions or indications.

The adaptive immune system can in theory generate an enormous diversity of T and B cell receptor CDR3 sequences— far more than are likely to be expressed in any one individual at any one time. Previous attempts to measure what fraction of this theoretical diversity is actually utilized in the adult αβ T cell repertoire, however, have not permitted accurate assessment of the diversity. What is described herein is the development of a novel approach to this question that is based on single molecule DNA sequencing, and in certain further embodiments, an analytic computational approach to estimation of repertoire diversity using diversity measurements in finite samples. The analysis demonstrated in the Examples herein show that the number of unique TCRβ CDR3 sequences in the adult repertoire significantly exceeds previous estimates, which were based on exhaustive capillary sequencing of small segments of the repertoire. The TCRβ chain diversity in the CD45RO⁻ population (enriched for naïve T cells) that was observed using the methods described herein was five-fold larger than previously reported. A major discovery is the number of unique TCRβ CDR3 sequences expressed in antigen-experienced CD45RO⁺ T cells—the results herein show that this number is between 10 and 20 times larger than expected based on previous results of others. The frequency distribution of CDR3 sequences in CD45RO⁺ cells suggests that the T cell repertoire contains a large number of clones that have a small clone size.

The results herein show that the realized set of TCR) chains are sampled non-uniformly from the huge potential space of sequences. In particular, the β chain sequences closer to germ line (few insertions and deletions at the V-D and D-J boundaries) appear to be created at a relatively high frequency. TCR sequences close to germ line are shared between different people because the germ line sequence for the Vs, Ds, and Js are shared, modulo a small number of polymorphisms, among the human population.

The T cell receptors expressed by mature αβ T cells are heterodimers whose two constituent chains are generated by independent rearrangement events of the TCR α and β chain variable loci. The a chain has less diversity than the β chain, so a higher fraction of αs are shared between individuals, and hundreds of exact TCR αβ receptors are shared between any pair of individuals.

Certain molecular biological techniques for use in the methods herein are known in the art and are described, for example, in Current Protocols in Molecular Biology, Second Edition, Ausubel et al. eds., John Wiley & Sons, 1992, or subsequent updates thereto; Current Protocols in Immunology (Edited by: John E. Coligan, Ada M. Kruisbeek, David H. Margulies, Ethan M. Shevach, Warren Strober 2001 John Wiley & Sons, NY, NY). Unless specific definitions are provided, the nomenclature utilized in connection with, and the laboratory procedures and techniques of, molecular biology, analytical chemistry, synthetic organic chemistry, and medicinal and pharmaceutical chemistry described herein are those well known and commonly used in the art. Standard techniques may be used for recombinant technology, molecular biological, microbiological, chemical syntheses, chemical analyses, pharmaceutical preparation, formulation, and delivery, and treatment of patients.

### Cells

B cells and T cells can be obtained in a biological sample, such as from a variety of tissue and biological fluid samples including marrow, thymus, lymph glands, lymph nodes, peripheral tissues and blood, but peripheral blood is most easily accessed. Any peripheral tissue can be sampled for the presence of B and T cells and is therefore contemplated for use in the methods described herein. Tissues and biological fluids from which adaptive immune cells may be obtained include, but are not limited to skin, epithelial tissues, colon, spleen, a mucosal secretion, oral mucosa, intestinal mucosa, vaginal mucosa or a vaginal secretion, cervical tissue, ganglia, saliva, cerebrospinal fluid (CSF), bone marrow, cord blood, serum, serosal fluid, plasma, lymph, urine, ascites fluid, pleural fluid, pericardial fluid, peritoneal fluid, abdominal fluid, culture medium, conditioned culture medium or lavage fluid. In certain embodiments, adaptive immune cells may be isolated from an apheresis sample. Peripheral blood samples may be obtained by phlebotomy from subjects. Peripheral blood mononuclear cells (PBMC) are isolated by techniques known to those of skill in the art, e.g., by Ficoll-Hypaque® density gradient separation. In certain embodiments, whole PBMCs are used for analysis.

In one embodiment, specific subpopulations of T or B cells are isolated prior to analysis using the methods described herein. Various methods and commercially available kits for isolating different subpopulations of T and B cells are known in the art and include, but are not limited to subset selection immunomagnetic bead separation or flow immunocytometric cell sorting using antibodies specific for one or more of any of a variety of known T and B cell surface markers. Illustrative markers include, but are not limited to, one or a combination of CD2, CD3, CD4, CD8, CD14, CD19, CD20, CD25, CD28, CD45RO, CD45RA, CD54, CD62, CD62L, CDw137 (41BB), CD154, G1TR, FoxP3, CD54, and CD28. For example, and as is known to the skilled person, cell surface markers, such as CD2, CD3, CD4, CD8, CD14, CD19, CD20, CD45RA, and CD45RO may be used to determine T, B, and monocyte lineages and subpopulations in flow cytometry. Similarly, forward light-scatter, side-scatter, and/or cell surface markers such as CD25, CD62L, CD54, CD137, CD154 may be used to determine activation state and functional properties of cells.

Illustrative combinations useful in certain of the methods described herein may include CD8⁺CD45RO⁺ (memory cytotoxic T cells), CD4⁺CD45RO⁺ (memory T helper), CD8⁺CD45RO⁻ (CD8⁺CD62L⁺CD45RA⁺ (naïve-like cytotoxic T cells); CD4⁺CD25⁺CD62L^{hi}GITR⁺FoxP3⁺ (regulatory T cells). Illustrative antibodies for use in immunomagnetic cell separations or flow immunocytometric cell sorting include fluorescently labeled anti-human antibodies, e.g., CD4 FITC (clone M-T466, Miltenyi Biotec), CD8 PE (clone RPA-T8, BD Biosciences), CD45RO ECD (clone UCHL-1, Beckman Coulter), and CD45RO APC (clone UCHL-G, BD Biosciences). Staining of total PBMCs may be done with the appropriate combination of antibodies, followed by washing cells before analysis. Lymphocyte subsets can be isolated by fluorescence activated cell sorting (FACS), e.g., by a BD FACSAria™ cell-sorting system (BD Biosciences) and by analyzing results with FlowJo™ software (Treestar Inc.), and also by conceptually similar methods involving specific antibodies immobilized to surfaces or beads.

### Nucleic Acid Extraction

Total genomic DNA is extracted from cells using methods known in the art and/or commercially available kits, e.g., by using the QIAamp® DNA blood Mini Kit (QIAGEN®). The approximate mass of a single haploid genome is 3 pg. Preferably, at least 100,000 to 200,000 cells are used for analysis of diversity, i.e., about 0.6 to 1.2 μg DNA from diploid T or B cells. Using PBMCs as a source, the number of T cells can be estimated to be about 30% of total cells. The number of B cells can also be estimated to be about 30% of total cells.

Alternatively, total nucleic acid can be isolated from cells, including both genomic DNA and mRNA. If diversity is to be measured from mRNA in the nucleic acid extract, the mRNA must be converted to cDNA prior to measurement. This can readily be done by methods of one of ordinary skill, for example, using reverse transcriptase according to known procedures.

### DNA Amplification

A multiplex PCR system is used to amplify rearranged adaptive immune cell loci from genomic DNA, preferably from a CDR3-encoding region. In certain embodiments, the CDR3-encoding region is amplified from a TCRα, TCRβ, TCRγ or TCRδ CDR3 region or from an IgH or IgL (lambda or kappa) locus.

In general, a multiplex PCR system may use at least 14, 15, 16, 17, 18, 19, 20, 21, 22, 23, 24, or 25, and in certain embodiments, at least 26, 27, 28, 29, 30, 31, 32, 33, 34, 35, 36, 37, 38, or 39, and in other embodiments 40, 41, 42, 43, 44, 45, 46, 47, 48, 49, or 50 or more “first” (e.g., “forward”) primers, in which each first or forward primer is capable of specifically hybridizing to a genomic DNAsequence (or to a cDNA sequence that has been reverse-transcribed from mRNA) corresponding to one or more V region-encoding segments. Illustrative V region primers for amplification of the TCRβ are shown in SEQ ID NOS: 114-248. Illustrative TCRγ V region primers are provided in SEQ ID NOs:485-488. Illustrative IgH V region primers are provided in SEQ ID NOs:505-588.

The multiplex PCR system also uses at least 3, 4, 5, 6, or 7, and in certain embodiments, 8, 9, 10, 11, 12 or 13 “second” (e.g., “reverse”) primers, in which each second or reverse primer is capable of specifically hybridizing to a genomic DNA sequence (or a cDNA sequence) corresponding to one or more J region-encoding segments. Illustrative TCRβ J segment primers are provided in SEQ ID NOS:249-261. Illustrative TCRγ J segment primers are provided in SEQ ID NOs:493-496. Illustrative IgH J segment primers are provided in SEQ ID NOs:499-504. In one embodiment, there is a J segment primer for every J segment.

Oligonucleotides or polynucleotides that are capable of specifically hybridizing or annealing to a target nucleic acid sequence by nucleotide base complementarity may do so under moderate to high stringency conditions. For purposes of illustration, suitable moderate to high stringency conditions for specific PCR amplification of a target nucleic acid sequence would be between 25 and 80 PCR cycles, with each cycle consisting of a denaturation step (e.g., about 10-30 seconds (s) at greater than about 95° C.), an annealing step (e.g., about 10-30s at about 60-68° C.), and an extension step (e.g., about 10-60s at about 60-72° C.), optionally according to certain embodiments with the annealing and extension steps being combined to provide a two-step PCR. As would be recognized by the skilled person, other PCR reagents may be added or changed in the PCR reaction to increase specificity of primer annealing and amplification, such as altering the magnesium concentration, optionally adding DMSO, and/or the use of blocked primers, modified nucleotides, peptide-nucleic acids, and the like.

In certain embodiments, nucleic acid hybridization techniques may be used to assess hybridization specificity of the primers described herein. Hybridization techniques are well known in the art of molecular biology. For purposes of illustration, suitable moderately stringent conditions for testing the hybridization of a polynucleotide as provided herein with other polynucleotides include prewashing in a solution of 5×SSC, 0.5% SDS, 1.0 mM EDTA (pH 8.0); hybridizing at 50° C.-60° C., 5×SSC, overnight; followed by washing twice at 65° C. for 20 minutes with each of 2×, 0.5× and 0.2×SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C.-70° C.

In certain embodiments, the primers are designed not to hybridize to genomic DNA across an intron/exon boundary. The first (forward) primers may comprise V-segment primers that in certain embodiments anneal (e.g., specifically hybridize) to the polynucleotide sequence encoding an adaptive immune receptor (TCR or Ig) V-region polypeptide (e.g., a V-segment) in a polynucleotide region of relatively strong sequence conservation between V-regions, so as to maximize the conservation of sequence among these primers. Accordingly, this oligonucleotide primer design strategy may, according to non-limiting theory, minimize the potential for each different primer to have significantly different annealing properties (e.g., for a candidate primer to exhibit a significantly increased or significantly decreased degree of detectable annealing to a complementary target sequence and amplification, relative to the degree of detectable annealing of a structurally unrelated control primer to its complementary target sequence and amplification, under comparable annealing and extension conditions). Further according to these and related embodiments, the amplified region between V and J primers may contain sufficient TCR or Ig V sequence information to permit identification of the specific V gene segment used, based on known genomic sequences for adaptive immune receptor (TCR and Ig) gene loci.

In certain embodiments, the “second” (e.g., reverse) J segment primers hybridize to a polynucleotide sequence encoding a conserved element of the adaptive immune receptor J-region polypeptide (J segment), and have similar annealing strength. In one embodiment, all J segment primers anneal to the same conserved framework region motif. The forward and reverse primers are both preferably modified at their 5′ ends with a universal forward primer sequence that is compatible with a DNA sequencer (e.g., Illumina GeneAnalyzer™2 (GA2) system, available from Illumina, Inc., San Diego, Calif.).

In particular embodiments, oligonucleotide primers for use in the compositions and methods described herein may comprise or consist of a nucleic acid of at least about 15 nucleotides long that has the same sequence as, or is complementary to, a 15 nucleotide long contiguous sequence of the target V- or J-segment (i.e., portion of genomic polynucleotide encoding a V-region or J-region polypeptide). Longer primers, e.g., those of about 16, 17, 18, 19, 20, 21, 22, 23, 24, 25, 26, 27, 28, 29, 30, 31, 32, 33, 34, 35, 36, 37, 38, 39, 40, 45, or 50, nucleotides long that have the same sequence as, or sequence complementary to, a contiguous sequence of the target V- or J-region encoding polynucleotide segment, will also be of use in certain embodiments. All intermediate lengths of the presently described oligonucleotide primers are contemplated for use herein. As would be recognized by the skilled person, the primers may have additional sequence added (e.g., nucleotides that may not be the same as or complementary to the target V- or J-region encoding polynucleotide segment), such as restriction enzyme recognition sites, adaptor sequences for sequencing, bar code sequences, and the like (see e.g., primer sequences provided in the Tables and sequence listing herein). Therefore, the length of the primers may be longer, such as about 55, 56, 57, 58, 59, 60, 61, 62, 63, 64, 65, 66, 67, 68, 69, 70, 71, 72, 73, 74, 75, 80, 85, 90, 95, 100 or more nucleotides in length or more, depending on the specific use or need.

Also contemplated for use in certain embodiments are adaptive immune receptor V-segment or J-segment oligonucleotide primer variants that may share a high degree of sequence identity to the oligonucleotide primers for which nucleotide sequences are presented herein, including those set forth in the Sequence Listing. Thus, in these and related embodiments, adaptive immune receptor V-segment or J-segment oligonucleotide primer variants may have substantial identity to the adaptive immune receptor V-segment or J-segment oligonucleotide primer sequences disclosed herein, for example, such oligonucleotide primer variants may comprise at least 70% sequence identity, preferably at least 75%, 80%, 85%, 90%, 91%, 92%, 93%, 94%, 95%, 96%, 97%, 98%, or 99% or higher sequence identity compared to a reference polynucleotide sequence such as the oligonucleotide primer sequences disclosed herein, using the methods described herein (e.g., BLAST analysis using standard parameters). One skilled in this art will recognize that these values can be appropriately adjusted to determine corresponding ability of an oligonucleotide primer variant to anneal to an adaptive immune receptor segment-encoding polynucleotide by taking into account codon degeneracy, reading frame positioning and the like. Typically, oligonucleotide primer variants will contain one or more substitutions, additions, deletions and/or insertions, preferably such that the annealing ability of the variant oligonucleotide is not substantially diminished relative to that of an adaptive immune receptor V-segment or J-segment oligonucleotide primer sequence that is specifically set forth herein. As also noted elsewhere herein, in preferred embodiments adaptive immune receptor V-segment and J-segment oligonucleotide primers are designed to be capable of amplifying a rearranged TCR or IGH sequence that includes the coding region for CDR3.

A multiplex PCR system may use 45 forward primers, each specific to a functional TCR or Ig V-region encoding segment, e.g., a TCR Vβ segment, (see e.g., the TCR primers as shown in Table 1), and thirteen reverse primers, each specific to a TCR or Ig J-region encoding segment, such as TCR Jβ segment (see e.g., Table 2). In another embodiment, a multiplex PCR reaction may use four forward primers each specific to one or more functional TCRγ V-region encoding segment and four reverse primers each specific for one or more TCRγ J-region encoding segments (see e.g., Table 15). In another embodiment, a multiplex PCR reaction may use four forward primers each specific to one or more functional V-region encoding segments and six reverse primers each specific for one or more J-region encoding segments (see e.g., IgH amplification primers provided in Table 17). With regard to the illustrative primers provided in the tables herein, Xn and Yn correspond to polynucleotides of lengths n and m, respectively, which comprise sequences that are specific to a single-molecule sequencing technology being employed, for example the GA2 system (Illumina, Inc., San Diego, Calif.) or other suitable sequencing suite of instrumentation, reagents and software.

TABLE 1
TCR-VB Forward primer sequences

| | SEQ | |
|---|---|---|
| TRBV gene | ID | |
| segment(s) | NO: | Primer sequence* |
| TRBV2 | 1 | XnTCAAATTTCACTCTGAAGATCCGGTCCAC |
| | | AA |
| TRBV3-1 | 2 | XnGCTCACTTAAATCTTCACATCAATTCCCT |
| | | GG |
| TRBV4-1 | 3 | XnCTTAAACCTTCACCTACACGCCCTGC |
| TRBV(4-2, 4-3) | 4 | XnCTTATTCCTTCACCTACACACCCTGC |
| TRBV5-1 | 5 | XnGCTCTGAGATGAATGTGAGCACCTTG |
| TRBV5-3 | 6 | XnGCTCTGAGATGAATGTGAGTGCCTTG |
| TRBV(5-4, 5-5, | 7 | XnGCTCTGAGCTGAATGTGAACGCCTTG |
| 5-6, 5-7, 5-8) | | |
| TRBV6-1 | 8 | XnTCGCTCAGGCTGGAGTCGGCTG |
| TRBV(6-2, 6-3) | 9 | XnGCTGGGGTGGAGTCGGCTG |
| TRBV6-4 | 10 | XnCCCTCACGTTGGCGTCTGCTG |
| TRBV6-5 | 11 | XnGCTCAGGCTGCTGTCGGCTG |
| TRBV6-6 | 12 | XnCGCTCAGGCTGGAGTTGGCTG |
| TRBV6-7 | 13 | XnCCCCTCAAGCTGGAGTCAGCTG |
| TRBV6-8 | 14 | XnCACTCAGGCTGGTGTCGGCTG |
| TRBV6-9 | 15 | XnCGCTCAGGCTGGAGTCAGCTG |
| TRBV7-1 | 16 | XnCCACTCTGAAGTTCCAGCGCACAC |
| TRBV7-2 | 17 | XnCACTCTGACGATCCAGCGCACAC |
| TRBV7-3 | 18 | XnCTCTACTCTGAAGATCCAGCGCACAG |
| TRBV7-4 | 19 | XnCCACTCTGAAGATCCAGCGCACAG |
| TRBV7-6 | 20 | XnCACTCTGACGATCCAGCGCACAG |
| TRBV7-7 | 21 | XnCCACTCTGACGATTCAGCGCACAG |
| TRBV7-8 | 22 | XaCCACTCTGAAGATCCAGCGCACAC |
| TRBV7-9 | 23 | XnCACCTTGGAGATCCAGCGCACAG |
| TRBV9 | 24 | XnGCACTCTGAACTAAACCTGAGCTCTCTG |
| TRBV10-1 | 25 | XnCCCCTCACTCTGGAGTCTGCTG |
| TRBV10-2 | 26 | XnCCCCCTCACTCTGGAGTCAGCTA |
| TRBV10-3 | 27 | XnCCTCCTCACTCTGGAGTCCGCTA |
| TRBV(11-1, 11-3) | 28 | XnCCACTCTCAAGATCCAGCCTGCAG |
| TRBV11-2 | 29 | XnCTCCACTCTCAAGATCCAGCCTGCAA |
| TRBV(12-3, 12-4, | 30 | XnCCACTCTGAAGATCCAGCCCTCAG |
| 12-5) | | |
| TRBV13 | 31 | XnCATTCTGAACTGAACATGAGCTCCTTGG |
| TRBV14 | 32 | XnCTACTCTGAAGGTGCAGCCTGCAG |
| TRBV15 | 33 | XnGATAACTTCCAATCCAGGAGGCCGAACA |
| TRBV16 | 34 | XnCTGTAGCCTTGAGATCCAGGCTACGA |
| TRBV17 | 35 | XnCTTCCACGCTGAAGATCCATCCCG |
| TRBV18 | 36 | XnGCATCCTGAGGATCCAGCAGGTAG |
| TRBV19 | 37 | XnCCTCTCACTGTGACATCGGCCC |
| TRBV20-1 | 38 | XnCTTGTCCACTCTGACAGTGACCAGTG |
| TRBV23-1 | 39 | XnCAGCCTGGCAATCCTGTCCTCAG |
| TRBV24-1 | 40 | XnCTCCCTGTCCCTAGAGTCTGCCAT |
| TRBV25-1 | 41 | XnCCCTGACCCTGGAGTCTGCCA |
| TRBV27 | 42 | XnCCCTGATCCTGGAGTCGCCCA |
| TRBV28 | 43 | XnCFCCCTGATTCTGGAGTCCGCCA |
| TRBV29-1 | 44 | XnCTAACATTCTCAACTCTGACTGTGAGCAA |
| | | CA |
| TRBV30 | 45 | XnCGGCAGTTCATCCTGAGTTCTAAGAAGC |

TABLE 2
TCR-Jβ Reverse Primer Sequences

| TRBJ gene | SEQ | |
|---|---|---|
| segment | ID NO: | Primer sequence* |
| TRBJ1-1 | 46 | YmTTACCTACAACTGTGAGTCTGGTGCCTTGTCC |
| | | AAA |
| TRBJI-2 | 47 | YmACCTACAACGGTTAACCTGGTCCCCGAACCGAA |
| TRBJ1-3 | 48 | YmACCTACAACAGTGAGCCAACTTCCCTCTCCAAA |
| TRBJ1-4 | 49 | YmCCAAGACAGAGAGCTGGGTTCCACTGCCAAA |
| TRBJ1-5 | 483 | YmACCTAGGATGGAGAGTCGAGTCCCATCACCAAA |
| TRBJ1-6 | 50 | YmCTGTCACAGTGAGCCTGGTCCCGTTCCCAAA |
| TRBJ2-1 | 51 | YmCGGTGAGCCGTGTCCCTGGCCCGAA |
| TRBJ2-2 | 52 | YmCCAGTACGGTCAGCCTAGAGCCTTCTCCAAA |
| TRBJ2-3 | 53 | YmACTGTCAGCCGGGTGCCTGGGCCAAA |
| TRBJ2-4 | 54 | YmAGAGCCGGGTCCCGGCGCCGAA |
| TRBJ2-5 | 55 | YmGGAGCCGCGTGCCTGGCCCGAA |
| TRBJ2-6 | 56 | YmGTCAGCCTGCTGCCGGCCCCGAA |
| TRBJ2-7 | 57 | YmGTGAGCCTGGTGCCCGGCCCGAA |

The 45 forward PCR primers of Table 1 are each complementary to one or more of the 48 functional TCR variable region-encoding (V) gene segments (referred to as TRBV in Table 1), and the thirteen reverse PCR primers of Table 2 are each complementary to one or more of the functional TCR joining region-encoding (J) gene segments from the TCRB locus (referred to as TRBJ in Table 2). The TCRB V region segments are identified in the Sequence Listing at SEQ ID NOS:114-248 and the TCRB J region segments are at SEQ ID NOS:249-261. Polynucleotide sequences of the TCRG J region segments are set forth in SEQ ID NOs:595-600. Polynucleotide sequences of the TCRG V region segments are set forth in SEQ ID NOs:601-618. Polynucleotide sequences of the IgH J region segments are set forth in SEQ ID NOs:619-634. Polynucleotide sequences of the IgH V region segments are set forth in SEQ ID NOs:635-925.

In certain preferred embodiments, the V-segment and J-segment oligonucleotide primers as described herein are designed to include nucleotide sequences such that adequate information is present within the sequence of an amplification product of a rearranged adaptive immune receptor (TCR or Ig) gene to identify uniquely both the specific V and the specific J genes that give rise to the amplification product in the rearranged adaptive immune receptor locus (e.g., at least 1, 2, 3, 4, 5, 6, 7, 8, 9, 10, 11, 12, 13, 14, 15, 16, 17, 18, 19 or 20 base pairs of sequence upstream of the V gene recombination signal sequence (RSS), preferably at least about 22, 24, 26, 28, 30, 32, 34, 35, 36, 37, 38, 39 or 40 base pairs of sequence upstream of the V gene recombination signal sequence (RSS), and in certain preferred embodiments greater than 40 base pairs of sequence upstream of the V gene recombination signal sequence (RSS), and at least 1, 2, 3, 4, 5, 6, 7, 8, 9, 10, 11, 12, 13, 14, 15, 16, 17, 18, 19 or 20 base pairs downstream of the J gene RSS, preferably at least about 22, 24, 26, 28 or 30 base pairs downstream of the J gene RSS, and in certain preferred embodiments greater than 30 base pairs downstream of the J gene RSS).

This feature stands in contrast to oligonucleotide primers described in the art for amplification of TCR-encoding or Ig-encoding gene sequences, which rely primarily on the amplification reaction merely for detection of presence or absence of products of appropriate sizes for V and J segments (e.g., the presence in PCR reaction products of an amplicon of a particular size indicates presence of a V or J segment but fails to provide the sequence of the amplified PCR product and hence fails to confirm its identity, such as the common practice of spectratyping).

Alternative primers to those described herein may be selected by a person of ordinary skill based on the present disclosure and knowledge in the art regarding published gene sequences for the V- and J-encoding regions of the genes for each TCR and Ig subunit (see e.g., SEQ ID NOs:114-261 and 595-925). Reference Genbank entries for human adaptive immune receptor sequences include: TCRα (TCRA/D): NC_{—}000014.8 (chr14:22090057..23021075); TCRβ: (TCRB): NC_{—}000007.13 (chr7:141998851..142510972); TCRγ: (TCRG): NC_{—}000007.13 (chr7:38279625..38407656); immunoglobulin heavy chain, IgH (IGH): NC_{—}000014.8 (chr14: 106032614..107288051); immunoglobulin light chain-kappa, IgLκ (IGK): NC_{—}000002.11 (chr2: 89156874..90274235); and immunoglobulin light chain-lambda, IgLλ (IGL): NC_{—}000022.10 (chr22: 22380474..23265085). Reference Genbank entries for mouse adaptive immune receptor loci sequences include: TCRβ: (TCRB): NC_{—}000072.5 (chr6: 40841295..41508370), and immunoglobulin heavy chain, IgH (IGH): NC_{—}000078.5 (chr12:114496979..117248165).

Primer design analyses and target site selection considerations can be performed, for example, using the OLIGO primer analysis software and/or the BLASTN 2.0.5 algorithm software (Altschul et al., Nucleic Acids Res. 1997, 25(17):3389-402), or other similar programs available in the art. Accordingly, based on the present disclosure and in view of these known adaptive immune receptor gene sequences and primer design methodologies, it is within the art to design V region-specific and J region-specific primers that are capable of annealing to substantially all V genes and substantially all J genes in a given adaptive immune receptor-encoding locus (e.g., a human TCR or IgH locus) and that permit generation in multiplexed (e.g., using multiple forward and reverse primer pairs) PCR of PCR amplification products that have a first end that is encoded by a rearranged V region-encoding gene segment and a second end that is encoded by a J region-encoding gene segment. Typically such amplification products will include a CDR3-encoding sequence. The primers may be preferably designed to yield amplification products having sufficient portions of V and J sequences such that by sequencing the products (amplicons), it is possible to identify on the basis of sequences that are unique to each gene segment (i) the particular V gene, and (ii) the particular J gene in the proximity of which the V gene underwent productive rearrangement to yield a functional adaptive immune receptor-encoding gene. Typically, and in preferred embodiments, the PCR amplification products will not be more than 600 base pairs in size, which according to non-limiting theory will exclude amplification products from non-rearranged adaptive immune receptor genes.

The forward primers described herein may be modified at the 5′ end with the universal forward primer sequence compatible with the DNA sequencer (Xn of Table 1). Similarly, the reverse primers may be modified with a universal reverse primer sequence (Ym of Table 2). Examples of such universal primers are shown in Tables 3 and 4, for the Illumina GAII single-end read sequencing system. As would be recognized by the skilled person, in certain embodiments, other modifications may be made to the primers, such as the addition of restriction enzyme sites, fluorescent tags, and the like, depending on the specific application.

For TCRβ chain sequences, the 45 TCR Vβ-segment forward primers anneal to the complementary Vβ-region encoding gene segments in a region of relatively strong sequence conservation between Vβ segments, so as to permit maximization of the conservation of sequence among these primers.

TABLE 3
TCR-Vβ Forward primer sequences

| TRBV | | |
|---|---|---|
| gene | SEQ | |
| segment(s) | ID NO: | primer sequence* |
| TRBV2 | 58 | CAAGCAGAAGACGGCATACGAGCTCTT |
| | | CCGATCTTCAAATTTCACTCTGAAGATCC |
| | | GGTCCACAA |
| TRBV3-1 | 59 | CAAGCAGAAGACGGCATACGAGCTCTT |
| | | CCGATCTGCTCACTTAAATCTTCACATCA |
| | | ATTCCCTGG |
| TRBV4-1 | 60 | CAAGCAGAAGACGGCATACGAGCTCTT |
| | | CCGATCTCTTAAACCTTCACCTACACGCC |
| | | CTGC |
| TRBV(4-2, | 61 | CAAGCAGAAGACGGCATACGAGCTCTT |
| 4-3) | | CCGATCTCTTATTCCTTCACCTACACACC |
| | | CTGC |
| TRBV5-1 | 62 | CAAGCAGAAGACGGCATACGAGCTCTT |
| | | CCGATCTGCTCTGAGATGAATGTGAGCAC |
| | | MG |
| TRBV5-3 | 63 | CAAGCAGAAGACGGCATACGAGCTCTT |
| | | CCGATCTGCTCTGAGATGAATGTGAGTGC |
| | | CTTG |
| TRBV (5-4, | 64 | CAAGCAGAAGACGGCATACGAGCTCTT |
| 5-5, 5-6, | | CCGATCTGCTCTGAGCTGAATGTGAACGC |
| 5-7, 5-8) | | CTTG |
| TRBV6-1 | 65 | CAAGCAGAAGACGGCATACGAGCTCTT |
| | | CCGATCTTCGCTCAGGCTGGAGTCGGCTG |
| TRBV(6-2,) | 66 | CAAGCAGAAGACGGCATACGAGCTCTT |
| 6-3 | | CCGATCTGCTGGGGTTGGAGTCGGCTG |
| TRBV6-4 | 67 | CAAGCAGAAGACGGCATACGAGCTCTT |
| | | CCGATCTCCCTCACGTTGGCGTCTGCTG |
| TRBV6-5 | 68 | CAAGCAGAAGACGGCATACGAGCTCTT |
| | | CCGATCTGCTCAGGCTGCTGTCGGCTG |
| TRBV6-6 | 69 | CAAGCAGAAGACGGCATACGAGCTCTT |
| | | CCGATCTCGCTCAGGCTGGAGTTGGCTG |
| TRBV6-7 | 70 | CAAGCAGAAGACGGCATACGAGCTCTT |
| | | CCGATCTCCCCTCAAGCTGGAGTCAGCTG |
| TR13V6-8 | 71 | CAAGCAGAAGACGGCATACGAGCTerr |
| | | CCGATCTCACTCAGGCTGGTGTCGGCTG |
| TRBV6-9 | 72 | CAAGCAGAAGACGGCATACGAGCTCTT |
| | | CCGATCTCGCTCAGGCTGGAGTCAGCTG |
| TRBV7-1 | 73 | CAAGCAGAAGACGGCATACGAGCTCTT |
| | | CCGATCTCCACTCTGAACTTTCCAGCGCAC |
| | | AC |
| TRBV7-2 | 74 | CAAGCAGAAGACGGCATACGAGCTCTT |
| | | CCCATCTCACTCTGACGATCCAGCGCACA |
| | | C |
| TRBV7-3 | 75 | CAAGCAGAAGACGGCATACGAGCTCTT |
| | | CCGATCTCTCTACTCTGAAGATCCAGCGC |
| | | ACAG |
| TRBV7-4 | 76 | CAAGCAGAAGACGGCATACGAGCTCTT |
| | | CCGATCTCCACTCTGAAGATCCAGCGCAC |
| | | AG |
| TRBV7-6 | 77 | CAAGCAGAAGACGGCATACGAGCTCTT |
| | | CCGATCTCACTCTGACGATCCAGCGCACA |
| | | G |
| TRBV7-7 | 78 | CAAGCAGAAGACGGCATACGAGCTCTT |
| | | CCGATCTCCACTCTGACGATTCAGCGCAC |
| | | AG |
| TRBV7-8 | 79 | CAAGCAGAAGACGGCATACGAGCTCTT |
| | | CCGATCTCCACTCTGAAGATCCAGCGCAC |
| | | AC |
| TRBV7-9 | 80 | CAAGCAGAAGACGGCATACGAGCTCTT |
| | | CCGATCTCACCTTGGAGATCCAGCGCACA |
| | | G |
| TRBV9 | 81 | CAAGCAGAAGACGGCATACGAGCTCTT |
| | | CCGATCTGCACTCTGAACTAAACCTGAGC |
| | | TCTCTG |
| TRBV10-1 | 82 | CAAGCAGAAGACGGCATACGAGCTCTT |
| | | CCGATCTCCCCTCACTCTGGAGTCTGCTG |
| TRBV10-2 | 83 | CAAGCAGAAGACGGCATACGAGCTCTT |
| | | CCGATCTCTTCCACTCTGGAGTCACTCT |
| | | A |
| TRBV10-3 | 84 | CAAGCAGAAGACGGCATACGAGCTCTT |
| | | CCGATCTCCTCCTCACTCTGGAGTCCGCT |
| | | A |
| TRBV(11-1, | 85 | CAAGCAGAAGACGGCATACGAGCTCTT |
| 11-3) | | CCGATCTCCACTCTCAAGATCCAGCCTGC |
| | | AG |
| TRBV11-2 | 86 | CAAGCAGAAGACGGCATACGAGCTCTT |
| | | CCGATCTCTCCACTCTCAAGATCCAGCCT |
| | | GCAA |
| TRBV(12-3, | 87 | CAAGCAGAAGACGGCATACGAGCTCTT |
| 12-4, 12-5) | | CCGATCTCCACTCTGAAGATCCAGCCCTC |
| | | AG |
| TRBV13 | 88 | CAAGCAGAAGACGGCATACGAGCTCTT |
| | | CCGATCTCATTCTGAACTGAACATGAGCT |
| | | CCTTGG |
| TRBV14 | 89 | CAAGCAGAAGACGGCATACGAGCTCTT |
| | | CCGATCTCTACTCTGAAGGTGCAGCCTGC |
| | | AG |
| TRBV15 | 90 | CAAGCAGAAGACGGCATACGAGCTCTT |
| | | CCGATCTGATAACTTCCAATCCAGGAGGC |
| | | CGAACA |
| TRBV16 | 91 | CAAGCAGAAGACGGCATACGAGCTCTT |
| | | CCGATCTCTGTAGCCTTGAGATCCAGGCT |
| | | ACGA |
| TRBV17 | 92 | CAAGCAGAAGACGGCATACGAGCTCTT |
| | | CCGATCTCTTCCACGCTGAAGATCCATCC |
| | | CG |
| TRBV18 | 93 | CAAGCAGAAGACGGCATACGAGCTCTT |
| | | CCGATCTGCATCCTGAGGATCCAGCAGGT |
| | | AG |
| TRBV19 | 94 | CAAGCAGAAGACGGCATACGAGCTCTT |
| | | CCGATCTCCTCTCACTGTGACATCGGCCC |
| TRBV20-1 | 95 | CAAGCAGAAGACGGCATACGAGCTCTT |
| | | CCGATCTCTTGTCCACTCTGACAGTGACC |
| | | TAGTG |
| TRBV23-1 | 96 | CAAGCAGAAGACGGCATACGAGCTCTT |
| | | CCGATCTCAGCCTGGCAATCCTGTCCTCA |
| | | G |
| TRBV24-1 | 97 | CAAGCAGAAGACGGCATACGAGCTCTT |
| | | CCGATCTCTCCCTGTCCCTAGAGTCTGCC |
| | | AT |
| TRBV25-1 | 98 | CAAGCAGAAGACGGCATACGAGCTCTT |
| | | CCGATCTCCCTGACCCTGGAGTCTGCCA |
| TRBV27 | 99 | CAAGCAGAAGACGGCATACGAGCTCTT |
| | | CCGATCTCCCTGATCCTGGAGTCGCCCA |
| TRBV28 | 100 | CAAGCAGAAGACGGCATACGAGCTCTT |
| | | CCGATCTCTCCCTGATTCTGGAGTCCGCC |
| | | A |
| TRBV29-1 | 101 | CAAGCAGAAGACGGCATACGAGCTCTT |
| | | CCGATCTCTAACATTCTCAACTCTGACTG |
| | | TGAGCAACA |
| TRBV30 | 102 | CAAGCAGAAGACGGCATACGAGCTCTT |
| | | CCGATCTCGGCAGTTCATCCTGAGTTCTA |
| | | AGAAGC |

TABLE 4
TCR-Jβ Reverse Primer Sequences

| TRBJ | SEQ | |
|---|---|---|
| gene | ID | |
| segment | NO: | Primer sequence* |
| TRBJ1-1 | 103 | AATGATACGGCGACCACCGAGATCTTTACCT |
| | | ACAACTGTGAGTCTGGTGCCTTGTCCAAA |
| TRBJ1-2 | 468 | AATGATACGGCGACCACCGAGATCTACCTA |
| | | CAACGGTTAACCTGGTCCCCGAACCGAA |
| TRBJ1-3 | 104 | AATGATACGGCGACCACCGAGATCTACCTA |
| | | CAACAGTGAGCCAACTTCCCTCTCCAAA |
| TRBJ1-4 | 105 | AATGATACGGCGACCACCGAGATCTCCAAG |
| | | ACAGAGAGCTGGGTTCCACTGCCAAA |
| TRBJ1-5 | 484 | AATGATACGGCGACCACCGAGATCTACCTA |
| | | GGATGGAGAGTCGAGTCCCATCACCAAA |
| TRBJ1-6 | 106 | AATGATACGGCGACCACCGAGATCTCTGTC |
| | | ACAGTGAGCCTGGTCCCGTTCCCAAA |
| TRBJ2-1 | 107 | AATGATACGGCGACCACCGAGATCTCGGTG |
| | | AGCCGTGTCCCTGGCCCGAA |
| TRBJ2-2 | 108 | AATGATACGGCGACCACCGAGATCTCCAGT |
| | | ACGGTCAGCCTAGAGCCTTCTCCAAA |
| TRBJ2-3 | 109 | AATGATACGGCGACCACCGAGATCTACTGT |
| | | CAGCCGGGTGCCTGGGCCAAA |
| TRBJ2-4 | 110 | AATGATACGGCGACCACCGAGATCTAGAGC |
| | | CGGGTCCCGGCGCCGAA |
| TRBJ2-5 | 111 | AATGATACGGCGACCACCGAGATCTGGAGC |
| | | CGCGTGCCTGGCCCGAA |
| TRBJ2-6 | 112 | AATGATACGGCGACCACCGAGATCTGTCAG |
| | | CCTGCTGCCGGCCCCGAA |
| TRBJ2-7 | 113 | AATGATACGGCGACCACCGAGATCTGTGAG |
| | | CCTGGTGCCCGGCCCGAA |

*bold sequence indicates universal R oligonucleotide for the sequence analysis

The lengths of the amplified PCR products generated using the methods described herein will vary depending on several factors, including the specific placement of the primers (e.g., the position within the V region of the V-gene segment to which the V-segment oligonucleotide primer specifically hybridizes by nucleotide base complementarity) and the particular adaptive immune receptor (TCR or Ig) locus that is being amplified. In certain embodiments, the length of the amplified PCR product may be at least about 50, 55, 60, 65, 70, 75, 80, 85, 90, 95, 100, 110, 120, 130, 140, 150, 160, 170, 180, 190, 200, 210, 220, 230, 240 or 250 base pairs long. For example, in certain embodiments described herein the total PCR product for a rearranged TCRβCDR3 region using the methods described herein may be approximately 200 bp long. Genomic templates are PCR amplified using a pool of the combined TCR or Ig V Forward primers (the “VF pool”) and a pool of the combined TCR or Ig J R primers (the “JR pool”).

In certain embodiments, the present disclosure provides IGH primer sets designed to accommodate the potential for somatic hypermutation within the rearranged IGH genes, as is observed after initial stimulation of native B cells. In certain embodiments, such primers may be designed to to anchor the 3′ end of each primer by annealing to complementary highly conserved sequences of three or more contiguous nucleotides that, by virtue of their high degree of conservation among multiple V and J genes, are believed to be resistant to both functional and non-functional somatic mutations. Thus, in these and related embodiments IgH V- and J-segment primers may desirably be of slightly greater length than those described elsewhere herein, for example, V-segment and/or J-segment oligonucleotide primers may be 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55 or more nucleotides in length (see, e.g., Table 17). For example, certain illustrative IGHJ reverse primers described herein were designed to anchor the 3′ end of each PCR primer on a highly conserved GGGG sequence motif within the IGHJ-region encoding segment.

Exemplary sequences are shown in Table 5. Underlined sequences complementary to a portion of the IgHJ-region encoding sequence are located ten base pairs internal to the position of the recombination signal sequence (RSS), which may be deleted. These sequences may therefore be excluded from certain embodiments in which oligonucleotide sequence design includes an identifier tag sequence sometimes referred to as a “barcode”. Bold sequences in Table 5 represent the reverse complement of the IGH J reverse PCR primers. Italicized sequences represent exemplary barcode for J-region identity (eight barcodes reveal six genes, and two alleles within genes). Further sequences within underlined segments may reveal additional allelic identities.

TABLE 5

| | SEQ | |
|---|---|---|
| | ID | |
| IgH J segment | NO: | Sequence |
| >IGHJ4*01/1-48 | 452 | ACTACTTTGACTACTGGGGCCAAGGAACCCTGG |
| | | TCACCGTCTCCTCAG |
| >IGHJ4*03/1-48 | 453 | GCTACTTTGACTACTGGGGCCAAGGGACCCTGG |
| | | TCACCGTCTCCTCAG |
| >IGHJ4*02/1-48 | 454 | ACTACTTTGACTACTGGGGCCAGGGAACCCTGG |
| | | TCACCGTCTCCTCAG |
| >IGHJ3*01/1-50 | 455 | TGATGCTTTTGATGTCTGGGGCCAAGGGACAAT |
| | | GGTCACCGTCTCTTCAG |
| >IGHJ3*02/1-50 | 456 | TGATGCTTTTGATATCTGGGGCCAAGGGACAAT |
| | | GGTCACCGTCTCTTCAG |
| >IGHJ6*01/1-63 | 457 | ATTACTACTACTACTACGGTATGGACGTCTGGG |
| | | GGCAAGGGACCACGGTCACCGTCTCCTCAG |
| >IGHJ6*02/1-62 | 458 | ATTACTACTACTACTACGGTATGGACGTCTGGG |
| | | GCCAAGGGACCACGGTCACCGTCTCCTCAG |
| >IGHJ6*04/1-63 | 459 | ATTACTACTACTACTACGGTATGGACGTGGGG |
| | | CAAAGGGACCACGGTCACCGTCTCTCCTCAG |
| >IGHJ6*03/1-62 | 460 | ATTACTACTACTACTACTACATGGACGTCTGGG |
| | | GCAAAGGGACCACGGTCACCGTCTCCTCAG |
| >IGHJ2*01/1-53 | 461 | CTACTGGTACTTCGATCTCTGGGGCCGTGGCAC |
| | | CCTGGTCACTGTCTCCTCAG |
| >IGHJ5*01/1 -51 | 462 | ACAACTGGTTCGACTCCTGGGGCCAAGGAACCC |
| | | TGGTCACCGTCTCCTCAG |
| >IGHJ5*02/1-51 | 463 | ACAACTGGTTCGACCCCTGGGGCCAGGGAACC |
| | | CTGGTCACCGTCTCCTCAG |
| >IGHJ1*01/1-52 | 464 | GCTGAATACTTCCAGCACTGGGGCCAGGGCACC |
| | | CTGGTCACCGTCTCCTCAG |
| >IGHJ2P*01/1-61 | 465 | CTACAAGTGCTTGGAGCACTGGGGCAGGGCAGC |
| | | CCGGACACCGTCTCCCTGGGAACGTCAG |
| >IGHJ1P*01/1-54 | 466 | AAAGGTGCTGGGGGTCCCCTGAACCCGACCCGC |
| | | CCTGAGACCGCAGCCACATCA |
| >IGHJ3P*01/1-52 | 467 | CTTGCGGTTGGACTTCCCAGCCGACAGTGGTGG |
| | | TCTGGCTTCTGAGGGGTCA |

TABLE 6

| | SEQ | |
|---|---|---|
| IgH J | ID | |
| segment | NO: | Sequence |
| >IGHJ4_1 | 421 | TGAGGAGACGGTGACCAGGGTTCCTTGGCCC |
| >IGHJ4_3 | 422 | TGAGGAGACGGTGACCAGGGTCCCTTGGCCC |
| >IGHJ4_2 | 423 | TGAGGAGACGGTGACCAGGGTTCCCTGGCCC |
| >IGHJ3_12 | 424 | CTGAAGAGACGGTGACCATTGTCCCTTGGCCC |
| >IGHJ6_1 | 425 | CTGAGGAGACGGTGACCGTGGTCCCTTGCCCC |
| >IGHJ6_2 | 426 | TGAGGAGACGGTGACCGTGGTCCCTTGGCCC |
| >IGHJ6_34 | 427 | CTGAGGAGACGGTGACCGTGGTCCCTTTGCCC |
| >IGHJ2_1 | 428 | CTGAGGAGACAGTGACCAGOGTGCCACGGCCC |
| >IGHJ5_1 | 429 | CTGAGGAGACGGTGACCAGGGTTCCTTGGCCC |
| >IGHJ5_2 | 430 | CTGAGGAGACGGTGACCAGGGTTCCCTGGCCC |
| >IGHJ1_1 | 431 | CTGAGGAGACGGTGACCAGGGTGCCCTGGCCC |

The IgHV-segment primers described herein were designed to hybridize to coding sequences for a conserved region of the second framework domain (FR2), at a location situated between the two conserved tryptophan (W) codons of FR2. The primer sequences are anchored at the 3′ end on a tryptophan codon for all IGHV families that conserve this codon. This allows for the last three nucleotides (tryptophan's TGG) to anchor on sequence that is expected to be resistant to somatic hypermutation, providing a 3′ anchor of five out of six nucleotides for each primer. The upstream sequence is extended further than normal, and includes degenerate nucleotides to allow for mismatches induced by hypermutation (or between closely relate IGH V families) without dramatically changing the annealing characteristics of the primer, as shown in Table 7. The sequences of the IgHV gene segments are SEQ ID NOS:262-420.

TABLE 7

| | SEQ | |
|---|---|---|
| IgH V | ID | |
| segment | NO: | Sequence |
| >IGHV1 | 443 | TGGGTGCACCAGGTCCANGNACAAGGGCTTGAGTGG |
| >IGHV2 | 444 | TGGGTGCGACAGGCTCGNGNACAACGCCTTGAGTGG |
| >IGHV3 | 445 | TGGGTGCGCCAGATGCCNGNGAAAGGCCTGGAGTGG |
| >IGHV4 | 446 | TGGGTCCGCCAGSCYCCNGNGAAGGGGCTGGAGTGG |
| >IGHV5 | 447 | TGGGTCCGCCAGGCTCCNGNAAAGGGGCTGGAGTGG |
| >IGHV6 | 448 | TGGGTCTGCCAGGCTCCNGNGAAGGGGCAGGAGTGG |
| >IGH7_ | 449 | TGTGTCCGCCAGGCTCCAGGGAATGGGCTGGAGTTG |
| 3.25p | | G |
| >IGH8_ | 450 | TCAGATTCCCAAGCTCCAGGGAAGGGGCTGGAGTGA |
| 3.54p | | G |
| >IGH9_ | 451 | TGGGTCAATGAGACTCTAGGGAAGGGGCTGGAGGGA |
| 3.63p | | G |

Thermal cycling conditions may follow methods of those skilled in the art. For example, using a PCR Express thermal cycler (Hybaid, Ashford, UK), the following cycling conditions may be used: 1 cycle at 95° C. for 15 minutes, 25 to 40 cycles at 94° C. for 30 seconds, 59° C. for 30 seconds and 72° C. for 1 minute, followed by one cycle at 72° C. for 10 minutes. As will be recognized by the skilled person, thermal cycling conditions may be optimized, for example, by modifying annealing temperatures and extension times. As described further in the Examples, for amplification of the TCRβ CDR3, 50 μl PCR reactions may be used with 1.0 μM VF pool (22 nM for each unique TCR Vβ F primer), 1.0 μM JR pool (77 nM for each unique TCRBJR primer), 1× QIAGEN Multiple PCR master mix (QIAGEN part number 206145), 10% Q-solution (QIAGEN), and 16 ng/ul gDNA. As would be recognized by the skilled person, the amount of primer and other PCR reagents used, as well as PCR parameters (e.g., annealing temperature, extension times and cycle numbers), may be optimized to achieve desired PCR amplification efficiency.

### Sequencing

Sequencing may be performed using any of a variety of available high through-put single molecule sequencing machines and systems. Illustrative sequence systems include sequence-by-synthesis systems such as the Illumina Genome Analyzer and associated instruments (Illumina, Inc., San Diego, Calif.), Helicos Genetic Analysis System (Helicos BioSciences Corp., Cambridge, Mass.), Pacific Biosciences PacBio RS (Pacific Biosciences, Menlo Park, Calif.), or other systems having similar capabilities. Sequencing is achieved using a set of sequencing oligonucleotides that hybridize to a defined region within the amplified DNA molecules. The sequencing oligonucleotides are designed such that the V- and J-encoding gene segments can be uniquely identified by the sequences that are generated, based on the present disclosure and in view of known adaptive immune receptor gene sequences that appear in publicly available databases.

The term “gene” means the segment of DNA involved in producing a polypeptide chain such as all or a portion of a TCR or Ig polypeptide (e.g., a CDR3-containing polypeptide); it includes regions preceding and following the coding region “leader and trailer” as well as intervening sequences (introns) between individual coding segments (exons), and may also include regulatory elements (e.g., promoters, enhancers, repressor binding sites and the like), and may also include recombination signal sequences (RSSs) as described herein.

The nucleic acids of the present embodiments, also referred to herein as polynucleotides, may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. A coding sequence which encodes a TCR or an immunoglobulin or a region thereof (e.g., a V region, a D segment, a J region, a C region, etc.) for use according to the present embodiments may be identical to the coding sequence known in the art for any given TCR or immunoglobulin gene regions or polypeptide domains (e.g., V-region domains, CDR3 domains, etc.), or may be a different coding sequence, which, as a result of the redundancy or degeneracy of the genetic code, encodes the same TCR or immunoglobulin region or polypeptide.

In certain embodiments, the amplified J-region encoding gene segments may each have a unique sequence-defined identifier tag of 2, 3, 4, 5, 6, 7, 8, 9, 10 or about 15, 20 or more nucleotides, situated at a defined position relative to a RSS site. For example, a four-base tag may be used, in the Jβ-region encoding segment of amplified TCRβ CDR3-encoding regions, at positions +11 through +14 downstream from the RSS site. However, these and related embodiments need not be so limited and also contemplate other relatively short nucleotide sequence-defined identifier tags that may be detected in 3-region encoding gene segments and defined based on their positions relative to an RSS site. These may vary between different adaptive immune receptor encoding loci.

The recombination signal sequence (RSS) consists of two conserved sequences (heptamer, 5′-CACAGTG-3′, and nonamer, 5′-ACAAAAACC-3′), separated by a spacer of either 12+/−1 bp (“12-signal”) or 23+/−1 bp (“23-signal”). A number of nucleotide positions have been identified as important for recombination including the CA dinucleotide at position one and two of the heptamer, and a C at heptamer position three has also been shown to be strongly preferred as well as an A nucleotide at positions 5, 6, 7 of the nonamer. (Ramsden et. al 1994; Akamatsu et. al. 1994; Hesse et. al. 1989). Mutations of other nucleotides have minimal or inconsistent effects. The spacer, although more variable, also has an impact on recombination, and single-nucleotide replacements have been shown to significantly impact recombination efficiency (Fanning et. al. 1996, Larijani et. al 1999; Nadel et. al. 1998). Criteria have been described for identifying RSS polynucleotide sequences having significantly different recombination efficiencies (Ramsden et. al 1994; Akamatsu et. al. 1994; Hesse et. al. 1989 and Cowell et. al. 1994). Accordingly, the sequencing oligonucleotides may hybridize adjacent to a four base tag within the amplified J-encoding gene segments at positions +11 through +14 downstream of the RSS site. For example, sequencing oligonucleotides for TCRB may be designed to anneal to a consensus nucleotide motif observed just downstream of this “tag”, so that the first four bases of a sequence read will uniquely identify the J-encoding gene segment (Table 8).

TABLE 8
Sequencing oligonucleotides

| Sequencing | SEQ | |
|---|---|---|
| oligo- | ID | |
| nucleotide | NO: | Oligonucleotide sequence |
| Jseq 1-1 | 470 | ACAACTGTGAGTCTGGTGCCTTGTCCAAAG |
| | | AAA |
| Jseq 1-2 | 471 | ACAACGGTTAACCTGGTCCCCGAACCGAAG |
| | | GTG |
| Jseq 1-3 | 472 | ACAACAGTGAGCCAACTTCCCTCTCCAAAA |
| | | TAT |
| Jseq 1-4 | 473 | AAGACAGAGAGCTGGGTTCCACTGCCAAAA |
| | | AAC |
| Jseq 1-5 | 474 | AGGATGGAGAGTCGAGTCCCATCACCAAAA |
| | | TGC |
| Jseq 1-6 | 475 | GTCACAGTGAGCCTGGTCCCGTTCCCAAAG |
| | | TGG |
| Jseq 2-1 | 476 | AGCACGGTGAGCCGTGTCCCTGGCCCGAAG |
| | | AAC |
| Jseq 2-2 | 477 | AGTACGGTCAGCCTAGAGCCTTCTCCAAAA |
| | | AAC |
| Jseq 2-3 | 478 | AGCACTGTCAGCCGGGTGCCTGGGCCAAAA |
| | | TAC |
| Jseq 2-4 | 479 | AGCACTGAGAGCCGGGTCCCGGCGCCGAAG |
| | | TAC |
| Jseq 2-5 | 480 | AGCACCAGGAGCCGCGTGCCTGGCCCGAAG |
| | | TAC |
| Jseq 2-6 | 481 | AGCACGGTCAGCCTGCTGCCGGCCCCGAAA |
| | | GTC |
| Jseq 2-7 | 482 | GTGACCGTGAGCCTGGTGCCCGGCCCGAAG |
| | | TAC |

The information used to assign identities to the J- and V-encoding segments of a sequence read is entirely contained within the amplified sequence, and does not rely upon the identity of the PCR primers. In particular, the methods described herein allow for the amplification of all possible V-J combinations at a TCR or Ig locus and sequencing of the individual amplified molecules allows for the identification and quantitation of the uniquely rearranged DNA encoding the CDR3 regions. The diversity of the adaptive immune cells of a given sample can be inferred from the sequences generated using the methods and algorithms described herein. One surprising advantage provided in certain preferred embodiments by the compositions and methods of the present disclosure was the ability to amplify successfully all possible V-J combinations of an adaptive immune cell receptor locus in a single multiplex PCR reaction.

In certain embodiments, the sequencing oligonucleotides described herein may be selected such that promiscuous priming of a sequencing reaction for one J-encoding gene segment by an oligonucleotide specific to another distinct J-encoding gene segment generates sequence data starting at exactly the same nucleotide as sequence data from the correct sequencing oligonucleotide. In this way, promiscuous annealing of the sequencing oligonucleotides does not impact the quality of the sequence data generated.

The average length of the CDR3-encoding region, for the TCR, defined as the nucleotides encoding the TCR polypeptide between the second conserved cysteine of the V segment and the conserved phenylalanine of the J segment, is 35+/−3 nucleotides. Accordingly and in certain embodiments, PCR amplification using V-segment oligonucleotide primers with J-segment oligonucleotide primers that start from the J segment tag of a particular TCR or IgH J region (e.g., TCR Jβ, TCR Jγ or IgH JH as described herein) will nearly always capture the complete V-D-J junction in a 50 base pair read. The average length of the IgH CDR3 region, defined as the nucleotides between the conserved cysteine in the V segment and the conserved phenylalanine in the J segment, is less constrained than at the TCR locus, but will typically be between about 10 and about 70 nucleotides. Accordingly and in certain embodiments, PCR amplification using V-segment oligonucleotide primers with J-segment oligonucleotide primers that start from the IgH J segment tag will capture the complete V-D-J junction in a 100 base pair read.

PCR primers that anneal to and support polynucleotide extension on mismatched template sequences are referred to as promiscuous primers. In certain embodiments, the TCR and Ig J-segment reverse PCR primers may be designed to minimize overlap with the sequencing oligonucleotides, in order to minimize promiscuous priming in the context of multiplex PCR. In one embodiment, the TCR and Ig J-segment reverse primers may be anchored at the 3′ end by annealing to the consensus splice site motif, with minimal overlap of the sequencing primers. Generally, the TCR and Ig V and J-segment primers may be selected to operate in PCR at consistent annealing temperatures using known sequence/primer design and analysis programs under default parameters.

For the sequencing reaction, the exemplary IGHJ sequencing primers extend three nucleotides across the conserved CAG sequences as shown in Table 9.

TABLE 9

| | SEQ | |
|---|---|---|
| | ID | |
| IgH J segment | No: | Sequence |
| >IGHJSEQ4_1 | 432 | TGAGGAGACGGTGACCAGGGTTCCTTGGCCCCAG |
| >IGHJSEQ4_3 | 433 | TGAGGAGACGGTGACCAGGGTCCCTTGGCCCCAG |
| >IGHJSEQ4_2 | 434 | TGAGGAGACGGTGACCAGGGTTCCCTGGCCCCAG |
| >IGHJSEQ3_12 | 435 | CTGAAGAGACGGTGACCATTGTCCCTTGGCCCCAG |
| >IGHJSEQ6_1 | 436 | CTGAGGAGACGGTGACCGTGGTCCCTTGCCCCCAG |
| >IGHJSEQ6_2 | 437 | TGAGGAGACGGTGACCGTGGTCCCTIGGCCCCAG |
| >IGHJSEQ6_34 | 438 | CTGAGGAGACGGTGACCGTGGTCCCTTTGCCCCAG |
| >IGHJSEQ2_1 | 439 | CTGAGGAGACAGTGACCAGGGTGCCACGGCCCCAG |
| >IGHJSEQ5_1 | 440 | CTGAGGAGACGGTGACCAGGGTTCCTTGGCCCCAG |
| >IGHJSEQ5_2 | 441 | CTGAGGAGACGGTGACCAGGGTTCCCTGGCCCCAG |
| >IGHJSEQ1_1 | 442 | CTGAGGAGACGGTGACCAGGGTGCCCTGGCCCCAG |

### Processing Sequence Data

As presently disclosed there are also provided methods for analyzing the sequences of the diverse pool of uniquely rearranged CDR3-encoding regions that are generated using the compositions and methods that are described herein. In particular, an algorithm is provided to correct for PCR bias, sequencing and PCR errors and for estimating true distribution of specific clonotypes (e.g., a TCR or Ig having a uniquely rearranged CDR3 sequence) in blood or in a sample derived from other peripheral tissue or bodily fluid. A preferred algorithm is described in further detail herein. As would be recognized by the skilled person, the algorithms provided herein may be modified appropriately to accommodate particular experimental or clinical situations.

The use of a PCR step to amplify the TCR or Ig CDR3 regions prior to sequencing could potentially introduce a systematic bias in the inferred relative abundance of the sequences, due to differences in the efficiency of PCR amplification of CDR3 regions utilizing different V and J gene segments. As discussed in more detail in the Examples, each cycle of PCR amplification potentially introduces a bias of average magnitude 1.5^{1/15}=1.027. Thus, the 25 cycles of PCR introduces a total bias of average magnitude 1.027²⁵=1.95 in the inferred relative abundance of distinct CDR3 region sequences.

Sequenced reads are filtered for those including CDR3 sequences. Sequencer data processing involves a series of steps to remove errors in the primary sequence of each read, and to compress the data. A complexity filter removes approximately 20% of the sequences that are misreads from the sequencer. Then, sequences were required to have a minimum of a six base match to both one of the TCR or Ig J-regions and one of V-regions. Applying the filter to the control lane containing phage sequence, on average only one sequence in 7-8 million passed these steps. Finally, a nearest neighbor algorithm is used to collapse the data into unique sequences by merging closely related sequences, in order to remove both PCR error and sequencing error.

Analyzing the data, the ratio of sequences in the PCR product are derived working backward from the sequence data before estimating the true distribution of clonotypes (e.g., unique clonal sequences) in the blood. For each sequence observed a given number of times in the data herein, the probability that that sequence was sampled from a particular size PCR pool is estimated. Because the CDR3 regions sequenced are sampled randomly from a massive pool of PCR products, the number of observations for each sequence are drawn from Poisson distributions. The Poisson parameters are quantized according to the number of T cell genomes that provided the template for PCR. A simple Poisson mixture model both estimates these parameters and places a pairwise probability for each sequence being drawn from each distribution. This is an expectation maximization method which reconstructs the abundances of each sequence that was drawn from the blood.

To estimate the total number of unique adaptive immune receptor CDR3 sequences that are present in a sample, a computational approach employing the “unseen species” formula may be employed (Efron and Thisted, 1976 Biometrika 63, 435-447). This approach estimates the number of unique species (e.g., unique adaptive immune receptor sequences) in a large, complex population (e.g., a population of adaptive immune cells such as T cells or B cells), based on the number of unique species observed in a random, finite sample from a population (Fisher et al., 1943 J. Anim. Ecol. 12:42-58; Ionita-Laza et al., 2009 Proc. Nat. Acad Sci. USA 106:5008). The method employs an expression that predicts the number of “new” species that would be observed if a second random, finite and identically sized sample from the same population were to be analyzed. “Unseen” species refers to the number of new adaptive immune receptor sequences that would be detected if the steps of amplifying adaptive immune receptor-encoding sequences in a sample and determining the frequency of occurrence of each unique sequence in the sample were repeated an infinite number of times. By way of non-limiting theory, it is operationally assumed for purposes of these estimates that adaptive immune cells (e.g., T cells, B cells) circulate freely in the anatomical compartment of the subject that is the source of the sample from which diversity is being estimated (e.g., blood, lymph, etc.).

To apply this formula, unique adaptive immune receptors (e.g., TCRβ, TCRα, TCRγ, TCRδ, IgH) clonotypes takes the place of species. The mathematical solution provides that for S, the total number of adaptive immune receptors having unique sequences (e.g., TCRβ, TCRγ, IgH “species” or clonotypes, which may in certain embodiments be unique CDR3 sequences), a sequencing experiment observes xₛ copies of sequence s. For all of the unobserved clonotypes, xₛ equals 0, and each TCR or Ig clonotype is “captured” in the course of obtaining a random sample (e.g., a blood draw) according to a Poisson process with parameter. The number of T or B cell genomes sequenced in the first measurement is defined as 1, and the number of T or B cell genomes sequenced in the second measurement is defined as t.

Because there are a large number of unique sequences, an integral is used instead of a sum. If G(λ) is the empirical distribution function of the parameters λ₁, . . . , λ_{S}, and n_{κ} is the number of clonotypes (e.g., unique TCR or Ig sequences, or unique CDR3 sequences) observed exactly x times, then the total number of clonotypes, i.e., the measurement of diversity E, is given by the following formula (I):

$$E ( {n}_{x} ) = S {\int }_{0}^{∞} ( \frac{{e}^{- λ} {λ}^{x}}{x !} ) d G ( λ ) . \qquad ( I )$$

Accordingly, formula (I) may be used to estimate the total diversity of species in the entire source from which the identically sized samples are taken. Without wishing to be bound by theory, the principle is that the sampled number of clonotypes in a sample of any given size contains sufficient information to estimate the underlying distribution of clonotypes in the whole source. The value for Δ(t), the number of new clonotypes observed in a second measurement, may be determined, preferably using the following equation (I):

$$Δ ( t ) = {\sum }_{x} {E ( {n}_{x} )}_{msmt 1 + msmt 2} - {\sum }_{x} {E ( {n}_{x} )}_{msmt 1} = S {\int }_{0}^{∞} {e}^{- λ} ( 1 - {e}^{- λ t} ) d G ( λ ) \qquad ( II )$$

in which msmt1 and msmt2 are the number of clonotypes from measurements 1 and 2, respectively. Taylor expansion of 1-e^{-λt} and substitution into the expression for Δ(t) yields:

α(t)=E(x₁)t−E(x₂)t²+E(x₃)t³− . . . , (III)

which can be approximated by replacing the expectations (E(nₓ)) with the actual numbers sequences observed exactly x times in the first sample measurement. The expression for Δ(t) oscillates widely as t goes to infinity, so Δ(t) is regularized to produce a lower bound for Δ(∞), for example, using the Euler transformation (Efron et al., 1976 Biometrika 63:435).

As described in the Examples, using the numbers observed in a first measurement of TCRβ sequence diversity in a blood sample, this formula (II) predicted that 1.6*10⁵ new unique sequences should be observed in a second measurement. The actual value of the second measurement was 1.8*10⁵ new TCRβ sequences, which suggested according to non-limiting theory that the prediction provided a valid lower bound on total TCRβ sequence diversity in the subject from whom the sample was drawn.

### Using a Measurement of Adaptive Immune Receptor Diversity

Determination of adaptive immune receptor sequence diversity as described herein will find uses in a variety of settings. As non-limiting examples, the methods for quantifying structural diversity of adaptive immune receptors (TCR, Ig) as described herein may be used to detect and/or diagnose a disease or to determine a risk for having or a predisposition to a disease, to characterize the effects of a therapeutic, palliative or other treatment on adaptive immune receptor diversity in the adaptive immune system of a subject (e.g., a patient), or to monitor the effectiveness of a therapeutic, palliative or other treatment.

For instance, T cell and/or B adaptive immune cell receptor repertoires can be measured in cancer patients at various time points, e.g., before and/or after hematopoietic stem cell transplant (HSCT) treatment for leukemia, or before and/or after chemotherapy, radiotherapy, immunotherapy or a bone marrow transplant. Both the change in diversity and the overall diversity of TCR and/or Ig (e.g., TCRB, TCRG, IGH) repertoire can be determined using the compositions and methods described herein to assess immunocompetence. In this regard, changes (e.g., statistically significant increases or decreases in the number of unique adaptive immune receptor sequences, or in the frequency of representation in a sample of one or more adaptive immune receptor sequences) in the adaptive immune receptor CDR3-encoding sequences that can be identified in a sample from a subject at discrete points in time, changes over time in relative levels of any one or more unique adaptive immune receptor CDR3-encoding sequences that may be identified in a sample from a subject at discrete points in time using the compositions and methods described herein, and the overall diversity (e.g., the number of unique adaptive immune receptor CDR3-encoding sequences identified) can be quantified using the compositions and methods of the present disclosure. As would be understood by the skilled artisan, appropriate control samples can be used to establish pre-determined normal or baseline control values for overall adaptive immune receptor diversity and corresponding immunocompetence. Overall diversity of test samples can then be compared to such pre-determined control values where a statistically significant decrease in overall adaptive immune receptor diversity (e.g., structural diversity such as sequence diversity) as compared to a pre-determined control value indicates immunodeficiency or a lack of immune reconstitution. Similarly, overall adaptive immune receptor diversity can be measured over time in an individual, for example, during or following treatment, where a statistically significant increase in overall diversity from a first time point during or following treatment as compared to a second or subsequent (later) time point indicates improvement in adaptive immune receptor immune diversity and partial or, in certain embodiments, full immune reconstitution.

A standard for the expected rate of immune reconstitution after transplant can be utilized. The rate of change in adaptive immune receptor diversity between any two time points may be used to actively modify treatment. The overall adaptive immune receptor diversity at a fixed time point is also an important measure, as this standard can be used to compare adaptive immune receptor diversity and, optionally one or more other appropriate clinical indicia including any of a number of art accepted indicia of immune status, between different patients. In particular, overall adaptive immune receptor diversity may in certain preferred embodiments correlate with a clinical definition of immune reconstitution. This information may be used to modify prophylactic drug regimens of antibiotics, antivirals, and antifungals, e.g., after HSCT.

As another non-limiting example, assessment of immune reconstitution in a subject after allogeneic hematopoietic cell transplantation may also be determined by measuring changes (e.g., statistically significant increases or decreases in the number of unique adaptive immune receptor sequences, or in the frequency of representation in a sample of one or more adaptive immune receptor sequences) in adaptive immune receptor diversity. These and related approaches will also enhance analysis of age-related declines in lymphocyte diversity, for example, as determined by analysis of T cell responses to vaccination. In other related embodiments, the present compositions and methods may also provide a means to evaluate investigational therapeutic agents (e.g., immunomodulatory or other immunotherapeutic agents such as cytokines, chemokines, interleukins, etc., for example, interleukin-2 (IL-2), IL-7, IL-12, IL-17, IL-21, interferon-γ, TNF-α, etc.) that may have a direct effect on the generation, growth, and development of particular lymphocyte subpopulations such as αβ T cells, γδ T cells, B cells or other lymphocyte subsets such as those exemplified below. Similarly, other related embodiments contemplate application of the herein described compositions and methods to the study of thymic T cell populations, to characterize adaptive immune receptor (e.g., TCR) diversity in the processes of T cell receptor gene rearrangement, and positive and negative selection of thymocytes.

As will be recognized by the skilled person, numerous methodologies that are known in the art for assessing functional immunocompetence may also be used in conjunction with the compositions and methods for quantifying adaptive immune receptor diversity as described herein, to monitor, characterize and/or confirm immune reconstitution. For example, cellular assays may be performed to measure T and B cell responses to one or more specific antigens or to polyclonal T and B cell stimulators. Such assays may include but need not be limited to lymphoproliferation assays, cytotoxic T cell assays, mixed lymphocyte reaction (MLR), cytokine (including lymphokines, chemokines or other soluble mediators) release assays, intracellular cytokine staining (ICS) by flow cytometry, ELISPOT, ELISA, and the like.

In certain other embodiments, the presently disclosed compositions and methods may be used to measure adaptive immune receptor diversity in newborn subjects (e.g., newborn human patients). A newborn may typically be immunodeficient where maternally transmitted antibodies are present but the immune system is not fully functioning, and thus may be susceptible to a number of diseases until the adaptive immune system autonomously develops. Assessment of the adaptive immune system by quantifying adaptive immune receptor structural diversity using the present compositions and methods will likely prove useful for diagnosis and treatment of newborn patients.

Lymphocyte diversity as detected by quantifying adaptive immune receptor diversity using the compositions and methods described herein may also be assessed in other states of congenital or acquired immunodeficiency. For instance, an AIDS patient with a failed or failing immune system may be monitored to determine the degree or stage of disease progression, and/or to measure a patient's response to therapies that are intended to reconstitute immunocompetence.

Another application of the present compositions and methods may be to provide diagnostic assessment of adaptive immune receptor diversity in solid organ transplant recipients undergoing treatment to inhibit rejection of donated organs, such as immunosuppressive regimens. Monitoring adaptive immune receptor diversity in such subjects as an indicator of their immunocompetence may usefully be conducted before and after transplantation.

Individuals exposed to radiation or chemotherapeutic drugs are subject to bone marrow transplantations or otherwise require replenishment of T cell populations, along with associated immunocompetence. The present compositions and methods provide a means for qualitatively and quantitatively assessing the bone marrow graft, or reconstitution of lymphocytes in the course of these treatments.

One manner of determining diversity is by comparing at least two samples of genomic DNA, in one embodiment in which one sample of genomic DNA is from a patient and the other sample is from a normal subject, or alternatively, in which one sample of genomic DNA is from a patient at a first time point before or during a therapeutic treatment and the other sample is from the patient at a second, later time point, during or after treatment, or in which the two samples of genomic DNA are from the same patient at different times during treatment. Another manner of diagnosis may be based on the comparison of diversity among the samples of genomic DNA, e.g., in which the immunocompetence of a human patient is assessed by the comparison.

### Biomarkers

Certain embodiments based on the present disclosure contemplate exploitation of the observation of TCR sequences that are shared among two or more individuals represent as a new class of biomarkers for a variety of diseases, including cancers, autoimmune diseases, and infectious diseases. T cells expressing such shared TCRs have been referred to as public T cells and have been described in a number of human diseases (e.g., Venturi et al., 2008 J Immunol 181, 7853-7862; Venturi et al., 2008 Nature Rev. 8, 231-238). T cells propagate via clonal expansion, through rapid cell division to yield a progeny population expressing the same rearranged TCR sequences as the progenitor T cell. Following such clonal expansion, the TCRs may be readily detected using the herein described compositions and methods to quantify TCR diversity, even where the disease burden is small (e.g., an early stage tumor). In other embodiments, specific TCRs may also find uses as biomarkers in diseases to which T cells contribute causally. For example, T cell activity is associated with the pathogenesis of certain autoimmune disorders, e.g., multiple sclerosis, Type I diabetes, and rheumatoid arthritis. According to certain related embodiments, T cells may themselves comprise targets for drug therapy, including therapies that may be designed to target specific, sequence-defined TCRs.

The practice of certain embodiments of the present invention will employ, unless indicated specifically to the contrary, conventional methods in microbiology, molecular biology, biochemistry, molecular genetics, cell biology, virology and immunology techniques that are within the skill of the art, and reference to several of which is made below for the purpose of illustration. Such techniques are explained fully in the literature. See, e.g., Sambrook, et al. Molecular Cloning: A Laboratory Manual (3^{rd} Edition, 2001); Maniatis et al. Molecular Cloning: A Laboratory Manual (3^{rd} Ed., 2001); DNA Cloning: A Practical Approach, vol. I & II (D. Glover, ed., 2^{nd} Edition, 195, Oxford Univ. Press USA); Oligonucleotide Synthesis (N. Gait, ed., 1984 Oxford Univ. Press USA); Nucleic Acid Hybridization (B. Hames & S. Higgins, eds., 1995, IRL Press); Transcription and Translation (B. Hames & S. Higgins, eds., 1984, IRL Press); Animal Cell Culture (R. Freshney, ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3^{rd} Edition, 2010 Human Press).

Unless the context requires otherwise, throughout the present specification and claims, the word “comprise” and variations thereof, such as, “comprises” and “comprising” are to be construed in an open, inclusive sense, that is, as “including, but not limited to”. By “consisting of” is meant including, and typically limited to, whatever follows the phrase “consisting of.” By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are required and may or may not be present depending upon whether or not they affect the activity or action of the listed elements.

In this specification and the appended claims, the singular forms “a,” “an” and “the” include plural references unless the content clearly dictates otherwise. As used herein, in particular embodiments, the terms “about” or “approximately” when preceding a numerical value indicates the value plus or minus a range of 5%, 6%, 7%, 8% or 9%. In other embodiments, the terms “about” or “approximately” when preceding a numerical value indicates the value plus or minus a range of 10%, 11%, 12%, 13% or 14%. In yet other embodiments, the terms “about” or “approximately” when preceding a numerical value indicates the value plus or minus a range of 15%, 16%, 17%, 18%, 19% or 20%.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

### Example 1

### Sample Acquisition, PBMC Isolation, FACS Sorting and Genomic DNA Extraction

Peripheral blood samples from two healthy male donors aged 35 and 37 were obtained with written informed consent using forms approved by the Institutional Review Board of the Fred Hutchinson Cancer Research Center (FHCRC). Peripheral blood mononuclear cells (PBMC) were isolated by Ficoll-Hypaque® density gradient separation. The T-lymphocytes were flow sorted into four compartments for each subject: CD8⁺CD45RO^{+/-} and CD4⁺CD45RO^{+/-}. For the characterization of lymphocytes the following conjugated anti-human antibodies were used: CD4 FITC (clone M-T466, Miltenyi Biotec), CD8 PE (clone RPA-T8, BD Biosciences), CD45RO ECD (clone UCHL-1, Beckman Coulter), and CD45RO APC (clone UCHL-1, BD Biosciences). Staining of total PBMCs was done with the appropriate combination of antibodies for 20 minutes at 4° C., and stained cells were washed once before analysis. Lymphocyte subsets were isolated by FACS sorting in the BD FACSAria™ cell-sorting system (BD Biosciences). Data were analyzed with FlowJo software (Treestar Inc.).

Total genomic DNA was extracted from sorted cells using the QIAamp® DNA blood Mini Kit (QIAGEN®). The approximate mass of a single haploid genome is 3 pg. In order to sample millions of rearranged TCRB in each T cell compartment, 6 to 27 micrograms of template DNA were obtained from each compartment (see Table 10).

TABLE 10

| | CD8+/CD45RO− | CD8+/CD45RO+ | CD4+/CD45RO− | CD4+/CD45RO+ | Donor |
|---|---|---|---|---|---|
| cells (×10⁶) | 9.9 | 6.3 | 6.3 | 10 | 2 |
| DNA (μg) | 27 | 13 | 19 | 25 | |
| PCR cycles | 25 | 25 | 30 | 30 | |
| clusters | 29.3 | 27 | 102.3* | 118.3* | |
| (K/tile) | | | | | |
| VJ sequences | 3.0 | 2.0 | 4.4 | 4.2 | |
| (×10⁶) | | | | | |
| Cells | 4.9 | 4.8 | 3.3 | 9 | 1 |
| DNA | 12 | 13 | 6.6 | 19 | |
| PCR cycles | 30 | 30 | 30 | 30 | |
| Clusters | 116.3 | 121 | 119.5 | 124.6 | |
| VJ sequences | 3.2 | 3.7 | 4.0 | 3.8 | |
| Cells | NA | NA | NA | 0.03 | PCR Bias |
| DNA | NA | NA | NA | 0.015 | assessment |
| PCR cycles | NA | NA | NA | 25 + 15 | |
| clusters | NA | NA | NA | 1.4/23.8 | |
| VJ sequences | NA | NA | NA | 1.6 | |

### Example 2

### Virtual T Cell Receptor β Chain Spectratyping

Virtual TCR β chain spectratyping was performed as follows. Complementary DNA was synthesized from RNA extracted from sorted T cell populations and used as template for multiplex PCR amplification of the rearranged TCR β chain CDR3 region. Each multiplex reaction contained a 6-FAM-labeled antisense primer specific for the TCR β chain constant region, and two to five TCR β chain variable (TRBV) gene-specific sense primers. All 23 functional Vβ families were studied. PCR reactions were carried out on a Hybaid PCR Express thermal cycler (Hybaid, Ashford, UK) under the following cycling conditions: 1 cycle at 95° C. for 6 minutes, 40 cycles at 94° C. for 30 seconds, 58° C. for 30 seconds, and 72° C. for 40 seconds, followed by 1-cycle at 72° C. for 10 minutes. Each reaction contained cDNA template, 500 μM dNTPs, 2 mM MgCl₂ and 1 unit of AmpliTaq Gold DNA polymerase (Perkin Elmer) in AmpliTaq Gold buffer, in a final volume of 20 μl. After completion, an aliquot of the PCR product was diluted 1:50 and analyzed using a DNA analyzer. The output of the DNA analyzer was converted to a distribution of fluorescence intensity vs. length by comparison with the fluorescence intensity trace of a reference sample containing known size standards.

### Example 3

### Multiplex PCR Amplification of TCRβ CDR3 Regions

The CDR3 junction region was defined operationally, as follows. The junction begins with the second conserved cysteine of the V-region and ends with the conserved phenylalanine of the J-region. Taking the reverse complements of the observed sequences and translating the flanking regions, the amino acids defining the junction boundaries were identified. The number of nucleotides between these boundaries determined the length and therefore the frame of the CDR3 region. In order to generate the template library for sequencing, a multiplex PCR system was selected to amplify rearranged TCR loci from genomic DNA. The multiplex PCR system used 45 forward primers (Table 3), each specific to a functional TCR Vβ segment, and thirteen reverse primers (Table 4), each specific to a TCR Jβ segment. The primers were selected to provide that adequate information was present within the amplified sequence to identify both the V and J genes uniquely (>40 base pairs of sequence upstream of the V gene recombination signal sequence (RSS), and >30 base pairs downstream of the J gene RSS).

The forward primers were modified at the 5′ end with the universal forward primer sequence compatible with the llumina GA2 cluster station solid-phase PCR. Similarly, all of the reverse primers were modified with the GA2 universal reverse primer sequence. The 3′ end of each forward primer was anchored at position −43 in the Vβ segment, relative to the recombination signal sequence (RSS), thereby providing a unique Vβ tag sequence within the amplified region. The thirteen reverse primers specific to each Jβ segment were anchored in the 3′ intron, with the 3′ end of each primer crossing the intron/exon junction. Thirteen sequencing primers complementary to the Jβ segments were designed that were complementary to the amplified portion of the Jβ segment, such that the first few bases of sequence generated captured the unique Jβ tag sequence.

On average J deletions were 4 bp+/−2.5 bp, which implied that J deletions greater than 10 nucleotides occurred in less than 1% of sequences. The thirteen different TCR Jβ gene segments each had a unique four base tag at positions +11 through +14 downstream of the RSS site. Thus, sequencing oligonucleotides were designed to anneal to a consensus nucleotide motif observed just downstream of this “tag”, so that the first four bases of a sequence read would uniquely identify the J segment (Table 5).

The information used to assign the J and V segment of a sequence read was entirely contained within the amplified sequence, and did not rely upon the identity of the PCR primers. These sequencing oligonucleotides were selected such that promiscuous priming of a sequencing reaction for one J segment by an oligonucleotide specific to another J segment would generate sequence data starting at exactly the same nucleotide as sequence data from the correct sequencing oligonucleotide. In this way, promiscuous annealing of the sequencing oligonucleotides did not impact the quality of the sequence data generated.

The average length of the CDR3 region, defined following convention as the nucleotides between the second conserved cysteine of the V segment and the conserved phenylalanine of the J segment, was 35+/−3 nucleotides, so sequences starting from the Jβ segment tag would nearly always capture the complete VNDNJ junction in a 50 bp read.

TCR βJ gene segments were roughly 50 bp in length. PCR primers that anneal and extend to mismatched sequences are referred to as promiscuous primers. Because of the risk of promiscuous priming in the context of multiplex PCR, especially in the context of a gene family, the TCR Jβ Reverse PCR primers were designed to minimize overlap with the sequencing oligonucleotides. Thus, the 13 TCR Jβ reverse primers were anchored at the 3′ end on the consensus splice site motif, with minimal overlap of the sequencing primers. The TCR Jβ primers were designed for a consistent annealing temperature (58° C. in 50 mM salt) using the OligoCalc program under default parameters (http://www.basic.northwestern.edu/biotools/oligocalc.html).

The 45 TCR Vβ forward primers were designed to anneal to the Vβ segments in a region of relatively strong sequence conservation between VD segments, for two express purposes. First, maximizing the conservation of sequence among these primers minimized the potential for differential annealing properties of each primer. Second, the primers were chosen such that the amplified region between V and J primers contained sufficient TCR Vβ sequence information to identify the specific Vβ gene segment used. This obviated the risk of erroneous TCR V gene segment assignment, in the event of promiscuous priming by the TCR Vβ primers. TCR Vβ forward primers were designed for all known non-pseudogenes in the TCRβ locus.

The total PCR product for a successfully rearranged TCRβ CDR3 region using this system was expected to be approximately 200 bp long. Genomic templates were PCR amplified using an equimolar pool of the 45 TCR Vβ F primers (the “VF pool”) and an equimolar pool of the thirteen TCR Jβ R primers (the “JR pool”). 50 μl PCR reactions were set up at 1.0 μM VF pool (22 nM for each unique TCR Vβ F primer), 1.0 μM JR pool (77 nM for each unique TCRBJR primer), 1× QIAGEN Multiple PCR master mix (QIAGEN part number 206145), 10% Q-solution (QIAGEN), and 16 ng/ul gDNA. The following thermal cycling conditions were used in a PCR Express thermal cycler (Hybaid, Ashford, UK) under the following cycling conditions: 1 cycle at 95° C. for 15 minutes, 25 to 40 cycles at 94° C. for 30 seconds, 59° C. for 30 seconds and 72° C. for 1 minute, followed by one cycle at 72° C. for 10 minutes. 12-20 wells of PCR were performed for each library, in order to sample hundreds of thousands to millions of rearranged TCRβ CDR3 loci.

### Example 4

### Pre-Processing of Sequence Data

Sequencer data processing involved a series of steps to remove errors in the primary sequence of each read, and to compress the data. First, a complexity filter removed approximately 20% of the sequences which were misreads from the sequencer. Then, sequences were required to have a minimum of a six base match to both one of the thirteen J-regions and one of 54 V-regions. Applying the filter to the control lane containing phage sequence, on average only one sequence in 7-8 million passed these steps without false positives. Finally, a nearest neighbor algorithm was used to collapse the data into unique sequences by merging closely related sequences, in order to remove both PCR error and sequencing error (see Table 10).

### Example 5

### Estimating Relative CDR3 Sequence Abundance in PCR Pools and Blood Samples

After collapsing the data, the underlying distribution of T-cell sequences in the blood reconstructing were derived from the sequence data. The procedure used three steps; 1) flow sorting T-cells drawn from peripheral blood, 2) PCR amplification, and 3) sequencing. Analyzing the data, the ratio of sequences in the PCR product was derived working backward from the sequence data before estimating the true distribution of clonotypes in the blood.

For each sequence observed a given number of times in the data generated as described herein, the probability that that sequence was sampled from a particular size PCR pool was estimated. Because the CDR3 regions sequenced were sampled randomly from a massive pool of PCR products, the number of observations for each sequence was drawn from Poisson distributions. The Poisson parameters were quantized according to the number of T cell genomes that provided the template for PCR. A simple Poisson mixture model both estimated these parameters and placed a pairwise probability for each sequence being drawn from each distribution. This was an expectation maximization method which reconstructed the abundances of each sequence that was drawn from the blood.

### Example 6

### Unseen Species Model for Estimation of True Diversity

A mixture model can reconstruct the frequency of each TCRβ CDR3 species drawn from the blood, but the larger question was: how many unique CDR3 species were present in the donor? This question was raised where the available sample was limited in each donor, and was pertinent where the herein described techniques were extrapolated to the smaller volumes of blood that could reasonably be drawn from patients undergoing treatment.

To estimate the total number of unique adaptive immune receptor CDR3 sequences that are present in a sample, a computational approach employing the “unseen species” formula was employed (Efron and Thisted, 1976 Biometrika 63, 435-447). This approach estimated the number of unique species (e.g. unique adaptive immune receptor sequences) in a large, complex population of T cells, based on the number of unique species observed in a random, finite sample from a population (Fisher et al., 1943 J. Anim. Ecol. 12:42-58; Ionita-Laza et al., 2009 Proc. Nat. Acad Sci. USA 106:5008). The method employed an expression that predicted the number of “new” species that would be observed if a second random, finite and identically sized sample from the same population were to be analyzed. “Unseen” species refers to the number of new adaptive immune receptor sequences that would be detected if the steps of amplifying adaptive immune receptor-encoding sequences in a sample and determining the frequency of occurrence of each unique sequence in the sample were repeated an infinite number of times. By way of non-limiting theory, it is operationally assumed for purposes of these estimates that adaptive immune cells (e.g., T cells) circulated freely in the anatomical compartment of the subject that was the source of the sample from which diversity is being estimated (e.g., blood).

To apply this formula, unique adaptive immune receptors (e.g., TCRβ) clonotypes were regarded as species. The mathematical solution provided that for S, the total number of adaptive immune receptors having unique sequences (e.g., TCRβ “species” or clonotypes), a sequencing experiment observed xₛ copies of sequence s. For all of the unobserved clonotypes, xₛ equalled 0, and each TCR or Ig clonotype was “captured” in the course of obtaining a random sample (e.g., a blood draw) according to a Poisson process with parameter λₛ. The number of T cell genomes sequenced in the first measurement was defined as I, and the number of T cell genomes sequenced in the second measurement was defined as t.

Because there were a large number of unique sequences, an integral was used instead of a sum. If G(λ) was the empirical distribution function of the parameters λ₁, . . . , λ_{S}, and n was the number of clonotypes (e.g., unique TCR sequences, or unique CDR3 sequences) observed exactly x times, then the total number of clonotypes, i.e., the measurement of diversity E, was given by the following formula (I):

$$E ( {n}_{x} ) = S {\int }_{0}^{∞} ( \frac{{e}^{- λ} {λ}^{x}}{x !} ) d G ( λ ) . \qquad ( I )$$

Accordingly, formula (I) was used to estimate the total diversity of species in the entire source from which the identically sized samples were taken. Without wishing to be bound by theory, the principle is that the sampled number of clonotypes in a sample of any given size contains sufficient information to estimate the underlying distribution of clonotypes in the whole source. The value for Δ(t), the number of new clonotypes observed in a second measurement, was determined, using the following equation (II):

$$Δ ( t ) = {\sum }_{x} {E ( {n}_{x} )}_{msmt 1 + msmt 2} - {\sum }_{x} {E ( {n}_{x} )}_{msmt 1} = S {\int }_{0}^{∞} {e}^{- λ} ( 1 - {e}^{- λ t} ) d G ( λ ) \qquad ( II )$$

in which msmt1 and msmt2 were the number of clonotypes from measurements 1 and 2, respectively. Taylor expansion of 1−e^{-λt} and substitution into the expression for Δ(t) yielded:

Δ(t)=E(x₁)t−E(x₂)t²+E(x₃)t³− . . . , (III)

which could be approximated by replacing the expectations (E(nₓ) with the actual numbers sequences observed exactly x times in the first sample measurement. The expression for Δ(t) oscillated widely as t goes to infinity, so Δ(t) was regularized to produce a lower bound for Δ(∞) using the Euler transformation (Efron et al., 1976 Biometrika 63:435).

From the numbers observed in the first measurement, this computational approach predicted that 1.6*10⁵ new sequences should have been observed in the second measurement. The actual value of the second measurement was 1.8*10⁵ new TCRβ sequences, which implied that the prediction provided a valid lower bound on total diversity.

### Example 7

### Error Correction and Bias Assessment

Sequence error in the primary sequence data deriveD primarily from two sources: (1) nucleotide misincorporation that occurRED during the amplification by PCR of TCRβ CDR3 template sequences, and (2) errors in base calls introduced during sequencing of the PCR-amplified library of CDR3 sequences. The large quantity of data allowed implementation of a straightforward error correcting code to correct most of the errors in the primary sequence data that were attributable to these two sources. After error correction, the number of unique, in-frame CDR3 sequences and the number of observations of each unique sequence were tabulated for each of the four flow-sorted T cell populations from the two donors. The relative frequency distribution of CDR3 sequences in the four flow cytometrically-defined populations demonstrated that antigen-experienced CD45RO⁺ populations contained significantly more unique CDR3 sequences with high relative frequency than the CD45RO⁻ populations. Frequency histograms of TCRβ CDR3 sequences observed in four different T cell subsets distinguished by expression of CD4, CD8, and CD45RO and present in blood showed that ten unique sequences were each observed 200 times in the CD4⁺CD45RO⁺ (antigen-experienced) T cell sample, which was more than twice as frequent as that observed in the CD4⁺CD45RO⁻ populations.

The use of a PCR step to amplify the TCRβ CDR3 regions prior to sequencing could potentially have introduced a systematic bias in the inferred relative abundance of the sequences, due to differences in the efficiency of PCR amplification of CDR3 regions utilizing different Vβ and Jβ gene segments. To estimate the magnitude of any such bias, the TCRβ CDR3 regions from a sample of approximately 30,000 unique CD4⁺CD45RO⁺ T lymphocyte genomes were amplified through 25 cycles of PCR, at which point the PCR product was split in half. Half was set aside, and the other half of the PCR product was amplified for an additional 15 cycles of PCR, for a total of 40 cycles of amplification. The PCR products amplified through 25 and 40 cycles were then sequenced and compared. Over 95% of the 25 cycle sequences were also found in the 40-cycle sample: a linear correlation was observed when the frequency of sequences between these samples were compared. For sequences observed a given number of times in the 25 cycle lane, a combination of PCR bias and sampling variance accounted for the variance around the mean of the number of observations at 40 cycles. Conservatively attributing the mean variation about the line (1.5-fold) entirely to PCR bias, each cycle of PCR amplification potentially introduced a bias of average magnitude 1.5^{1/15}=1.027. Thus, the 25 cycles of PCR introduced a total bias of average magnitude 1.027²⁵=1.95 in the inferred relative abundance of distinct CDR3 region sequences.

### Example 8

### J_{β} Gene Segment Usage

The CDR3 region in each TCR β chain included sequence derived from one of the thirteen J_{β} gene segments. Analysis of the CDR3 sequences in the four different T cell populations from the two donors demonstrated that the fraction of total sequences which incorporated sequences derived from the thirteen different J_{β} gene segments varied more than 20-fold. Jβ utilization among four different T flow cytometrically-defined T cells from a single donor was relatively constant within a given donor. Moreover, the J_{β} usage patterns observed in two donors, which were inferred from analysis of genomic DNA from T cells sequenced using the Illumina GA2, were qualitatively similar to those observed in T cells from umbilical cord blood and from healthy adult donors, both of which were inferred from analysis of cDNA from T cells sequenced using exhaustive capillary-based techniques.

### Example 9

### Nucleotide Insertion Bias

Much of the diversity at the CDR3 junctions in TCR α and μ chains was created by non-templated nucleotide insertions by the enzyme Terminal Deoxynucleotidyl Transferase (TdT). However, in vivo, selection plays a significant role in shaping the TCR repertoire giving rise to unpredictability. The TdT nucleotide insertion frequencies, independent of selection, were calculated using out of frame TCR sequences. These sequences were non-functional rearrangements that were carried on one allele in T cells where the second allele had a functional rearrangement. The mono-nucleotide insertion bias of TdT favored C and G (Table 11).

TABLE 11
Mono-nucleotide bias in out of frame data

| | A | C | G | T |
|---|---|---|---|---|
| Lane 1 | 0.24 | 0.294 | 0.247 | 0.216 |
| Lane 2 | 0.247 | 0.284 | 0.256 | 0.211 |
| Lane 3 | 0.25 | 0.27 | 0.268 | 0.209 |
| Lane 4 | 0.255 | 0.293 | 0.24 | 0.21 |

Similar nucleotide frequencies were observed in the in frame sequences (Table 12).

TABLE 12
Mono-nucleotide bias in in-frame data

| | A | C | G | T |
|---|---|---|---|---|
| Lane 1 | 0.21 | 0.285 | 0.275 | 0.228 |
| Lane 2 | 0.216 | 0.281 | 0.266 | 0.235 |
| Lane 3 | 0.222 | 0.266 | 0.288 | 0.221 |
| Lane 4 | 0.206 | 0.294 | 0.228 | 0.27 |

The N regions from the out-of-frame TCR sequences were used to measure the di-nucleotide bias. To isolate the marginal contribution of a di-nucleotide bias, the di-nucleotide frequencies were divided by the mononucleotide frequencies of each of the two bases. The measure was:

$$m = \frac{f ( {n}_{1} {n}_{2} )}{f ( {n}_{1} ) f ( {n}_{2} )} .$$

The matrix for m is found in Table 13.

TABLE 13
Di-nucleotide odd ratios for out of frame data

| | A | C | G | T |
|---|---|---|---|---|
| A | 1.198 | 0.938 | 0.945 | 0.919 |
| C | 0.988 | 1.172 | 0.88 | 0.931 |
| G | 0.993 | 0.701 | 1.352 | 0.964 |
| T | 0.784 | 1.232 | 0.767 | 1.23 |

Many of the dinucleotides were under or over represented. As an example, the odds of finding a GG pair were very high. Since the codons GGN translated to glycine, many glycines were expected in the CDR3 regions.

### Example 10

### Amino Acid Distributions in the CDR3 Regions

The distribution of amino acids in the CDR3 regions of TCRβ chains are shaped by the germline sequences for V, D, and J regions, the insertion bias of TdT, and selection. The distribution of amino acids in this region for the four different T cell sub-compartments is very similar between different cell subtypes. Separating the sequences into β chains of fixed length, a position dependent distribution was determined among amino acids, which were grouped by the six chemical properties: small, special, and large hydrophobic, neutral polar, acidic and basic. The distributions were virtually identical except for the CD8+ antigen experienced T cells, which used a higher proportion of acidic bases, particularly at position 5.

Of particular interest was the comparison between CD8⁺ and CD4⁺TCR sequences, as they are known to bind to peptides presented by class I and class II HLA molecules, respectively. The CD8⁺ antigen experienced T cells had a few positions with a higher proportion of acidic amino acids. This may have been due to binding with a basic residue found on HLA Class I molecules, but not on Class II.

### Example 11

### TCR β Chains with Identical Amino Acid Sequences Found in Different People

The TCR β chain-encoding DNA sequences determined in samples from two unrelated human subjects were translated to amino acid sequences and then compared pairwise between the two donors. Many thousands of exact sequence matches were observed. For example, comparing the CD4⁺CD45RO⁻ sub-compartments, approximately 8,000 of the 250,000 unique amino acid sequences from donor 1 were exact matches to donor 2. Many of these matching sequences at the amino acid level had multiple nucleotide differences at third codon positions. Following the example mentioned above, 1,500/8,000 identical amino acid matches had >5 nucleotide mismatches. Between any two T cell sub-types, 4-5% of the unique TCRβ sequences were found to have identical amino acid matches.

Two possibilities were examined: 1) that selection during TCR development was responsible for producing these common sequences and 2) that the large bias in nucleotide insertion frequency by TdT created similar nucleotide sequences. The in-frame pairwise matches were compared to the out-of-frame pairwise matches (see Examples 1-4, above). Changing frames preserved all of the features of the genetic code and so the same number of matches should have been found if the sequence bias was responsible for the entire observation. However, almost twice as many in-frame matches as out-of-frame matches were found, suggesting that selection at the protein level played a significant role.

To confirm this finding of thousands of identical TCR β chain amino acid sequences, two donors were compared with respect to the CD8⁺ CD62L⁺ CD45RA⁺ (naïve T cell-like) TCRs from a third donor, a 44 year old CMV⁺ Caucasian female. Identical pairwise matches of many thousands of sequences at the amino acid level between the third donor and each of the original two donors were found. In contrast, 460 sequences were shared between all three donors. The large variation in total number of unique sequences between the donors was a product of the starting material and variations in loading onto the sequencer, and was not representative of a variation in true diversity in the blood of the donors.

### Example 12

### Higher Frequency Clonotypes are Closer to Germline

The variation in copy number between different sequences within every T cell sub-compartment ranged by a factor of over 10,000-fold. The only property that correlated with copy number was the sum: (the number of insertions plus the number of deletions), which inversely correlated. Results of the analysis showed that deletions played a smaller role than did insertions in the inverse correlation with copy number.

Sequences with fewer insertions and deletions have receptor sequences closer to germ line. One possibility for the increased number of sequences closer to germ line is that they were created multiple times during T cell development. Since germ line sequences are shared between people, shared TCR chains are likely created by TCRs with a small number of insertions and deletions.

### Example 13

### “Spectratype” Analysis of TCRβ CDR3 Sequences by V Gene Segment Utilization and CDR3 Length

TCR diversity has commonly been assessed using the technique of TCR spectratyping, an RT-PCR-based technique that does not assess TCR CDR3 diversity at the sequence level, but rather evaluates the diversity of TCRα or TCRβ CDR3 lengths expressed as mRNA in subsets of u T cells that use the same V_{α} or V_{β} gene segment. The spectratypes of polyclonal T cell populations with diverse repertoires of TCR CDR3 sequences, such as are seen in umbilical cord blood or in peripheral blood of healthy young adults typically contain CDR3 sequences of 8-10 different lengths that are multiples of three nucleotides, reflecting the selection for in-frame transcripts. Spectratyping also provides roughly quantitative information about the relative frequency of CDR3 sequences with each specific length. To assess whether direct sequencing of TCRβ CDR3 regions from T cell genomic DNA using the sequencer could faithfully capture all of the CDR3 length diversity that is identified by spectratyping, “virtual” TCR spectratypes (see Examples above) were generated from the sequence data and compared with TCRβ spectratypes generated using conventional PCR techniques. The virtual spectratypes contained all of the CDR3 length and relative frequency information present in the conventional spectratypes. Direct TCRβ CDR3 sequencing captured all of the TCR diversity information present in a conventional spectratype. A comparison was made of standard TCRβ spectratype data and calculated TCRβ CDR3 length distributions for sequences utilizing representative TCR VP gene segments and present in CD4⁺CD45RO⁺ cells from donor 1. Reducing the information contained in the sequence data to a frequency histogram of the unique CDR3 sequences with different lengths within each Vβ family readily reproduced all of the information contained in the spectratype data. In addition, the virtual spectratypes revealed the presence within each V_{β} family of rare CDR3 sequences with both very short and very long CDR3 lengths that were not detected by conventional PCR-based spectratyping.

### Example 14

### Estimation of Total CDR3 Sequence Diversity

After error correction, the number of unique CDR3 sequences observed in each lane of the sequencer flow cell routinely exceeded 1×10⁵. Given that the PCR products sequenced in each lane were necessarily (due to sample size) derived from a small fraction of the T cell genomes present in each of the two donors, the actual total number of unique TCRβ CDR3 sequences in the entire T cell repertoire of each individual was likely to be far higher. Estimating the number of unique sequences in the entire repertoire, therefore, involved an estimate of the number of additional unique CDR3 sequences that existed in the blood but were not observed in the sample. The estimation of total species diversity in a large, complex population using measurements of the species diversity present in a finite sample has historically been called the “unseen species problem” (also discussed above). The solution started with determining the number of new species, or TCRβ CDR3 sequences, that were observed if the experiment were repeated, i.e., if the sequencing were repeated on an identical sample of peripheral blood T cells, e.g., an identically prepared library of TCRβ CDR3 PCR products was run in a different lane of the sequencer flow cell and the number of new CDR3 sequences was counted. For CD8⁺CD45RO⁻ cells from donor 2, the predicted and observed number of new CDR3 sequences in a second lane were within 5% (see above), suggesting that this analytic solution could, in fact, be used to estimate the total number of unique TCRβ CDR3 sequences in the entire repertoire.

The resulting estimates of the total number of unique TCRβ CDR3 sequences in the four flow cytometrically-defined T cell compartments are shown in Table 14.

TABLE 14
TCR repertoire diversity

| Donor | CD8 | CD4 | CD45RO | Diversity |
|---|---|---|---|---|
| 1 | + | − | + | 6.3 * 10⁵ |
| | + | − | − | 1.24 * 10⁶ |
| | − | + | + | 8.2 * 10⁵ |
| | − | + | − | 1.28 * 10⁶ |
| | Total T cell diversity | | | 3.97 * 10⁶ |
| 2 | + | − | + | 4.4 * 10⁵ |
| | + | − | − | 9.7 * 10⁵ |
| | − | + | + | 8.7 * 10⁵ |
| | − | + | − | 1.03 * 10⁶ |

| | |
|---|---|
| Total T cell diversity | 3.31 * 10⁶ |

Of note, the total TCRβ diversity in these populations was between 3-4 million unique sequences in the peripheral blood. Surprisingly, the CD45RO⁺, or antigen-experienced, compartment constituted approximately 1.5 million of these sequences. This is at least an order of magnitude larger than expected. This discrepancy was likely attributable to the large number of these sequences observed at low relative frequency, which could only be detected through deep sequencing. The estimated TCRβ CDR3 repertoire sizes of each compartment in the two donors are within 20% of each other.

The results herein demonstrated that the realized TCRβ receptor diversity was at least five-fold higher than previous estimates (˜4*10⁶ distinct CDR3 sequences), and, in particular, suggested far greater TCRβ diversity among CD45RO⁺ antigen-experienced a T cells than has previously been reported (˜1.5*10⁶ distinct CDR3 sequences). However, bioinformatic analysis of the TCR sequence data showed strong biases in the mono- and di-nucleotide content, implying that the utilized TCR sequences were sampled from a distribution much smaller than the theoretical size. With the large diversity of TCRβ chains in each person sampled from a severely constricted space of sequences, overlap of the TCR sequence pools was expected between each person. In fact, the results showed about 5% of CD8⁺ naïve TCRβ chains with exact amino acid matches were shared between each pair of three different individuals. As the TCRα pool has been previously measured to be substantially smaller than the theoretical TCRβ diversity, these results demonstrated that hundreds to thousands of truly public αβ TCRs can be found.

### Example 15

### Measurement of the Diversity of TCRγ Repertoire

### Sample Preparation

The diversity of the TCRγ repertoire was measured in the oral T cells of saliva, circulating T cells in peripheral blood, and T cells from tissue biopsies which were frozen (skin) or formalin fixed and embedded in paraffin (FFPE). For the peripheral blood, genomic DNA was isolated from 42 ml of sample obtained by venous puncture, from which the mononuclear cells were isolated by Ficoll Hypaque density gradient separation. For saliva, the genomic DNA was isolated from 5 ml of sample. To extract DNA from the biopsies, the tissues were lysed by overnight proteinase K digests at 70° C. followed by affinity chromatography of the lysates to purify the DNA. The DNA extractions were performed using Qiagen Maxiprep™ (Qiagen, Valencia, Calif.) to isolate 8.5 to 11.4 μg of high molecular weight DNA.

### Library Generation

To generate a library of TCR molecules for sequencing, a multiplex PCR reaction to amplify all possible combinations of TCRγ V and J segments from the genomic DNA was designed. The primer design for TCRγ used a minimal set of primers to capture the multitude of V/J segments. The first primer listed in Table 15 below was universally recognized by six of the nine possible Vγ segments in the TCRγ. Similarly, the first Jγ primer in Table 15 below recognized 2 of the 5 possible Jγ segments. The multiplex PCR reaction consisted of 800 ng genomic DNA, 1.0 micromolar each of an equimolar pool of TCRγ V and J primers, and Phusion TAQ polymerase in the presence of A, T, C, and G deoxynucleotides, betaine and buffer. The pool of TCRγ primers is described in Table 15.

TABLE 15
TCRγ PCR and sequencing primers

| | SEQ | | |
|---|---|---|---|
| | ID | 5′ | |
| Primer | NO: | Adapter | Sequence |
| TRGV123458 | 485 | L1 | GGAGGGGAAGGCCCCACAGTGTCTTC |
| TRGV10_1 | 486 | L1 | CCAAATCAGGCTTTGGAGCACCTGAT |
| | | | CT |
| TRGV11_1 | 487 | L1 | CAAAGGCTTAGAATATTTATTACATG |
| | | | T |
| TRGV9_1 | 488 | L1 | TGAAGTCATACAGTTCCTGGTGTCCA |
| | | | T |
| TRGJ1_1/2 | 493 | L2 | ATCACGAGTGTTGTTCCACTGCCAAA |
| | | | GAGTTTC |
| TRGJP_1 | 494 | L2 | ATCACGAGCTTTGTTCCGGGACCAAA |
| | | | TACCTTG |
| TRGJP1_1 | 495 | L2 | ATCACGCTTAGTCCCTTCAGCAAATA |
| | | | TCTTGAA |
| TRGJP2_1 | 496 | L2 | ATCACGCCTAGTCCCTTTTGCAAACG |
| | | | TCTTGAT |
| TRGJSeq1_1/2 | 489 | — | AGTGTTGTTCCACTGCCAAAGAGTTT |
| | | | CTTAT |
| TRGJSeqP_1 | 490 | — | AGCTTTGTTCCGCTGACCAAATACCT |
| | | | TGATTT |
| TRGJSeqP1_1 | 491 | — | CTTAGTCCCTTCAGCAAATATCTTGA |
| | | | ACCA |
| TRGJSeqP2_1 | 492 | — | CCTAGTCCCTTTTGCAAACGTCTTGA |
| | | | TCCA |
| L1 Adapter | 497 | | CAAGCAGAAGACGGCATACGAGCTCT |
| | | | TCCGATCT |
| L2 Adapter | 498 | | AATGATACGGCGACCACCGAGATCT |

Eight PCR reactions from a single DNA sample were combined and concentrated by affinity chromatography to generate a TCRγ library for sequencing. The library of TCRγ molecules was quantitated by spectrophotometry using a NanoDrop1000 then assessed qualitatively by gel electrophoresis.

### Sequencing Strategy

To determine the DNA sequences encoding millions of TCRγ molecules, TCRγ libraries were amplified from genomic T cell DNA and analyzed on an Illumina GAIIx, which generated 60 bp of sequence per molecule, sufficient to capture the J and V segments and the entire CDR3 coding region. The TCRγ V and J primers were modified to contain the Illumina adaptor sequences (indicated by L1 and L2 in Table 15, above) on the 5′ end to accommodate the Illumina sequencing chemistry. The TCRγ V and J primers were positioned such that sufficient sequence around the CDR3-encoding region was present to allow unique V and J identification. The JSeq sequencing primers were designed to provide additional specificity by extending four bases into the J segment from the end of the PCR primer. This specificity of the sequencing primer design prevented generating any sequence data from molecules in the library that were present as a result of the amplification of unintended targets, allowing a highly quantitative measurement of the V and J pairings in the TCRγ repertoire. In a typical run 7 million sequences were generated from PCR products that were amplified from 6.4 micrograms of genomic DNA. From an estimation that 10% of the genomic DNA extracted was from TCRγ expressing T cells, then the input of the PCR reaction was approximately 200,000 TCRγ copies. Therefore, in the 7 million, 60-base sequences that were generated, nearly 35× coverage of the TCR library was obtained.

### TCRγ Repertoire: Data Preprocessing

The data preprocessing consisted of an initial step to apply an error-correcting algorithm to identify and correct the PCR errors generated during the amplification, and a second step to remove sequences that could not be recognized as TCRγ. Error-correcting algorithms exist in the art; one such algorithm is described in Robins et al., Blood Vol. 114, No. 19, pages 4099-4107, 5 Nov. 2009, herein incorporated by reference. The 60 bases of TCRγ sequence were then analyzed to identify the component V and J sequences and productive versus non-productive rearrangements (sequences that were out-of-frame or contained a stop codon). Tabular data were then summarized in a custom database, which provided for graphical comparison of the repertoire samples.

### TCRγ Repertoire: Analysis

Blood

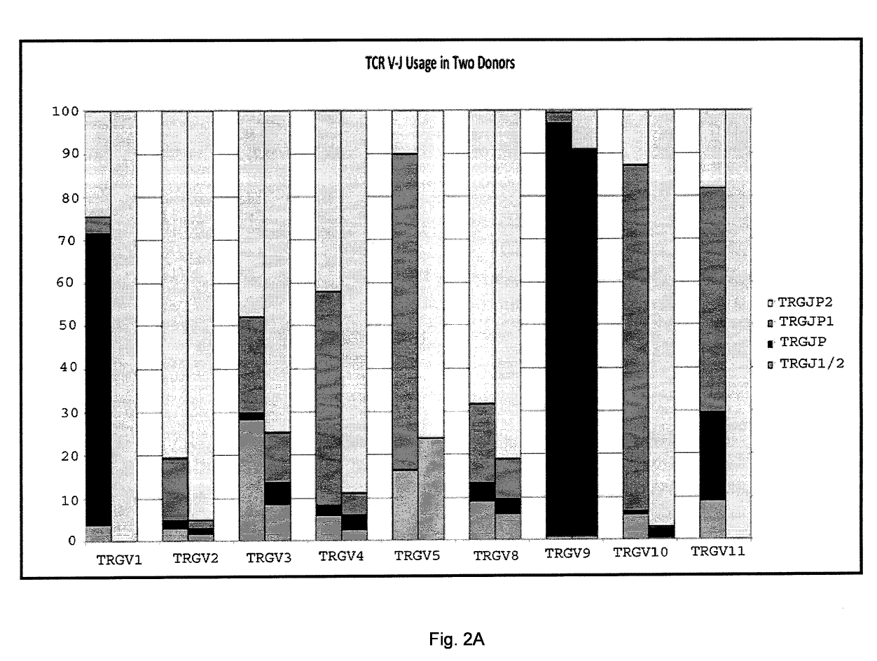

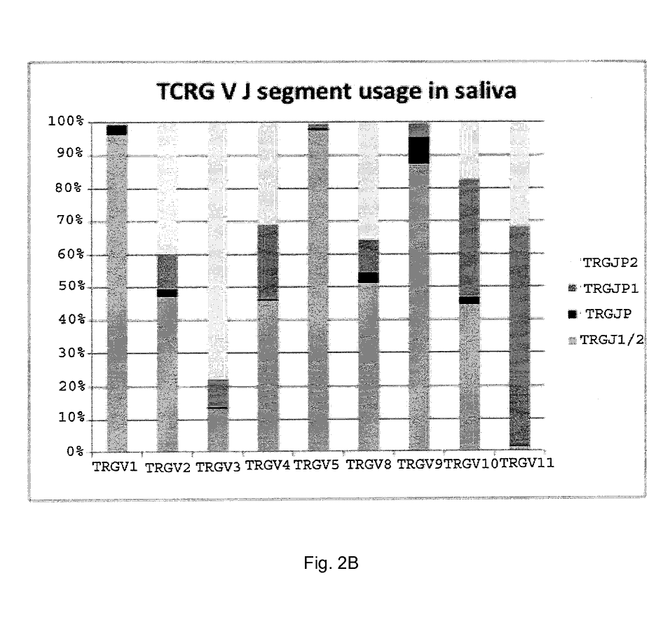

TCRγ libraries amplified from peripheral blood from two unrelated female donors were generated and compared. As a result of the comparison, it was noted that there existed diversity between the TCRγ V and J pairings between the two donors as exemplified in FIG. 2A.

This result was contrary to reports in the literature that the TCRγ in peripheral blood was restricted to a single dominant V9-JP pair. It was observed that there were 35 pairings, including 32 in the bottom five percent of all sequences. These previously unseen rare V-J pairings in the blood illustrated the sensitivity of the methods described herein for detecting TCRγ, such as potential TCRγ biomarkers for disease states.

Saliva

To demonstrate the TCRγ diversity in a peripheral tissue, TCRγ DNA library was amplified and sequenced from saliva as exemplified in FIG. 2B. The V-J pairings in the saliva TCRγ were distinct from the pattern observed in the blood, specifically a bias in pairings between V1-J1/2, V5-J1/2, and V11-JP1. These results suggested the diversity of the TCRγ repertoire in peripheral tissues exposed to the external environment could harbor signals that can be used to monitor a disease state, such as an autoimmune disease or an environmentally induced disease.

Skin

The diversity of TCRγ in skin was determined from DNA extracted from a frozen 1 mm diameter punch biopsy that contained approximately 3 mm of dermal tissue. The most common V-J pairing observed in skin was V9-JP, similar to blood (FIG. 2A) and saliva (FIG. 2B). The V9-J1 pairing was also found at significant levels in skin, but was not observed in high levels in blood and saliva.

Colon

The TCRγ repertoire from colon tissue was generated from a 10 mg formalin fixed, paraffin embedded (FFPE) tissue biopsy. The diversity of the TCRγ sequences in colon was distinct from the other tissues that were examined in that the most prevalent TCRγ V segment observed in colon was the TCRγ V10 segment, and more V-J combinations were observed in colon than in blood, skin, or saliva (Table 16).

The number of TCR sequences identified by this inventive methodology far exceeded the number of all previously known TCR sequences in any adaptive immune receptor repertoire that had been reported prior to this disclosure.

For example, in the four tissues examined, the TCRγ repertoire was characterized by determining the total number of sequences obtained from a sample, and determining the number of unique sequences represented in that total (Table 16). The set of unique sequences was comprised of individual sequences and the number of times they were seen in the total sequence count. The difference between the set of unique sequences and the set of total sequences reflected the amount of clonal expansion present in the sample, which contributed to the underlying diversity of the sequences identified, thus demonstrating the ability of this methodology to detect and quantify varying degrees of TCR, and hence T-cell, diversity. As described herein, identification and quantification of specific and significant TCRγ sequences among the millions of rearranged TCRγ sequences demonstrated the ability to detect candidate diagnostic TCRγ sequences, for use as biomarkers, predictors of a disease state, therapeutic targets, and/or indicators for monitoring a therapeutic response. The present compositions and methods may be further applicable to identifying the diversity of TCRγ in tissue samples from patients with a specific disease relative to a panel of non-disease state control samples to identify the biomarkers specific to the disease state. These biomarkers could then be used as therapeutic or predictive indicators to guide appropriate therapies. Yet another application would be use of TCRγ biomarkers to predict disease susceptibility, such as in autoimmune disease or an environmentally associated disease, such as cancer. By profiling the diversity of the TCRγ sequences the present disclosure provides a means to identify useful predictive and therapeutic biomarkers.

TABLE 16
Summary of the diversity of TCRγ sequences observed
in blood, saliva, skin and colon tissue.

| | Total | Unique | % Unique |
|---|---|---|---|
| | Sequences | Sequences | Sequences |
| Skin | 6,084,524 | 28,501 | 0.5% |
| Colon | 16,043,278 | 32,329 | 0.2% |
| Blood | 333,392 | 19,788 | 5.9% |
| Saliva | 6,976,949 | 12,068 | 0.2% |

### Example 16

### Measurement of the Diversity of the IGH Repertoire

### Sample Preparation

The IGH repertoire of naïve B cells was measured from genomic DNA which was prepared from peripheral blood using standard methods known in the art. Specifically, PBMC were FACS sorted using commercially available reagents to isolate the CD19+CD27-mature, naïve B cell population.

### Library Generation

A library of IGH-encoding DNA molecules for sequencing was prepared by designing a multiplex PCR reaction to amplify all possible combinations of productively rearranged, CDR3-containing IGHV, D and J encoding segments from the genomic DNA. A minimal set of primers was designed to amplify all known alleles of the 46 IGHV segments and the 6 IGHJ segments such that the 26 D segments were also captured by the amplified CDR3 regions. In generating this library, the IGHV primers were positioned in conserved codons to maximize primer binding affinity. The IGHJ primers were designed to anneal to the 3′ end of the shorter J segments to capture sufficient residual sequence to permit a unique identification. The IGH V and J primers were modified at the 5′ end to contain the Illumina adapter sequences (indicated by L1 and L2 in Table 17, below) to make the library compatible with the sequencing platform. A multiplex PCR reaction utilizing an equimolar pool of IGHV and IGHJ primers as well as standard additional reagents was used to generate library molecules. The pool of IGHV and IGHJ primers is presented in Table 17.

TABLE 17
IGH PCR and sequencing primers

| | SEQ | | |
|---|---|---|---|
| | ID | 5′ | |
| Primer | NO: | Adapter | Sequence |
| IGHJ1 | 499 | L2 | GCTCCCCGCTATCCCCAGACAGCAGAC |
| IGHJ2 | 500 | L2 | AGACTGGGAGGGGGCTGCAGTGGGACT |
| IGHJ3 | 501 | L2 | AGAGAAAGGAGGCAGAAGGAAAGCCAT |
| | | | C |
| IGHJ4 | 502 | L2 | CTTCAGAGTTAAAGCAGGAGAGAGGTT |
| | | | G |
| IGHJ5 | 503 | L2 | TCCCTAAGTGGACTCAGAGAGGGGGTG |
| | | | G |
| IGHJ6 | 504 | L2 | GAAAACAAAGGCCCTAGAGTGGCCATT |
| | | | C |
| IGHV1-2_03 | 505 | L1 | TGGGTGCNACAGGCCCCTGGACAAGGG |
| | | | CTTGAGTGG |
| IGHV1-24_01 | 506 | L1 | TGGGTGCGACAGGCTCCTGGAAAAGGG |
| | | | CTTGAGTGG |
| IGHV1-3_01 | 507 | L1 | TGGGTGCGCCAGGCCCCCGGACAAAGG |
| | | | CTTGAGTGG |
| IGHV1-45_01 | 508 | L1 | TGGGTGCGACAGGCCCCCGGACAAGCG |
| | | | CTTGAGTGG |
| IGHV1-45_03 | 509 | L1 | TGGGTGCGACAGGCCCCCAGACAAGCG |
| | | | CTTGAGTGG |
| IGHV1-58_01 | 510 | L1 | TGGGTGCGACAGGCTCGTGGACAACGC |
| | | | CTTGAGTGG |
| IGHV1-68_ | 511 | L1 | TGGTTGCAACAGGCCCCTGGACAAGGG |
| | | | CTTGAAAGG |
| IGHV1-8_01 | 512 | L1 | TGGGTGCGACAGGCCACTGGACAAGGG |
| | | | CTTGAGTGG |
| IGHV1-c_01 | 513 | L1 | TGGGTGCAACAGTCCCCTGGACAAGGG |
| | | | CTTGAGTGG |
| IGHV1-f_01 | 514 | L1 | TGGGTGCAACAGGCCCCTGGAAAAGGG |
| | | | CTTGAGTGG |
| IGHV1-NL1_1 | 515 | L1 | TGGGTGTGACAAAGCCCTGGACAAGGG |
| | | | CATNAGTGG |
| IGHV1p15-11 | 516 | L1 | TGGGTGCGACAGGCCCCTGGACAAGAG |
| | | | CTTGGGTGG |
| IGHV1p15-12 | 517 | L1 | TGGGTGTGACAGGCCCCTGAACAAGGG |
| | | | CTTGAGTGG |
| IGHV1p15-21 | 518 | L1 | TGGATGCGCCAGGCCCCTGGACAAAGG |
| | | | CTTGAGTGG |
| IGHV1p15-31 | 519 | L1 | TGGATGCGCCAGGCCCCTGGACAAGGC |
| | | | TTCGAGTGG |
| IGHV1p15-32 | 520 | L1 | TGGGTGTGACAGGCCCCTGGACAAGGA |
| | | | CTTGAGTGG |
| IGHV1p15-33 | 521 | L1 | TGGGTGCACCAGGTCCATGCACAAGGG |
| | | | CTGACTTGG |
| IGHV1p15-41 | 522 | L1 | TGGGTGCGCCAGGTCCATGCACAAGGG |
| | | | CTTGAGTGG |
| IGHV1p15-51 | 523 | L1 | TGGGTGTGCCAGGCCCATGCACAAGGG |
| | | | CTTGAGTGG |
| IGHV2-10_01 | 524 | L1 | TAGATCTGTCAGCCCTCAGCAAAGGCC |
| | | | CTGGAGTGG |
| IGHV2-26_01 | 525 | L1 | TGGATCCGTCAGCCCCCAGGGAAGGCC |
| | | | CTGGAGTGG |
| IGHV2-5_01 | 526 | L1 | TGGATCCGTCAGCCCCCAGGAAAGGCC |
| | | | CTGGAGTGG |
| IGHV2-70_70 | 527 | L1 | TGGATCCGTCAGCCCCCGGGGAAGGCC |
| | | | CTGGAGTGG |
| IGHV3-07_02 | 528 | L1 | TGGGTCCGCCAGGCTCCAGGGAAAGGG |
| | | | CTGGAGTGG |
| IGHV3-09_01 | 529 | L1 | TGGGTCCGGCAAGCTCCAGGGAAGGGC |
| | | | CTGGAGTGG |
| IGHV3-11_01 | 530 | L1 | TGGATCCGCCAGGCTCCAGGGAAGGGG |
| | | | CTGGAGTGG |
| IGHV3-13_01 | 531 | L1 | TGGGTCCGCCAAGCTACAGGAAAAGGT |
| | | | CTGGAGTGG |
| IGHV3-15_01 | 532 | L1 | TGGGTCCGCCAGGCTCCAGGGAAGGGG |
| | | | CTGGAGTGG |
| IGHV3-16_01 | 533 | L1 | TGGGCCCGCAAGGCTCCAGGAAAGGGG |
| | | | CTGGAGTGG |
| IGHV3-19_01 | 534 | L1 | TGGGTCCGCCAGGCTCCAGGAAAGGGG |
| | | | CTGGAGTGG |
| IGHV3-20_01 | 535 | L1 | TGGGTCCGCCAAGCTCCAGGGAAGGGG |
| | | | CTGGAGTGG |
| IGHV3-22_01 | 536 | L1 | GGGGTCCGCCAGGCTCCCGGGAAGGGG |
| | | | CTGGAATGG |
| IGHV3-25_01 | 537 | L1 | TGTGTCCGCCAGGCTCCAGGGAATGGG |
| | | | CTGGAGTTG |
| IGHV3-30_01 | 538 | L1 | TGGGTCCGCCAGGCTCCAGGCAAGGGG |
| | | | CTAGAGTGG |
| IGHV3-30_02 | 539 | L1 | TGGGTCCGCCAGGCTCCAGGCAAGGGG |
| | | | CTGGAGTGG |
| IGHV3-30_16 | 540 | L1 | TGGGTCCGCCAGGCCCCAGGCAAGGGG |
| | | | CTAGAGTGG |
| IGHV3-30_17 | 541 | L1 | TGGGTCCGCCAGGCTCCGGGCAAGGGG |
| | | | CTAGAGTGG |
| IGHV3-32_01 | 542 | L1 | CGAGTTCACCAGTCTCCAGGCAAGGGG |
| | | | CTGGAGTGA |
| IGHV3-35_01 | 543 | L1 | TGGGTCCATCAGGCTCCAGGAAAGGGG |
| | | | CTGGAGTGG |
| IGHV3-43_01 | 544 | L1 | TGGGTCCGTCAAGCTCCGGGGAAGGGT |
| | | | CTGGAGTGG |
| IGHV3-43_02 | 545 | L1 | TGGGTCCGTCAAGCTCCAGGGAAGGGT |
| | | | CTGGAGTGG |
| IGHV3-47_01 | 546 | L1 | TGGGTTCGCCGGGCTCCAGGGAAGGGT |
| | | | CTGGAGTGG |
| IGHV3-47_02 | 547 | L1 | TGGGTTCGCCGGGCTCCAGGGAAGGGT |
| | | | CCGGAGTGG |
| IGHV3-49_01 | 548 | L1 | TGGTTCCGCCAGGCTCCAGGGAAGGGG |
| | | | CTGGAGTGG |
| IGHV3-52_01 | 549 | L1 | TGGGTCTCCCAGGCTCCGGAGAAGGGG |
| | | | CTGGAGTGG |
| IGHV3-52_02 | 550 | L1 | TGGGTCTGCCAGGCTCCGGAGAAGGGG |
| | | | CAGGAGTGG |
| IGHV3-53_03 | 551 | L1 | TGGGTCCGCCAGCCTCCAGGGAAGGGG |
| | | | CTGGAGTGG |
| IGHV3-54_01 | 552 | L1 | TCAGATTCCCAAGCTCCAGGGAAGGGG |
| | | | CTGGAGTGA |
| IGHV3-54_02 | 553 | L1 | TCAGATTCCCAGGCTCCAGGGAAGGGG |
| | | | CTGGAGTGA |
| IGHV3-62_01 | 554 | L1 | TGGGTCCGCCAGGCTCCAAGAAAGGGT |
| | | | TTGTAGTGG |
| IGHV3-63_01 | 555 | L1 | TGGGTCAATGAGACTCTAGGGAAGGGG |
| | | | CTGGAGGGA |
| IGHV3-64_01 | 556 | L1 | TGGGTCCGCCAGGCTCCAGGGAAGGGA |
| | | | CTGGAATAT |
| IGHV3-71_01 | 557 | L1 | TGGGTCCGCCAGGCTCCCGGGAAGGGG |
| | | | CTGGAGTGG |
| IGHV3-73_01 | 558 | L1 | TGGGTCCGCCAGGCTTCCGGGAAAGGG |
| | | | CTGGAGTGG |
| IGHV3-74_01 | 559 | L1 | TGGGTCCGCCAAGCTCCAGGGAAGGGG |
| | | | CTGGTGTGG |
| IGHV3-d_01 | 560 | L1 | TGGGTCCGCCAGGCTCCAGGGAAGGGT |
| | | | CTGGAGTGG |
| IGHV3p15-7 | 561 | L1 | TGGGTCCGCCAGGCTCAAGGGAAAGGG |
| | | | CTAGAGTTG |
| IGHV3p16-08 | 562 | L1 | TGGGTCCGCCAGGCTCCAGGGAAGGGA |
| | | | CTGGAGTGG |
| IGHV3p16-10 | 563 | L1 | TGGGTTCGCCAGGCTCCAGGAAAAGGT |
| | | | CTGGAGTGG |
| IGHV3p16-12 | 564 | L1 | TGGATCCACCAGGCTCCAGGGAAGGGT |
| | | | CTGGAGTGG |
| IGHV3p16-13 | 565 | L1 | TGGGTCCGCCAATCTCCAGGGAAGGGG |
| | | | CTGGTGTGA |
| IGHV3p16-15 | 566 | L1 | TGGGTCCTCTAGGCTCCAGGAAAGGGG |
| | | | CTGGAGTGG |
| IGHV4-28_01 | 567 | L1 | TGGATCCGGCAGCCCCCAGGGAAGGGA |
| | | | CTGGAGTGG |
| IGHV4-30-21 | 568 | L1 | TGGATCCGGCAGCCACCAGGGAAGGGC |
| | | | CTGGAGTGG |
| IGHV4-30-41 | 569 | L1 | TGGATCCGCCAGCCCCCAGGGAAGGGC |
| | | | CTGGAGTGG |
| IGHV4-30-45 | 570 | L1 | TGGATCCGCCAGCNCCCAGGGAAGGGC |
| | | | CTGGAGTGG |
| IGHV4-30-46 | 571 | L1 | TGGATCCGCCAGCACCCAGGGAAGGGC |
| | | | CTGGAGTGG |
| IGHV4-34_01 | 572 | L1 | TGGATCCGCCAGCCCCCAGGGAAGGGG |
| | | | CTGGAGTGG |
| IGHV4-34_05 | 573 | L1 | TGGATCCGCCAGCCCCTAGGGAAGGGG |
| | | | CTGGAGTGG |
| IGHV4-34_09 | 574 | L1 | TGGATCCGCCAGCCCCCAGGGAAGGGA |
| | | | CTGGAGTGG |
| IGHV4-34_11 | 575 | L1 | TGGATCCGGCAGCCCCCAGGGAAGGGG |
| | | | CTGGAGTGG |
| IGHV4-4_01 | 576 | L1 | TGGGTCCGCCAGCCCCCAGGGAAGGGG |
| | | | CTGGAGTGG |
| IGHV4-4_07 | 577 | L1 | TGGATCCGGCAGCCCGCCGGGAAGGGA |
| | | | CTGGAGTGG |
| IGHV4-59_05 | 578 | L1 | TGGATCCGGCAGCCGCCGGGGAAGGGA |
| | | | CTGGAGTGG |
| IGHV4-59_06 | 579 | L1 | TGGATCCGGCAGCCCGCTGGGAAGGGC |
| | | | CTGGAGTGG |
| IGHV4-59_10 | 580 | L1 | TGGATCCGGCAGCCCGCCGGGAAGGGG |
| | | | CTGGAGTGG |
| IGHV5-51_01 | 581 | L1 | TGGGTGCGCCAGATGCCCGGGAAAGGC |
| | | | CTGGAGTGG |
| IGHV5-51_02 | 582 | L1 | TGGGTGCGCCAGATGCCCGGGAAAGGC |
| | | | TTGGAGTGG |
| IGHV5-51_05 | 583 | L1 | TGGGTGCGCCAGATGCCCAGGAAAGGC |
| | | | CTGGAGTGG |
| IGHV5-78_01 | 584 | L1 | TGGGTGCGCCAGATGCCCGGGAAAGAA |
| | | | CTGGAGTGG |
| IGHV6-1_01 | 585 | L1 | TGGATCAGGCAGTCCCCATCGAGAGGC |
| | | | CTTGAGTGG |
| IGHV7-4-1_0 | 586 | L1 | TGCGACAGGCCCCTGGACAAGGGCTTG |
| | | | AGTGGATGG |
| IGHV7-40_03 | 587 | L1 | TGGGTATGATAGACCCCTGGACAGGGC |
| | | | TTTGAGTGG |
| IGHV7-81 | 588 | L1 | TGGGTGCCACAGGCCCCTGGACAAGGG |
| | | | CTTGAGTGG |
| IGHJ1seq | 589 | — | CTGAGGAGACGGTGACCAGGGT |
| IGHJ2seq | 590 | — | CTGAGGAGACAGTGACCAGGGT |
| IGHJ3seq | 591 | — | CTGAAGAGACGGTGACCATTGT |
| IGHJ4seq | 592 | — | CTGAGGAGACGGTGACCAGGGT |
| IGHJ5seq | 593 | — | CTGAGGAGACGGTGACCAGGGT |
| IGHJ6seq | 594 | — | CTGAGGAGACGGTGACCGTGGT |

### Sequencing Strategy

The DNA sequences of the IGH molecules amplified from the naïve B cell DNA were determined using an Illumina HiSeq2000 to capture 100 bases of IGH sequence per molecule, sufficient to capture and identify the V, D, and J segments and random N nucleotides of the splice junctions that comprised the CDR3 coding regions. The sequencing primers were designed to provide additional specificity by extending into the J segment from the end of the PCR primer. This specificity of the sequencing primer design prevented generating any sequence data from the amplification of unintended targets, allowing a highly quantitative measurement of the IGHV and IGHJ pairings. Sequencing of this library resulted in 29.7 million IGH sequences, amplified from 1.2 micrograms of genomic DNA (see Table 18), including 652,252 unique sequences illustrating the diversity of the IGH repertoire in naïve B cells.

### IGH Repertoire: Data Preprocessing

The preprocessing and error correcting of the IGH sequences was performed essentially as described above for the preprocessing of the TCRγ libraries with specific modifications for the IGH sequences. The IGH V and J segments were used for alignment. Due to the possibility of somatic hypermutation, the number of mismatches allowed to pass the filter was increased. The total allowed number of mismatches ranged from 0-30% of the nucleotides.

TABLE 18
Summary of all IGH sequences generated from 29.8 million sequences.

| | | | Unique | Percent of all |
|---|---|---|---|---|
| | Total sequences | Percent of all | sequences | Unique |
| | observed | sequences | observed | sequences |
| Productive | 25,846,735 | 86.79% | 560,268 | 85.90% |
| Out of frame | 3,254,162 | 10.93% | 73,323 | 11.24% |
| Has stop | 681,695 | 2.29% | 18,634 | 2.86% |
| Total | 29,782,592 | | 652,225 | |

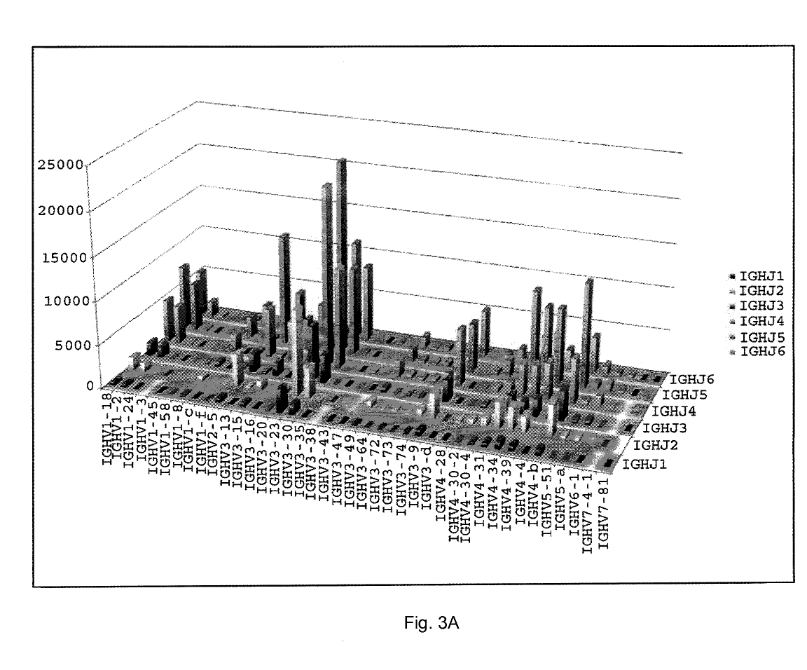

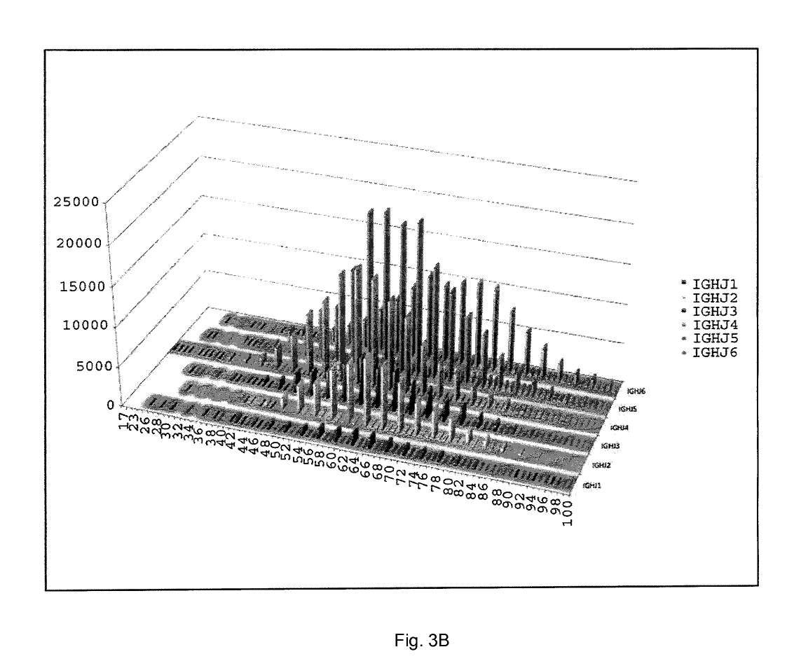

Structural diversity of the IgH repertoire was thus characterized at the level of individual adaptive immune receptor sequence representation in the population. A three dimensional representation of the IGHV and IGHJ usage in 28 million sequences from B cells was plotted (FIG. 3A). The V segments are listed on the X axis, the J segments are listed on the Y axis and the number of observations of each pairing are shown on the Z axis. For all IGHV/IGHJ pairings, the lengths of the CDR3 sequences were compared (FIG. 3B). The CDR3 length is shown on the X axis, the IGHJ segment is listed on the Y axis and the number of observations is listed on Z axis.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1-40. (canceled)

41. A method of generating a sequence-based distribution of T cell clonotypes, the method comprising the steps of:
extracting genomic DNA from a sample comprising T cells;
combining a plurality of V segment primers and a plurality of J segment primers with genomic DNA from the sample, each V segment primer comprising a sequence that binds to a different V segment at least one base pair upstream of a V gene recombination signal sequence and each J segment primer comprising a sequence that binds to a different J segment at least one base pair downstream of a J gene recombination signal sequence, so that either
(a) the sequence of each V segment primer is at least ninety percent identical to a member of the group consisting of SEQ ID NOS: 601-618 or has a sequence selected from the group consisting of SEQ ID NOS: 485-488, and the sequence of each J segment primer is at least ninety percent identical to a member of the group consisting of SEQ ID NOS: 493-496 and 595-600 or has a sequence selected from the group consisting of SEQ ID NOS: 489-496, or
(b) the sequence of each V segment primer is at least ninety percent identical to a member of the group consisting of SEQ ID NOS: 1-45 and 58-102, and the sequence of each J segment primer is at least ninety percent identical to a member of the group consisting of SEQ ID NOS: 46-57, 103-113, 468 and 483-484;
amplifying in a multiplex polymerase chain reaction (PCR) rearranged TCR genes using the plurality of V segment primers and the plurality of J segment primers to produce amplified rearranged DNA molecules less than 600 nucleotides in length;
immobilizing amplified rearranged DNA molecules on a solid surface and performing solid phase PCR to form template clusters on the solid surface;
sequencing using reversible dye-termination chemistry DNA molecules of the template clusters to produce sequences reads including a 60 bp interval that encompasses a CDR3-encoding region thereof;
removing PCR and sequencing errors by merging closely related sequence reads to form clonotypes; and
determining the relative abundance of clonotypes to form a sequence-based clonotype distribution of the sample.

42. The method of claim 41 wherein each of said V segment primers has a 5′ end with a universal forward primer sequence and each of said J segment primers has a 5′ end with a universal reverse primer sequence.

43. The method of claim 41 wherein said sample comprises at least 100,000 T cells.

44. The method of claim 41 wherein said clonotypes are T cell receptor γ (TCRγ) clonotypes and said sequence of each V segment primer is at least ninety percent identical to a member of the group consisting of SEQ ID NOS: 601-618 or has a sequence selected from the group consisting of SEQ ID NOS: 485-488, and said sequence of each J segment primer is at least ninety percent identical to a member of the group consisting of SEQ ID NOS: 493-496 and 595-600 or has a sequence selected from the group consisting of SEQ ID NOS: 489-496.

45. The method of claim 44 wherein said sample is from marrow, thymus, lymph glands, lymph nodes, or peripheral blood.

46. The method of claim 44 wherein said sample is from a human subject.

47. The method of claim 44 wherein said step of removing includes merging closely related sequence reads whenever the sequence reads are less than a given Hamming distance.

48. The method of claim 41 wherein said clonotypes are T cell receptor β (TCRβ) clonotypes and said sequence of each V segment primer is at least ninety percent identical to a member of the group consisting of SEQ ID NOS: 1-45 and 58-102, and said sequence of each J segment primer is at least ninety percent identical to a member of the group consisting of SEQ ID NOS: 46-57, 103-113, 468 and 483-484.

49. The method of claim 48 wherein said sample is from marrow, thymus, lymph glands, lymph nodes, or peripheral blood.

50. The method of claim 48 wherein said sample is from a human subject.

51. The method of claim 48 wherein said step of removing includes merging closely related sequence reads whenever the sequence reads are less than a given Hamming distance.

52. The method of claim 48 wherein each said V segment primer and each said J segment primer are selected so that each V segment and each J segment are identifiable by sequences amplified therebetween.

53. The method of claim 52 wherein said V segment primer and each said J segment primer are selected so that said amplified rearranged DNA molecules include sequences located at positions 11 through 14 downstream of said J gene recombination signal sequence.

54. A method of generating a sequence-based distribution of B cell clonotypes, the method comprising the steps of:
extracting genomic DNA from a sample comprising B cells;
combining a plurality of V segment primers and a plurality of J segment primers with genomic DNA from the sample, each V segment primer comprising a sequence that binds to a different V segment at least one base pair upstream of a V gene recombination signal sequence and each J segment primer comprising a sequence that binds to a different J segment at least one base pair downstream of a J gene recombination signal sequence, so that each V segment and each J segment are identifiable by sequences amplified therebetween and so that the sequence of each V segment primer is at least ninety percent identical to a member of the group consisting of SEQ ID NOS: 443-451, 505-588 and 635-925, and the sequence of each J segment primer is at least ninety percent identical to a member of the group consisting of SEQ ID NOS: 421-431, 452-467, 499-504 and 619-634;
amplifying in a multiplex polymerase chain reaction (PCR) rearranged immunoglobulin heavy chain genes using the plurality of V segment primers and the plurality of J segment primers to produce amplified rearranged DNA molecules less than 600 nucleotides in length;
immobilizing amplified rearranged DNA molecules on a solid surface and performing solid phase PCR to form template clusters on the solid surface;
sequencing using reversible dye-termination chemistry DNA molecules of the template clusters to produce sequences reads including 60 bp that encompass a CDR3-encoding region thereof;
removing PCR and sequencing errors by merging closely related sequence reads to form clonotypes; and
determining the relative abundance of clonotypes to form a sequence-based clonotype distribution of the sample.

55. The method of claim 54 wherein each of said V segment primers has a 5′ end with a universal forward primer sequence and each of said J segment primers has a 5′ end with a universal reverse primer sequence.

56. The method of claim 54 wherein said sample comprises at least 100,000 B cells.

57. The method of claim 54 wherein said sample is from marrow, thymus, lymph glands, lymph nodes, or peripheral blood.

58. The method of claim 54 wherein said sample is from a human subject.

59. The method of claim 54 wherein said step of removing includes merging closely related sequence reads whenever the sequence reads are less than a given Hamming distance.
